(19) United States
(12) Patent Application Publication
(10) Pub. No.: US 2004/0185440 A9
(43) Pub. Date: Sep. 23, 2004

(54) 125 human secreted proteins

(76) Inventors: Ping Feng, Gaithersburg, MD (US); Steven M. Ruben, Olney, MD (US); Craig A. Rosen, Laytonsville, MD (US); Reinhard Ebner, Gaithersburg, MD (US); Henrik S. Olsen, Gaithersburg, MD (US); Jian Ni, Rockville, MD (US); Ying-Fei Wei, Berkeley, CA (US); Daniel R. Soppet, Centreville, VA (US); Paul A. Moore, Germantown, MD (US); Hla Kyaw, Frederick, MD (US); David W. LaFleur, Washington, DC (US); Yanggu Shi, Gaithersburg, MD (US); Fouad Janat, Westerly, RI (US); Gregory A. Endress, Potomac, MD (US); Kenneth C. Carter, North Potomac, MD (US)

Correspondence Address: HUMAN GENOME SCIENCES INC, INTELLECTUAL PROPERTY DEPT., 14200 SHADY GROVE ROAD, ROCKVILLE, MD, 20850, US

(21) Appl. No.: 09/818,683

(22) Filed: Mar. 28, 2001

Related U.S. Application Data
(63) Continuation of application No. 09/305,736, filed on May 5, 1999, now abandoned.
Application No. 09/305,736 is a continuation-in-part of application No. PCT/US98/23435, filed on Nov. 4, 1998.
(60) Provisional application No. 60/064,911, filed on Nov. 7, 1997.
(60) Provisional application No. 60/064,912, filed on Nov. 7, 1997.
(60) Provisional application No. 60/064,983, filed on Nov. 7, 1997.
(60) Provisional application No. 60/064,900, filed on Nov. 7, 1997.
(60) Provisional application No. 60/064,988, filed on Nov. 7, 1997.
(60) Provisional application No. 60/064,987, filed on Nov. 7, 1997.
(60) Provisional application No. 60/064,908, filed on Nov. 7, 1997.
(60) Provisional application No. 60/064,984, filed on Nov. 7, 1997.
(60) Provisional application No. 60/064,985, filed on Nov. 7, 1997.
(60) Provisional application No. 60/066,094, filed on Nov. 17, 1997.
(60) Provisional application No. 60/066,100, filed on Nov. 17, 1997.
(60) Provisional application No. 60/066,089, filed on Nov. 17, 1997.
(60) Provisional application No. 60/066,095, filed on Nov. 17, 1997.
(60) Provisional application No. 60/066,090, filed on Nov. 17, 1997.

(51) Int. Cl. C12Q 1/68; C12P 21/06; A01N 37/18; C07H 21/00; A61K 38/00

(52) U.S. Cl. 435/6; 435/69.1; 536/23.1; 530/300; 514/2

(57) ABSTRACT

The present invention relates to novel human secreted proteins and isolated nucleic acids containing the coding regions of the genes encoding such proteins. Also provided are vectors, host cells, antibodies, and recombinant methods for producing human secreted proteins. The invention further relates to diagnostic and therapeutic methods useful for diagnosing and treating disorders related to these novel human secreted proteins.

### FIELD OF THE INVENTION

This invention relates to newly identified polynucleotides and the polypeptides encoded by these polynucleotides, uses of such polynucleotides and polypeptides, and their production.

### BACKGROUND OF THE INVENTION

Unlike bacterium, which exist as a single compartment surrounded by a membrane, human cells and other eucaryotes are subdivided by membranes into many functionally distinct compartments. Each membrane-bounded compartment, or organelle, contains different proteins essential for the function of the organelle. The cell uses “sorting signals,” which are amino acid motifs located within the protein, to target proteins to particular cellular organelles.

One type of sorting signal, called a signal sequence, a signal peptide, or a leader sequence, directs a class of proteins to an organelle called the endoplasmic reticulum (ER). The ER separates the membrane-bounded proteins from all other types of proteins. Once localized to the ER, both groups of proteins can be further directed to another organelle called the Golgi apparatus. Here, the Golgi distributes the proteins to vesicles, including secretory vesicles, the cell membrane, lysosomes, and the other organelles.

Proteins targeted to the ER by a signal sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins are stored in secretory vesicles (or secretory granules) until exocytosis is triggered. Similarly, proteins residing on the cell membrane can also be secreted into the extracellular space by proteolytic cleavage of a “linker” holding the protein to the membrane.

Despite the great progress made in recent years, only a small number of genes encoding human secreted proteins have been identified. These secreted proteins include the commercially valuable human insulin, interferon, Factor VIII, human growth hormone, tissue plasminogen activator, and erythropoeitin. Thus, in light of the pervasive role of secreted proteins in human physiology, a need exists for identifying and characterizing novel human secreted proteins and the genes that encode them. This knowledge will allow one to detect, to treat, and to prevent medical disorders by using secreted proteins or the genes that encode them.

### SUMMARY OF THE INVENTION

The present invention relates to novel polynucleotides and the encoded polypeptides. Moreover, the present invention relates to vectors, host cells, antibodies, and recombinant methods for producing the polypeptides and polynucleotides. Also provided are diagnostic methods for detecting disorders related to the polypeptides, and therapeutic methods for treating such disorders. The invention further relates to screening methods for identifying binding partners of the polypeptides.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” protein refers to those proteins capable of being directed to the ER, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of the coding sequence, but do not comprise all or a portion of any intron. In another embodiment, the nucleic acid comprising the coding sequence does not contain coding sequences of a genomic flanking gene (i.e., 5′ or 3′ to the gene in the genome).

As used herein, a “polynucleotide” refers to a molecule having a nucleic acid sequence contained in SEQ ID NO:X or the cDNA contained within the clone deposited with the ATCC. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

In the present invention, the full length sequence identified as SEQ ID NO:X was often generated by overlapping sequences contained in multiple clones (contig analysis). A representative clone containing all or most of the sequence for SEQ ID NO:X was deposited with the American Type Culture Collection (“ATCC”). As shown in Table 1, each clone is identified by a cDNA Clone ID (Identifier) and the ATCC Deposit Number. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

A “polynucleotide” of the present invention also includes those polynucleotides capable of hybridizing, under stringent hybridization conditions, to sequences contained in SEQ ID NO:X, the complement thereof, or the cDNA within the clone deposited with the ATCC. “Stringent hybridization conditions” refers to an overnight incubation at 42° C. in a solution comprising 50% formamide, 5× SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6× SSPE (20× SSPE=3M NaCl; 0.2M NaH₂PO₄; 0.02M EDTA, pH 7.4), 0.5% SDS, 30% formamide, 100 ug/ml salmon sperm blocking DNA; followed by washes at 50° C. with 1× SSPE, 0.1% SDS. In addition, to achieve even lower stringency, washes performed following stringent hybridization can be done at higher salt concentrations (e.g. 5× SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:X” refers to a polynucleotide sequence while “SEQ ID NO:Y” refers to a polypeptide sequence, both sequences identified by an integer specified in Table 1.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

Polynucleotides and Polypeptides of the Invention

Features of Protein Encoded by Gene No: 1

The translation product of this gene shares sequence homology with transcytosis-associated protein (TAP), which is thought to be important in the docking of transport vesicles with their target membrane. This protein is believed to be the human homolog of the TAP protein.

The gene encoding the disclosed cDNA is thought to reside on chromosome 4. Accordingly, polynucleotides related to this invention are useful as a marker in linkage analysis for chromosome 4.

This gene is expressed primarily in developing brain, other embryonic tissue and placental tissue.

Therefore, polynucleotides and polypeptides of the invention are useful as reagents for differential identification of the tissue(s) or cell type(s) present in a biological sample and for diagnosis of diseases and conditions which include, but are not limited to, developmental and neurodegenerative diseases and/or disorders of the brain, as well as, other developmental anomalies or fetal deficiencies. Similarly, polypeptides and antibodies directed to these polypeptides are useful in providing immunological probes for differential identification of the tissue(s) or cell type(s). For a number of disorders of the above tissues or cells, particularly of the brain, expression of this gene at significantly higher or lower levels may be routinely detected in certain tissues or cell types (e.g., embryonic, developmental, neural, cancerous and wounded tissues) or bodily fluids (e.g., lymph, serum, plasma, urine, amniotic fluid, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 139 as residues: Pro-51 to Arg-56, Lys-89 to Gln-94, Glu-144 to Gln-151, Gln-178 to Gln-183, Leu-224 to Gln-229, Tyr-284 to Pro-298, Lys-324 to Lys-334.

The tissue distribution in developing brain and placental tissues, and the homology to transcytosis-associated protein (TAP), indicates that polynucleotides and polypeptides corresponding to this gene are useful for a host of conditions which arise as a result of a failure of, or deficiency in, the secretory or endocytic pathway (i.e., neurotransmitters, etc.). In addition, the expression in brain would suggest a role in the detection and/or treatment of brain tumors, developmental and behavioral disorders such as mania, depression, paranoia, addictive behavior and sleep disorders. Moreover, the expression within embryonic tissue and other cellular sources marked by proliferating cells indicates this protein may play a role in the regulation of cellular division, and may show utility in the diagnosis, treatment, and/or prevention of developmental diseases and disorders, cancer, and other proliferative conditions.

Representative uses are described in the “Hyperproliferative Disorders” and “Regeneration” sections below and elsewhere herein. Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases.

The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:11 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 996 of SEQ ID NO:11, b is an integer of 15 to 1010, where both a and b correspond to the positions of nucleotide residues shown in SEQ ID NO:11, and where b is greater than or equal to a +14.

Features of Protein Encoded by Gene No: 2

This gene is expressed primarily in human adrenal gland tumor, and, to a lesser extent, in smooth muscle.

Therefore, polynucleotides and polypeptides of the invention are useful as reagents for differential identification of the tissue(s) or cell type(s) present in a biological sample and for diagnosis of diseases and conditions which include, but are not limited to, endocrine and vascular diseases and/or disorders, particularly adrenal gland tumors. Similarly, polypeptides and antibodies directed to these polypeptides are useful in providing immunological probes for differential identification of the tissue(s) or cell type(s). For a number of disorders of the above tissues or cells, particularly of the adrenal gland, expression of this gene at significantly higher or lower levels may be routinely detected in certain tissues or cell types (e.g., endocrine, adrenal gland, placental, smooth muscle, vascular, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in adrenal gland tumor tissue indicates that polynucleotides and polypeptides corresponding to this gene are useful for the diagnosis or treatment of endocrine diseases and/or disorders, particularly adrenal gland tumors. Representative uses are described in the “Biological Activity”, “Hyperproliferative Disorders”, and “Binding Activity” sections below, in Example 11, 17, 18, 19, 20 and 27, and elsewhere herein. Briefly, polynucleotides and polypeptides corresponding to this gene are useful for the detection, treatment, and/or prevention of various endocrine disorders and cancers, particularly Addison's disease, Cushing's Syndrome, and disorders and/or cancers of the pancrease (e.g., diabetes mellitus), adrenal cortex, ovaries, pituitary (e.g., hyper-, hypopituitarism), thyroid (e.g., hyper-, hypothyroidism), parathyroid (e.g., hyper-, hypoparathyroidism), hypothallamus, and testes.

Moreover, the protein is useful in the detection, treatment, and/or prevention of a variety of vascular disorders and conditions, which include, but are not limited to miscrovascular disease, vascular leak syndrome, aneurysm, stroke, embolism, thrombosis, coronary artery disease, arteriosclerosis, and/or atherosclerosis. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues. tissues.

Many polynucleotide sequences, such as EST sequences, are publicly available and accessible through sequence databases. Some of these sequences are related to SEQ ID NO:12 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1545 of SEQ ID NO:12, b is an integer of 15 to 1559, where both a and b correspond to the positions of nucleotide residues shown in SEQ ID NO:12, and where b is greater than or equal to a +14.

Features of Protein Encoded by Gene No: 3

When tested against U937 Myeloid cell lines, supernatants removed from cells containing this gene activated the GAS assay. Thus, it is likely that this gene activates myeloid cells, including their progenitors and other immune and hematopoietic cells and tissues, through the Jak-STAT signal transduction pathway. The gamma activating sequence (GAS) is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| GRAFALRTMLPVVSSVFALPFYLNFRIYYFKILSYLNVIHFSSTNFEYHSFVLL | (SEQ ID NO:267) |
| DLHSLRSWGAKLGLRF GGFRSRVLSGGSASNADWRFCSNAFASSAH, | |
| LPVVSSVFALPFYLNERIYYF, | (SEQ ID NO:268) |
| FKILSYLNVIHFSSTNFEYHS, | (SEQ ID NO:271) |
| SFVLLDLHSLRSWGAKLGLRF, and/or | (SEQ ID NO:269) |
| FGGFRSRVLSGGSASNADWR. | (SEQ ID NO:270) |

Polynucleotides encoding these polypeptides are also provided.

This gene is expressed primarily in small intestine.

Therefore, polynucleotides and polypeptides of the invention are useful as reagents for differential identification of the tissue(s) or cell type(s) present in a biological sample and for diagnosis of diseases and conditions which include, but are not limited to, a variety of gastrointestinal disorders including duodenal uclers. Similarly, polypeptides and antibodies directed to these polypeptides are useful in providing immunological probes for differential identification of the tissue(s) or cell type(s). For a number of disorders of the above tissues or cells, particularly of the gastrointestinal system, expression of this gene at significantly higher or lower levels may be routinely detected in certain tissues or cell types (e.g., gastrointestinal, smooth muscle, endothelial, and cancerous and wounded tissues) or bodily fluids (e.g., bile, lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 141 as residues: Gln-77 to Pro-86.

The tissue distribution in small intestine indicates that the translation product of this gene is useful for the diagnosis and/or treatment of a number of disorders having to do with the gastrointestinal system, and specifically the small intestine. Representative uses are described elsewhere herein. Briefly, polynucleotides and polypeptides corresponding to this gene are useful in the detection, treatment, and/or prevention of obstructions of the ileum, meckel's diverticulum, Crohn's disease, celiac sprue, tropical sprue, and lymphoma. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:13 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1575 of SEQ ID NO:13, b is an integer of 15 to 1589, where both a and b correspond to the positions of nucleotide residues shown in SEQ ID NO:13, and where b is greater than or equal to a +14.

Features of Protein Encoded by Gene No: 4

The translation product of this gene shares sequence homology with the mouse astrotactin protein, which is thought to be important in supporting neuronal migration along glial fibers (See Genbank Accession No. gi|1293559). Additionally, astrotactin is thought to act as a ligand for neuron-glial binding during neuronal migration (See, for example, Science 272 (5260), 417-419 (1996) and PCT application WO9740155, which are hereby incorporated by reference herein).

Preferred polypeptides of the invention comprise the following amino acid sequence:

| | |
|---|---|
| GAGKRPQVLTFPEYITSLSDSGTKRMAAGVRMECQSKGRCPSSCPLCHVTSSP | (SEQ ID NO:272) |
| DTPAEPVLLEVTKAAPIYELVTNNQTQRLLQEATMSSLWCSGTGDVTEDWCR | |
| CDSTAFGADGLPTCAPLPQPVYGSLSLFQHYSGNR, | |
| TFPEYITSLSDSGTKRMAAG, | (SEQ ID NO:273) |
| GVRMECQSKGRCPSSCPLCHV, | (SEQ ID NO:274) |
| VTSSPDTPAEPVLLEVTKAAP, | (SEQ ID NO:275) |
| PIYELVTNNQTQRLLQEATM | (SEQ ID NO:276) |
| CLSIALSNALHSLDGAT | (SEQ ID NO:277) |
| SRADFVALLDQFGNHYIQEAIYGFEESCSIWYPNKQVQRRLWLEYEDISKGNS | |
| PSDESEERERDPKC, and/or | |
| MSSLWCSGTGDVIEDWCRCDS. | (SEQ ID NO:278) |

Polynucleotides encoding these polypeptides are also provided.

The gene encoding the disclosed cDNA is thought to reside on chromosome 1. Accordingly, polynucleotides related to this invention are useful as a marker in linkage analysis for chromosome 1.

This gene is expressed primarily in brain tissue from a patient with Alzheimer's disease.

Therefore, polynucleotides and polypeptides of the invention are useful as reagents for differential identification of the tissue(s) or cell type(s) present in a biological sample and for diagnosis of diseases and conditions which include, but are not limited to, neural or CNS disorders, particularly neurodegenerative disorders such as Alzheimer's disease. Similarly, polypeptides and antibodies directed to these polypeptides are useful in providing immunological probes for differential identification of the tissue(s) or cell type(s). For a number of disorders of the above tissues or cells, particularly of the central nervous system, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 142 as residues: Gln43 to Trp-53, Arg-69 to Ser-76.

The tissue distribution in brain, combined with the homology to mouse astrotactin, indicates that polynucleotides and polypeptides corresponding to this gene are useful for the diagnosis and/or treatment of CNS diseases and disorders. Representative uses are described in the “Regeneration” and “Hyperproliferative Disorders” sections below, in Example 11, 15, and 18, and elsewhere herein. Briefly, the uses include, but are not limited to the detection, treatment, and/or prevention of neurodegenerative disease states, behavioural disorders, or inflamatory conditions such as Alzheimer's disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival.

Moreover, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:14 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1241 of SEQ ID NO:14, b is an integer of 15 to 1255, where both a and b correspond to the positions of nucleotide residues shown in SEQ ID NO:14, and where b is greater than or equal to a +14.

Features of Protein Encoded by Gene No: 5

The translation product of this gene shares sequence homology with transporter protein, which is thought to be important in metabolic and respiratory functions. Based on the sequence similarity, the translation product of this gene is expected to share biological activities with transporter proteins. Such activities are known in the art, some of which are described elsewhere herein.

Preferred polypeptides of the invention comprise the following amino acid sequence:

| | |
|---|---|
| NSARAEAEELSPLLSNELHRQRSPGVSFGLSVFNLMNAIMGSGILGLAYV, | (SEQ ID NO:279) |
| LSPLLSNELHRQRSPGVSFGL, and/or | (SEQ ID NO:280) |
| LSVFNLMNAIMGSGILGLAYV. | (SEQ ID NO:281) |

Polynucleotides encoding these polypeptides are also provided.

The gene encoding the disclosed cDNA is believed to reside on chromosome 14. Accordingly, polynucleotides related to this invention are useful as a marker in linkage analysis for chromosome 14.

This gene is expressed primarily in T-cell lymphoma and dendritic cells, and to a lesser extent in placental tissue.

Therefore, polynucleotides and polypeptides of the invention are useful as reagents for differential identification of the tissue(s) or cell type(s) present in a biological sample and for diagnosis of diseases and conditions which include, but are not limited to, haemopoietic and immune diseases and/or disorders, particularly cancers, and including T-cell lymphoma and disorders associated with embryogenesis. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, developmental, reproductive, and cancerous and wounded tissues) or bodily fluids (e.g., lymph, serum, plasma, amniotic fluid, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 143 as residues: Thr-87 to Trp-94.

The tissue distribution in T-cell lymphoma and dendritic cells, combined with the homology to transporter proteins, indicates that polynucleotides and polypeptides corresponding to this gene are useful for the treatment and/or diagnosis of haemopoietic disorders such as cancer, particularly T-cell lymphoma and disorders associated with embryogenesis. Representative uses are described in the “Regeneration” and “Hyperproliferative Disorders” sections below, in Example 11, 15, and 18, and elsewhere herein. Briefly, this gene product may play a role in the survival, proliferation, and/or differentiation of hematopoieitic lineages. Expression of this gene product in T cells and primary dendritic cells also strongly indicates a role for this protein in immune function and immune surveillance. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:15 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1177 of SEQ ID NO:15, b is an integer of 15 to 1191, where both a and b correspond to the positions of nucleotide residues shown in SEQ ID NO:15, and where b is greater than or equal to a +14.

Features of Protein Encoded by Gene No: 6

Preferred polypeptides of the invention comprise the following amino acid sequence: HLGRGFVPGILGHWLGFEERSQYLPGCR (SEQ ID NO: 282). Polynucleotides encoding these polypeptides are also provided.

This gene is expressed primarily in the liver, and, to a lesser extent, in testis.

Therefore, polynucleotides and polypeptides of the invention are useful as reagents for differential identification of the tissue(s) or cell type(s) present in a biological sample and for diagnosis of diseases and conditions which include, but are not limited to, hepatic, reproductive, or endocrine disorders, particularly hepatoma or male infertility. Similarly, polypeptides and antibodies directed to these polypeptides are useful in providing immunological probes for differential identification of the tissue(s) or cell type(s). For a number of disorders of the above tissues or cells, particularly of the immune and hematopoetic systems, expression of this gene at significantly higher or lower levels may be routinely detected in certain tissues or cell types (e.g., hepatic, reproductive, endocrine, testical, immune, and cancerous and wounded tissues) or bodily fluids (e.g., lymph, serum, serminal fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 144 as residues: Ser-21 to Trp-34, Cys-68 to Gly-89, Cys-122 to Phe-133.

The tissue distribution in liver tissue indicates that polynucleotides and polypeptides corresponding to this gene are useful for the diagnosis and treatment of liver disorders, particularly those affecting the immune and hematopoetic systems, including hepatomas. Representative uses are described in the “Hyperproliferative Disorders”, “Infectious Disease”, and “Binding Activity” sections below, in Example 11, and 27, and elsewhere herein. Briefly, the protein can be used for the detection, treatment, and/or prevention of hepatoblastoma, jaundice, hepatitis, or liver metabolic diseases and conditions that are attributable to the differentiation of hepatocyte progenitor cells. Furthermore, the expression within testis indicates that the protein may show utility in the treatment and/or detection of a variety of reproductive disorders such as male infertility, impotence, and may even be useful as a contraceptive. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:16 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1172 of SEQ ID NO:16, b is an integer of 15 to 1186, where both a and b correspond to the positions of nucleotide residues shown in SEQ ID NO:16, and where b is greater than or equal to a +14.

Features of Protein Encoded by Gene No: 7

The translation product of this gene shares sequence homology with urokinase receptor which is thought to be important in cell matrix remodeling and cell movement. This gene also has good homology with the mouse hematopoietic stem cell antigen (Sca-2), Ly-6 and CD59 (protectin, MACIF—membrane attack complex inhibit factor). They are members of a new family of cell-surface protein, Ly6 superfamily (ly6SF). Sca-2 is highly expressed in early thymic precusor cells. The progeny of the intrathymic precusor population contiune to express Sca-2 until the transition from blast cells to small cells.

Mature thymocytes and peripheral T cells do not express detectable levels of Sca-2, whereas peripheral B cells are Sca-2 positive. It seems very likely that Sca-2 play a very importantnt role in thymocyte maturation and differention, and Sca-2 may be a receptor for a unknown cytokine involving thymocyte maturation and differention. CD59 is a recently discovered complement regulation protein (also knomn as protectin, MACIF—membrane attack complex inhibiting factor). Recent studies show that CD59 may prevent damage from complement C5b-9 and protect astrocytes during inflammatory and infectious disorders of the nervous system.

Expression of recombinant human CD59 on porcine donor organs have been shown to prevent complement-mediated lysis and activation of endothelial cells that leads to hyperacute rejection. Our new gene may have similar functions. This new gene also has good homology with a recently patented TGF-alpha inhibiting protein (all cysteines and spacing are conserved). The TGF-alpha inhibiting protein has anti-inflammatory, anti-coagulant and anti-tumoral properties. Recently, transgenic pigs were engineered to express the human CD59 as complement inhibitor. The expression of CD59 in transgenic pigs renders xenogeneic.

Organs resistant to hyperacute rejection (PNAS, 91:11153, 1994, Alexion Pharmaceuticals). The same company also reported (Blood, 1994, 84:2604) that expression of recombinant transmembrane CD59 in Paroxysmal Nocturnal Hemoglobinuria (PNH) B-cells confers resistance to human complement. PNH is an acquired hematopoietic disorder characterized by complement-mediated hemolytic anemia, pancytopenia, and venous thrombosis. They suggest that retroviral gene therapy with this molecule could provide a treatment for PNH patients. All references cited above are hereby incorporated herein by reference. Preferred polynucleotides of the invention comprise the following nucleic acid sequence:

| | |
|---|---|
| GGGTCGACCCACGCGTCCGGTAAAATATAAAGAAACTGAACCAGTGTGTC | (SEQ ID NO:287) |
| TTTTCACCATAGATATAAGAGTTCGGACCGCCCAGCACACAAGGTCAGCA | |
| TGCTGCTCCTCTGTCACGCTCTCGCTATAGCTGTTGTCCAGATCGTTATCTT | |
| CTCAGAAAGCTGGGCATTTGCCAAGAACATCAACTTCTATAATGTGAGGC | |
| CTCCTCTCGACCCTACACCATTTCCAAATAGCTTCAAGTGCTTTACTTGTG | |
| AAAACGCAGGGGATAATTATAACTGCAATCGATGGGCAGAAGACAAATG | |
| GTGTCCACAAAATACACAGTACTGTTTGACAGTTCATCACTTCACCAGCCA | |
| CGGAAGAAGCACATCCATCACCAAAAAGTGTGCCTCCAGAAGTGAATGTC | |
| ATTTTGTCGGTTGCCACCACAGCCGAGATTCTGAACATACGGAGTGTAGG | |
| TCTTGCTGTGAAGGAATGATCTGCAATGTAGAATTACCCACCAATCACAC | |
| TAATGCAGTGTTTGCCGTAATGCACGCTCAGAGAACATCTGGCAGCAGTG | |
| CCCCCACACTCTACCTACCAGTGCTTGCCTGGGTCTTTGTGCTTCCATTGCT | |
| GTGATGCCACCATTCCTAGGAGAGGCAGAGACCAGCCTCTAAAGCACAAG | |
| CCAAAAACTGTGTGAACGGTGAACTTTGGAGTGAAGATCAATCTTGCACT | |
| TGGTGAAGAGTGCACATTGGACCTCAAGGCGAAAGCCAGTGGTTTGCTTG | |
| GATAAAATGTTCCCGCATGAGGCCACAGGACTGAGGATGGGAATTTGGCA | |
| GGGCCTGAGAAGATGGTCTGACTTCCAGGCTTCCTGGTCAAAGAGAGCTA | |
| CGTTTGGGCAGTTCTGCAGAGAGGATCCTGGCAACTAGTCCCACCTGACT | |
| AGGCCTTTAGCTGAAAAGGATTTCTTGACCTCCTTGACTGCCTCAGAGGCT | |
| GCCAGGTCAAACCCTCTTGTTTATGTGATTAGCTCAGAGCATCTCTATGAA | |
| ATCTAACCCTTCCCCTCATGAGAAAGCAGTTTTCCCCACCAACAGCATAGT | |
| CAATGAGAAAGGCAACTGTACGAAGAAAACTTCCAGTGGAACTAATATG | |
| AAATCTATTTGCAAATTATGGGGGGAAATAAAGCTTTTAAATTATACAAT | |
| GTAAAAAAAAAAAAAAAAAAAAAAAAAAAA. | |

Preferred polypeptides of the invention comprise the following amino acid sequence:

| | |
|---|---|
| FYIADHSFTARPTLRMFRISAVVATDKMTFTSGGTLFGDGCASSVAGEVMNC | (SEQ ID NO:283) |
| QTVLCILWTPFVECPSIAVIIIPCVETSKALEATWKWCRVERRPHIWVDVLGKC | |
| PAF, | |
| RPTLRMFRISAVVATDKMTFTSGGT, | (SEQ ID NO:284) |
| PSIAVIIIIPCVFTSKALEAIWKWCRVER, | (SEQ ID NO:285) |
| TSVSFHHRYKSSDRPAHKVS, | (SEQ ID NO:286) |
| MLLLCHALAIAVVQIVTFSESWAFAKMNFYNVRPPLDPTPFPNSFKCFTCENA | (SEQ ID NO:288) |
| GDNYNCNRWAEDKWCPQNTQYCLTVKHFTSHGRSTSITKKCASRSECHFVG | |
| CHHSRDSEHTECRSCCEGMICNVELPTNHTNAVFAVMHAQRTSGSSAPTLYL | |
| PVLAWVFVLPLL, | |
| IAVVQIVTFSESWAEAKNINF, | (SEQ ID NO:289) |
| FYNVRPPLDPTPFPNSFKCET, | (SEQ ID NO:290) |
| TCENAGDNYNCNRWAEDKWCP, | (SEQ ID NO:291) |
| PQNTQYCLTVHHFTSHGRSTS | (SEQ ID NO:292) |
| SITKKCASRSECHEVGCHHSR, and/or | (SEQ ID NO:293) |
| RDSEHTECRSCCEGMICNVEL. | (SEQ ID NO:294) |

Polynucleotides encoding these polypeptides are also provided.

This gene is expressed primarily in fetal lung, breast, and Hodgkin's Lymphoma II.

Therefore, polynucleotides and polypeptides of the invention are useful as reagents for differential identification of the tissue(s) or cell type(s) present in a biological sample and for diagnosis of diseases and conditions which include, but are not limited to, pulmonary, reproductive, immune, or hematopoietic diseases and/or disorders, particularly cell growth and differentiation conditions. Similarly, polypeptides and antibodies directed to these polypeptides are useful in providing immunological probes for differential identification of the tissue(s) or cell type(s). For a number of disorders of the above tissues or cells, particularly of the fetal lung, breast, and tissues involved in Hodgkin's Lymphoma II expression of this gene at significantly higher or lower levels may be routinely detected in certain tissues and cell types (e.g., pulmonary, immune, reproductive, and cancerous and wounded tissues) or bodily fluids (e.g., lymph, amniotic fluid, pulmonary surfactant or sputum, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 145 as residues: Asn-32 to Asp-38, Thr-40 to Phe-46, Asn-53 to Gln-74, Ser-84 to Ile-91, Cys-95 to Glu-100, Ser-109 to Cys-121.

The tissue distribution in proliferating and differentiating tissues, combined with the homology to a urokinase receptor, indicates that polynucleotides and polypeptides corresponding to this gene are useful for the diagnosis and treatment of cell growth and differentiation disorders, particularly of the lung, renal, breast, immune and endothelial tissues. Representative uses are described in the “Immune Activity” and “Infectious Disease” sections below, in Example 11, 13, 14, 16, 18, 19, 20, and 27, and elsewhere herein. Briefly, the expression within fetal tissue and other cellular sources marked by proliferating cells indicates that this protein may play a role in the regulation of cellular division, and may show utility in the diagnosis and treatment of cancer and other proliferative disorders.

Similarly, developmental tissues rely on decisions involving cell differentiation and/or apoptosis in pattern formation. Thus this protein may also be involved in apoptosis or tissue differentiation and could again be useful in cancer therapy. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:17 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1168 of SEQ ID NO:17, b is an integer of 15 to 1182, where both a and b correspond to the positions of nucleotide residues shown in SEQ ID NO:17, and where b is greater than or equal to a +14.

Features of Protein Encoded by Gene No: 8

The translation product of this gene shares sequence homology with cell adhesion molecules, which are implicated in cell migration, axonal guidance and fasiculation, and growth and tumorogenesis.

When tested against U937 cell lines, supernatants removed from cells containing this gene activated the GAS (gamma activating sequence) promoter element. Thus, it is likely that this gene activates myeloid cells, myeloid progenitors, and to a lesser extent, in other cells and tissue cell types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| RHNDFNKLSYTECNNMNKRMAKPEKKKGSVKSSLGIELGPNCHLISSLFLFSV | (SEQ ID NO:295) |
| SLYPFATQF | |
| PFHYVLILEIIQAFGLCLPLTERQEAKSGLGGLCPDYTWPCPCLLVSCLSLLRL, | |
| CEVFSWHEPWSKLSPHLFLVSFLCIPLSLCHTVSFSLCSNIYNPGLRTMLAPHR | (SEQ ID NO:296) |
| ETGGQVWAGWALSRLHVALPMSLGVLSLPAPTVTVVRMEGGDWKVCEQLG | |
| QCTYSHRMTK, | |
| KRMAKPEKKKGSVKSSLGTFLGP, and/or | (SEQ ID NO:297) |
| YNPGLRTMLAPHRETGGQVWAGWALSRLHVA. | (SEQ ID NO:298) |

Polynucleotides encoding these polypeptides are also provided.

This gene is expressed primarily in fetal heart, meningima, melanocytes, and, to a lesser extent, in breast.

Therefore, polynucleotides and polypeptides of the invention are useful as reagents for differential identification of the tissue(s) or cell type(s) present in a biological sample and for diagnosis of diseases and conditions which include, but are not limited to, neurodegenerative disease states and behavioral disorders, in addition to integumentary, cardiovascular, or reproductive diseases and/or disorders, particularly of the breast. Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues and cell types (e.g., neural, integumentary, breast, reproductive, cardiovascular, developmental, and cancerous and wounded tissues) or bodily fluids (e.g., lymph, serum, plasma, urine, amniotic fluid, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 146 as residues: Asn-71 to Asp-79.

The tissue distribution in menigima combined with the homology to cell adhesion molecules and the detected GAS biological activity indicates that polynucleotides and polypeptides corresponding to this gene are useful for the treatment and/or detection of neurodegenerative disease states and behavioural disorders. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimer's Disease, Parkinson's Disease, Huntington's Disease, schizophrenia, mania, dementia, paranoia, obsessive compulsive disorder and panic disorder.

The expression within melanocytes and breast tissue indicates polynucleotides and polypeptides corresponding to this gene are useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. In addition, such disorders may predispose (i.e., increase an individuals susceptibility) to viral and bacterial infections of the skin (i.e. cold sores, warts, chickenpox, molluscum contagiosum, herpes zoster, boils, cellulitis, erysipelas, impetigo, tinea, althletes foot, and ringworm).

Moreover, the protein product of this gene may also be useful for the treatment or diagnosis of various connective tissue disorders such as arthritis, trauma, tendonitis, chrondomalacia and inflammation, autoimmune disorders such as rheumatoid arthritis, lupus, scleroderma, and dermatomyositis as well as dwarfism, spinal deformation, and specific joint abnormalities as well as chondrodysplasias (ie. spondyloepiphyseal dysplasia congenita, familial osteoarthritis, Atelosteogenesis type II, metaphyseal chondrodysplasia type Schmid). Furthermore, This protein may show utility in modulating the immune systems response to various degenerative neural conditions based upon the detected GAS biological activity. The protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:18 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1157 of SEQ ID NO:18, b is an integer of 15 to 1171, where both a and b correspond to the positions of nucleotide residues shown in SEQ ID NO:18, and where b is greater than or equal to a +14.

Features of Protein Encoded by Gene No: 9

Preferred polypeptides of the invention comprise the following amino acid sequence: SCKTENLLE (SEQ ID NO:299). Polynucleotides encoding these polypeptides are also provided.

This gene is expressed primarily in fetal liver and spleen, and infant brain.

Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, neural, and developmental disorders. Similarly, polypeptides and antibodies directed to these polypeptides are useful in providing immunological probes for differential identification of the tissue(s) or cell type(s). For a number of disorders of the above tissues or cells, particularly of the immune and developmental systems, expression of this gene at significantly higher or lower levels may be routinely detected in certain tissues and cell types (e.g., immune, hematopoietic, neural, developmental, and cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 147 as residues: Thr-187 to Lys-192, Asn-255 to Leu-262.

The tissue distribution of this gene in fetal liver spleen indicates a key role in the development of the immune system. Representative uses are described in the “Immune Activity” and “Infectious Disease” sections below, in Example 11, 13, 14, 15, 16, 18, 19, 20, and 27, and elsewhere herein. Briefly, this gene could be used in the treatment and/or detection of immune disorders including arthritis, asthma, immunodeficiency diseases and leukemia. Expression in infant brain also indicates a role in the treatment and/or detection of neurodegenerative disease states and behavioural disorders such as Alzheimers Disease, Parkinsons Disease, Huntintons Disease, schizophrenia, mania, dementia, paranoia, obsessive compulsive disorder and panic disorder. Moreover, expression within fetal tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:19 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1323 of SEQ ID NO:19, b is an integer of 15 to 1337, where both a and b correspond to the positions of nucleotide residues shown in SEQ ID NO:19, and where b is greater than or equal to a +14.

Features of Protein Encoded by Gene No: 10

Preferred polypeptides of the invention comprise the following amino acid sequence:

| | |
|---|---|
| ECGSWAGFHTSSFPRPSALALAAWRRWGSICHLHTAGFIFGAAPRGNKCR, | (SEQ ID NO:300) |
| TSSFPRPSALALAAWRRWGSI, and/or | (SEQ ID NO:301) |
| ICHLHTAGFIFGAAPRGNKCR. | (SEQ ID NO:302) |

Polynucleotides encoding these polypeptides are also provided.

This gene is expressed primarily in breast tissue, and to a lesser extent in liver tissue.

Therefore, polynucleotides and polypeptides of the invention are useful as reagents for differential identification of the tissue(s) or cell type(s) present in a biological sample and for diagnosis of diseases and conditions which include, but are not limited to, breast cancer, hepatoblastoma, hepatitis, liver metabolic diseases, and conditions that are attributable to the differentiation of hepatocyte progenitor cells. Similarly, polypeptides and antibodies directed to these polypeptides are useful in providing immunological probes for differential identification of the tissue(s) or cell type(s). For a number of disorders of the above tissues or cells, particularly of the breast and liver, expression of this gene at significantly higher or lower levels may be routinely detected in certain tissues or cell types (e.g., breast, liver, cancerous and wounded tissues) or bodily fluids (e.g., lymph, serum, plasma, breast milk, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 148 as residues: Gln-29 to Gly-38, Lys-57 to Asp-62.

The tissue distribution indicates that polynucleotides and polypeptides corresponding to this gene are useful for the detection and/or treatment of liver disorders and cancers (e.g., hepatoblastoma, jaundice, hepatitis, liver metabolic diseases), and conditions that are attributable to the differentiation of hepatocyte progenitor cells. Representative uses are described in the “Hyperproliferative Disorders”, “Infectious Disease”, and “Binding Activity” sections below, in Example 11, and 27, and elsewhere herein. In addition, the expression in breast would suggest a possible role in the detection and treatment of breast cancer. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:20 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1148 of SEQ ID NO:20, b is an integer of 15 to 1162, where both a and b correspond to the positions of nucleotide residues shown in SEQ ID NO:20, and where b is greater than or equal to a +14.

Features of Protein Encoded by Gene No: 11

Preferred polypeptides of the invention comprise the following amino acid sequence: PDTLDKSPLAPGSSLVDPQISLWVL (SEQ ID NO:303). Polynucleotides encoding these polypeptides are also provided.

This gene is expressed primarily in brain, frontal cortex, and retinal tissues.

Therefore, polynucleotides and polypeptides of the invention are useful as reagents for differential identification of the tissue(s) or cell type(s) present in a biological sample and for diagnosis of diseases and conditions which include, but are not limited to, developmental, degenerative and behavioral diseases and/or disorders of the brain such as depression, schizophrenia, Alzheimer's disease, Parkinson's disease, Huntington's disease, specific brain tumors, aphasia, mania, depression, dementia, paranoia, addictive behavior and sleep disorders as well as conditions that affect vision and function of the eye, such as retinoblastoma and cataracts. Similarly, polypeptides and antibodies directed to these polypeptides are useful in providing immunological probes for differential identification of the tissue(s) or cell type(s). For a number of disorders of the above tissues or cells, particularly of the brain and eye, expression of this gene at significantly higher or lower levels may be routinely detected in certain tissues or cell types (e.g., brain, retina, visual, and cancerous and wounded tissues) or bodily fluids (e.g., lymph, serum, plasma, urine, aqueous human, vitreous humor, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 149 as residues: Pro-46 to Gln-60, Pro-68 to Gly-75, Leu-78 to Ala-86, Gln-93 to Asp-98.

The tissue distribution indicates that polynucleotides and polypeptides corresponding to this gene are useful for the treatment and/or diagnosis of developmental, degenerative and behavioral diseases, and conditions of the brain such as aphasia, depression, schizophrenia, Alzheimer's disease, Parkinson's disease, Huntington's disease, specific brain tumors, mania, depression, dementia, paranoia, addictive behavior and sleep disorders. Representative uses are described in the “Regeneration” and “Hyperproliferative Disorders” sections below, in Example 11, 15, and 18, and elsewhere herein. In addition, the expression in retina would also suggest a role for this gene product in the diagnosis and treatment of conditions that affect vision and function of the eye such as retinoblastoma, myopia, hyperopia and cataracts. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:21 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1823 of SEQ ID NO:21, b is an integer of 15 to 1837, where both a and b correspond to the positions of nucleotide residues shown in SEQ ID NO:21, and where b is greater than or equal to a +14.

Features of Protein Encoded by Gene No: 12

Preferred polypeptides of the invention comprise the following amino acid sequence:

| | |
|---|---|
| MSPYASQGFPFLPPYPPQEANRSITSLSVADTVSSSTTSHTTAKPAAPSFGVLS | (SEQ ID NO:304) |
| NLPLPIPTVDASTPTSQNGFGYKMPDVPDAEPELSELSVSQLTDMNEQEEVLLE | |
| QFLTLPQLKQIITDKDDLVKSTEELARKNLLLEPSLEAKRQTVLDKYELLTQM | |
| KSTFEKKMQRQHELSESCSASALQARLKVAAHIEAEEESDNIAEDFLEGKMEI | |
| DDFLSSFMEKRTICHCRRAKEEKLQQAIAMHSQFHAPL, | |
| LPPYPPQEANRSITSLSVADTVS, | (SEQ ID NO:305) |
| TAKPAAPSFGVLSNLPLPIPTVDASIP, | (SEQ ID NO:306) |
| PDVPDAFPELSELSVSQLTDMNEQE, | (SEQ ID NO:307) |
| QFLTLPQLKQIITDKDDLVKSILEELARKN, | (SEQ ID NO:308) |
| RQTVLDKYELLTQMKSTFEKKMQRQ, | (SEQ ID NO:309) |
| ASALQARLKVAAHEAEEESDNIAEDFLE, | (SEQ ID NO:310) |
| MEKRTICHCRRAKEEKLQQAIAMHSQF, and/or | (SEQ ID NO:311) |
| LLLQQHZFLIYTVTQVGCLL. | (SEQ ID NO:312) |

Polynucleotides encoding these polypeptides are also provided.

The gene encoding the disclosed cDNA is thought to reside on chromosome 8. Accordingly, polynucleotides related to this invention are useful as a marker in linkage analysis for chromosome 8.

This gene is expressed primarily in breast, placenta, and testis tissues, and to a lesser extent in a variety of other tissues and cell types.

Therefore, polynucleotides and polypeptides of the invention are useful as reagents for differential identification of the tissue(s) or cell type(s) present in a biological sample and for diagnosis of diseases and conditions which include, but are not limited to, breast and endometrial cancers as well as pre-natal and reproductive disorders and deficiencies. Similarly, polypeptides and antibodies directed to these polypeptides are useful in providing immunological probes for differential identification of the tissue(s) or cell type(s). For a number of disorders of the above tissues or cells, particularly of the breast and reproductive system, expression of this gene at significantly higher or lower levels may be routinely detected in certain tissues or cell types (e.g., breast, reproductive, placental, tesicular, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in breast and endometrial tissue indicates that polynucleotides and polypeptides corresponding to this gene are useful for the detection and/or treatment of breast cancer, ovarian and other endometrial cancers, infertility and pre-natal disorders. Representative uses are described elsewhere herein. Furthermore, the tissue distribution indicates that polynucleotides and polypeptides corresponding to this gene are useful for treating female infertility. The protein product is likely involved in preparation of the endometrium of implantation and could be administered either topically or orally.

Alternatively, this gene could be transfected in gene-replacement treatments into the cells of the endometrium and the protein products could be produced. Similarly, these treatments could be performed during artificial insemination for the purpose of increasing the likelyhood of implantation and development of a healthy embryo. In both cases this gene or its gene product could be administered at later stages of pregnancy to promote heathy development of the endometrium. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:22 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1040 of SEQ ID NO:22, b is an integer of 15 to 1054, where both a and b correspond to the positions of nucleotide residues shown in SEQ ID NO:22, and where b is greater than or equal to a +14.

Features of Protein Encoded by Gene No: 13

Preferred polypeptides of the invention comprise the following amino acid sequence: EFGTRKSKSKINIKEE (SEQ ID NO: 313). Polynucleotides encoding these polypeptides are also provided.

This gene is expressed primarily in retina, and, to a lesser extent, in anergic T-cells.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly autoimmune disorders such as lupus and degenerative visual disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, visual, and cancerous and wounded tissues) or bodily fluids (e.g., lymph, serum, plasma, urine, aqueous humor, vitreous humor, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 151 as residues: Lys-49 to Gln-57, Arg-63 to Ala-69.

The tissue distribution in T-cells indicates that the polypeptides or polynucleotides are useful for the treatment, prophylaxis, and diagnosis of immune and autoimmune diseases, such as lupus, transplant rejection. allergic reactions, arthritis, asthma, immunodeficiency diseases, leukemia, and AIDS. Representative uses are described in the “Immune Activity”, and “Infectious Disease” sections below, in Example 11, 13, 14, 16, 18, 19, 20, and 27, and elsewhere herein. Briefly, polynucleotides or polypeptides of this gene are important in treating and/or detecting hematopoietic disorders, such as graft versus host reaction, graft versus host disease, transplant rejection, myelogenous leukemia, bone marrow fibrosis, and myeloproliferative disease. The polypeptides or polynucleotides are also useful to enhance or protect proliferation, differentiation, and functional activation of hematopoietic progenitor cells (e.g., bone marrow cells), useful in treating cancer patients undergoing chemotherapy or patients undergoing bone marrow transplantation. The polypeptides or polynucleotides are also useful to increase the proliferation of peripheral blood leukocytes, which can be used in the combat of a range of hematopoietic disorders, including immunodeficiency diseases, leukemia, and septicemia. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:23 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1052 of SEQ ID NO:23, b is an integer of 15 to 1066, where both a and b correspond to the positions of nucleotide residues shown in SEQ ID NO:23, and where b is greater than or equal to a +14.

Features of Protein Encoded by Gene No: 14

The translation product of this gene shares sequence homology with a drought-induced protease inhibitor from soybean. As a result, the protein product of this gene may show utility in the treatment and/or prevention of a variety of proliferative disorders (e.g., for inhibition of key proteolytic events during cellular metabolism of the tumor which may lead to cessation of mitosis) or for the treatment of degenerative conditions where the inhibition of aberrant proteolysis may lead to cessation of degeneration and ultimately in immune protection.

Preferred polypeptides of the invention comprise the following amino acid sequence: GTSSKVVTQKVHLSSVEFPF (SEQ ID NO:314). Polynucleotides encoding these polypeptides are also provided.

This gene is expressed primarily in the kidney cortex.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the kidney. Similarly, polypeptides and antibodies directed to these polypeptides are useful in providing immunological probes for differential identification of the tissue(s) or cell type(s). For a number of disorders of the above tissues or cells, particularly of the urogenital system, expression of this gene at significantly higher or lower levels may be routinely detected in certain tissues or cell types (e.g., kidney, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 152 as residues: Glu-48 to Arg-56, Ser-61 to Gly-66.

The tissue distribution in kidney tissue, combined with the homology to a protease inhibitor, indicates that polynucleotides and polypeptides corresponding to this gene are useful for the diagnosis and treatment of disorders affecting the kidney. Representative uses are described elsewhere herein. Briefly, the uses include, but are not limited to the detection, treatment, and/or prevention of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilms Tumor Disease, and congenital kidney abnormalities such as horseshoe kidney, polycystic kidney, and Falconi's syndrome. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:24 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 914 of SEQ ID NO:24, b is an integer of 15 to 928, where both a and b correspond to the positions of nucleotide residues shown in SEQ ID NO:24, and where b is greater than or equal to a +14.

Features of Protein Encoded by Gene No: 15

When tested against U937 cell lines, supernatants removed from cells containing this gene activated the GAS (gamma activating sequence) promoter element. Thus, it is likely that this gene activates myeloid cells, and, to a lesser extent, in other immune and hematopoietic cells and cell types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| TRPVFLSMTPLKGIKSVILPQVFLCAYMAAFNSTNGNRSYTCKPLERSLLMAG | (SEQ ID NO:315) |
| AVASSTFLGVIPQFVQ, | |
| PLKGIKSVILPQVFLCAYMAA, and/or | (SEQ ID NO:316) |
| AFNSINGNRSYTCKPLERSLL. | (SEQ ID NO:317) |

Polynucleotides encoding these polypeptides are also provided.

The gene encoding the disclosed cDNA is believed to reside on chromosome 10. Accordingly, polynucleotides related to this invention are useful as a marker in linkage analysis for chromosome 10.

This gene is expressed primarily in B cell and T cell lymphomas.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders, particularly B cell and T cell lymphomas, infections, multiple myeloma, immunodeficiencies, and inflammatory conditions. Similarly, polypeptides and antibodies directed to these polypeptides are useful in providing immunological probes for differential identification of the tissue(s) or cell type(s). For a number of disorders of the above tissues or cells, particularly immune or hematopoietic disorders, such as B- and T-cell lymphomas, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 153 as residues: Phe-85 to Gly-96, Glu-133 to Thr-143.

The tissue distribution in B- and T-cell lymphomas, combined with the detected GAS biological activity, indicates that polynucleotides and polypeptides corresponding to this gene are useful for the diagnosis and treatment of a variety of immune disorders, particularly proliferative conditions such as cancer and leukemias. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:25 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 952 of SEQ ID NO:25, b is an integer of 15 to 966, where both a and b correspond to the positions of nucleotide residues shown in SEQ ID NO:25, and where b is greater than or equal to a +14.

Features of Protein Encoded by Gene No: 16

The protein product of this gene was found to have homology to the Poly(A) polymerase of Bos taurus, which is known to be important in the creation of the 3′ poly(A) tail of rnRNA's (See Genbank Accession No.gi|1377872; all references available through the cited accession number are hereby incorporated herein by reference, see for example, Mol. Cell. Biol. 16 (5), 2378-2386 (1996)).

The gene encoding the disclosed cDNA is believed to reside on chromosome 14. Accordingly, polynucleotides related to this invention are useful as a marker in linkage analysis for chromosome 14.

This gene is expressed primarily in brain, and, to a lesser extent, in prostate.

Therefore, polynucleotides and polypeptides of the invention are useful as reagents for differential identification of the tissue(s) or cell type(s) present in a biological sample and for diagnosis of diseases and conditions which include, but are not limited to, neural disorders, such as neurodegenerative disease states and behavioral conditions, in addition to reproductive disorders, particularly of the prostate. Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues and cell types (e.g., neural, reproductive, and cancerous and wounded tissues) or bodily fluids (e.g., lymph, serum, seminal fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 154 as residues: Glu-47 to Ser-52.

The tissue distribution in brain indicates that polynucleotides and polypeptides corresponding to this gene are useful for the detection/treatment of neurodegenerative disease states and behavioural disorders. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimers Disease, Parkinsons Disease, Huntingtons Disease, schizophrenia, mania, dementia, paranoia, obsessive compulsive disorder and panic disorder. Moreover, expression of the gene in prostate indicates that polynucleotides and polypeptides corresponding to this gene are useful for the detection or treatment of prostate disorders including benign prostate hyperplasia, prostate cancer, and metabolic disorders. The homology to the PAP polyA polymerase indicates that the protein product of this gene, antibodies directed to this protein, or the gene encoding this protein via a gene therapy approach, may show utility as a preventative therapy for proliferative conditions. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:26 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1132 of SEQ ID NO:26, b is an integer of 15 to 1146, where both a and b correspond to the positions of nucleotide residues shown in SEQ ID NO:26, and where b is greater than or equal to a +14.

Features of Protein Encoded by Gene No: 17

Preferred polypeptides of the invention comprise the following amino acid sequence: PESPVYPRRRTFSPNPSPI (SEQ ID NO:318). Polynucleotides encoding these polypeptides are also provided.

This gene is expressed primarily in epididymus.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the reproductive organs. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, testicular, and cancerous and wounded tissues) or bodily fluids (e.g., lymph, serum, plasma, urine, seminal fluid, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 155 as residues: Met-l to Pro-6, Glu-58 to Cys-63, Glu-65 to Gly-72, Thr-74 to Val-87.

The tissue distribution in epididymus indicates that polynucleotides and polypeptides corresponding to this gene are useful for the diagnosis and/or treatment of disorders of the epididymus and reproductive organs. Representative uses are described elsewhere herein. Furthermore, the tissue distribution indicates that polynucleotides and polypeptides corresponding to this gene are useful for the treatment and diagnosis of conditions concerning proper testicular function (e.g., endocrine function, sperm maturation), as well as cancer. Therefore, this gene product is useful in the treatment of male infertility and/or impotence. This gene product is also useful in assays designed to identify binding agents as such agents (antagonists) are useful as male contraceptive agents. Similarly, the protein is believed to by useful in the treatment and/or diagnosis of testicular cancer. The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:27 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 788 of SEQ ID NO:27, b is an integer of 15 to 802, where both a and b correspond to the positions of nucleotide residues shown in SEQ ID NO:27, and where b is greater than or equal to a +14.

Features of Protein Encoded by Gene No: 18

Preferred polypeptides of the invention comprise the following amino acid sequence: NVSANLNFHVH (SEQ ID NO:319). Polynucleotides encoding these polypeptides are also provided.

This gene is expressed primarily in synovium and rhabdomyosarcoma.

Therefore, polynucleotides and polypeptides of the invention are useful as reagents for differential identification of the tissue(s) or cell type(s) present in a biological sample and for diagnosis of diseases and conditions which include, but are not limited to, musculo-skeletal system diseases and/or disorders, including cancer. Similarly, polypeptides and antibodies directed to these polypeptides are useful in providing immunological probes for differential identification of the tissue(s) or cell type(s). For a number of disorders of the above tissues or cells, particularly of the musculo-skeletal system, expression of this gene at significantly higher or lower levels may be routinely detected in certain tissues or cell types (e.g., musculo-skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 156 as residues: Trp-30 to Val-35, Lys-44 to Arg-49.

The tissue distribution in synovium and rhabdomyosarcoma tissue indicates that polynucleotides and polypeptides corresponding to this gene are useful for the treatment and/or diagnosis of disorders of the musculo-skeletal system, and cancer. Representative uses are described elsewhere herein. Furthermore, the expression of this gene product in synovium would suggest a role in the detection and treatment of disorders and conditions afflicting the skeletal system, in particular osteoporosis, bone cancer, connective tissue disorders (e.g. arthritis, trauma, tendonitis, chrondomalacia and inflammation). The protein is also useful in the diagnosis or treatment of various autoimmune disorders (i.e., rheumatoid arthritis, lupus, scleroderma, and dermatomyositis), dwarfism, spinal deformation, joint abnormalities, and chondrodysplasias (i.e. spondyloepiphyseal dysplasia congenita, familial osteoarthritis, Atelosteogenesis type II, metaphyseal chondrodysplasia type Schmid, etc.). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:28 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1155 of SEQ ID NO:28, b is an integer of 15 to 1169, where both a and b correspond to the positions of nucleotide residues shown in SEQ ID NO:28, and where b is greater than or equal to a +14.

Features of Protein Encoded by Gene No: 19

The gene encoding the disclosed cDNA is thought to reside on chromosome 5. Accordingly, polynucleotides related to this invention are useful as a marker in linkage analysis for chromosome 5.

This gene is expressed primarily in fetal liver/spleen, and, to a lesser extent, in tonsils.

Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, or hepatic disorders, particularly mutiple myeloma, immunodeficiencies, and cancers. Similarly, polypeptides and antibodies directed to these polypeptides are useful in providing immunological probes for differential identification of the tissue(s) or cell type(s). For a number of disorders of the above tissues or cells, particularly of the hepatic system, expression of this gene at significantly higher or lower levels may be routinely detected in certain tissues or cell types (e.g., immune, cancerous and wounded tissues) or bodily fluids (e.g., lymph, serum, bile, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 157 as residues: Asp-27 to Ser-36.

The tissue distribution in fetal liver and tonsil tissue indicates that polynucleotides and polypeptides corresponding to this gene are useful for the diagnosis and treatment of a variety of immune system disorders. Representative uses are described in the “Immune Activity” and “Infectious Disease” sections below, in Example 11, 13, 14, 16, 18, 19, 20, and 27, and elsewhere herein. Briefly, the protein product of this gene may play a role in regulatingproliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses). Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis.

Expression of this gene at either the RNA or protein level indicates that polynucleotides and polypeptides of the present invention could be used as a diagnostic indicator of hepatic cancer. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:29 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1452 of SEQ ID NO:29, b is an integer of 15 to 1466, where both a and b correspond to the positions of nucleotide residues shown in SEQ ID NO:29, and where b is greater than or equal to a +14.

Features of Protein Encoded by Gene No: 20

This gene is expressed primarily in human brain.

Therefore, polynucleotides and polypeptides of the invention are useful as reagents for differential identification of the tissue(s) or cell type(s) present in a biological sample and for diagnosis of diseases and conditions which include, but are not limited to, disorders or diseases of the central nervous system. Similarly, polypeptides and antibodies directed to these polypeptides are useful in providing immunological probes for differential identification of the tissue(s) or cell type(s). For a number of disorders of the above tissues or cells, particularly of the brain, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in brain tissue indicates that polynucleotides and polypeptides corresponding to this gene are useful for the diagnosis and the treatment of CNS disorders. Representative uses are described in the “Regeneration” and “Hyperproliferative Disorders” sections below, in Example 11, 15, and 18, and elsewhere herein. Briefly, the uses include, but are not limited to the detection, treatment, and/or prevention of of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:30 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1212 of SEQ ID NO:30, b is an integer of 15 to 1226, where both a and b correspond to the positions of nucleotide residues shown in SEQ ID NO:30, and where b is greater than or equal to a +14.

Features of Protein Encoded by Gene No: 21

Preferred polypeptides of the invention comprise the following amino acid sequence:

| | |
|---|---|
| MSDFEKVDISVHQHIHVGPLLLMTTESWGPSCAPSPALLSGHTAASFTHTLGG | (SEQ ID NO:320) |
| VLGCPPYHKEYSS | |
| AHTSDHRKETNKVEEGRWVDVTRSLGNFNFRRKFFCVSELLICGIELDSSWKL | |
| QINSNDCKVL, | |
| VGPLLLMTTESWGPSCAPSPALLSGHTAAS, | (SEQ ID NO:321) |
| ETNKVEEGRWVDVTRSLGNFNERRKFF, and/or | (SEQ ID NO:322) |
| QSPRVRSLGD. | (SEQ ID NO:323) |

Polynucleotides encoding these polypeptides are also provided.

This gene is expressed primarily in fetal spleen or liver, adult spleen, and, to a lesser extent, in activated T-cells.

Therefore, polynucleotides and polypeptides of the invention are useful as reagents for differential identification of the tissue(s) or cell type(s) present in a biological sample and for diagnosis of diseases and conditions which include, but are not limited to, immune and hematopoietic diseases and/or disorders, particularly abnormal proliferation or activation of hematopoietic cells, particularly of T-cells and their progenitors. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 159 as residues: Arg-19 to Phe-24, Ala-44 to Asp-51, Glu-60 to Ile-66.

The tissue distribution in spleen tissues and T-cells indicates that polynucleotides and polypeptides corresponding to this gene are useful for modulating or detecting the abnormal proliferation or activation of T-cells and immune cell precursor cells. Representative uses are described in the “Immune Activity” and “Infectious Disease” sections below, in Example 11, 13, 14,16, 18, 19, 20, and 27, and elsewhere herein. Briefly, the expression within fetal spleen indicates that polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types.

Similarly, this gene product may be involved in the regulation of cytokine production, antigen presentation, or other processes that may also suggest a usefulness in the treatment of cancer (e.g., by boosting immune responses). Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:31 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1080 of SEQ ID NO:31, b is an integer of 15 to 1094, where both a and b correspond to the positions of nucleotide residues shown in SEQ ID NO:31, and where b is greater than or equal to a +14.

Features of Protein Encoded By Gene No: 22

Contact of cells with supernatant expressing the product of this gene has been shown to increase the permeability of the plasma membrane of THP-1 cells to calcium. Thus it is likely that the product of this gene is involved in a signal transduction pathway that is initiated when the product binds a receptor on the surface of the plasma membrane of both monocytes. Thus, polynucleotides and polypeptides have uses which include, but are not limited to, activating monocytes, and, to a lesser extent, in other tissues and cell types.

Preferred polypeptides of the invention comprise the following amino acid sequence:

(SEQ ID NO:324)

GPMKDCEYSQISTHSSSPMESPHKKKKIAARRKWEVFPGRNKFFCNGRI,

(SEQ ID NO:325)

SQISTHSSSPMESPHKKKKIA, and/or

(SEQ ID NO:326)

AARRKWEVFPGRNKFFCNGRI.

Polynucleotides encoding these polypeptides are also provided.

This gene is expressed primarily in the amygdala.

Therefore, polynucleotides and polypeptides of the invention are useful as reagents for differential identification of the tissue(s) or cell type(s) present in a biological sample and for diagnosis of diseases and conditions which include, but are not limited to, developmental, degenerative and behavioral diseases of the brain such as depression, schizophrenia, Alzheimer's disease, Parkinson's disease, Huntington's disease, specific brain tumors, aphasia, mania, depression, dementia, paranoia, addictive behavior and sleep disorders. Similarly, polypeptides and antibodies directed to these polypeptides are useful in providing immunological probes for differential identification of the tissue(s) or cell type(s). For a number of disorders of the above tissues or cells, particularly of the brain, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 160 as residues: Pro-94 to Ala-107.

The tissue distribution in amygdala, combined with the detected calcium flux activity, indicates that polynucleotides and polypeptides corresponding to this gene are useful for the treatment and/or diagnosis of developmental, degenerative and behavioral diseases and conditions of the brain. Representative uses are described in the “Regeneration” and “Hyperproliferative Disorders” sections below, in Example 11, 15, and 18, and elsewhere herein. Briefly, the uses include, but are not limited to the detection, treatment, and/or prevention of aphasia, depression, schizophrenia, Alzheimer's disease, Parkinson's disease, Huntington's disease, specific brain tumors, mania, depression, dementia, paranoia, addictive behavior and sleep disorders. Furthermore, the protein may also be used to determine biological activity, to raise antibodies, as tissue markers, to isolate cognate ligands or receptors, to identify agents that modulate their interactions, in addition to its use as a nutritional supplement. Thr protein may modulate the immune response to aberrant polypeptides, as may be present in proliferative tissues and cells (i.e., brain tumor tissue, etc.). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:32 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1023 of SEQ ID NO:32, b is an integer of 15 to 1037, where both a and b correspond to the positions of nucleotide residues shown in SEQ ID NO:32, and where b is greater than or equal to a +14.

Features of Protein Encoded by Gene No: 23

The translation product of this gene shares sequence homology with octaprenyltransferase, which is thought to be important in the biosynthesis of ubiquitin, and may be essential for cellular function and metabolism. When tested against fibroblast cell lines, supernatants removed from cells containing this gene activated the EGR1 assay. Thus, it is likely that this gene activates fibroblast cells, and to a lesser extent, other tissues and cell types, through a signal transduction pathway. Early growth response 1 (EGR1) is a promoter associated with certain genes that induces various tissues and cell types upon activation, leading the cells to undergo differentiation and proliferation.

Preferred polypeptides of the invention comprise the following amino acid sequence: PPFPHPETGQLCLVDSAPRPLQPYLRL (SEQ ID NO:327). Polynucleotides encoding these polypeptides are also provided.

The gene encoding the disclosed cDNA is thought to reside on chromosome 4. Accordingly, polynucleotides related to this invention are useful as a marker in linkage analysis for chromosome 4.

This gene is expressed primarily in synovium, liver cells, dendritic cells and stromal cells.

Therefore, polynucleotides and polypeptides of the invention are useful as reagents for differential identification of the tissue(s) or cell type(s) present in a biological sample and for diagnosis of diseases and conditions which include, but are not limited to, metabolic, developmental, and immune diseases and/or disorders. Similarly, polypeptides and antibodies directed to these polypeptides are useful in providing immunological probes for differential identification of the tissue(s) or cell type(s). For a number of disorders of the above tissues or cells, particularly of the metabolic processes and the immune system, expression of this gene at significantly higher or lower levels may be routinely detected in certain tissues or cell types (e.g., immune, developmental, metabolic, liver, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 161 as residues: Asp-54 to Asn-69, His-176 to Asp-181, Phe-194 to Trp-201, Ser-220 to Pro-225, Arg-248 to Trp-253, Trp-276 to Ile-288.

The tissue distribution in liver and immune tissue and cells, combined with the homology to octaprenyltransferase, indicates that polynucleotides and polypeptides corresponding to this gene are useful for the treatment and/or diagnosis of metabolic and respiratory disorders. Representative uses are described elsewhere herein. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:33 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1362 of SEQ ID NO:33, b is an integer of 15 to 1376, where both a and b correspond to the positions of nucleotide residues shown in SEQ ID NO:33, and where b is greater than or equal to a +14.

Features of Protein Encoded by Gene No: 24

Preferred polypeptides of the invention comprise the following amino acid sequence: HPMCAKVADPELSSCPHCGLTAQPGPESGNISHSLREGSPRTLFVDSTSQASV PAAECPGHREGTP FSGASTSQAF (SEQ ID NO:328). Polynucleotides encoding these polypeptides are also provided.

This gene is expressed primarily in activated T cells and in the spleen from a patient suffering from lymphocytic leukemia.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly immunodeficiencies, multiple myeloma, and leukemias. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in T-cells and spleen tissue indicates that polynucleotides and polypeptides corresponding to this gene are useful for the diagnosis or treatment of leukemia. Representative uses are described in the “Immune Activity” and “Infectious Disease” sections below, in Example 11, 13, 14, 16, 18, 19, 20, and 27, and elsewhere herein. Briefly, the tissue distribution indicates that the polypeptides or polynucleotides are useful for treatment, prophylaxis, and diagnosis of immune and autoimmune diseases, such as lupus, transplant rejection, allergic reactions, arthritis, asthma, immunodeficiency diseases, leukemia, and AIDS. The expression observed predominantly in hematopoietic cells also indicates that the polynucleotides or polypeptides are important in treating and/or detecting hematopoietic disorders, such as graft versus host reaction, graft versus host disease, transplant rejection, myelogenous leukemia, bone marrow fibrosis, and myeloproliferative disease.

The polypeptides or polynucleotides are also useful to enhance or protect proliferation, differentiation, and functional activation of hematopoietic progenitor cells (e.g., bone marrow cells), useful in treating cancer patients undergoing chemotherapy or patients undergoing bone marrow transplantation. The polypeptides or polynucleotides are also useful to increase the proliferation of peripheral blood leukocytes, which can be used in the combat of a range of hematopoietic disorders, including immmunodeficiency diseases, leukemia, and septicemia. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:34 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1206 of SEQ ID NO:34, b is an integer of 15 to 1220, where both a and b correspond to the positions of nucleotide residues shown in SEQ ID NO:34, and where b is greater than or equal to a +14.

Features of Protein Encoded by Gene No: 25

The translation product of this gene was shown to have homology to the human krueppel family zinc finger protein (See Genbank Accession No. gi|2384653; all references available through this accession no. are hereby incorporated herein by reference) which is thought to be involved in gene regulation.

Preferred polypeptides of the invention comprise the following amino acid sequence: TPLLSPCLQPLPGV (SEQ ID NO: 329). Polynucleotides encoding these polypeptides are also provided.

This gene is expressed primarily in bone marrow.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly disorders afflicting stem cell or myeloid progenitors, and in particular multiple myeloma, immunodeficiencies, or SCID. Similarly, polypeptides and antibodies directed to these polypeptides are useful in providing immunological probes for differential identification of the tissue(s) or cell type(s). For a number of disorders of the above tissues or cells, particularly of the immune and hematopoetic systems, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in bone marrow indicates that polynucleotides and polypeptides corresponding to this gene are useful for the diagnosis and treatment of disorders affecting the immune and hematopoetic systems. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the diagnosis and/or treatment of hematopoietic disorders. Furthermore, this gene product is primarily expressed in hematopoietic cells and tissues, suggesting that it plays a role in the survival, proliferation, and/or differentiation of hematopoieitic lineages. This is particularly supported by the expression of this gene product in bone marrow, which is a primary sites of definitive hematopoiesis. The uses include bone marrow cell ex vivo culture, bone marrow transplantation, bone marrow reconstitution, radiotherapy or chemotherapy of neoplasia.

The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:35 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1332 of SEQ ID NO:35, b is an integer of 15 to 1346, where both a and b correspond to the positions of nucleotide residues shown in SEQ ID NO:35, and where b is greater than or equal to a +14.

Features of Protein Encoded by Gene No: 26

Preferred polypeptides of the invention comprise the following amino acid sequence: TRRSCSSQVSS (SEQ ID NO: 330). Polynucleotides encoding these polypeptides are also provided.

This gene is expressed primarily in the neutrophils.

Therefore, polynucleotides and polypeptides of the invention are useful as reagents for differential identification of the tissue(s) or cell type(s) present in a biological sample and for diagnosis of diseases and conditions which include, but are not limited to, disorders of the immune systems, such as AIDS. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 164 as residues: His-17 to Ser-24, Glu-53 to Asn-58, Glu-66 to Lys-72.

The tissue distribution in immune cells indicates that polynucleotides and polypeptides corresponding to this gene are useful for the diagnosis and treatment of a variety of immune system disorders. Representative uses are described in the “Immune Activity” and “Infectious Disease” sections below, in Example 11, 13, 14, 16, 18, 19, 20, and 27, and elsewhere herein. Briefly, the the expression of this gene product indicates a role in regulatingproliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:36 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1012 of SEQ ID NO:36, b is an integer of 15 to 1026, where both a and b correspond to the positions of nucleotide residues shown in SEQ ID NO:36, and where b is greater than or equal to a +14.

Features of Protein Encoded by Gene No: 27

The translation product of this gene shares sequence homology with glucan synthetase which is thought to be important in modifying carbohydrate moieties on extracellular molecules.

This gene is expressed primarily in T-cells, and, to a lesser extent, in human embryo and retina.

Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, and developmental diseases and/or disorders, particularly autoimmune diseases and inflammation. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, developmental, visual, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 165 as residues: Gly-33 to Leu-39, Thr-69 to Ser-77, Arg-102 to Thr-109.

The tissue distribution in T-cells, combined with the homology to glucan synthetase, indicates that polynucleotides and polypeptides corresponding to this gene are useful for modifying the response to and production of active cytokines by T cells, in modulating cell-cell interactions, or cell-tissue interactions, and in inflammatory conditions. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. The protein, antibodies directed to the protein, or polynucleotides encoding the disclosed protein, are useful in modulating the immune response to a variety of conditions (i.e., through the inhibition of cellular adhesion and migration via loss of function of glucan synthetase, etc.). The protein, antibodies directed to the protein, or polynucleotides encoding the protein, also useful in the treatment or prevention of proliferative conditions, particularly in inhibiting metastasis. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:37 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 818 of SEQ ID NO:37, b is an integer of 15 to 832, where both a and b correspond to the positions of nucleotide residues shown in SEQ ID NO:37, and where b is greater than or equal to a +14.

Features of Protein Encoded by Gene No: 28

Preferred polypeptides of the invention comprise the following amino acid sequence:

(SEQ ID NO:331)

GRGDKPPQDRPASLRLKGPPSCQAPASHSSTLSSHCPCSLFACGSVWPGS
LGSGIFARLSQLLPSPASWGWDFLTLRQAQQMLGPSLCPGHSTSAHQHYG
AYVLPRDLCSFLLTSTVQGTAPLKNSRVTCLIGSQQVPLC,

(SEQ ID NO:332)

AEVTSPAKTDLQVFVSRDLPHARPLPLTAAPFPLIVPVPFLPVDLFGQGP
WGQEYLQDSASSFPAQPLGAGTFSPCGRHNRCWDPVSAQVTAQVHISTMG
PMSCPETSAPSCSHPQFRARRPSRTPESPVSSAPSKCLFV YDVPLL,

(SEQ ID NO:333)

SLRLKGPPSCQAPASHSSTLSSHCPCSLFA,

(SEQ ID N0:334)

QQMLGPSLCPGHSTSAHQHYGAYVLPRDLC,

(SEQ ID NO:335)

DLQVFVSRDLPHARPLPLTAAPFPLIVPVPF,

(SEQ ID NO:336)

AQVHISTMGPMSCPETSAPSCSHPQFRARRPSRTPESPV,
and/or

(SEQ ID NO:337)

QAPPRQTCKSSSQGTSL.

Polynucleotides encoding these polypeptides are also provided.

This gene is expressed primarily in endometrial tumors, fetal spleen, and, to a lesser extent, in activated monocytes and T-cells.

Therefore, polynucleotides and polypeptides of the invention are useful as reagents for differential identification of the tissue(s) or cell type(s) present in a biological sample and for diagnosis of diseases and conditions which include, but are not limited to, reproductive, immune, hematopoietic disorders, particularly pregnancy defects. Similarly, polypeptides and antibodies directed to these polypeptides are useful in providing immunological probes for differential identification of the tissue(s) or cell type(s). For a number of disorders of the above tissues or cells, particularly of the reproductive and immune systems, expression of this gene at significantly higher or lower levels may be routinely detected in certain tissues and cell types (e.g., reproductive, endometrial, immune, hematopoietic, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 166 as residues: Ser-66 to Thr-75.

The tissue distribution in endometrial tissue indicates that the protein product of this gene could be used in the teatment and/or detection of pregnancy associated disorders including miscarriage, and endometriosis. Representative uses are described in the “Immune Activity”, “Regeneration”, “Hyperproliferative Disorders”, and “Infectious Disease” sections below, in Example 11, 13, 14, 16, 18, 19, 20, and 27, and elsewhere herein. Briefly, expression in hematopoietic cells indicates that polynucleotides and polypeptides corresponding to this gene are useful for the treatment and/or detection of immune system related diseases including arthritis, asthma, immunodeficiency diseases and leukemia. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:38 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 692 of SEQ ID NO:38, b is an integer of 15 to 706, where both a and b correspond to the positions of nucleotide residues shown in SEQ ID NO:38, and where b is greater than or equal to a +14.

Features of Protein Encoded by Gene No: 29

Preferred polypeptides of the invention comprise the following amino acid sequence:

(SEQ ID NO:338)

AALRPSGSLAGPEWPWQHWCGCWREHXVKPQQVDLHSARLWAAPAAVGPA
HAGGSPGMPPGGTAPHARRHSLPSPTAQSHLWHVHGLRQRGPKAVPLDLA
QLVTTTTPLFXLALSALLLGRRHHPLQLAAMGPLCLGAACSLAGEFRTPP
TGCGFLLAATCLRGLKSVQQSALLQEERLDAVTLLYATSLPSFCLLAGAA
LVLEAGVAPPPTAGDSRLWACILLSCLLSVLYNLASFSLLALTSALTVHV
LGNLTVVGNLILSRLLFGSRLSALSYVGIA LTLSGMFLYHNCEFVASWA
ARRGLWRRDQPSKGL,

(SEQ ID NO:339)

GQPSGPPAAWPGPSGHGSTGVAAGGSTXSSLNKWIFTVHGFGRPLLLSAL
HMLVAALACHRGARRP,

(SEQ ID NO:340)

WPGPSGHGSTGVAAGGSTXSS,

(SEQ ID NO:341)

EWPWQHWCGCWREHXVKPQQVDLHSA,

(SEQ NO NO:342)

QQSALLQEERLDAVTLLYATSLPSFCLL,

(SEQ ID NO:343)

ACILLSCLLSVLYNLASFSLLALTSAL,
and/or

(SEQ ID NO:344)

SLNKWIFTVHGFGRPLLLSAL.

Polynucleotides encoding these polypeptides are also provided.

This gene is expressed primarily in brain tissue from a patient suffering from Alzheimer's disease (spongy change), and, to a lesser extent, in human umbilical vein and human pancreas tumor tissues.

Therefore, polynucleotides and polypeptides of the invention are useful as reagents for differential identification of the tissue(s) or cell type(s) present in a biological sample and for diagnosis of diseases and conditions which include, but are not limited to, developmental, immune, metabolic, digestive or neural diseases and/or disorders, such as Alzheimer's disease, in addition to cancers and tumors. Similarly, polypeptides and antibodies directed to these polypeptides are useful in providing immunological probes for differential identification of the tissue(s) or cell type(s). For a number of disorders of the above tissues or cells, particularly of the immune and secretory systems, expression of this gene at significantly higher or lower levels may be routinely detected in certain tissues and cell types (e.g., developmental, immune, metabolic, digestive, cancerous and wounded tissues) or bodily fluids (e.g., lymph, bile, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in brain tissue indicates that polynucleotides and polypeptides corresponding to this gene are useful for the diagnosis and/or treatment of Alzheimer's disease, and immune and secretory system disorders such as cancers. Representative uses are described in the “Regeneration” and “Hyperproliferative Disorders” sections below, in Example 11, 15, and 18, and elsewhere herein. Briefly, polynucleotides and polypeptides corresponding to this gene are useful for the detection/treatment of neurodegenerative disease states, behavioural disorders, or inflammatory conditions such as Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception.

In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:39 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1333 of SEQ ID NO:39, b is an integer of 15 to 1347, where both a and b correspond to the positions of nucleotide residues shown in SEQ ID NO:39, and where b is greater than or equal to a +14.

Features of Protein Encoded by Gene No: 30

Preferred polypeptides of the invention comprise the following amino acid sequence: EFGTSRARLQLKKNKKKERNIPGTLLSI (SEQ ID NO:345). Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, immune and hematopoietic diseases and/or disorders, particularly infection and inflammation. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 168 as residues: Asn-43 to Ala-49.

The tissue distribution in neutrophils indicates that polynucleotides and polypeptides corresponding to this gene are useful for the diagnosis and/or treatment of infection and inflammation related immune diseases. Furthermore, the gene product may also be involved in lymphopoiesis, therefore, it can be used in immune disorders such as infection, inflammation, allergy, immunodeficiency, etc. In addition, this gene product may have commercial utility in the expansion of stem cells and committed progenitors of various blood lineages, and in the differentiation and/or proliferation of various cell types. Additionally, expression of this gene product in neutrophils also strongly indicates a role for this protein in immune function and immune surveillance. Representative uses are described in the “Immune Activity” and “Infectious Disease” sections below, in Example 11, 13, 14, 16, 18, 19, 20, and 27, and elsewhere herein. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:40 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1453 of SEQ ID NO:40, b is an integer of 15 to 1467, where both a and b correspond to the positions of nucleotide residues shown in SEQ ID NO:40, and where b is greater than or equal to a +14.

Features of Protein Encoded by Gene No: 31

The translation product of this gene shares sequence homology with Ly6C antigen, in addition to the NG24 protein of Mus musculus, which are thought to be important in T- and B-cell activation.

Contact of cells with supernatant expressing the product of this gene has been shown to increase the permeability of the plasma membrane of THP-1 cells to calcium. Thus it is likely that the product of this gene is involved in a signal transduction pathway that is initiated when the product binds a receptor on the surface of the plasma membrane of monocytes. Thus, polynucleotides and polypeptides have uses which include, but are not limited to, activating monocytes, and to a lesser extent, in other cell-lines or tissue cell types. Binding of a ligand to a receptor is known to alter intracellular levels of small molecules, such as calcium, potassium and sodium, as well as alter pH and membrane potential. Alterations in small molecule concentration can be measured to identify supernatants which bind to receptors of a particular cell.

Preferred polypeptides of the invention comprise the following amino acid sequence:

(SEQ ID NO:346)

KSTLSAAVVATILRTLA,

(SEQ ID NO:347)

GDHSEQCLIKEMGARERRFCKARGYRDTGREAQAKAGGRRGSQWNEWQCS
SQRPRPAKEVRKTRPRAGVGRGPALLQLSLLQQVVLYVRPSLRLVWLKAS,

(SEQ ID NO:348)

MERGEYGGWGTYGSLDLGSQLCTVRSSGPCGSLHWGQHRSPISGPDPNPS
SSRGQQSIGSKVGSPSRSQWRSWKEVGRDPEKGE,

(SEQ ID NO:349)

QAKAGGRRGSQWNESQCSSQRPR,

(SEQ ID NO:350)

VGRGPALLQLSLLQQVVLYVRPSLRL,

(SEQ ID NO:351)

YGSLDLGSQLCTVRSSGPCGSL,
and/or

(SEQ ID NO:352)

KVGSPSRSQWRSWKEVGRDP.

Polynucleotides encoding these polypeptides are also provided.

The gene encoding the disclosed cDNA is believed to reside on chromosome 6. Accordingly, polynucleotides related to this invention are useful as a marker in linkage analysis for chromosome 6.

This gene is expressed primarily in bone cancer, fetal brain, lung, and adipose tissues.

Therefore, polynucleotides and polypeptides of the invention are useful as reagents for differential identification of the tissue(s) or cell type(s) present in a biological sample and for diagnosis of diseases and conditions which include, but are not limited to, skeletal, developmental, pulmonary, or metabolic diseases and/or disorders, particular disorders in the immune responses to the above conditions, such as in autoimmunities. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., skeletal, developmental, pulmonary, metabolic, and cancerous and wounded tissues) or bodily fluids (e.g., lymph, amniotic fluid, pulmonary surfactant or sputum, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 169 as residues: Gln-37 to Gln-45, Phe-76 to Leu-83, Thr-89 to Thr-105.

The tissue distribution, combined with the homology to the Ly6C T-cell activation antigen and detected calcium flux biological activity, indicates that polynucleotides and polypeptides corresponding to this gene are useful for the diagnosis and/or intervention of immune related disorders. The tissue distribution in tissues particularly active in immune reaction, for example bone cancer, indicates that this gene may also be involved in T-cell activation. Representative uses are described in the “Immune Activity” and “Infectious Disease” sections below, in Example 11, 13, 14, 16, 18, 19, 20, and 27, and elsewhere herein. Briefly, the gene product can be used either for the development of immune suppressants, or modulators, for immune responses. Moreover, the expression within brain tissue indicates that the protein is useful for the treatment and/or prevention of neurodegenerative disorders, particularly, but not limited to, Alzheimer's or Parkinson's disease.

Alternatively, the expression within fetal tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:41 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 900 of SEQ ID NO:41, b is an integer of 15 to 914, where both a and b correspond to the positions of nucleotide residues shown in SEQ ID NO:41, and where b is greater than or equal to a +14.

Features of Protein Encoded by Gene No: 32

Preferred polypeptides of the invention comprise the following amino acid sequence: ARGFFFYILITRLTPIKYDVNLILTAVTGSVGG (SEQ ID NO:353). Polynucleotides encoding these polypeptides are also provided.

The gene encoding the disclosed cDNA is thought to reside on chromosome 12. Accordingly, polynucleotides related to this invention are useful as a marker in linkage analysis for chromosome 12.

This gene is expressed primarily in brain, keratinocytes and fibroblasts.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the brain and epidermal system. Similarly, polypeptides and antibodies directed to these polypeptides are useful in providing immunological probes for differential identification of the tissue(s) or cell type(s). For a number of disorders of the above tissues or cells, particularly of the epidermal and neural systems, expression of this gene at significantly higher or lower levels may be routinely detected in certain tissues or cell types (e.g., skin, brain, cancerous and wounded tissues) or bodily fluids (e.g., lymph, serum, plasma, urine, synovial fluid and spinal fluid) or another tissue or cell sample taken from an individual having such a disorder, relative to the standard gene expression level (i.e., the expression level in healthy tissue or bodily fluid from an individual not having the disorder).

The tissue distribution in brain tissue indicates that polynucleotides and polypeptides corresponding to this gene are useful for the treatment and/or diagnosis of diseases of the neural and epidermal systems. Representative uses are described in the “Regeneration” and “Hyperproliferative Disorders” sections below, in Example 11, 15, and 18, and elsewhere herein. Briefly, the tissue distribution indicates that polynucleotides and polypeptides corresponding to this gene are useful for the detection/treatment of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. Additionally, the tissue distribution indicates that polynucleotides and polypeptides corresponding to this gene are useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma.

Moreover, such disorders may predispose (i.e., increase an individuals susceptibility) to viral and bacterial infections of the skin (i.e. cold sores, warts, chickenpox, molluscum contagiosum, herpes zoster, boils, cellulitis, erysipelas, impetigo, tinea, althletes foot, and ringworm). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:42 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1117 of SEQ ID NO:42, b is an integer of 15 to 1131, where both a and b correspond to the positions of nucleotide residues shown in SEQ ID NO:42, and where b is greater than or equal to a +14.

Features of Protein Encoded by Gene No: 33

The translation product of this gene shares sequence homology with a sodium dependent sulfate transporter which is thought to be important in sulfate uptake by cells (See Genbank Accession No. gi|310183, and Proc. Natl. Acad. Sci. U.S.A. 90, 8073-8077 (1993), which are hereby incorporated by reference herein).

Preferred polypeptides of the invention comprise the following amino acid sequence:

(SEQ ID NO:354)

MPQSLSSLASSSSSFQRXKPCFGKKNDGENQEHSLGTEPITWKDFQKTMP
WEIVILVGGGYALASGSKSSGLSTWIGNQMLSLSSLPPWAVTLLACIL
VSIVTEFVSNPATITIFLPILCSLSETLHINPLYTLIPVTMCISFAVMLP
VGNPPNAIVFSYGHCQIKDMVKAGLGVNVIGLVIVMVAINTWGVSLFHLD
TYPAWARVSNITDQA,

(SEQ ID NO:355)

NDGENQEHSLGTEPIITWKDFQK,

(SEQ ID NO:356)

IGNQMLSLSSLPPWAVTLLACILV,

(SEQ ID NO:357)

ATITIFLPILCSLSETLHINPLYTLIP,

(SEQ ID NO:358)

LPVGNPPNAIVFSYGHCQIKDMVKAG,

(SEQ ID NO:359)

LVIVMVAINTWGVSLFHLDTYPAWARVSN,

(SEQ ID NO:360)

LEHFNNQYPAAEVVNFGTWFLFSFPISLIMLVVSWFWMHWLFLGCNFKET
CSLSKKKKTKREQLSEKXXQ EEYEKLGDISYPE,
and/or

(SEQ ID NO:361)

QELWPLYMDWEPDVVPEQPPTVGCHPAGMHPRVHCH.

Polynucleotides encoding these polypeptides are also provided.

The gene encoding the disclosed cDNA is thought to reside on chromosome 7. Accordingly, polynucleotides related to this invention are useful as a marker in linkage analysis for chromosome 7.

This gene is expressed primarily in placenta, and, to a lesser extent, in infant brain and spinal cord.

Therefore, polynucleotides and polypeptides of the invention are useful as reagents for differential identification of the tissue(s) or cell type(s) present in a biological sample and for diagnosis of diseases and conditions which include, but are not limited to, metabolic, reproductive, vascular, or central nervous system disorders. Similarly, polypeptides and antibodies directed to these polypeptides are useful in providing immunological probes for differential identification of the tissue(s) or cell type(s). For a number of disorders of the above tissues or cells, particularly of the central nervous system, expression of this gene at significantly higher or lower levels may be routinely detected in certain tissues or cell types (e.g., CNS, reproductive, metabolic, vascular, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in placental and neural tissues, combined with the homology to a sodium dependent sulfate transporter, indicates that polynucleotides and polypeptides corresponding to this gene are useful for the treatment of metabolic disorders involving sodium and sulfate metabolism and CNS disorders involving neuronal signalling abnormalities. Representative uses are described in the “Regeneration” and “Hyperproliferative Disorders” sections below, in Example 11, 15, and 18, and elsewhere herein.

Briefly, polynucleotides and polypeptides corresponding to this gene are useful for the detection/treatment of neurodegenerative disease states, behavioural disorders, or inflammatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival.

Moreover, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues. Many polynucleotide sequences, such as EST sequences, are publicly available and accessible through sequence databases. Some of these sequences are related to SEQ ID NO:43 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1319 of SEQ ID NO:43, b is an integer of 15 to 1333, where both a and b correspond to the positions of nucleotide residues shown in SEQ ID NO:43, and where b is greater than or equal to a +14.

Features of Protein Encoded by Gene No: 34

Contact of cells with supernatant expressing the product of this gene increases the permeability of bovine chondrocyte cells to calcium. Thus, it is likely that the product of this gene is involved in a signal transduction pathway that is initiated when the product of this gene binds a receptor on the surface of the chondrocyte cell. Thus, polynucleotides and polypeptides have uses which include, but are not limited to, activating chondrocyte cells.

This gene is expressed primarily in CD34 positive cells.

Therefore, polynucleotides and polypeptides of the invention are useful as reagents for differential identification of the tissue(s) or cell type(s) present in a biological sample and for diagnosis of diseases and conditions which include, but are not limited to, immune, reproductive, or skeletal disorders, particularly diseases related to lymphocytes. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., bone, immune, hematopoietic, reproductive, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 172 as residues: Leu-26 to Arg-32, Asn-40 to Ser-46.

The tissue distribution in CD34 positive cells, combined with the detected calcium flux activity, indicates that polynucleotides and polypeptides corresponding to this gene are useful for the diagnosis or treatment of the diseases of the immune system particularly those related to T lymphocytes. Representative uses are described elsewhere herein. Polynucleotides and polypeptides corresponding to this gene are useful for the diagnosis and/or treatment of bone and hematopoietic disorders. The ability of the translation product of this gene to induce a calcium flux in chondrocytes indicates that it may play a role in the survival, proliferation, and/or growth of bone. Therefore, it may be useful in influencing bone mass in such conditions as osteoporosis. More generally, as evidenced by expression in CD34 positive cells, this gene may play a role in the survival, proliferation, and/or differentiation of hematopoietic cells, and may be of use in the augmentation of the numbers of stem cells and committed progenitors. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:44 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 990 of SEQ ID NO:44, b is an integer of 15 to 1004, where both a and b correspond to the positions of nucleotide residues shown in SEQ ID NO:44, and where b is greater than or equal to a +14.

Features of Protein Encoded by Gene No: 35

Contact of cells with supernatant expressing the product of this gene has been shown to increase the permeability of the plasma membrane of THP-1 cells to calcium. Thus it is likely that the product of this gene is involved in a signal transduction pathway that is initiated when the product binds a receptor on the surface of the plasma membrane of monocytes, and, to a lesser extent, in other cell-lines or tissue cell types. Thus, polynucleotides and polypeptides have uses which include, but are not limited to, activating monocytes.

Preferred polypeptides of the invention comprise the following amino acid sequence: STHASGGGRRGRGPRGEETQPRGWHARPGPGPRSTGA (SEQ ID NO:362). Polynucleotides encoding these polypeptides are also provided.

The gene encoding the disclosed cDNA is thought to reside on chromosome 9. Accordingly, polynucleotides related to this invention are useful as a marker in linkage analysis for chromosome 9.

This gene is expressed primarily in the brain, and, to a lesser extent, in liver.

Therefore, polynucleotides and polypeptides of the invention are useful as reagents for differential identification of the tissue(s) or cell type(s) present in a biological sample and for diagnosis of diseases and conditions which include, but are not limited to, disorders affecting the brain, central nervous system, or liver, including cancer. Similarly, polypeptides and antibodies directed to these polypeptides are useful in providing immunological probes for differential identification of the tissue(s) or cell type(s). For a number of disorders of the above tissues or cells, particularly of the immune, hematopoetic, or central nervous systems, expression of this gene at significantly higher or lower levels may be routinely detected in certain tissues or cell types (e.g., brain, liver, immune, hematopoietic, and cancerous and wounded tissues) or bodily fluids (e.g., lymph, serum, plasma, bile, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in brain and liver tissue, combined with the detected calcium flux activity indicates that polynucleotides and polypeptides corresponding to this gene are useful for the diagnosis and treatment of disorders affecting the immune, hematopoetic, or central nervous systems. Representative uses are described in the “Regeneration” and “Hyperproliferative Disorders” sections below, in Example 11, 15, and 18, and elsewhere herein. Briefly, the polynucleotides and polypeptides corresponding to this gene are useful for the detection/treatment of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, the gene or gene product may also play a role in the treatment and/or detection of developmental disorders associated with the developing embryo.

Alternatively, the expression within hepatic tissue indicates polynucleotides and polypeptides corresponding to this gene are useful for the detection and treatment of liver disorders and cancers (e.g., hepatoblastoma, jaundice, hepatitis, liver metabolic diseases and conditions that are attributable to the differentiation of hepatocyte progenitor cells). The protein is useful in modulating the immune response to aberrant proteins, for example, such proteins may be present in proliferative tissues. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:45 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1480 of SEQ ID NO:45, b is an integer of 15 to 1494, where both a and b correspond to the positions of nucleotide residues shown in SEQ ID NO:45, and where b is greater than or equal to a +14.

Features of Protein Encoded by Gene No: 36

When tested against U937 and Jurkat cell lines, supernatants removed from cells containing this gene activated the GAS (gamma activating sequence) promoter element. Thus, it is likely that this gene activates myeloid and T-cells, and to a lesser extent, other cells and tissue cell-types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

(SEQ ID NO:363)

ETCPSNGIELRQAPTSLYILLLHIQPTPTHPMLGRSYVLPAFSXNXEHGG
LPNQIPKGDRNGNIRHSRITFPCSSSTLQPESHLGFIRSKLHGLVRPGKD
LRGRRSLQLSKHSLSTCYMLRWETYKQVSYTAV,

(SEQ ID NO:364)

QRHQENDKRNVHRFLHTCVHMPMCTHTHTWAVLSTWEGQFSNVASFTSLK
RIPLSIIYIHSSHSPRRFVKVCQLRQEKALELTEVYVSASLKLQLYHLHC
HFHTAV,

(SEQ ID NO:365)

RQAPTSLYILLLHIQPTPTHPMLG,

(SEQ ID NO:366)

SHLGFIRSKLHGLVRPGKDLRGRRS,

(SEQ ID NO:367)

RNVHRFLHTCVHMPMCTHTHTQ,
and/or

(SEQ ID NO:368)

QLRQEKALELTEVYVSASLKLQLYH.

Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the immune system, particularly neutropenia, cancer, inflammatory diseases and allergies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoieic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils, combined with the detected GAS biological activity indicates that polynucleotides and polypeptides corresponding to this gene are useful for treatment/diagnosis of diseases of the immune system. Representative uses are described in the “Immune Activity” and “Infectious Disease” sections below, in Example 11, 13, 14, 16, 18, 19, 20, and 27, and elsewhere herein. Briefly, since expression is primarily in neutrophils, the protein may be useful as a growth factor for the differentiation or proliferation of neutrophils for the treatment of neutropenia following chemotherapy or may be useful in the treatment of immune dysfunction or anti-inflamatory by inhibiting infiltration of neutrophils to the site of injury or distress. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:46 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1152 of SEQ ID NO:46, b is an integer of 15 to 1166, where both a and b correspond to the positions of nucleotide residues shown in SEQ ID NO:46, and where b is greater than or equal to a +14.

Features of Protein Encoded by Gene No: 37

Preferred polypeptides of the invention comprise the following amino acid sequence:

(SEQ ID NO:369)

PRVRGRKEPGCLGPGRAGGDSQKEIGSWQQM,

(SEQ ID NO:370)

LSKGNRIMAADDDNGDGTSLFDVFSASPLKNNDEGSLDIYAGLDSAVSDS
ASKSCVPSRNCLDLYEEILTEEGTAKEATYNDLQVEYGKCQLQMKELMKK
FKEIQTQNFSLINENQSLKKNISALIKTARVEINRKDEEISNLHQKIVLS
FHIFEIIIKLQGHLIQLKQKILNLKLHIWMIVQRLITRAKSDVSKDVHHS
TSLPNLEKEGKPHSDKRSTSHLPTSVEKHCTNGVWSRSHYQVGEGSSNED
SRRGRKDIRHS QFNRGTERVRKDLSTGCGDGEPRILEASQRLQGTS,

(SEQ ID NO:371)

NRIMAADDDNGDGTSLFDVFSASPLKN,

(SEQ ID NO:372)

CLDLYEEILTEEGTAKEATYNDL,

(SEQ ID NO:373)

DEEISNLHQKIVLSFHIFEIIIKLQG,

(SEQ ID NO:374)

EKEGKPHSDKRSTSHLPTSVEK,
and/or

(SEQ ID NO:375)

TERVRKDLSTGCGDGEPRILEASQRL.

Polynucleotides encoding these polypeptides are also provided.

This gene is expressed primarily in activated T cells.

Therefore, polynucleotides and polypeptides of the invention are useful as reagents for differential identification of the tissue(s) or cell type(s) present in a biological sample and for diagnosis of diseases and conditions which include, but are not limited to, immune and inflammatory diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in T-cells indicates that polynucleotides and =polypeptides corresponding to this gene are useful for the diagnosis and treatment of immune and inflammatory disorders. Representative uses are described in the “Immune Activity” and “Infectious Disease” sections below, in Example 11, 13, 14, 16, 18, 19, 20, and 27, and elsewhere herein. Briefly, expression of this gene product in tonsils indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:47 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1522 of SEQ ID NO:47, b is an integer of 15 to 1536, where both a and b correspond to the positions of nucleotide residues shown in SEQ ID NO:47, and where b is greater than or equal to a +14.

Features of Protein Encoded by Gene No: 38

Contact of cells with supernatant expressing the product of this gene has been shown to increase the permeability of the plasma membrane of chondrocytes to calcium. Thus it is likely that the product of this gene is involved in a signal transduction pathway that is initiated when the product binds a receptor on the surface of the plasma membrane of both chondrocytes, in addition to other cell lines or tissue cell types. Thus, polynucleotides and polypeptides have uses which include, but are not limited to, activating chondrocytes, and to a lesser extent, other cells and tissue cell-types. Binding of a ligand to a receptor is known to alter intracellular levels of small molecules, such as calcium, potassium and sodium, as well as alter pH and membrane potential. Alterations in small molecule concentration can be measured to identify supernatants which bind to receptors of a particular cell.

Preferred polypeptides of the invention comprise the following amino acid sequence:

(SEQ ID NO:376)

KSYFRTMGGTKRGIKKLVNVCLKHPKNTSLSQQLVFAKINKILISKTTKS
TNLKGLKCLPPLSVSIHPTFIYYKHNTTLRIVFGTYFDFFPYRKNKDQAF
EGEDWESSLNVSDAW,

(SEQ ID NO:377)

TKRGIKKLVNVCLKHPKNTSLS,
and/or

(SEQ ID NO:378)

SIHPTFIYYKHNTTLRIVFGTYFDFF,

Polynucleotides encoding these polypeptides are also provided.

The gene encoding the disclosed cDNA is believed to reside on chromosome 3. Accordingly, polynucleotides related to this invention are useful as a marker in linkage analysis for chromosome 3.

This gene is expressed primarily in resting T-cells, and, to a lesser extent, in retina and placenta.

Therefore, polynucleotides and polypeptides of the invention are useful as reagents for differential identification of the tissue(s) or cell type(s) present in a biological sample and for diagnosis of diseases and conditions which include, but are not limited to, immune, reproductive, or eye disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, eye, reproductive, and cancerous and wounded tissues) or bodily fluids (e.g., lymph, serum, amniotic fluid, aqueous humor, vitreous humor, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 176 as residues: Met-1 to Pro-12.

The tissue distribution of this gene predominantly in T-cells and placenta, combined with the detected calcium flux activity, indicates that the gene could be important for the treatment or detection of immune or hematopoietic disorders including arthritis, asthma, immunodeficiency diseases and leukemia. Representative uses are described in the “Immune Activity”, “Hyperproliferative Disorders”, and “Infectious Disease” sections below, in Example 11, 13, 14, 16, 18, 19, 20, and 27, and elsewhere herein. Expression of the gene at high levels in the retina indicates a role in the treatment and/or detection of eye disorders including color blindness, blindness, vision defects, and light sensitivity. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:48 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1024 of SEQ ID NO:48, b is an integer of 15 to 1038, where both a and b correspond to the positions of nucleotide residues shown in SEQ ID NO:48, and where b is greater than or equal to a +14.

Features of Protein Encoded by Gene No: 39

This gene is expressed primarily in brain, retina, fetal heart, and pericardium tissues, and to a lesser extent in embryonic tissue.

Therefore, polynucleotides and polypeptides of the invention are useful as reagents for differential identification of the tissue(s) or cell type(s) present in a biological sample and for diagnosis of diseases and conditions which include, but are not limited to, developmental, cardiovascular, and neural diseases and/or disorders, particularly behavioral diseases of the brain such as depression, schizophrenia, Alzheimer's disease, Parkinson's disease, Huntington's disease, specific brain tumors, aphasia, mania, depression, dementia, paranoia, addictive behavior and sleep disorders. Similarly, polypeptides and antibodies directed to these polypeptides are useful in providing immunological probes for differential identification of the tissue(s) or cell type(s). For a number of disorders of the above tissues or cells, particularly of the brain, expression of this gene at significantly higher or lower levels may be routinely detected in certain tissues or cell types (e.g., developmental, cardiovacular, brain, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 177 as residues: Pro-35 to Met-42.

The tissue distribution in brain, heart, and fetal tissue indicates that polynucleotides and polypeptides corresponding to this gene are useful for the treatment and/or diagnosis of developmental, cardiovascular, and neural diseases. Representative uses are described in the “Regeneration”, “Infectious Diseases”, and “Hyperproliferative Disorders” sections below, in Example 11, 15, and 18, and elsewhere herein. Briefly, the uses include, but are not limited to the detection, treatment, and/or prevention of aphasia, depression, schizophrenia, Alzheimer's disease, Parkinson's disease, Huntington's disease, specific brain tumors, mania, depression, dementia, paranoia, addictive behavior and sleep disorders. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:49 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1162 of SEQ ID NO:49, b is an integer of 15 to 1176, where both a and b correspond to the positions of nucleotide residues shown in SEQ ID NO:49, and where b is greater than or equal to a +14.

Features of Protein Encoded by Gene No: 40

Preferred polypeptides of the invention comprise the following amino acid sequence:

(SEQ ID NO:379)

TRPRRHLGGQPGALHGQAACVHVPCLVPLCPPPANLTGSPHNSALQKQPL
GGRGKK,

(SEQ ID NO:380)

QPGALHGQAACVHVPCLVPLC,
and/or

(SEQ ID NO:381)

CPPPANLTGSPHNSALQKQPL.

Polynucleotides encoding these polypeptides are also provided.

The gene encoding the disclosed cDNA is thought to reside on chromosome 17. Accordingly, polynucleotides related to this invention are useful as a marker in linkage analysis for chromosome 17.

This gene is expressed primarily in infant brain tissue, and to a lesser extent in synovium.

Therefore, polynucleotides and polypeptides of the invention are useful as reagents for differential identification of the tissue(s) or cell type(s) present in a biological sample and for diagnosis of diseases and conditions which include, but are not limited to, neural, developmental, and musculo-skeletal system diseases and/or disorders. Similarly, polypeptides and antibodies directed to these polypeptides are useful in providing immunological probes for differential identification of the tissue(s) or cell type(s). For a number of disorders of the above tissues or cells, particularly of the musclulo-skeletal system, expression of this gene at significantly higher or lower levels may be routinely detected in certain tissues or cell types (e.g., neural, developmental, and synovium, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 178 as residues: Pro-15 to Cys-29, Gly-40 to Tyr-54, Pro-72 to His-79.

The tissue distribution in infant brain tissue indicates that polynucleotides and polypeptides corresponding to this gene are useful for the detection, treatment, and/or prevention of neurodegenerative disease states, behavioral disorders, or inflammatory conditions. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors.

Furthermore, the expression of this gene product in synovium would indicatesuggest a role in the detection and treatment of disorders and conditions afflicting the skeletal system, in particular osteoporosis, bone cancer, connective tissue disorders (e.g. arthritis, trauma, tendonitis, chrondomalacia and inflammation). The protein is also useful in the diagnosis or treatment of various autoimmune disorders (i.e., rheumatoid arthritis, lupus, scleroderma, and dermatomyositis), dwarfism, spinal deformation, joint abnormalities, and chondrodysplasias (i.e. spondyloepiphyseal dysplasia congenita, familial osteoarthritis, Atelosteogenesis type II, metaphyseal chondrodysplasia type Schmid, etc.). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:50 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 717 of SEQ ID NO:50, b is an integer of 15 to 731, where both a and b correspond to the positions of nucleotide residues shown in SEQ ID NO:50, and where b is greater than or equal to a +14.

Features of Protein Encoded by Gene No: 41

The translation product of this gene shares sequence homology with Enoyl-CoA hydratase, which is an RNA binding protein with intrinsic enzymatic activity thought to be important in metabolic disorders. Moreover, the protein product of this gene also has homology to carnitine racemase, which is thought to play an important role in fatty acid metabolism (See Geneseq Accession No. R80283; all references available through this accession number are hereby incorporated herein by reference, for example, Proc. Natl. Acad. Sci. U.S.A. 92 (6), 2051-2055 (1995)).

Preferred polypeptides of the invention comprise the following amino acid sequence: PDAGTASSQREPRRCRAGEAPSLPACAP (SEQ ID NO:382). Polynucleotides encoding these polypeptides are also provided.

The gene encoding the disclosed cDNA is thought to reside on chromosome 1. Accordingly, polynucleotides related to this invention are useful as a marker in linkage analysis for chromosome 1.

This gene is expressed primarily in fetal liver tissue.

Therefore, polynucleotides and polypeptides of the invention are useful as reagents for differential identification of the tissue(s) or cell type(s) present in a biological sample and for diagnosis of diseases and conditions which include, but are not limited to, metabolic disorders, liver disorders and cancer. Similarly, polypeptides and antibodies directed to these polypeptides are useful in providing immunological probes for differential identification of the tissue(s) or cell type(s). For a number of disorders of the above tissues or cells, particularly of the hepatic and metabolic systems, expression of this gene at significantly higher or lower levels may be routinely detected in certain tissues or cell types (e.g., liver, cancerous and wounded tissues) or bodily fluids (e.g., lymph, serum, plasma, urine, bile, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 179 as residues: Pro-10 to Arg-15, Leu-96 to Ser-103, Gly-172 to Pro-178, Gln-213 to Asp-218, Asn-268 to Leu-275, Arg-282 to Phe-289.

The tissue distribution in fetal liver, combined with the homology to Enoyl-CoA hydratase and carnitine racemase, indicates that polynucleotides and polypeptides corresponding to this gene are useful for the treatment and/or diagnosis of metabolic and liver diseases and cancer. Representative uses are described elsewhere herein. Furthermore, the tissue distribution indicates that polynucleotides and polypeptides corresponding to this gene are useful for the detection and/or treatment of liver disorders and cancers (e.g., hepatoblastoma, jaundice, hepatitis, liver metabolic diseases and conditions that are attributable to the differentiation of hepatocyte progenitor cells). The protein is useful in the detection, treatment, and/or prevention of neural diseases and/or disorders, particularly those conditions which may occur secondary to aberrations in fatty-acid metabolism. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:51 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1423 of SEQ ID NO:51, b is an integer of 15 to 1437, where both a and b correspond to the positions of nucleotide residues shown in SEQ ID NO:51, and where b is greater than or equal to a +14.

Features of Protein Encoded by Gene No: 42

Preferred polypeptides of the invention comprise the following amino acid sequence: FLIHLEVIWELGCFSPKAKAIASTPVIKGSLQIYFPCRSE (SEQ ID NO:383). Polynucleotides encoding these polypeptides are also provided.

This gene is expressed primarily in rhabdomyosarcoma tissue.

Therefore, polynucleotides and polypeptides of the invention are useful as reagents for differential identification of the tissue(s) or cell type(s) present in a biological sample and for diagnosis of diseases and conditions which include, but are not limited to, disorders of the musculo-skeletal system and cancer. Similarly, polypeptides and antibodies directed to these polypeptides are useful in providing immunological probes for differential identification of the tissue(s) or cell type(s). For a number of disorders of the above tissues or cells, particularly of the musculo-skeletal system, expression of this gene at significantly higher or lower levels may be routinely detected in certain tissues or cell types (e.g., musculo-skeletal, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in rhabdomyosarcoma tissue indicates that polynucleotides and polypeptides corresponding to this gene are useful for the treatment and/or diagnosis of disorders of the musculo-skeletal system and cancer. Representative uses are described elsewhere herein. Furthermore, the tissue distribution indicates a role in the detection and/or treatment of disorders and conditions affecting the musculo-skeletal system, in particular rhabdomyosarcomas as well as related cancers. The protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:52 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1355 of SEQ ID NO:52, b is an integer of 15 to 1369, where both a and b correspond to the positions of nucleotide residues shown in SEQ ID NO:52, and where b is greater than or equal to a +14.

Features of Protein Encoded by Gene No: 43

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils indicates that polynucleotides and polypeptides corresponding to this gene are useful for the diagnosis and treatment of aberrant immune responses to foreign antigens. Representative uses are described in the “Immune Activity” and “Infectious Disease” sections below, in Example 11, 13, 14, 16, 18, 19, 20, and 27, and elsewhere herein. Briefly, the expression of this gene product in neutrophils indicates a role in regulating the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:53 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1023 of SEQ ID NO:53, b is an integer of 15 to 1037, where both a and b correspond to the positions of nucleotide residues shown in SEQ ID NO:53, and where b is greater than or equal to a +14.

Features of Protein Encoded by Gene No: 44

Contact of cells with supernatant expressing the product of this gene has been shown to increase the permeability of the plasma membrane of AML-193 cells to calcium. Thus it is likely that the product of this gene is involved in a signal transduction pathway that is initiated when the product binds a receptor on the surface of the plasma membrane of myeloid leukemia cells, and to a lesser extent, other immune and hematopoietic cell-lines or tissue cell types. Thus, polynucleotides and polypeptides have uses which include, but are not limited to, activating myeloid cells.

Preferred polypeptides of the invention comprise the following amino acid sequence: HESKEKCPPGPLHQRCVFNSSGAGRVMATRKR (SEQ ID NO:384). Polynucleotides encoding these polypeptides are also provided.

This gene is expressed primarily in neutrophils induced with IL-1 and LPS.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders, particularly in aberrant neutrophil responses to infection. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 182 as residues: Lys-36 to Cys-42.

The tissue distribution in neutrophils, combined with the detected calcium flux activity, indicates that polynucleotides and polypeptides corresponding to this gene are useful for the diagnosis and treatment of a lack of immune response to infection. Representative uses are described in the “Immune Activity” and “Infectious Disease” sections below, in Example 11, 13, 14, 16, 18, 19, 20, and 27, and elsewhere herein. Briefly, the expression of this gene product in neutrophils indicates a role in regulatingproliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:54 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1359 of SEQ ID NO:54, b is an integer of 15 to 1373, where both a and b correspond to the positions of nucleotide residues shown in SEQ ID NO:54, and where b is greater than or equal to a +14.

Features of Protein Encoded by Gene No: 45

Preferred polypeptides of the invention comprise the following amino acid sequence: KRTLLQRLDWSYWVDSWEHQHSLHNGW (SEQ ID NO:385). Polynucleotides encoding these polypeptides are also provided.

This gene is expressed primarily in frontal cortex and bone marrow.

Therefore, polynucleotides and polypeptides of the invention are useful as reagents for differential identification of the tissue(s) or cell type(s) present in a biological sample and for diagnosis of diseases and conditions which include, but are not limited to, CNS and immune diseases and/or disorders. Similarly, polypeptides and antibodies directed to these polypeptides are useful in providing immunological probes for differential identification of the tissue(s) or cell type(s). For a number of disorders of the above tissues or cells, particularly of the central nervous system (CNS), expression of this gene at significantly higher or lower levels may be routinely detected in certain tissues or cell types (e.g., brain, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in frontal cortex tissue indicates that polynucleotides and polypeptides corresponding to this gene are useful for the treatment and/or diagnosis of disorders of the central nervous system. Representative uses are described in the “Regeneration” and “Hyperproliferative Disorders” sections below, in Example 11, 15, and 18, and elsewhere herein. Briefly, the elevated expression of this gene product within the frontal cortex of the brain indicates that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's. The protein is useful in modulating the immune response to aberrant polypeptides, as may be present in proliferative cells and tissues (i.e., brain cancer tissues, etc.). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:55 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1333 of SEQ ID NO:55, b is an integer of 15 to 1347, where both a and b correspond to the positions of nucleotide residues shown in SEQ ID NO:55, and where b is greater than or equal to a +14.

Features of Protein Encoded by Gene No: 46

Preferred polypeptides of the invention comprise the following amino acid sequence: GPRGVGDGGVSS (SEQ ID NO:386). Polynucleotides encoding these polypeptides are also provided.

This gene is expressed primarily in spleen.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders, particularly those affecting the spleen, such as in T- and B-cell maturation and their resulting efficacy in the immune response. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, spleen, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 184 as residues: Ser-20 to Ser-34, Thr-40 to Ser-46.

The tissue distribution in spleen indicates that polynucleotides and polypeptides corresponding to this gene are useful for the diagnosis and treatment of disorders affecting the spleen and immune system. Representative uses are described in the “Immune Activity” and “Infectious Disease” sections below, in Example 11, 13, 14, 16, 18, 19, 20, and 27, and elsewhere herein. Briefly, this gene may play a role in the survival, proliferation, and/or differentiation of hematopoietic cells in general, and may be of use in augmenting the number of stem cells and committed progenitors. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:56 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 808 of SEQ ID NO:56, b is an integer of 15 to 822, where both a and b correspond to the positions of nucleotide residues shown in SEQ ID NO:56, and where b is greater than or equal to a +14.

Features of Protein Encoded by Gene No: 47

Preferred polypeptides of the invention comprise the following amino acid sequence:

(SEQ ID NO:387)

QRPHPQPWXPMTLMGTGIPVFAHKMLPFDPPCHLSCTHINPKPXXPQGDE
QKSQGTEEWCDREGKKRRSI,

(SEQ ID NO:388)

PMTLMGTGIPVFAHKMLPFDP,

(SEQ ID NO:389)

PPCHLSCTHINPKPXXPQGDE,

(SEQ ID NO:390)

EQKSQGTEEWCDREGKKRRSI,

(SEQ ID NO:391)

DEWGAGRRMEWEDNLPLEFSCPVTKLLSVPSWTPLDAQMLLLFFPSLSHH
SSVPWLFCSSPCGXXGLGFI,

(SEQ ID NO:392)

EWEDNLPLEFSCPVTKLLSVP,

(SEQ ID NO:393)

PSWTPLDAWMLLLFFPSLSHH,
and/or

(SEQ ID NO:394)

HSSVPWLFCSSPCGXXGLGFI.

Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the immune system, including neutropenia, cancer, inflammatory diseases and allergies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils indicates that polynucleotides and polypeptides corresponding to this gene are useful for treatment/diagnosis of diseases of the immune system. Representative uses are described in the “Immune Activity” and “Infectious Disease” sections below, in Example 11, 13, 14, 16, 18, 19, 20, and 27, and elsewhere herein. Briefly, the expression primarily in neutrophils indicates that the protein may be useful as a growth factor for the differentiation or proliferation of neutrophils for the treatment of neutropenia following chemotherapy or may be useful in the treatment of immune dysfunction or anti-inflamatory by inhibiting infiltration of neutrophils to the site of injury or distress. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:57 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 522 of SEQ ID NO:57, b is an integer of 15 to 536, where both a and b correspond to the positions of nucleotide residues shown in SEQ ID NO:57, and where b is greater than or equal to a +14.

Features of Protein Encoded by Gene No: 48

This gene is expressed primarily in prostate, brain and T-cells.

Therefore, polynucleotides and polypeptides of the invention are useful as reagents for differential identification of the tissue(s) or cell type(s) present in a biological sample and for diagnosis of diseases and conditions which include, but are not limited to, disorders of the reproductive, central nervous system (CNS) and immune system. Similarly, polypeptides and antibodies directed to these polypeptides are useful in providing immunological probes for differential identification of the tissue(s) or cell type(s). For a number of disorders of the above tissues or cells, particularly of the reproductive, CNS and immune system, expression of this gene at significantly higher or lower levels may be routinely detected in certain tissues or cell types (e.g., immune, brain, prostate, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 186 as residues: Asp-26 to Gly-32, Ile-37 to Trp-44.

The tissue distribution indicates that polynucleotides and polypeptides corresponding to this gene are useful for the treatment and/or diagnosis of disorders of the reproductive, CNS and immune systems. Furthermore, the tissue distribution indicates that polynucleotides and polypeptides corresponding to this gene are useful for the detection and/or treatment of neurodegenerative disease states and behavioural disorders such as Alzheimers Disease, Parkinsons Disease, Huntingtons Disease, Tourette Syndrome, mania, dementia, paranoia, obsessive compulsive disorder, panic disorder, learning disabilities, ALS, psychoses, autism, and altered behaviors, including disorders in feeding, sleep patterns, balance, and perception. Additionally, the tissue distribution indicates that polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. Expression of this gene product in T cells strongly indicates a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:58 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1248 of SEQ ID NO:58, b is an integer of 15 to 1262, where both a and b correspond to the positions of nucleotide residues shown in SEQ ID NO:58, and where b is greater than or equal to a +14.

Features of Protein Encoded by Gene No: 49

This gene is expressed primarily in frontal cortex of schizophrenics.

Therefore, polynucleotides and polypeptides of the invention are useful as reagents for differential identification of the tissue(s) or cell type(s) present in a biological sample and for diagnosis of diseases and conditions which include, but are not limited to, central nervous system (CNS) diseases and Schizophrenia. Similarly, polypeptides and antibodies directed to these polypeptides are useful in providing immunological probes for differential identification of the tissue(s) or cell type(s). For a number of disorders of the above tissues or cells, particularly of the central nervous system, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution indicates that polynucleotides and polypeptides corresponding to this gene are useful for the treatment and/or diagnosis of disorders of the CNS and schizophrenia. Furthermore, the tissue distribution indicates that polynucleotides and polypeptides corresponding to this gene are useful for the diagnosis and/or treatment of disorders of the brain and nervous system. Elevated expression of this gene product within the frontal cortex of the brain indicates that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's.

Many polynucleotide sequences, such as EST sequences, are publicly available and accessible through sequence databases. Some of these sequences are related to SEQ ID NO:59 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1255 of SEQ ID NO:59, b is an integer of 15 to 1269, where both a and b correspond to the positions of nucleotide residues shown in SEQ ID NO:59, and where b is greater than or equal to a +14.

Features of Protein Encoded by Gene No: 50

Preferred polypeptides of the invention comprise the following amino acid sequence: ITEVRKDDLKVVRI (SEQ ID NO:395). Polynucleotides encoding these polypeptides are also provided.

This gene is expressed primarily in the testes.

Therefore, polynucleotides and polypeptides of the invention are useful as reagents for differential identification of the tissue(s) or cell type(s) present in a biological sample and for diagnosis of diseases and conditions which include, but are not limited to, reproductive or endocrine disorders, particularly male infertility and testicular cancer. Similarly, polypeptides and antibodies directed to these polypeptides are useful in providing immunological probes for differential identification of the tissue(s) or cell type(s). For a number of disorders of the above tissues or cells, particularly of the male reproductive system, expression of this gene at significantly higher or lower levels may be routinely detected in certain tissues or cell types (e.g., reproductive, testicular, and cancerous and wounded tissues) or bodily fluids (e.g., lymph, seminal fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 188 as residues: His-62 to Ser-74, Leu-99 to Gln-104.

The tissue distribution in testes indicates that polynucleotides and polypeptides corresponding to this gene are useful for treating male infertility. The protein product is likely involved in sperm development and could be administered by injection or related techniques. Representative uses are described elsewhere herein. Briefly, the uses include, but are not limited to the detection, treatment, and/or prevention of testicular cancer and aberrant testicular function. this gene could be transfected in gene-replacement treatments into the cells of the testes and the protein products could be produced. The presence of expression of this gene at either the RNA or protein level could be used as a diagnostic in testicular cancer. Furthermore, the tissue distribution indicates that polynucleotides and polypeptides corresponding to this gene are useful for the treatment and diagnosis of conditions concerning proper testicular function (e.g., endocrine function, sperm maturation), as well as cancer. Therefore, this gene product is useful in the treatment of male infertility and/or impotence. This gene product is also useful in assays designed to identify binding agents as such agents (antagonists) are useful as male contraceptive agents.

Similarly, the protein is believed to be useful in the treatment and/or diagnosis of testicular cancer. The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:60 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1815 of SEQ ID NO:60, b is an integer of 15 to 1829, where both a and b correspond to the positions of nucleotide residues shown in SEQ ID NO:60, and where b is greater than or equal to a +14.

Features of Protein Encoded by Gene No: 51

Preferred polypeptides of the invention comprise the following amino acid sequence: QGLSHIFW NEQTLK (SEQ ID NO:396). Polynucleotides encoding these polypeptides are also provided.

This gene is expressed primarily in activated T-cells.

Therefore, polynucleotides and polypeptides of the invention are useful as reagents for differential identification of the tissue(s) or cell type(s) present in a biological sample and for diagnosis of diseases and conditions which include, but are not limited to, immune disorders, particularly acute inflammatory conditions or autoimmune disease. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in activated T-cells indicates that polynucleotides and polypeptides corresponding to this gene are useful for modulating the response of activated T-cells to treat inflammation or autoimmune diseases. Representative uses are described in the “Immune Activity” and “Infectious Disease” sections below, in Example 11, 13, 14, 16, 18, 19, 20, and 27, and elsewhere herein. Briefly, the expression of this gene product indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:61 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1098 of SEQ ID NO:61, b is an integer of 15 to 1112, where both a and b correspond to the positions of nucleotide residues shown in SEQ ID NO:61, and where b is greater than or equal to a +14.

Features of Protein Encoded by Gene No: 52

When tested against U937 cell lines, supernatants removed from cells containing this gene activated the GAS (gamma activating sequence) promoter element. Thus, it is likely that this gene activates myeloid cells, and to a lesser extent, other cells and tissue cell-types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| TLVCLGVSSEEGSCPRDVTGPGCCFSLTLTGF, | (SEQ ID NO:397) |
| ADLIVLWHHHPLWPQHLALPSSGASHDHVELTVYPKTVAASWLLELSRPPIF | (SEQ ID NO:398) |
| CLFTXPALTXHGLDRVAALVECTIWXXXGMWYRRRYSCCQFRDRDIRDVFP | |
| EAVMLQQHLRHLAVATYRCRRRSPCKAPTVEEAEGGKPRAVPSGTGFQKHG | |
| QEPGGSTSPHWFWGHLQLLVLSVNNRQLFVQGRAGYLEMTGLPCPKLLLTL | |
| LRGLTPGVGHGLCAYRRGCLAWRLDXAS, | |
| ILWRQAPEAPHCSQDSVSSSPRLQEDLAHVTQVTRHPHFRSLPSAWCSHSSLL | (SEQ ID NO:399) |
| PVSLPRHALATKSPNMXXSSPILHLIQFTGQISSPLGGXVQPPGQTASPICTQP | |
| MSHPRRQASQQCEQQLWTGQTSHLQIPCPALNKELPVVDTQDKELQMSPEP | |
| MWGCGPSRLLPMLLESCA, | |
| MLQQHLRHLAVATYRCRRRSPCKAPTVEEAEGGK, | (SEQ ID NO:400) |
| VTQVTRHPHFRSLPSAWCSHSSLLPVSLP, and/or | (SEQ ID NO:401) |
| GQTASPICTQPMSHPRRQASQQCEQQLW. | (SEQ ID NO:402) |

Polynucleotides encoding these polypeptides are also provided.

This gene is expressed primarily in activated T-cells.

Therefore, polynucleotides and polypeptides of the invention are useful as reagents for differential identification of the tissue(s) or cell type(s) present in a biological sample and for diagnosis of diseases and conditions which include, but are not limited to, immune diseases and/or disorders, particularly autoimmune diseases and inflammation. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 190 as residues: Ser-25 to Lys-33.

The tissue distribution in neutrophils, combined with the detected GAS biological activity indicates that polynucleotides and polypeptides corresponding to this gene are useful for modulating the response of activated T-cells and other cells of the immune system involved in inflammation and autoimmune diseases. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:62 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1660 of SEQ ID NO:62, b is an integer of 15 to 1674, where both a and b correspond to the positions of nucleotide residues shown in SEQ ID NO:62, and where b is greater than or equal to a +14.

Features of Protein Encoded by Gene No: 53

Preferred polypeptides of the invention comprise the following amino acid sequence:

| | |
|---|---|
| FITLRLGPKNMAGVLWRHSNLQTPHYISWCPLLNYRETGNCLLHVSGFLNSR | (SEQ ID NO:403) |
| LLANCSGEASGKVIQTLLWPGEISAVA, | |
| KIRTFLFSGHRLFSTQGQSLTVKAHTAFMLIVKNLRYFIAFKFLMGISDSSEIGL | (SEQ ID NO:404) |
| VMQPLQKPHTVILIRGIEFLSPGGVLP, | |
| MAGVLWRHSNLQTPHYISWCPLLNYR, | (SEQ ID NO:405) |
| YFIAFKFLMGISDSSEIGLVMQPLQKPHT, and/or | (SEQ ID NO:406) |
| PFGLLVLP. | (SEQ ID NO:407) |

Polynucleotides encoding these polypeptides are also provided.

The gene encoding the disclosed cDNA is believed to reside on chromosome 12. Accordingly, polynucleotides related to this invention are useful as a marker in linkage analysis for chromosome 12.

This gene is expressed primarily in spleen, and, to a lesser extent, in bone marrow and B-cells.

Therefore, polynucleotides and polypeptides of the invention are useful as reagents for differential identification of the tissue(s) or cell type(s) present in a biological sample and for diagnosis of diseases and conditions which include, but are not limited to, immune and hematopoietic diseases and/or disorders, particularly mutiple myeloma, immunodeficiencies, and infections. Similarly, polypeptides and antibodies directed to these polypeptides are useful in providing immunological probes for differential identification of the tissue(s) or cell type(s). For a number of disorders of the above tissues or cells, particularly of the immune and hematopoietic disorders, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution of this gene predominantly in hematopoietic cell types and immune tissues indicates that the gene could be important for the treatment or detection of immune or hematopoietic disorders including arthritis, asthma, immunodeficiency diseases and leukemia. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:63 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1031 of SEQ ID NO:63, b is an integer of 15 to 1045, where both a and b correspond to the positions of nucleotide residues shown in SEQ ID NO:63, and where b is greater than or equal to a +14.

Features of Protein Encoded by Gene No: 54

The translation product of this gene shares very weak sequence homology with follicle-stimulating hormone beta subunit, which is thought to be important in hormonal regulation. When tested against K562 leukemia cell lines, supernatants removed from cells containing this gene activated the ISRE assay. Thus, it is likely that this gene activates leukemia cells through the Jak-STAT signal transduction pathway. The interferon-sensitive response element is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the ISRE element, can be used to indicate proteins involved in the proliferation and differentiation of cells.

The gene encoding the disclosed cDNA is thought to reside on chromosome 4. Accordingly, polynucleotides related to this invention are useful as a marker in linkage analysis for chromosome 4. The translation product of this gene is believed to be a Type 1b transmembrane protein. The transmembrane domain is shown as residues about 82 to about 98 and the intracellular domain is shown as residues about 99 to about 174, in the amino acid sequence in the sequence listing corresponding to this gene. The extracellular domain is believed to comprise residues from about 31 to about 81 of said sequence, however, the reading frame is open well upstream of the start predicted start methionine described in Table 1 indicating the possibility that this cDNA gene is not full-length. Accordingly, preferred polypeptides of the invention comprise the extracellular domain alone, the transmembrane domain alone, the intracellular domain alone, or any combinantion thereof linked by peptide bonds.

Preferred polypeptides of the invention comprises the following amino acid sequences:

| | |
|---|---|
| GFSRDTSVLSHFAFNSASPPKSYIRGKLGLEEYAVFYPPNGVIPFHGFSMYVAP | (SEQ ID NO:408) |
| LCFLYHEPSKLYQIFREMYVRFFFRLHSISSHPSGIVSLCLLFETLLQTYLPQLF | |
| YHLREIGAQPLRISFKWMVRAFSGYLATDQLLLLQDRILGYNS. | |

Polynucleotides encoding all of the aforementioned polypeptides are also provided.

This gene is expressed primarily in adult brain and adipocytes.

Therefore, polynucleotides and polypeptides of the invention are useful as reagents for differential identification of the tissue(s) or cell type(s) present in a biological sample and for diagnosis of diseases and conditions which include, but are not limited to, endocrine diseases. Similarly, polypeptides and antibodies directed to these polypeptides are useful in providing immunological probes for differential identification of the tissue(s) or cell type(s). For a number of disorders of the above tissues or cells, particularly of the endocrine system, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 192 as residues: Ser-139 to Ser-144, Phe-153 to Leu-159, Gln-162 to Ser-170.

The tissue distribution in brain tissue, and the homology to follicle stimulating hormone, indicates that polynucleotides and polypeptides corresponding to this gene are useful as a hormone for the diagnosis and/or treatment of endocrine disorders. The brain is a major site for secreting various hormones that regulate a wide range of body physiology. The secretory molecule encoded by this gene has very weak homology with FSH, and further indicates that it may serves as an endocrine. Endocrines can often be used in hormonal treatment of pathological disorders or change of physiology under certain circumstances such as in the treatment of reproductive disorders.

Many polynucleotide sequences, such as EST sequences, are publicly available and accessible through sequence databases. Some of these sequences are related to SEQ ID NO:64 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1037 of SEQ ID NO:64, b is an integer of 15 to 1051, where both a and b correspond to the positions of nucleotide residues shown in SEQ ID NO:64, and where b is greater than or equal to a +14.

Features of Protein Encoded by Gene No: 55

The translation product of this gene shares homology with a number of C. elegans proteases, which are thought to be important in programmed cell death.

This gene is expressed primarily in activated T-cells, and to a lesser extent in human stomach tissue.

Therefore, polynucleotides and polypeptides of the invention are useful as reagents for differential identification of the tissue(s) or cell type(s) present in a biological sample and for diagnosis of diseases and conditions which include, but are not limited to, immune disorders or stomach diseases. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 193 as residues: Lys-41 to Arg-47, Asp-125 to Lys-139, Ser-177 to Glu-185.

The tissue distribution in activated T-cells and stomach tissue indicates that polynucleotides and polypeptides corresponding to this gene are useful for the diagnosis and/or treatment of immune disorders, transplantation or stomach diseases. Particularly, the expression of the gene by activated T-cells can be used for the development of therapeutic agents as immune suppressants or immune modulators. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:65 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1168 of SEQ ID NO:65, b is an integer of 15 to 1182, where both a and b correspond to the positions of nucleotide residues shown in SEQ ID NO:65, and where b is greater than or equal to a +14.

Features of Protein Encoded by Gene No: 56

The translation product of this gene shares sequence homology with CD53 tetraspan transmembrane molecule, which is thought to be important in leukocyte activation.

The gene encoding the disclosed cDNA is thought to reside on chromosome 7. Accordingly, polynucleotides related to this invention are useful as a marker in linkage analysis for chromosome 7.

This gene is expressed primarily in KMH2 and activated T-cells, and to a lesser extent in tonsils.

Therefore, polynucleotides and polypeptides of the invention are useful as reagents for differential identification of the tissue(s) or cell type(s) present in a biological sample and for diagnosis of diseases and conditions which include, but are not limited to, infection, inflammation and other immune disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 194 as residues: Lys-99 to Arg-107.

The tissue distribution, and homology to CD53, indicates that polynucleotides and polypeptides corresponding to this gene are useful for the diagnosis and/or development of therapeutic agents for immune disorders including infection, allergy, inflammation, transplantation and immune deficiencies. Furthermore, expression of this gene product in tonsils indicates a role in regulating the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Expression of this gene product in T cells strongly indicates a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:66 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 661 of SEQ ID NO:66, b is an integer of 15 to 675, where both a and b correspond to the positions of nucleotide residues shown in SEQ ID NO:66, and where b is greater than or equal to a +14.

Features of Protein Encoded by Gene No: 57

The gene encoding the disclosed cDNA is thought to reside on chromosome 17. Accordingly, polynucleotides related to this invention are useful as a marker in linkage analysis for chromosome 17.

This gene is expressed primarily in fetal liver tissue, and to a lesser extent in neutrophils and keratinocytes.

Therefore, polynucleotides and polypeptides of the invention are useful as reagents for differential identification of the tissue(s) or cell type(s) present in a biological sample and for diagnosis of diseases and conditions which include, but are not limited to, inflammation, autoimmune and skin defects. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., liver, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 195 as residues: Pro-41 to Gln-50.

The tissue distribution indicates that polynucleotides and polypeptides corresponding to this gene are useful for the study, detection and/or treatment of inflammatory, general immune, and skin disorders. Furthermore, the tissue distribution indicates that polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. This is particularly supported by the expression of this gene product in fetal liver, which is a primary site of definitive hematopoiesis. Expression of this gene product in neutrophils also strongly indicates a role for this protein in immune function and immune surveillance.

Many polynucleotide sequences, such as EST sequences, are publicly available and accessible through sequence databases. Some of these sequences are related to SEQ ID NO:67 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1091 of SEQ ID NO:67, b is an integer of 15 to 1105, where both a and b correspond to the positions of nucleotide residues shown in SEQ ID NO:67, and where b is greater than or equal to a +14.

Features of Protein Encoded by Gene No: 58

This gene is expressed primarily in induced neutrophils.

Therefore, polynucleotides and polypeptides of the invention are useful as reagents for differential identification of the tissue(s) or cell type(s) present in a biological sample and for diagnosis of diseases and conditions which include, but are not limited to, immune and haemopoietic disorders. Similarly, polypeptides and antibodies directed to these polypeptides are useful in providing immunological probes for differential identification of the tissue(s) or cell type(s). For a number of disorders of the above tissues or cells, particularly of the haemopoietic and immune systems, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution indicates that polynucleotides and polypeptides corresponding to this gene are useful for the treatment and/or diagnosis of disorders of the haemopoietic and immune systems, such as those described elsewhere herein. This gene product may be involved in the regulation of cytokine production, antigen presentation, or other processes that may also suggest a usefulness in the treatment of cancer (e.g., by boosting immune responses). Since the gene is expressed in cells of lymphoid origin, the gene or protein, as well as, antibodies directed against the protein may show utility as a tumor marker and/or immunotherapy targets for the above listed tissues. Expression of this gene product in neutrophils also strongly indicates a role for this protein in immune function and immune surveillance.

Many polynucleotide sequences, such as EST sequences, are publicly available and accessible through sequence databases. Some of these sequences are related to SEQ ID NO:68 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1265 of SEQ ID NO:68, b is an integer of 15 to 1279, where both a and b correspond to the positions of nucleotide residues shown in SEQ ID NO:68, and where b is greater than or equal to a +14.

Features of Protein Encoded by Gene No: 59

Preferred polypeptides of the invention comprise the following amino acid sequence: LCQRGWAGQPGILTDGHPLPGQAASRSHQGPVGPGFSAN (SEQ ID NO:409), and/or QPGILTDGHPLPGQAASRSHQ (SEQ ID NO:410). Polynucleotides encoding these polypeptides are also provided.

This gene is expressed primarily in the endometrium, parathyroid tumor, and, to a lesser extent, in testis.

Therefore, polynucleotides and polypeptides of the invention are useful as reagents for differential identification of the tissue(s) or cell type(s) present in a biological sample and for diagnosis of female infertility or reproductive and endocrine diseases and/or disorders. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, endometrium, testicular, endocrine, and cancerous and wounded tissues) or bodily fluids (e.g., lymph, serum, amniotic fluid, plasma, seminal fluid, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in endometrium indicates that polynucleotides and polypeptides corresponding to this gene are useful for treating female infertility. Representative uses are described elsewhere herein. Briefly, the uses include, but are not limited to the preparation of the endometrium for implantation and could be administered either topically or orally. Alternatively, this gene could be transfected in gene-replacement treatments into the cells of the endometrium and the protein products could be produced. Similarly, these treatments could be performed during artificial insemination for the purpose of increasing the likelihood of implantation and development of a healthy embryo. In both cases this gene or its gene product could be administered at later stages of pregnancy to promote heathy development of the endometrium. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:69 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1624 of SEQ ID NO:69, b is an integer of 15 to 1638, where both a and b correspond to the positions of nucleotide residues shown in SEQ ID NO:69, and where b is greater than or equal to a +14.

Features of Protein Encoded by Gene No: 60

Preferred polypeptides of the invention comprise the following amino acid sequence: LLRPIL (SEQ ID NO:411). Polynucleotides encoding these polypeptides are also provided.

This gene is expressed primarily in the cells of the immune system, such as eosinophils, T-cells, dendritic cells, and tonsils.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseass and/or disorders, such as AIDS, inflammatory conditions, multiple myeloma, or SCID. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types or cell type (e.g., immune, hemaopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in various immune cells and tissues indicates that polynucleotides and polypeptides corresponding to this gene are useful for the diagnosis and treatment of immune system disorders, such as AIDS. Representative uses are described in the “Immune Activity” and “Infectious Disease” sections below, in Example 11, 13, 14, 16, 18, 19, 20, and 27, and elsewhere herein. Briefly, the expression of this gene product in tonsils and other immune cells indicates a role in regulatingproliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:70 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 873 of SEQ ID NO:70, b is an integer of 15 to 887, where both a and b correspond to the positions of nucleotide residues shown in SEQ ID NO:70, and where b is greater than or equal to a +14.

Features of Protein Encoded by Gene No: 61

The translation product of this gene shares homology with human stannin, which is thought to play a role in the toxic effects of organotins (See Genbank Accession No. gi|3378097, and Mamm. Genome 9 (7), 556-564 (1998), which are hereby incorporated by reference herein). Moreover, the protein product of this gene may also show utility in the treatment, and/or prevention of a variety of defects in the regulation and metabolism of calcium, and/or other ions.

Preferred polypeptides of the invention comprise the following amino acid sequence:

| | |
|---|---|
| ARADRARGAAAGRSGRAAAAPWTPVSSLSSSLTEWPPPLCCQPRKPPALTMSI, | (SEQ ID NO:412) |
| AAAGRSGRAAAAPWTPVSSLS, and/or | (SEQ ID NO:413) |
| SSSLTEWPPPKCCQPRKPPAL. | (SEQ ID NO:414) |

Polynucleotides encoding these polypeptides are also provided.

This gene is expressed primarily in GM-CSF treated macrophages.

Therefore, polynucleotides and polypeptides of the invention are useful as reagents for differential identification of the tissue(s) or cell type(s) present in a biological sample and for diagnosis of diseases and conditions which include, but are not limited to, immune or skeletal diseases and/or disorders, particularly in the treatment or amelioration of abberant immune response to tumor or foreign antigens, and in phagocytosis. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, skeletal, developmental, and cancerous and wounded tissues) or bodily fluids (e.g., lymph, serum, plasma, urine, amniotic fluid, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 199 as residues: Gly-43 to Gly-55.

The tissue distribution in macrophages indicates that polynucleotides and polypeptides corresponding to this gene are useful for the treatment and diagnosis of immune disorders. Representative uses are described in the “Immune Activity” and “Infectious Disease” sections below, in Example 11, 13, 14, 16, 18, 19, 20, and 27, and elsewhere herein. Briefly, the tissue distribution indicates that polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. Expression of this gene product in macrophage also strongly indicates a role for this protein in immune function and immune surveillance.

The protein product may even serve to stimulate the immune response, or may be used to inhibit such a response which may be useful during host versus graft disease or autoimmune disorders. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:71 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 850 of SEQ ID NO:71, b is an integer of 15 to 864, where both a and b correspond to the positions of nucleotide residues shown in SEQ ID NO:71, and where b is greater than or equal to a +14.

Features of Protein Encoded by Gene No: 62

This gene is expressed primarily in activated monocytes.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in monocytes indicates that polynucleotides and polypeptides corresponding to this gene are useful for diagnosing and/or treating immune or hematopoietic disorders. This gene product is primarily expressed in hematopoietic cells and tissues, suggesting that it plays a role in the survival, proliferation, and/or differentiation of hematopoieitic lineages. Representative uses are described in the “Immune Activity” and “Infectious Disease” sections below, in Example 11, 13, 14, 16, 18, 19, 20, and 27, and elsewhere herein. Briefly, the expression of this gene product in monocytes also strongly indicates a role for this protein in immune function and immune surveillance.

Moreover, polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:72 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1203 of SEQ ID NO:72, b is an integer of 15 to 1217, where both a and b correspond to the positions of nucleotide residues shown in SEQ ID NO:72, and where b is greater than or equal to a +14.

Features of Protein Encoded by Gene No: 63

This gene is expressed primarily in activated monocytes and helper T-cells.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 201 as residues: Met-i to Gly-6.

The tissue distribution in monocytes and helper T-cells indicates that polynucleotides and polypeptides corresponding to this gene are useful for diagnosing and/or treating immune or hematopoietic disorders. This gene product is primarily expressed in hematopoietic cells and tissues, suggesting that it plays a role in the survival, proliferation, and/or differentiation of hematopoieitic lineages. Representative uses are described in the “Immune Activity” and “Infectious Disease” sections below, in Example 11, 13, 14, 16, 18, 19, 20, and 27, and elsewhere herein. Briefly, the expression of this gene product in monocytes also strongly indicates a role for this protein in immune function and immune surveillance. Moreover, polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia.

The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:73 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1703 of SEQ ID NO:73, b is an integer of 15 to 1717, where both a and b correspond to the positions of nucleotide residues shown in SEQ ID NO:73, and where b is greater than or equal to a +14.

Features of Protein Encoded by Gene No: 64

The translation product of this gene was shown to have homology to the conserved S.pombe−rad4+/cut5+product which is thought to function as a type II, DNA topoisomerase (See Genbank Accession No.gnI|PID|d1014079). The uses for such activity is well-known in the art and described elsewhere herein.

When tested against K562 leukemia cell lines, supernatants removed from cells containing this gene activated the ISRE assay. Thus, it is likely that this gene activates leukemia cells through the Jak-STAT signal transduction pathway. The interferon-sensitive response element is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the ISRE element, can be used to indicate proteins involved in the proliferation and differentiation of cells.

Preferred polypeptides of the invention comprise the following amino acid sequence:

| | |
|---|---|
| EYFLEFVFSLIWILSHCSILLSSAVCDPGNIRVTEAPKHPISEELETPIKDSHLIPT | (SEQ ID NO:415) |
| PQAPSIAFP | |
| LANPPVAPHPREKIITIEETHEELKKQYIFQLSSLNPQERIDYCHLIEKLGTSILL | |
| KSKMSHIITIFGSQ M, | |
| LIWILSHCSILLSSAVCDPGN, | (SEQ ID NO:416) |
| NIRVTEAPKHPISEELETPIK, | (SEQ ID NO:417) |
| KDSHLIPTPQAPSIAFP LAN, | (SEQ ID NO:418) |
| NPPVAPHPREKIITIEETHEE and/or | (SEQ ID NO:419) |
| ELKKQYIFQLSSLNPQERIDY. | (SEQ ID NO:420) |

Polynucleotides encoding these polypeptides are also provided.

The gene encoding the disclosed cDNA is thought to reside on chromosome 3. Accordingly, polynucleotides related to this invention are useful as a marker in linkage analysis for chromosome 3.

This gene is expressed primarily in spleen from a chronic lymphocytic leukemia patient, dendritic cells, and, to a lesser extent, in bone marrow cells.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly leukemias. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., spleen, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in immune cells, combined with the detected ISRE biological activity in K562 cell lines and homology to a putative topoisomerase homolog, indicates that polynucleotides and polypeptides corresponding to this gene are useful for the diagnosis and/or treatment of chronic lymphocytic leukemia. Representative uses are described in the “Immune Activity” and “Infectious Disease” sections below, in Example 11, 13, 14, 16, 18, 19, 20, and 27, and elsewhere herein. Briefly, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:74 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1262 of SEQ ID NO:74, b is an integer of 15 to 1276, where both a and b correspond to the positions of nucleotide residues shown in SEQ ID NO:74, and where b is greater than or equal to a +14.

Features of Protein Encoded by Gene No: 65

Preferred polypeptides of the invention comprise the following amino acid sequence: INICIY (SEQ ID NO:421). Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils indicates that polynucleotides and polypeptides corresponding to this gene are useful for the diagnosis and/or treatment of neutrophils inactivation and other immune system disorders. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. Expression of this gene product in neutrophils also strongly indicates a role for this protein in immune function and immune surveillance. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:75 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1130 of SEQ ID NO:75, b is an integer of 15 to 1144, where both a and b correspond to the positions of nucleotide residues shown in SEQ ID NO:75, and where b is greater than or equal to a +14.

Features of Protein Encoded by Gene No: 66

Preferred polypeptides of the invention comprise the following amino acid sequence:

| | |
|---|---|
| LQESAXQFSSS, | (SEQ ID NO:422) |
| NLHGCHGKFQEHNLKVNCMTLFCVSLTTTHSVSLKVTVYITVSILCMPDTQD | (SEQ ID NO:423) |
| SNFSFPLDTTYLVINFGSTYSTK, and/or | |
| LFCVSLTTTHSVSLKVTVYITVSILCMPDT. | (SEQ ID NO:424) |

Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly neutropenia. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils indicates that polynucleotides and polypeptides corresponding to this gene are useful for the diagnosis and/or treatment of immune system disorders. Representative uses are described in the “Immune Activity” and “Infectious Disease” sections below, in Example 11, 13, 14, 16, 18, 19, 20, and 27, and elsewhere herein. Briefly, the expression of this gene product in neutrophils also strongly indicates a role for this protein in immune function and immune surveillance. The protein may also be useful in the inhibition of neutrophil activation which may show utility in host-versus-graft disease and autoimmune disorders. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:76 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 904 of SEQ ID NO:76, b is an integer of 15 to 918, where both a and b correspond to the positions of nucleotide residues shown in SEQ ID NO:76, and where b is greater than or equal to a +14.

Features of Protein Encoded by Gene No: 67

When tested against U937 myeloid cell lines, supernatants removed from cells containing this gene activated the GAS promoter element. Thus, it is likely that this gene activates myeloid cells, myeloid progenitors, and to a lesser extent, in other cells and tissue cell-types, through the Jak-STAT signal transduction pathway. The gamma activating sequence (GAS) is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence: LLNPKASLHSA (SEQ ID NO:425). Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, such as neutropenia. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 205 as residues: Asp-23 to Trp-29.

The tissue distribution in neutrophils, combined with the detected GAS biological activity in myeloid cell lines indicates that polynucleotides and polypeptides corresponding to this gene are useful for the diagnosis and treatment of immune system disorders. Representative uses are described in the “Immune Activity” and “Infectious Disease” sections below, in Example 11, 13, 14, 16, 18, 19, 20, and 27, and elsewhere herein. Briefly, the tissue distribution indicates that polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages.

Expression of this gene product in neutrophils also strongly indicates a role for this protein in immune function and immune surveillance. The protein product of this gene may show utility in the inhibition of neutrophil activation which may show utility in host-versus-graft disease and in autoimmune disorders. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:77 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1051 of SEQ ID NO:77, b is an integer of 15 to 1065, where both a and b correspond to the positions of nucleotide residues shown in SEQ ID NO:77, and where b is greater than or equal to a +14.

Features of Protein Encoded by Gene No: 68

This gene is expressed primarily in neutrophils induced with IL-1 and LPS.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, such as neutropenia. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils indicates that polynucleotides and polypeptides corresponding to this gene are useful for the diagnosis and treatment of aberrant immune response to foreign antigens. Representative uses are described in the “Immune Activity” and “Infectious Disease” sections below, in Example 11, 13, 14, 16, 18, 19, 20, and 27, and elsewhere herein. Briefly, the tissue distribution indicates that polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. Expression of this gene product in neutrophils also strongly indicates a role for this protein in immune function and immune surveillance. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. The protein product of this gene may also show utility in the inactivation of neutrophils which may show utility in host-versus-graft disease or in autoimmune disorders, for example. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:78 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1112 of SEQ ID NO:78, b is an integer of 15 to 1126, where both a and b correspond to the positions of nucleotide residues shown in SEQ ID NO:78, and where b is greater than or equal to a +14.

Features of Protein Encoded by Gene No: 69

The translation product of this nucleotide sequence shares homology with a number of cysteine proteinases (See Genbank Accession No. gi|391621, and Geneseq Accession No. W53200. All references available through these accessions are hereby incorporated by reference herein (For example, J. Biol. Chem2. 273 (48), 32000-32008 (1998)).

Contact of cells with supernatant expressing the product of this gene increases the permeability of TF-1 Myeloid cells to calcium. Thus, it is likely that the product of this gene is involved in a signal transduction pathway that is initiated when the product of this gene binds a receptor on the surface of the myeloid cell. Thus, polynucleotides and polypeptides have uses which include, but are not limited to, activating myeloid cells, and to a lesser extent, in other cells and tissue cell-types.

Preferred polypeptides of the invention comprise the following amino acid sequence:

| | |
|---|---|
| DPRVRASVGRCVRAAGFXLA, | (SEQ ID NO:426) |
| PYRGGXPYHLPESPPKRVPWQEHAPRQVCWRLCPIRXGLEEKGGRHQSQEPG | (SEQ ID NO:427) |
| MXGSCWAFSXTGNVEGQWFLKQGPXLPLRXXXLGL, | |
| RPTRPRVRRSVRPGRRLRPRHGTLAAAAVXAGAAPGXRSRPAPPSSRRSGPG | (SEQ ID NO:428) |
| GGVPGAAGARPLRAGDVQ | |
| RPRGSRXAGDAGGRARSRPPGGRGVAVLPEGDPGGASLQRXHGVPAPCVXE | |
| TLLCSFEVLDELGKHMLLR | |
| RDCGPVDTKVTDDKNETLSSVLPLLNKEPLPQDFSVKMASIFKEFVTTYNRTY | |
| ESKEETQWRMSVFSNNM | |
| MRAQKIQALDRGTAQYGVTKFSDLTEEEFHTIYLNPLLREYHGKNMRLDKSA | |
| GDSAPSEWDWXXKGXVTK VKNQACXAPAGLSQSLVTWRASGS, | |
| TLAAAAVXAGAAPGXRSRPAPPSSRRSGPGGGVPGAAGARPLRAGDVQPRP | (SEQ ID NO:429) |
| GSRXAGDAGGRARSRPPGGRGVAVLPEGDPGGAS, and/or | |
| SFEVLDELGKHMLLRRDCGPVDTKVTDDKNETLSSVLPLLNKEPLPQDFSVK | (SEQ ID NO:430) |
| MASIFKEFVTTYNRTYESKEETQWRMSVFSNNMMRAQKIQALDRGTAQYGV | |
| TKFSDLTEEEFHTIYL. | |

Polynucleotides encoding these polypeptides are also provided.

This gene is expressed primarily in tissue from an ovarian tumor.

Therefore, polynucleotides and polypeptides of the invention are useful as reagents for differential identification of the tissue(s) or cell type(s) present in a biological sample and for diagnosis of diseases and conditions which include, but are not limited to, reproductive disorders, particularly ovarian cancer. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, immune, hematopoietic, ovarian, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The homology to proteins of the cysteine proteinase family, tissue distribution in ovarian tissues, combined with the detected calcium flux activity in myeloid cells indicates that the protein product of this gene may show utility in the treatment, and/or prevention of a variety of reproductive disorders, such as in ovarian cancer, or even in the modulation of the immune response. Thus, it is useful for the diagnosis and/or treatment of ovarian cancer. Representative uses are described in the “Immune Activity” and “Infectious Disease” sections below, in Example 11, 13, 14, 16, 18, 19, 20, and 27, and elsewhere herein. Briefly, the biological activity data, when compared to the tissue distribution, suggest that the translation product of this gene could be useful in activating the immune system to respond to cancerous growths, particularly those involving ovarian cancer. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:79 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 970 of SEQ ID NO:79, b is an integer of 15 to 984, where both a and b correspond to the positions of nucleotide residues shown in SEQ ID NO:79, and where b is greater than or equal to a +14.

Features of Protein Encoded by Gene No: 70

Preferred polypeptides of the invention comprise the following amino acid sequence: TSHPLGGGVER (SEQ ID NO:431). Polynucleotides encoding these polypeptides are also provided.

This gene is expressed primarily in anergic T-cells.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders, such as autoimmune disorders including lupus. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 208 as residues: Ser-26 to Lys-34.

The tissue distribution in T-cells indicates that polynucleotides and polypeptides corresponding to this gene are useful for the diagnosis and treatment of a variety of immune system disorders. Representative uses are described in the “Immune Activity” and “Infectious Disease” sections below, in Example 11, 13, 14, 16, 18, 19, 20, and 27, and elsewhere herein. Briefly, the expression of this gene product in T-cells indicates a role in regulating the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses).

Expression of this gene product in T cells also strongly indicates a role for this protein in immune function and immune surveillance. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:80 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1233 of SEQ ID NO:80, b is an integer of 15 to 1247, where both a and b correspond to the positions of nucleotide residues shown in SEQ ID NO:80, and where b is greater than or equal to a +14.

Features of Protein Encoded by Gene No: 71

This gene shares homology with the human adult heart neutral calponin, which is implicated in the regulation and modulation of smooth muscle contraction. It is capable of binding to actin, calmodulin, troponin C, and tropomyosin. The interaction of calponin with actin inhibits the actomyosin Mg-ATPase activity. Therefore, the protein product of this gene may be beneficial as a vasoconstrictor or vasodilator, a muscle relaxor, treatment for tetanus stimuli, or for the treatment of various cardiovascular disorders. Contact of cells with supernatant expressing the product of this gene has been shown to increase the permeability of the plasma membrane of AML-193 cells to calcium. Thus it is likely that the product of this gene is involved in a signal transduction pathway that is initiated when the product binds a receptor on the surface of the plasma membrane of myeloid leukemia cells, in addition to other cell-lines or tissue cell types. Thus, polynucleotides and polypeptides have uses which include, but are not limited to myeloid cells.

Preferred polypeptides of the invention comprise the following amino acid sequence: ACCCLEWAG (SEQ ID NO:432). Polynucleotides encoding these polypeptides are also provided. The gene encoding the disclosed cDNA is thought to reside on chromosome 19. Accordingly, polynucleotides related to this invention are useful as a marker in linkage analysis for chromosome 19.

This gene is expressed primarily in adrenal gland tumor and human 12 week embryo. Furthermore, the gene is expressed in cardiomyopathy tissue.

Therefore, polynucleotides and polypeptides of the invention are useful as reagents for differential identification of the tissue(s) or cell type(s) present in a biological sample and for diagnosis of diseases and disorders: endocrine, developmental, cardiovascular disorders, particularly diseases involving abnormal cellular proliferation such as cancers particularly of the adrenal gland, and disorders involving heart muscle, such as cardiomyopathy Similarly, polypeptides and antibodies directed to these polypeptides are useful in providing immunological probes for differential identification of the tissue(s) or cell type(s). For a number of disorders of the above tissues or cells, particularly of the adrenal gland, heart, expression of this gene at significantly higher or lower levels may be routinely detected in certain tissues or cell types (e.g., heart, muscle, endocrine, developmental, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 209 as residues: Ser-61 to Trp-67.

The tissue distribution in adrenal tumor tissue indicates that polynucleotides and polypeptides corresponding to this gene are useful for the diagnosis and treatment of abnormal cellular proliferation, such as tumors. Given the tissue distribution and the homology to human adult heart neutral calponin, the translation product of this gene is useful for detecting, identifying, and/or treating disorders involving the degeneration of the regulation and modulation of smooth muscle contraction, such as is seen with cardiomyopathies. Moreover, the expression within embryonic tissue and other cellular sources marked by proliferating cells indicates this protein may play a role in the regulation of cellular division, and may show utility in the diagnosis, treatment, and/or prevention of developmental diseases and disorders, cancer, and other proliferative conditions. Representative uses are described in the “Hyperproliferative Disorders” and “Regeneration” sections below and elsewhere herein. Briefly, developmental tissues rely on decisions involving cell differentiation and/or apoptosis in pattern formation.

Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:81 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 944 of SEQ ID NO:81, b is an integer of 15 to 958, where both a and b correspond to the positions of nucleotide residues shown in SEQ ID NO:81, and where b is greater than or equal to a +14.

Features of Protein Encoded by Gene No: 72

Preferred polypeptides of the invention comprise the following amino acid sequence: SAEQKTRLHLLYKTELYFSFIISRVAVLLVLIHWRGGIRTDVS (SEQ ID NO:433). Polynucleotides encoding these polypeptides are also provided.

This gene is expressed primarily in human bone marrow and 9 week embryo.

Therefore, polynucleotides and polypeptides of the invention are useful as reagents for differential identification of the tissue(s) or cell type(s) present in a biological sample and for diagnosis of diseases and conditions which include, but are not limited to, skeletal, immune, hemopoietic, or developmental disordes. Similarly, polypeptides and antibodies directed to these polypeptides are useful in providing immunological probes for differential identification of the tissue(s) or cell type(s). For a number of disorders of the above tissues or cells, particularly of the hematoplastic tissues, expression of this gene at significantly higher or lower levels may be routinely detected in certain tissues or cell types (e.g., immune, bone, cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 210 as residues: Ala-22 to Lys-36.

The tissue distribution in bone marrow and embryonic tissues indicates that polynucleotides and polypeptides corresponding to this gene are useful for the diagnosis and treatment of hemopoietic or immune diseases and/or disorders. Furthermore, it may be useful in influencing bone mass in such conditions as osteoporosis. Polynucleotides and polypeptides corresponding to this gene are useful for the treatment and diagnosis of hematopoietic related disorders such as anemia, pancytopenia, leukopenia, thrombocytopenia or leukemia since stromal cells are important in the production of cells of hematopoietic lineages. Representative uses are described in the “Immune Activity” and “Infectious Disease” sections below, in Example 11, 13, 14, 16, 18, 19, 20, and 27, and elsewhere herein. Briefly, the uses include bone marrow cell ex-vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc.

In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:82 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1378 of SEQ ID NO:82, b is an integer of 15 to 1392, where both a and b correspond to the positions of nucleotide residues shown in SEQ ID NO:82, and where b is greater than or equal to a +14.

Features of Protein Encoded by Gene No: 73

Preferred polypeptides of the invention comprise the following amino acid sequence: TLQNIYPLLIDASLYICVYIHTY (SEQ ID NO:434). Polynucleotides encoding these polypeptides are also provided.

This gene is expressed primarily in helper T cells.

Therefore, polynucleotides and polypeptides of the invention are useful as reagents for differential identification of the tissue(s) or cell type(s) present in a biological sample and for diagnosis of diseases and conditions which include, but are not limited to, disorders and/or diseases of the immune or hematopoietic systems, particularly immunodeficiencies or inflammatory conditions, such as AIDS, SCID, leukemias, or multiple myeloma. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 211 as residues: Asp-26 to Leu-36, Leu-42 to Phe-50.

The tissue distribution in T-cells indicates that polynucleotides and polypeptides corresponding to this gene are useful for the treatment of disorders of the immune system such as AIDS. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses). Since the gene is expressed in cells of lymphoid origin, the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Expression of this gene product in T cells also strongly indicates a role for this protein in immune function and immune surveillance. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:83 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1141 of SEQ ID NO:83, b is an integer of 15 to 1155, where both a and b correspond to the positions of nucleotide residues shown in SEQ ID NO:83, and where b is greater than or equal to a +14.

Features of Protein Encoded by Gene No: 74

When tested against U937 cell lines, supernatants removed from cells containing this gene activated the GAS (gamma activating sequence) promoter element. Thus, it is likely that this gene activates myeloid cells, including progenitors, and to a lesser extent, other tissues and cell-types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| DVLLPLLYLLVRKHINRAGIGNTFQGGANCI, | (SEQ ID NO:435) |
| MCCCLCCTSWSGSTSTERVSGTRFREVPTASCSSSAPAPSELGSSLSVAAAAL | (SEQ ID NO:436) |
| LSLPPRARLALPRLPRL PSQENLRNPKGPQGNFQAPGAFVLSSSVA, | |
| CAAASAVPPGPEAHQQSGYREHVSGRCQLHHVRPLHPRRPNSALLSLLLLLL | (SEQ ID NO:437) |
| FSASHQEPGWHSQGSRAF | |
| QARRISGIPRDPRGTSKHLELLSFLVLWHRCCLPGGRXFCESLXQGRSACLLH | |
| QKPPLLMLSAPLGEQLP | |
| TQLLLPPRSSGSKFXRYQRPGPRVGVHLHKGSSEIREAGGPQLWPQCPHPVDL | |
| DVLRTTQHCLQSEGPTS VHLSSV, | |
| EVEEAELAAALPMEPRASIAGASGAADMHFCPAXGTHRXA | (SEQ ID NO:438) |
| YPQEGSTYATELERTKAPGAWKFPWGPLGFLRFSWLGRRGSLGSASRALGG | |
| RLRRAAAATEREEPSSDGA | |
| GAEDEHDAVGTSLKRVPDTRSVDVLPDQEVQQRQQHI, | |
| RRISGIPRDPRGRSKHLELLSFLVLWHRCCL, | (SEQ ID NO:439) |
| RTKAPGAWKFPWGPLGFLRFSWFGRRGSL, and/or | (SEQ ID NO:440) |
| DVLLPLLYLLVRKHINRAGIGNTFQGGANCI. | (SEQ ID NO:441) |

Polynucleotides encoding these polypeptides are also provided.

This gene is expressed primarily in smooth muscle, and, to a lesser extent, in melanocytes.

Therefore, polynucleotides and polypeptides of the invention are useful as reagents for differential identification of the tissue(s) or cell type(s) present in a biological sample and for diagnosis of diseases and conditions which include, but are not limited to, disorders of smooth muscle tissue, particularly vascular disorders, such as vasculositis, microvascular disease, atherosclerosis, stroke, aneurysm, and embolism. Similarly, polypeptides and antibodies directed to these polypeptides are useful in providing immunological probes for differential identification of the tissue(s) or cell type(s). For a number of disorders of the above tissues or cells, particularly of smooth muscle tissue, expression of this gene at significantly higher or lower levels may be routinely detected in certain tissues and cell types (e.g., smooth muscle, vascular, integumentary, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 212 as residues: Ser-23 to Glu-54.

The tissue distribution in smooth muscle, combined with the detected GAS biological activity indicates that polynucleotides and polypeptides corresponding to this gene are useful for the diagnosis and/or treatment of vascular or cardiopulmonary disorders. In addition, the protein may show utility in the modulation of the immune system in response to various vascular disorders, particularly in the early stages of atherosclerosis, embolism, thrombosis, and stroke. Representative uses are described in the “Biological Activity”, “Hyperproliferative Disorders”, and “Binding Activity” sections below, in Example 11, 17, 18, 19, 20 and 27, and elsewhere herein. Briefly, the protein may be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO: 84 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1359 of SEQ ID NO:84, b is an integer of 15 to 1373, where both a and b correspond to the positions of nucleotide residues shown in SEQ ID NO:84, and where b is greater than or equal to a +14.

Features of Protein Encoded by Gene No: 75

When tested against NIH3T3 cell lines, supernatants removed from cells containing this gene activated the EGR1 (early growth response gene 1) promoter element. Thus, it is likely that this gene activates fibroblast cells, and to a lesser extent, other cells and tissue cell-types, through the EGR1 signal transduction pathway. EGR1 is a separate signal transduction pathway from Jak-STAT, genes containing the EGR1 promoter are induced in various tissues and cell types upon activation, leading the cells to undergo differentiation and proliferation.

Preferred polypeptides of the invention comprise the following amino acid sequence:

| | |
|---|---|
| PRLAQLRLLSL, | (SEQ ID NO:442) |
| QSDFREMNQTNSTSNAAKAREAQQGRGRD | (SEQ ID NO:443) |
| REAIFSSSALEHLVCYLQAYKHTLLFIRSLNEHGLQQLLFQWRDGLFGNWYF | |
| RIPILLFFTGFHCYHLSC | |
| PHLPCAQRQSSRGTVPYVLCPHPHHHLHHYSWFPFLIPVLHTLPKLQPKFHGR | |
| PEQPLNLLQVKPTSGTI ASAEQVWVK. | |
| VCYLQAYKNTLLFIRSLNEHGLQQLLFQW, and/or | (SEQ ID NO:444) |
| VPYVLCPHPHHHLHHYSWFPFLIPVLHTLLPKS. | (SEQ ID NO:445) |

Polynucleotides encoding these polypeptides are also provided.

The gene encoding the disclosed cDNA is believed to reside on chromosome 1. Accordingly, polynucleotides related to this invention are useful as a marker in linkage analysis for chromosome 1.

This gene is expressed primarily in brain, ulcerative colitis, pancreas tumor, placenta, and, to a lesser extent, in thyroid, bone marrow stromal cells, B-cell lymphoma, and hemangiopericytoma.

Therefore, polynucleotides and polypeptides of the invention are useful as reagents for differential identification of the tissue(s) or cell type(s) present in a biological sample and for diagnosis of diseases and conditions which include, but are not limited to, tumors and degenerative conditions involving infiltration by the immune system, particularly in soft-tissues, in addition to, neural, gastrointestinal, metabolic, reproductive, endocrine, and hematopoietic, or immune disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., neural, gastrointestinal, metabolic, reproductive, endocrine, hematopoietic, immune disorders, and cancerous and wounded tissues) or bodily fluids (e.g., lymph, serum, bile, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 213 as residues: Lys-33 to Arg-51, Gly-64 to Gly-74.

The tissue distribution in brain tissues, combined with the detected EGR1 biological activity, indicates that polynucleotides and polypeptides corresponding to this gene are useful for treating the secondary effects of immune system involvement in diseases such as pancreatic tumors, ulcerative colitis, and Alzheimer's disease. Representative uses are described in the “Regeneration” and “Hyperproliferative Disorders” sections below, in Example 11, 15, and 18, and elsewhere herein. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:85 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1244 of SEQ ID NO:85, b is an integer of 15 to 1258, where both a and b correspond to the positions of nucleotide residues shown in SEQ ID NO:85, and where b is greater than or equal to a +14.

Features of Protein Encoded by Gene No: 76

When tested against PC12 cell lines, supernatants removed from cells containing this gene activated the EGR1 (early growth response gene 1) promoter element. Thus, it is likely that this gene activates sensory neuron cells, and to a lesser extent, other tissues and cell-types, through the EGR1 signal transduction pathway. EGR1 is a separate signal transduction pathway from Jak-STAT, genes containing the EGR1 promoter are induced in various tissues and cell types upon activation, leading the cells to undergo differentiation and proliferation.

Preferred polypeptides of the invention comprise the following amino acid sequence: ESERAVVYLITGALFIVSSCVLCFLPSSRRE (SEQ ID NO:446). Polynucleotides encoding these polypeptides are also provided.

The gene encoding the disclosed cDNA is believed to reside on chromosome 12. Accordingly, polynucleotides related to this invention are useful as a marker in linkage analysis for chromosome 12.

This gene is expressed primarily in activated T cells, tonsils, and activated monocytes.

Therefore, polynucleotides and polypeptides of the invention are useful as reagents for differential identification of the tissue(s) or cell type(s) present in a biological sample and for diagnosis of diseases and conditions which include, but are not limited to, immune and inflammatory diseases and/or disorders. Similarly, polypeptides and antibodies directed to these polypeptides are useful in providing immunological probes for differential identification of the tissue(s) or cell type(s). For a number of disorders of the above tissues or cells, particularly of the activated T cells, tonsils and activated monocytes, expression of this gene at significantly higher or lower levels may be routinely detected in certain tissues and cell types (e.g., immune, hematopoietic, neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in T-cells and immune tissues or cell types, combined with the detected EGR biological activity, indicates that polynucleotides and polypeptides corresponding to this gene are useful for the diagnosis and/or treatment of immune and inflammatory disorders. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:86 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1304 of SEQ ID NO:86, b is an integer of 15 to 1318, where both a and b correspond to the positions of nucleotide residues shown in SEQ ID NO:86, and where b is greater than or equal to a +14.

Features of Protein Encoded by Gene No: 77

When tested against fibroblast cell lines, supernatants removed from cells containing this gene activated the EGR1 assay. Thus, it is likely that this gene activates fibroblast cells through a signal transduction pathway. Early growth response I (EGR1) is a promoter associated with certain genes that induces various tissues and cell types upon activation, leading the cells to undergo differentiation and proliferation.

The gene encoding the disclosed cDNA is thought to reside on chromosome 16. Accordingly, polynucleotides related to this invention are useful as a marker in linkage analysis for chromosome 16.

Preferred polypeptides of the invention comprise the following amino acid sequence:

| | |
|---|---|
| HEARQGVSRGVKAAMNRVLCAPAAGAVRALRLIGWASRSLHPLPGSRDRAH | (SEQ ID NO:447) |
| PAAEEEDDPDRPIEFSSSKANPHRWSVHGTMGKGHQRPWWKVLPLSCFLVA | |
| LIIWCXLREESEADQWLRQVWGEVPEPSDRSEEPETPAAYRART | |

and fragments thereof as described generally elsewhere herein. Polynucleotides encoding such polypeptides and polypeptide fragments are also provided.

This gene is expressed primarily in eosinophils and activated T-cells, and to a lesser extent in lung and thymus stromal cells.

Therefore, polynucleotides and polypeptides of the invention are useful as reagents for differential identification of the tissue(s) or cell type(s) present in a biological sample and for diagnosis of diseases and conditions which include, but are not limited to, immune disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 215 as residues: Met-1 to Trp-10.

The tissue distribution indicates that polynucleotides and polypeptides corresponding to this gene are useful for the diagnosis and/or treatment of immune disorders, including infection, allergy, inflammation, graft rejection and immunodeficiency. Furthermore, the tissue distribution indicates that polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. Expression of this gene product in T cells and eosinophils also strongly indicates a role for this protein in immune function and immune surveillance.

Many polynucleotide sequences, such as EST sequences, are publicly available and accessible through sequence databases. Some of these sequences are related to SEQ ID NO:87 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 964 of SEQ ID NO:87, b is an integer of 15 to 978, where both a and b correspond to the positions of nucleotide residues shown in SEQ ID NO:87, and where b is greater than or equal to a +14.

Features of Protein Encoded by Gene No: 78

Preferred polypeptides of the invention comprise the following amino acid sequence:

| | |
|---|---|
| MWVXGEEVLGSHAASPAFLHRCFSEESCVSIPEVEGYVVVLQPDAPQILLSGT | (SEQ ID NO:448) |
| AHFARPAVDFEGTNGVPLFPDLQITCSISHQVEAKKDESWQGTVTDTRMSDEI | |
| VHNLDGCEISLVGDDLDPERESLLLDTTSLQQRGLELTNTSAYLTIAGVESITV | |
| YEEILRQARYRLRHGAALYTRKFRLSCSEMNGRYSSNEFIVEVNVLHSMNRV | |
| AHPSHVLSXQQFLHRGHQPPPEMAGHSLASSHRNSST, | |
| LGSHAASPAFLHRCFSEESCVSI, | (SEQ ID NO:449) |
| GYVVVLQPDAPQILLSGTAHFARPAVDFE, | (SEQ ID NO:450) |
| ITCSISHQVEAKKDESWQGTVTDTRM, | (SEQ ID NO:451) |
| NLDGCEISLVGDDLDPERESLLLDTTSLQ, | (SEQ ID NO:452) |
| SAYLTIAGVESITVYEEILRQAR, | (SEQ ID NO:453) |
| RLSCSEMNGRYSSNEFIVEVNVLHSM, and/or | (SEQ ID NO:454) |
| QQFLHRGHQPPPEMAGHSLASSHRN. | (SEQ ID NO:455) |

Polynucleotides encoding these polypeptides are also provided.

This gene is expressed primarily in brain and spleen tissues.

Therefore, polynucleotides and polypeptides of the invention are useful as reagents for differential identification of the tissue(s) or cell type(s) present in a biological sample and for diagnosis of diseases and conditions which include, but are not limited to, brain afflictions such as depression, schizophrenia, Alzheimer's disease, Parkinson's disease, Huntington's disease, specific brain tumors, aphasia, mania, depression, dementia, paranoia, addictive behavior and sleep disorders, as well as immune disorders such as leukemias, lymphomas, AIDS, arthritis and imflammation. Similarly, polypeptides and antibodies directed to these polypeptides are useful in providing immunological probes for differential identification of the tissue(s) or cell type(s). For a number of disorders of the above tissues or cells, particularly of the central nervous and immune systems, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 216 as residues: Gly-36 to Leu-44.

The tissue distribution indicates that polynucleotides and polypeptides corresponding to this gene are useful for the treatment and/or diagnosis of developmental, degenerative and behavioral diseases and conditions of the brain such as aphasia, depression, schizophrenia, Alzheimer's disease, Parkinson's disease, Huntington's disease, specific brain tumors, mania, depression, dementia, paranoia, addictive behavior and sleep disorders. In addition, the expression in spleen would suggest a possible role in the detection and treatment of immune disorders including: leukemias, lymphomas, auto-immunities, immunodeficiencies (e.g., AIDS), immunosupressive conditions (transplantation) and hematopoietic disorders as well as conditions of general microbial infection, inflammation or cancer.

Many polynucleotide sequences, such as EST sequences, are publicly available and accessible through sequence databases. Some of these sequences are related to SEQ ID NO:88 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1849 of SEQ ID NO:88, b is an integer of 15 to 1863, where both a and b correspond to the positions of nucleotide residues shown in SEQ ID NO:88, and where b is greater than or equal to a +14.

Features of Protein Encoded by Gene No: 79

When tested against K562 leukemia cell lines, supernatants removed from cells containing this gene activated the ISRE assay. Thus, it is likely that this gene activates leukemia cells through the Jak-STAT signal transduction pathway. The interferon-sensitive response element is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the ISRE element, can be used to indicate proteins involved in the proliferation and differentiation of cells. One embodiment of this invention comprises polypeptides comprising one of the following amino acid sequences:

| | |
|---|---|
| MADSETFISLEECRGHKRARKRTSMETALALEKLFPKQCQVLGIVTPGIVVXP | (SEQ ID NO:456) |
| MGSGSNRPQEIEIGESGFALLFPQIEGIKIQPFHFIKDPKNLTLERHQLTEVGLL | |
| DNPELRVVLVFGYNCCKVGASNYLQQVVSTFSDMNIILAGGQVDNLSSLTSE | |
| KNPLDIDASGVVGLSFSGHRIQSATVLLNEDVSDEKTAEAAMQRLKAANIPE | |
| HNTIGFMFACVGRGFQYYRAKGNVEADAFRKFFPSVPLFGFFGNGEIGCDRIV | |
| TGNFILRKCNEVKDDDLFHSYTTIMALIHLGSSK, | |
| HKRARKRTSMETALALEKLFP, | (SEQ ID NO:457) |
| MGSGSNRPQEIEIGESGFALLFPQ, | (SEQ ID NO:458) |
| FHFIKDPKNLTLERHQLTEVGL, | (SEQ ID NO:459) |
| FGYNCCKVGASNYLQQVVSTFSD, | (SEQ ID NO:460) |
| TSEKNPLDIDASGVVGLSFS, | (SEQ ID NO:461) |

| | |
|---|---|
| NEDVSDEKTAEAAMQRLKAANIPEHN, | (SEQ ID NO:462) |
| YYRAKGNVEADAFRKFFPSVPLFGF, and/or | (SEQ ID NO:463) |
| IGCDRIVTGNFILRKCNEVKDDDLFH. | (SEQ ID NO:464) |

An additional embodiment is the polynucleotides encoding these polypeptides.

This gene is expressed primarily in endothelial cells, and to a lesser extent in reproductive and various endocrine organs.

Therefore, polynucleotides and polypeptides of the invention are useful as reagents for differential identification of the tissue(s) or cell type(s) present in a biological sample and for diagnosis of diseases and conditions which include, but are not limited to, cancer, cardiovascular and immune defects. Similarly, polypeptides and antibodies directed to these polypeptides are useful in providing immunological probes for differential identification of the tissue(s) or cell type(s). For a number of disorders of the above tissues or cells, particularly of the immune, cardiovascular, and reproductive systems, expression of this gene at significantly higher or lower levels may be routinely detected in certain tissues or cell types (e.g., endothelial, reproductive, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 217 as residues: Ser-44 to Ala-50.

The tissue distribution indicates that polynucleotides and polypeptides corresponding to this gene are useful for the diagnosis and/or treatment of cancer, cardiovascular and reproductive disorders.

Many polynucleotide sequences, such as EST sequences, are publicly available and accessible through sequence databases. Some of these sequences are related to SEQ ID NO:89 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2072 of SEQ ID NO:89, b is an integer of 15 to 2086, where both a and b correspond to the positions of nucleotide residues shown in SEQ ID NO:89, and where b is greater than or equal to a +14.

Features of Protein Encoded by Gene No: 80

This gene is expressed primarily in human tongue and TNF-induced epithelium.

Therefore, polynucleotides and polypeptides of the invention are useful as reagents for differential identification of the tissue(s) or cell type(s) present in a biological sample and for diagnosis of diseases and conditions which include, but are not limited to, mucosal, oral, and inflammatory conditons. Similarly, polypeptides and antibodies directed to these polypeptides are useful in providing immunological probes for differential identification of the tissue(s) or cell type(s). For a number of disorders of the above tissues or cells, particularly of mucosal and epidermal tissues, expression of this gene at significantly higher or lower levels may be routinely detected in certain tissues or cell types (e.g., tongue, epithelial, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO:

218 as residues: Ser-39 to Leu-48, Ala-65 to Pro-75, Pro-81 to Cys-87.

The tissue distribution indicates that polynucleotides and polypeptides corresponding to this gene are useful for the study, detection and/or treatment of disorders of the oral and intestinal mucosa, inflammation, and other epithelial disorders.

Many polynucleotide sequences, such as EST sequences, are publicly available and accessible through sequence databases. Some of these sequences are related to SEQ ID NO:90 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 877 of SEQ ID NO:90, b is an integer of 15 to 891, where both a and b correspond to the positions of nucleotide residues shown in SEQ ID NO:90, and where b is greater than or equal to a +14.

Features of Protein Encoded by Gene No: 81

This gene is expressed primarily in activated neutrophils.

Therefore, polynucleotides and polypeptides of the invention are useful as reagents for differential identification of the tissue(s) or cell type(s) present in a biological sample and for diagnosis of diseases and conditions which include, but are not limited to, immune, autoimmune, and inflammatory conditions. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution indicates that polynucleotides and polypeptides corresponding to this gene are useful for the study, diagnosis and/or treatment of immune, autoimmune, and inflammatory disorders. Furthermore, this gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Expression of this gene product in neutrophils strongly indicates a role for this protein in immune function and immune surveillance.

Many polynucleotide sequences, such as EST sequences, are publicly available and accessible through sequence databases. Some of these sequences are related to SEQ ID NO:91 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1960 of SEQ ID NO:91, b is an integer of 15 to 1974, where both a and b correspond to the positions of nucleotide residues shown in SEQ ID NO:91, and where b is greater than or equal to a +14.

Features of Protein Encoded by Gene No: 82

Multiple endocrine neoplasia type 1 (MEN 1) is an inherited cancer syndrome in which affected individuals develop multiple parathyroid, enteropancreatic, and pituitary tumors. The locus for MENI is tightly linked to the marker PYGM on chromosome 11q13, and linkage analysis places the MEN1 gene within a 2-Mb interval flanked by the markers D11S1883 and D11S449. Loss of heterozygosity studies in MEN 1 and sporadic tumors suggest that the MEN1 gene encodes a tumor suppressor and have helped to narrow the location of the gene to a 600-kb interval between PYGM and D11S449. The transcript for this gene shares sequence identity with a transcript determined to map to the MEN-1 locus. (Genome Res. 1997 July;7(7):725-35).

When tested against NIH3T3 cell lines, supernatants removed from cells containing this gene activated the EGR1 (early growth response gene 1) promoter element. Thus, it is likely that this gene activates fibroblast cells, and to a lesser extent, other cells and tissue cell-types, through the EGR1 signal transduction pathway. EGR1 is a separate signal transduction pathway from Jak-STAT, genes containing the EGR1 promoter are induced in various tissues and cell types upon activation, leading the cells to undergo differentiation and proliferation.

Preferred polypeptides of the invention comprise the following amino acid sequence

| | |
|---|---|
| GTRYFLMELVWFRFLHLNLLPRGVCCGICVCVRRGMVLSEPTSCGQRALSCE | (SEQ ID NO:465) |
| GGCHSGRVQFRRP. | |

Polynucleotides encoding such polypeptides are also provided.

This gene is expressed primarily in primary dendritic cells, and to a lesser extent in neutrophils, monocytes, and osteoblasts.

Therefore, polynucleotides and polypeptides of the invention are useful as reagents for differential identification of the tissue(s) or cell type(s) present in a biological sample and for diagnosis of diseases and conditions which include, but are not limited to, immune and hematopoietic conditions. Similarly, polypeptides and antibodies directed to these polypeptides are useful in providing immunological probes for differential identification of the tissue(s) or cell type(s). For a number of disorders of the above tissues or cells, particularly of the immune and hematopoietic systems, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 220 as residues: Gly-47 to Arg-53.

The tissue distribution in dendritic cells, combined with the detected EGR1 biological activity, indicates that polynucleotides and polypeptides corresponding to this gene are useful for the study, diagnosis and/or treatment of immune, inflammatory and hematopoietic disorders. Furthermore, this gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Expression of this gene product in neutrophils and primary dendritic cells also strongly indicates a role for this protein in immune function and immune surveillance. The tissue distribution and sequence similarity to nucleic acid sequences derived from the MEN-1 region further indicate that this gene and its gene products are useful in the treatment of cancer, particularly the treatment of pancreatic, parathyroid and prostate cancers.

Many polynucleotide sequences, such as EST sequences, are publicly available and accessible through sequence databases. Some of these sequences are related to SEQ ID NO:92 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1409 of SEQ ID NO:92, b is an integer of 15 to 1423, where both a and b correspond to the positions of nucleotide residues shown in SEQ ID NO:92, and where b is greater than or equal to a +14.

Features of Protein Encoded by Gene No: 83

Preferred polypeptides of the invention comprise the following amino acid sequence:

| | |
|---|---|
| MPKRKVTFQGVGDEEDEDEIIVPKKKLVDPVAGSGGPGSRFKGKHSLDSDEE | (SEQ ID NO:466) |
| EDDDDGGSSKYDILASEDVEGQEAATLPSEGGVRITPFNLQEEMEEGHFDAD | |
| GNYFLNRDAQIRDSWLDNIDWVDIRERPPGQRQASDSEEEDSLGQTSMSAQA | |
| LLEGLLELLLPRETVAGALRRLGARGGGKGRKGPGQPSSPQRLDRLSGLADQ | |
| MVARGNLGVYQETRERLAMRLKGLGCQTLGPHNPTPPPSLDMFAEELAEEE | |
| LETPTPTQRGEAESRGDGLVDVMWEYKWENTGDAELTGPFTSAQMQTWVS | |
| EGYFPDGVYCRKLDPPGGQFYN SKRIDFDLYT, | |
| TFQGVGDEEDEDEIIVPKKKL VDP, | (SEQ ID NO:467) |
| PGSRFKGKHSLDSDEEEDDDDGGSSKY, | (SEQ ID NO:468) |
| EAATLPSEGGVRITPFNLQEEMEEG, | (SEQ ID NO:469) |
| FLNRDAQIRDSWLDNIDWVKIRERPPGQR, | (SEQ ID NO:470) |
| SLGQTSMSAQALLEGLLELLLPRETV, | (SEQ ID NO:471) |
| RGGGKGRKGPGQPSSPQRLDRLSGLADQ, | (SEQ ID NO:472) |
| QETRERLAMRLKGLGCQTLGPHNP, | (SEQ ID NO:473) |
| DMFAEELAEEELETPTPTQRGEAESRGD, and/or | (SEQ ID NO:474) |
| ELYGPFTSAQMQTWVSEGYFPDGVYCRKLD. | (SEQ ID NO:475) |

Polynucleotides encoding these polypeptides are also provided.

This gene is expressed primarily in fetal lung, stromal cells and lymphoma cells.

Therefore, polynucleotides and polypeptides of the invention are useful as reagents for differential identification of the tissue(s) or cell type(s) present in a biological sample and for diagnosis of diseases and conditions which include, but are not limited to, haemopoietic and respiratory disorders and cancer. Similarly, polypeptides and antibodies directed to these polypeptides are useful in providing immunological probes for differential identification of the tissue(s) or cell type(s). For a number of disorders of the above tissues or cells, particularly of the haemopoietic and respiratory systems, expression of this gene at significantly higher or lower levels may be routinely detected in certain tissues or cell types (e.g., lung, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 221 as residues: Met-I to Trp-15, Thr-52 to Met-58.

The tissue distribution indicates that polynucleotides and polypeptides corresponding to this gene are useful for the treatment and/or diagnosis of diseases of the haemopoietic and respiratory systems. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:93 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1351 of SEQ ID NO:93, b is an integer of 15 to 1365, where both a and b correspond to the positions of nucleotide residues shown in SEQ ID NO:93, and where b is greater than or equal to a +14.

Features of Protein Encoded by Gene No: 84

Preferred polypeptides of the invention comprise the following amino acid sequence:

| | |
|---|---|
| ARGWECEEGSPGPVFRGCASPRTPVSGNAVPSTFRACPPCGVAALLPGVISSE | (SEQ ID NO:483) |
| SFLHALFPPHVPPRALPTSVPWFGSSSPVRYGYPRVWS, | |
| PHSSRVSFLQSLSF, | (SEQ ID NO:476) |
| RGQPRPCVSGVCLSPHSRFWECCSFYLQGLPALRCSRTPPGCHFFRVFPSCPFS | (SEQ ID NO:477) |
| SSRSPSCFTHICPVVRIQFSRALWVSTCLVLAITPGKWLLPEDRALSLMLLASL | |
| QCCPPPFGAWWMQVLTHKGRQAGLGPGVSSRPL, | |
| SNIKSLPPTNSLSLLRAQTGTDCAVSPGLAGPCHQRGLEDTPGPRPACLPLCVS | (SEQ ID NO:478) |
| TCIHQAPKGGGQHWREASSIRDRALSSGRSHFPGVMAKTKHVDTHNARENW | |
| IRTTGQMWVKHEGEREEEKGHEGKTLKK, | |
| VCLSPHSRFWECCSFYLQGLPALRC, | (SEQ ID NO:479) |
| QFSRALWVSTCLVLAITPGKWLLPEDR, | (SEQ ID NO:480) |
| SLSLLRAQTGTDCAVSPGLAGPCHQRG, and/or | (SEQ ID NO:481) |
| SGRSHFPGVMAKTKHVDTHNARENWIRT. | (SEQ ID NO:482) |

Polynucleotides encoding these polypeptides are also provided. When tested against U937 cell lines, supernatants removed from cells containing this gene activated the GAS (gamma activating sequence) promoter element. Thus, it is likely that this gene activates myeloid cells, including their progenitors, through the Jak-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

This gene is expressed primarily in T-cells and lungs.

Therefore, polynucleotides and polypeptides of the invention are useful as reagents for differential identification of the tissue(s) or cell type(s) present in a biological sample and for diagnosis of diseases and conditions which include, but are not limited to, respiratory and immune diseases. Similarly, polypeptides and antibodies directed to these polypeptides are useful in providing immunological probes for differential identification of the tissue(s) or cell type(s). For a number of disorders of the above tissues or cells, particularly of the immune and respiratory systems, expression of this gene at significantly higher or lower levels may be routinely detected in certain tissues or cell types (e.g., pulmonary, immune, hematopoietic, and cancerous and wounded tissues) or bodily fluids (e.g., lymph, pulmponary surfactant or sputum, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 222 as residues: His-38 to Ala-43.

The tissue distribution in T-cells and lung tissue, combined with the detected GAS biological activity, indicates that polynucleotides and polypeptides corresponding to this gene are useful for the treatment and/or diagnosis of disorders of the respiratory and immune systems. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues. The protein may show utility in modulating the immune response to various pulmonary disorders or conditions, particularly in emphysema, or ARDS.

Many polynucleotide sequences, such as EST sequences, are publicly available and accessible through sequence databases. Some of these sequences are related to SEQ ID NO:94 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 742 of SEQ ID NO:94, b is an integer of 15 to 756, where both a and b correspond to the positions of nucleotide residues shown in SEQ ID NO:94, and where b is greater than or equal to a +14.

Features of Protein Encoded by Gene No: 85

Preferred polypeptides of the invention comprise the following amino acid sequence:

| | |
|---|---|
| ARVEVQGQGPGAKVDAGEGQ, | (SEQ ID NO:484) |
| WVVLSQLQAQGVAGMMCSYPEGQKKGKEATRSHRWVPRSLPGMGSXLAA | (SEQ ID NO:485) |
| PHSNPWLAPLALLEIPXPVLCEWKRKLIALEEVSECRPGVGGGGGFLSXCRRG | |
| HLSFLSGAPYPLFPISPLX, | |
| ELRHGGPRQVKDSFLDYMGYPDEDRAGPPSRWFPRERFLSPPTVVPLCVELR | (SEQ ID NO:486) |
| LGFESGMGWGVPGSSHSEGGPEARWPLIAPMYTVTQWFQRPNSGRGPQPPP | |
| QXRGEIGKRGYGAPERKLRWPLLXWERXPPPPPTPGRHSETSSSAISFLFHSQR | |
| TGWGISSSANGASQGLLWGAARXLPIPGRDLGTHLWDLVASFPFFCPSG, | |
| PEGQKKGKEATRSHRWVPRSLPGM, | (SEQ ID NO:487) |
| LRLGFESGMGWGVPGSSHSEGGPEAR, and/or | (SEQ ID NO:488) |
| HSQRTGWGISSSANGASQGLLWGA. | (SEQ ID NO:489) |

Polynucleotides encoding these polypeptides are also provided.

This gene is expressed primarily in eosinophils, dendritic cells, Jurkat cells and tonsils.

Therefore, polynucleotides and polypeptides of the invention are useful as reagents for differential identification of the tissue(s) or cell type(s) present in a biological sample and for diagnosis of diseases and conditions which include, but are not limited to, immune, or hematopoietic disorders, particularly inflammatory, autoimmune, allergy, and hypersensitivity conditions. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in a variety of immune and hematopoietic-specific cells and tissues indicates that polynucleotides and polypeptides corresponding to this gene are useful for modifying the response of the immune system in autoimmune diseases and inflammatory conditions. Moreover, polynucleotides and polypeptides corresponding to this gene are useful for the treatment and/or diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc.

In addition, this gene product may have commercial utility in the expansion of stem cells and committed progenitors of various blood lineages, and in the differentiation and/or proliferation of various cell types. It may also have a very wide range of biological acitivities. Typical of these are cytokine, cell proliferation/differentiation modulating activity or induction of other cytokines; immunostimulating/immunosuppressant activities (e.g., for treating human immunodeficiency virus infection, cancer, autoimmune diseases and allergy); regulation of hematopoiesis (e.g., for treating anaemia or as adjunct to chemotherapy); stimulation or growth of bone, cartilage, tendons, ligaments and/or nerves (e.g., for treating wounds, stimulation of follicle stimulating hormone (for control of fertility); chemotactic and chemokinetic activities (e.g., for treating infections, tumors); hemostatic or thrombolytic activity (e.g., for treating haemophilia, cardiac infarction etc.); anti-inflammatory activity (e.g., for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:95 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 924 of SEQ ID NO:95, b is an integer of 15 to 938, where both a and b correspond to the positions of nucleotide residues shown in SEQ ID NO:95, and where b is greater than or equal to a +14.

Features of Protein Encoded by Gene No: 86

Preferred polypeptides of the invention comprise the following amino acid sequence: DSLTIKSGSQPQYSPAITLW (SEQ ID NO:490). Polynucleotides encoding these polypeptides are also provided.

This gene is expressed primarily in cells from fibrosarcoma tumors.

Therefore, polynucleotides and polypeptides of the invention are useful as reagents for differential identification of the tissue(s) or cell type(s) present in a biological sample and for diagnosis of diseases and conditions which include, but are not limited to, muscle, or endothelial disorders, particularly fibrosarcomas and fibroids. Similarly, polypeptides and antibodies directed to these polypeptides are useful in providing immunological probes for differential identification of the tissue(s) or cell type(s). For a number of disorders of the above tissues or cells, particularly of the skeleto-muscular system, expression of this gene at significantly higher or lower levels may be routinely detected in certain tissues or cell types (e.g., skeleto-muscular, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in fibrosarcoma tissue indicates that polynucleotides and polypeptides corresponding to this gene are useful for the detection, treatment, and/or prevention of various muscle disorders, in particular fibrosarcomas. Representative uses are described elsewhere herein. Briefly, the uses include, but are not limited to the detection, treatment, and/or prevention of disorders and conditions afflicting the skeletal system, in particular osteoporosis, bone cancer, connective tissue disorders (e.g., arthritis, trauma, tendonitis, chrondomalacia and inflammation). The protein is also useful in the diagnosis or treatment of various autoimmune disorders (i.e., rheumatoid arthritis, lupus, scleroderma, and dermatomyositis), dwarfism, spinal deformation, joint abnormalities, and chondrodysplasias (i.e. spondyloepiphyseal dysplasia congenita, familial osteoarthritis, Atelosteogenesis type II, metaphyseal chondrodysplasia type Schmid, etc.). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:96 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 914 of SEQ ID NO:96, b is an integer of 15 to 928, where both a and b correspond to the positions of nucleotide residues shown in SEQ ID NO:96, and where b is greater than or equal to a +14.

Features of Protein Encoded by Gene No: 87

This gene is expressed primarily in helper T-Cells, cerebellum, and, to a lesser extent, in mesangial cells, fetal lung, fetal liver, cortex, and adipose tissue.

Therefore, polynucleotides and polypeptides of the invention are useful as reagents for differential identification of the tissue(s) or cell type(s) present in a biological sample and for diagnosis of diseases and conditions which include, but are not limited to, immune, or neural diseases and/or disorders, particularly, for modulation of immune responses to viral or bacterial infections, or neurodefeciencies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., renal, developmental, pulmonary, hepatic, neural, metabolic, immune, and cancerous and wounded tissues) or bodily fluids (e.g., lymph, serum, bile, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in helper T-cells indicates that polynucleotides and polypeptides corresponding to this gene are useful for modifying the immune response to foreign agents such as bacteria or virus. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Moreover, based upon the expression within the cerebellum and cortex, polynucleotides and polypeptides corresponding to this gene are useful for the detection/treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival.

Moreover, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:97 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1701 of SEQ ID NO:97, b is an integer of 15 to 1715, where both a and b correspond to the positions of nucleotide residues shown in SEQ ID NO:97, and where b is greater than or equal to a +14.

Features of Protein Encoded by Gene No: 88

When tested against U937 cell lines, supernatants removed from cells containing this gene activated the GAS (gamma activating sequence) promoter element. Thus, it is likely that this gene activates promyelocytic cells, and to a lesser extent, other tissues and cell-types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| FIMKLLYQLLMLTTSSSYSLITHLCYSIFLCSFYFHFPCNVSLFVLISEEFIYD, | (SEQ ID NO:491) |
| LMLTTSSSYSLITHLCYSIFL, | (SEQ ID NO:492) |
| LCSFYFHFPCNVSLFVLISEE, | (SEQ ID NO:493) |
| MRKNIFAILDKMLTCLIINELFRNQYDITNITREVKIKGTEENGIAQMSYKAI, | (SEQ ID NO:494) |
| DKMLTCLIINELFRNQYKETN, and/or | (SEQ ID NO:495) |
| NITREVKIKGTEENGIAQMSY. | (SEQ ID NO:496) |

Polynucleotides encoding these polypeptides are also provided.

This gene is expressed primarily in fetal heart and lung, cheek carcinoma, small intesine, and tonsil.

Therefore, polynucleotides and polypeptides of the invention are useful as reagents for differential identification of the tissue(s) or cell type(s) present in a biological sample and for diagnosis of diseases and conditions which include, but are not limited to, pulmonary and developmental diseases and/or disorders. Similarly, polypeptides and antibodies directed to these polypeptides are useful in providing immunological probes for differential identification of the tissue(s) or cell type(s). For a number of disorders of the above tissues or cells, particularly of the developmental and pulmonary systems, expression of this gene at significantly higher or lower levels may be routinely detected in certain tissues and cell types (e.g., pulmonary, developmental, cardiovascular, immune, hematopoietic, and cancerous and wounded tissues) or bodily fluids (e.g., lymph, serum, pulmonary surfactant or sputum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution of this gene only in fetal lung, combined with the detected GAS biological activity indicates that it plays a key role in development of the pulmonary system. This would suggest that misregulation of the expression of this protein product in the adult could lead to lymphoma or sarcoma formation, particularly in the lung. It may also be involved in the predisposition to certain pulmonary defects such as pulmonary edema and embolism, bronchitis and cystic fibrosis. Moreover, the protein product of this gene may be beneficial in the treatment of underdeveloped lung tissue, as exists in premature infants, both through the use of antibodies directed against the protein, through a gene therapy-based regimine, or through the action of the protein itself, either directly or indirectly. Moreover, the expression within fetal tissue and other cellular sources marked by proliferating cells (i.e., cheek carcinoma, etc.) indicates this protein may play a role in the regulation of cellular division, and may show utility in the diagnosis, treatment, and/or prevention of developmental diseases and disorders, cancer, and other proliferative conditions.

Representative uses are described in the “Hyperproliferative Disorders” and “Regeneration” sections below and elsewhere herein. Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:98 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 664 of SEQ ID NO:98, b is an integer of 15 to 678, where both a and b correspond to the positions of nucleotide residues shown in SEQ ID NO:98, and where b is greater than or equal to a +14.

Features of Protein Encoded by Gene No: 89

When tested against U937 cell lines, supernatants removed from cells containing this gene activated the GAS (gamma activating sequence) promoter element. Thus, it is likely that this gene activates myeloid cells, including their progenitors, through the Jak-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence: GISERKP (SEQ ID NO:497). Polynucleotides encoding these polypeptides are also provided.

This gene is expressed primarily in brain tissue.

Therefore, polynucleotides and polypeptides of the invention are useful as reagents for differential identification of the tissue(s) or cell type(s) present in a biological sample and for diagnosis of diseases and conditions which include, but are not limited to, neural or immune disorders. Similarly, polypeptides and antibodies directed to these polypeptides are useful in providing immunological probes for differential identification of the tissue(s) or cell type(s). For a number of disorders of the above tissues or cells, particularly of the central nervous system, expression of this gene at significantly higher or lower levels may be routinely detected in certain tissues or cell types (e.g., neural, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 227 as residues: Ile-40 to Trp-50.

The tissue distribution in brain tissue, combined with the detected GAS biological activity, indicates that polynucleotides and polypeptides corresponding to this gene are useful for the treatment and/or diagnosis of central nervous system disorders. Moreover, polynucleotides and polypeptides corresponding to this gene are useful for the detection and/or treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival.

Moreover, the gene or gene product may also play a role in the treatment and/or detection of developmental disorders associated with the developing embryo, sexually-linked disorders, or disorders of the cardiovascular system. Furthermore, the protein may show utility in modulating the immune response to various neurodegenerative conditions. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:99 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1527 of SEQ ID NO:99, b is an integer of 15 to 1541, where both a and b correspond to the positions of nucleotide residues shown in SEQ ID NO:99, and where b is greater than or equal to a +14.

Features of Protein Encoded by Gene No: 90

Preferred polypeptides of the invention comprise the following amino acid sequence:

| | |
|---|---|
| QSPAVSYTVTSQVPWGLGLLAGEKR, | (SEQ ID NO:498) |
| LPSHPLRPLTFSSAMCMHLPPPLCRRAALSAPFATQHRPWSVAAACLPRIHQN | (SEQ ID NO:499) |
| PLDAEYPSGCCRMSFLPAACSNIYSQECHYTLMSHSEASTLQXAQLL, | |
| MLLQAAGRKLMRQQPDGYSASRGFWWMRGRQAAATLHGRCWVAKGADS | (SEQ ID NO:500) |
| AALRQRGGGRCMHIADEKVRGLSGCDGS, | |
| LCRRAALSAPFATQHRPWSVAAACL, | (SEQ ID NO:501) |
| RGFWWMRGRQAAATLHGRCWVAKG, and/or | (SEQ ID NO:502) |
| QRGGGRCMHIAKEDVRGLSGCDG. | (SEQ ID NO:503) |

Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, inflammatory and immune conditions. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 228 as residues: Pro-34 to His-39, Pro-44 to His-54.

The tissue distribution in neutrophils indicates that polynucleotides and polypeptides corresponding to this gene are useful for the study, diagnosis, and/or treatment of inflammatory, general immune, and infectious diseases. Moreover, the expression of this gene indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:100 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 867 of SEQ ID NO:100, b is an integer of 15 to 881, where both a and b correspond to the positions of nucleotide residues shown in SEQ ID NO:100, and where b is greater than or equal to a +14.

Features of Protein Encoded by Gene No: 91

When tested against Jurkat cell lines, supernatants removed from cells containing this gene activated the GAS (gamma activating sequence) promoter element. Thus, it is likely that this gene activates myeloid cells through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

In addition, contact of cells with supernatant expressing the product of this gene has been shown to increase the permeability of the plasma membrane of stromal cells to calcium. Thus, it is likely that the product of this gene is involved in a signal transduction pathway that is initiated when the product binds a receptor on the surface of the plasma membrane of both stromal, in addition to other cell-lines or tissue cell types. Thus, polynucleotides and polypeptides have uses which include, but are not limited to, activating stromal cells. Binding of a ligand to a receptor is known to alter intracellular levels of small molecules, such as calcium, potassium and sodium, as well as alter pH and membrane potential. Alterations in small molecule concentration can be measured to identify supernatants which bind to receptors of a particular cell.

Preferred polypeptides of the invention comprise the following amino acid sequence:

| | |
|---|---|
| THPSHPSIVIQSTVSLCLTASSRRKKSDCLSLCQVSCSQRPGSHKTNVAWGFL | (SEQ ID NO:504) |
| MSRVHFSVRWVSGGRGI TGAICKESSLPCKEIQGKACYFCHHPAQQSTPFSHI, | |
| VIQSTVSLCLTASSRRKKSDCLSLCQV, and/or | (SEQ ID NO:505) |
| ICKESSLPCKEIQGKACYFCHHPAQQQ. | (SEQ ID NO:506) |

Polynucleotides encoding these polypeptides are also provided.

This gene is expressed primarily in neutrophils, and to a lesser extent in cord blood.

Therefore, polynucleotides and polypeptides of the invention are useful as reagents for differential identification of the tissue(s) or cell type(s) present in a biological sample and for diagnosis of diseases and conditions which include, but are not limited to, immune or developmental disorders, particularly inflammatory conditions. Similarly, polypeptides and antibodies directed to these polypeptides are useful in providing immunological probes for differential identification of the tissue(s) or cell type(s). For a number of disorders of the above tissues or cells, particularly of the immune and haemopoietic systems, expression of this gene at significantly higher or lower levels may be routinely detected in certain tissues or cell types (e.g., immune, developmental, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 229 as residues: Glu-32 to Arg-37.

The tissue distribution in neutrophils, combined with the detected GAS and calcium flux biological activities, indicates that polynucleotides and polypeptides corresponding to this gene are useful for the study, diagnosis and/or treatment of inflammatory, infectious, and haemopoietic disorders. Similarly, expression within cord blood indicates that this protein may play a role in the regulation of cellular division, and may show utility in the diagnosis and treatment of cancer and other proliferative disorders, particularly of the developing hematopoietic system. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:101 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 933 of SEQ ID NO:101, b is an integer of 15 to 947, where both a and b correspond to the positions of nucleotide residues shown in SEQ ID NO:101, and where b is greater than or equal to a +14.

Features of Protein Encoded by Gene No: 92

The translation product of this gene was shown to have homology to an DNA/RNA non-specific endoneuclease (See Genbank Accession No.gi|2105496) which may implicate this gene in playing a role in DNA repair and cellular metabolism.

Preferred polypeptides of the invention comprise the following amino acid sequence: PTRPPTRPAGK (SEQ ID NO:507). Polynucleotides encoding these polypeptides are also provided.

The gene encoding the disclosed cDNA is thought to reside on chromosome 15. Accordingly, polynucleotides related to this invention are useful as a marker in linkage analysis for chromosome 15.

This gene is expressed primarily in brain, macrophages, T cells, dendritic cells, testes and pancreas tumors.

Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, and neural diseases and/or disorders including testis and pancreas tumors. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, neural, metabolic, and cancerous and wounded tissues) or bodily fluids (e.g., lymph, bile, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 230 as residues: Gln-85 to Lys-91, Pro-106 to Ser-117, Pro-124 to Ala-130, Trp-154 to Trp-160.

The tissue distribution in immune cells and tissues, combined with the homology to an endonuclease, indicates that polynucleotides and polypeptides corresponding to this gene are useful for the diagnosis and treatment of immune disorders such as testes and pancreatic tumors. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the diagnosis and/or treatment of hematopoietic disorders. This gene product is primarily expressed in hematopoietic cells and tissues, suggesting that it plays a role in the survival, proliferation, and/or differentiation of hematopoieitic lineages. Expression of this gene product in T cells and primary dendritic cells also strongly indicates a role for this protein in immune function and immune surveillance.

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Alternatively, polynucleotides and polypeptides corresponding to this gene are useful for the detection, treatment, and/or prevention of neurodegenerative disease states, and behavioral disorders. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates it plays a role in normal neural function. Potentially, this gene product is involved in synapse formation, neurotransmission, learning, cognition, homeostasis, or neuronal differentiation or survival. The protein, in addition to fragments thereof, are useful in modulating apoptosis, DNA repair, transcription, and other cellular processes. Such a use has utility in inhibiting cell proliferation and indicates this protien is useful in treating and/or prevention cancer. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:102 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1355 of SEQ ID NO:102, b is an integer of 15 to 1369, where both a and b correspond to the positions of nucleotide residues shown in SEQ ID NO:102, and where b is greater than or equal to a +14.

Features of Protein Encoded by Gene No: 93

This gene is expressed primarily in brain tissue from a patient suffering from manic depression.

Therefore, polynucleotides and polypeptides of the invention are useful as reagents for differential identification of the tissue(s) or cell type(s) present in a biological sample and for diagnosis of diseases and conditions which include, but are not limited to, neural diseases and/or disorders, particularly manic depression. Similarly, polypeptides and antibodies directed to these polypeptides are useful in providing immunological probes for differential identification of the tissue(s) or cell type(s). For a number of disorders of the above tissues or cells, particularly of the brain, expression of this gene at significantly higher or lower levels may be routinely detected in certain tissues or cell types (e.g., brain, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in brain tissue indicates that polynucleotides and polypeptides corresponding to this gene are useful for diagnosis of manic depression and other disorders of the CNS. Representative uses are described in the “Regeneration” and “Hyperproliferative Disorders” sections below, in Example 11, 15, and 18, and elsewhere herein. Briefly, polynucleotides and polypeptides corresponding to this gene are useful for the detection/treatment of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:103 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1217 of SEQ ID NO:103, b is an integer of 15 to 1231, where both a and b correspond to the positions of nucleotide residues shown in SEQ ID NO:103, and where b is greater than or equal to a +14.

Features of Protein Encoded by Gene No: 94

Preferred polypeptides of the invention comprise the following amino acid sequence: SITKYCQGCRKIGALLPWWECNMVPDTTSILKLIC (SEQ ID NO:508). Polynucleotides encoding these polypeptides are also provided.

This gene is expressed primarily in anergic T-cells.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly autoimmune disorders such as lupus. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in T-cells indicates that polynucleotides and polypeptides corresponding to this gene are useful for the diagnosis and treatment of a variety of immune system disorders. Representative uses are described in the “Immune Activity” and “Infectious Disease” sections below, in Example 11, 13, 14, 16, 18, 19, 20, and 27, and elsewhere herein. Briefly, the protein product of this gene may play a role in regulating the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g., by boosting immune responses). Expression of this gene product in T cells also strongly indicates a role for this protein in immune function and immune surveillance.

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:104 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1228 of SEQ ID NO:104, b is an integer of 15 to 1242, where both a and b correspond to the positions of nucleotide residues shown in SEQ ID NO:104, and where b is greater than or equal to a +14.

Features of Protein Encoded by Gene No: 95

This gene is expressed primarily in neutrophils and the spinal cord.

Therefore, polynucleotides and polypeptides of the invention are useful as reagents for differential identification of the tissue(s) or cell type(s) present in a biological sample and for diagnosis of diseases and conditions which include, but are not limited to, neural disorders, particularly CNS, PNS, and a variety of congenital malformations of the spinal column and injuries of the spinal cord. Similarly, polypeptides and antibodies directed to these polypeptides are useful in providing immunological probes for differential identification of the tissue(s) or cell type(s) present in a biological sample. For a number of disorders of the above tissues or cells, particularly of the central nervous system, expression of this gene at significantly higher or lower levels may be routinely detected in certain tissues or cell types (e.g., CNS, immune. hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 233 as residues: Ser-44 to His-52.

The tissue distribution in spinal cord tissue indicates that polynucleotides and polypeptides corresponding to this gene are useful for the diagnosis and/or treatment of disorders of the brain and nervous system. Such involvement may impact many processes, such as learning and cognition. Representative uses are described in the “Regeneration” and “Hyperproliferative Disorders” sections below, in Example 11, 15, and 18, and elsewhere herein. Briefly, the protein product of this gene may also be useful in the treatment of neurodegenerative disorders as schizophrenia; ALS; or Alzheimer's. Polynucleotides and polypeptides corresponding to this gene are useful for the diagnosis and treatment of a variety of immune system disorders. Representative uses are described in the “Immune Activity” and “Infectious Disease” sections below, in Example 11, 13, 14, 16, 18, 19, 20, and 27, and elsewhere herein. Briefly, the expression of this gene product indicates a role in regulating the proliferation; survival; differentiation; and/or activation of hematopoietic cell lineages, including blood stem cells. This gene product may be involved in the regulation of cytokine production, antigen presentation, or other processes suggesting a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, and scleroderma.

Moreover, the protein may represent a secreted factor that influences the differentiation or behavior of other blood cells, or that recruits hematopoietic cells to sites of injury. Thus, this gene product is thought to be useful in the expansion of stem cells and committed progenitors of various blood lineages, and in the differentiation and/or proliferation of various cell types. Furthermore, the protein may also be used to determine biological activity, raise antibodies, as tissue markers, to isolate cognate ligands or receptors, to identify agents that modulate their interactions, in addition to its use as a nutritional supplement. Protein is useful in modulating the immune response to aberrant proteins, such as those present in proliferative cells and tissues (i.e., brain cancer tissue). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:105 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1137 of SEQ ID NO:105, b is an integer of 15 to 1151, where both a and b correspond to the positions of nucleotide residues shown in SEQ ID NO:105, and where b is greater than or equal to a +14.

Features of Protein Encoded by Gene No: 96

This gene is expressed primarily in smooth muscle, and early stage human.

Therefore, polynucleotides and polypeptides of the invention are useful as reagents for differential identification of the tissue(s) or cell type(s) present in a biological sample and for diagnosis of diseases and conditions which include, but are not limited to, muscular, vascular, or cardiopulmonary disorders, particularly a variety of diseases that include wasting and muscle mass loss including amyotropic lateral sclerosis, embolism, atherosclerosis, stroke, and aneurysm. Similarly, polypeptides and antibodies directed to these polypeptides are useful in providing immunological probes for differential identification of the tissue(s) or cell type(s). For a number of disorders of the above tissues or cells, particularly of the neuromuscular system, expression of this gene at significantly higher or lower levels may be routinely detected in certain tissues or cell types (e.g., muscle, developmental, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 234 as residues: Leu-37 to Trp-44.

The tissue distribution in smooth muscle indicates that polynucleotides and polypeptides corresponding to this gene are useful for the detection, treatment, and/or prevention of various muscle disorders, such as muscular dystrophy, cardiomyopathy, fibroids, myomas, vascular disorders, and rhabdomyosarcomas. Moreover, the expression within embryonic tissue and other cellular sources marked by proliferating cells indicates this protein may play a role in the regulation of cellular division, and may show utility in the diagnosis, treatment, and/or prevention of developmental diseases and disorders, cancer, and other proliferative conditions. Representative uses —are described in the “Hyperproliferative Disorders” and “Regeneration” sections below and elsewhere herein.

Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:106 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1614 of SEQ ID NO:106, b is an integer of 15 to 1628, where both a and b correspond to the positions of nucleotide residues shown in SEQ ID NO:106, and where b is greater than or equal to a +14.

Features of Protein Encoded by Gene No: 97

This gene is expressed primarily in the brain, and, to a lesser extent, in neutrophils.

Therefore, polynucleotides and polypeptides of the invention are useful as reagents for differential identification of the tissue(s) or cell type(s) present in a biological sample and for diagnosis of diseases and conditions which include, but are not limited to, disorders affecting the brain and central nervous system, such as Alzheimer's disease. Similarly, polypeptides and antibodies directed to these polypeptides are useful in providing immunological probes for differential identification of the tissue(s) or cell type(s). For a number of disorders of the above tissues or cells, particularly of the brain and central nervous system, expression of this gene at significantly higher or lower levels may be routinely detected in certain tissues or cell types (e.g., brain, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in brain tissue indicates that polynucleotides and polypeptides corresponding to this gene are useful for the detection/treatment of neurodegenerative disease states and behavioural disorders. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, the gene or gene product may also play a role in the treatment and/or detection of developmental disorders associated with the developing embryo. Furthermore, the protein may also be used to determine biological activity, to raise antibodies, as tissue markers, to isolate cognate ligands or receptors, to identify agents that modulate their interactions, in addition to its use as a nutritional supplement. The protein is useful in the modulation of the immune response to aberrant proteins, as may be present in rapidly proliferating cells and tissues (e.g., brain cancer, etc.). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:107 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1451 of SEQ ID NO:107, b is an integer of 15 to 1465, where both a and b correspond to the positions of nucleotide residues shown in SEQ ID NO:107, and where b is greater than or equal to a +14.

Features of Protein Encoded by Gene No: 98

Preferred polypeptides of the invention comprise the following amino acid sequence:

| | |
|---|---|
| SLQVLRTLGSKCGDFLRSRFCKDVLPKLAGSLVTQAPISARAGPVYSHTLAFK | (SEQ ID NO:509) |
| LQLAVLQGLGPLCERLDLGEGDLNKVADACLIYLSVKQPVKLQEAARSVFLH | |
| LMKVDPDSTWFLLNELYCPVQFTPPHPSLHPVQLXGASGQQNPXHDQRAPA | |
| AQGAAVTLLPHHRGHRSLPYCQPEAGLTPPRP, | |
| GADGNVSDFDNEEEEQSVPPKVDENDTRPDVEPPLPLQIQIAMDVMERCIHLL | (SEQ ID NO:510) |
| SDKNLQIRLKVLDVLDL | |
| CVVVLQSHKNQLLPLAHQAWPSLVHRLTRDAPLAVLRAFKFYVPWEASVVT | |
| FFAAGSAKMSCQSWLAP, | |
| TLGSKCGDFLRSRFCKDVLPKLAGSL, | (SEQ ID NO:511) |
| PVYSHTLAFKLQLAVLQGLGPLCERLDLG, | (SEQ ID NO:512) |
| SVPPKVDENDTRPDVEPPLPLQIQIAM, and/or | (SEQ ID NO:513) |
| WPSLVHRLTRDAPLAVLRAFKFYVPW. | (SEQ ID NO:514) |

Polynucleotides encoding these polypeptides are also provided.

This gene is expressed primarily in kidney cortex, hemangiopericytoma, fetal spleen, infant brain, and, to a lesser extent, in pancreas, lymph node, fetal liver, ovarian tumor, T-cells and other tissues.

Therefore, polynucleotides and polypeptides of the invention are useful as reagents for identification of the tissue(s) or cell type(s) present in a biological sample and for diagnosis of diseases and conditions which include, but are not limited to, renal, immune, neural, or developmental diseases and/or disorders, particularly tumors. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues and cell types (e.g., renal, immune, neural, developmental, reproductive, ovarian, hepatic, metabolic, and cancerous and wounded tissues) or bodily fluids (e.g., lymph, serum, bile, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 236 as residues: Pro-24 to Pro-37.

The tissue distribution in proliferating tissues and cells, combined with its distribution in developing tissues indicates that polynucleotides and polypeptides corresponding to this gene are useful for diagnosing and treating tumors. The expression within fetal tissue and other cellular sources marked by proliferating cells (i.e., ovarian tumor, etc.) indicates this protein may play a role in the regulation of cellular division, and may show utility in the diagnosis, treatment, and/or prevention of developmental diseases and disorders, cancer, and other proliferative conditions. Representative uses are described in the “Hyperproliferative Disorders” and “Regeneration” sections below and elsewhere herein.

Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:108 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1251 of SEQ ID NO:108, b is an integer of 15 to 1265, where both a and b correspond to the positions of nucleotide residues shown in SEQ ID NO:108, and where b is greater than or equal to a +14.

Features of Protein Encoded by Gene No: 99

Preferred polypeptides of the invention comprise the following amino acid sequence:

| | |
|---|---|
| SLGISTFGIMVFSVYFGGIMISIPYSGISFGNKKELNIDSCYNMVNLKNIMFSER | (SEQ ID NO:515) |
| SQT, | |
| HASGNNDPLWFLTYL, | (SEQ ID NO:516) |
| MVFSVYFGGIMISIPYSGISF, and/or | (SEQ ID NO:517) |
| FGNKKELNIDSCYNMVNLKN. | (SEQ ID NO:518) |

Polynucleotides encoding these polypeptides are also provided.

This gene is expressed primarily in T-cells, spleen, and pancreas islet cell tumor.

Therefore, polynucleotides and polypeptides of the invention are useful as reagents for differential identification of the tissue(s) or cell type(s) present in a biological sample and for diagnosis of diseases and conditions which include, but are not limited to, immune or endocrine diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, endocrine, pancreatic, cancerous and wounded tissues) or bodily fluids (e.g., lymph, serum, bile, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 237 as residues: Thr-24 to Arg-29.

The tissue distribution of this gene predominantly in cell types or tissues associated with the immune system indicates that the gene could be important for the treatment and/or detection of immune or hematopoietic disorders including, but not limited to, arthritis, asthma, immunodeficiency diseases and leukemia. Representative uses are described in the “Immune Activity” and “Infectious Disease” sections below, in Example 11, 13, 14, 16, 18, 19, 20, and 27, and elsewhere herein. Moreover, the expression within pancreatic tissues indicates that the protein product of this gene may be useful in the treatment or prevention of a variety of metabolic disorders, such as diabetes. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:109 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 992 of SEQ ID NO:109, b is an integer of 15 to 1006, where both a and b correspond to the positions of nucleotide residues shown in SEQ ID NO:109, and where b is greater than or equal to a +14.

Features of Protein Encoded by Gene No: 100

The gene encoding the disclosed cDNA is believed to reside on the X chromosome. Accordingly, polynucleotides related to this invention are useful as a marker in linkage analysis for the X chromosome.

Preferred polypeptides of the invention comprise the following amino acid sequence:

| | |
|---|---|
| MHQQKRQPELVEGNLPVFVFPTELIFYADDQSTHKQVLTLYNPYEFALDFKV | (SEQ ID NO:519) |
| LCTTPNKYVVVDAAGAVKPQCCVDIVIRHRDVRSCHYGVIDKFRLQVSEQSQ | |
| RKALGKKRGCCYSSPISKRTTKGRRGKKIKGTFNXXFIF | |

and fragements thereof, preferrably biologically active fragments thereof. Polynucleotides encoding such polypeptides and polypeptide fragments are also provided.

This gene is expressed primarily in urinary bladder carcinoma HSC172 cells, and to a lesser extent in human adult heart, lung, osteoclastoma, and liver tissues.

Therefore, polynucleotides and polypeptides of the invention are useful as reagents for differential identification of the tissue(s) or cell type(s) present in a biological sample and for diagnosis of diseases and conditions which include, but are not limited to, urogenital, or renal disorders, particularly urinary bladder carcinoma and other cancers. Similarly, polypeptides and antibodies directed to these polypeptides are useful in providing immunological probes for differential identification of the tissue(s) or cell type(s). For a number of disorders of the above tissues or cells, particularly of the bladder, expression of this gene at significantly higher or lower levels may be routinely detected in certain tissues or cell types (e.g., renal, cardiopulmonary, hepatic, and cancerous and wounded tissues) or bodily fluids (e.g., lymph, serum, bile, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 238 as residues: Gly-18 to Lys-23, Pro-31 to Gly-38.

The tissue distribution in urinary bladder carcinoma indicates that polynucleotides and polypeptides corresponding to this gene are useful for the diagnosis, treatment and/or therapeutic targeting of urinary bladder carcinoma, osteoclastoma, and other cancers. Additionally, the tissue distribution in heart, lung and osteocarcinoma indicates an indication for the use of this gene and gene product in the diagnosis and/or treatment of disorders in the heart and lung. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:110 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2200 of SEQ ID NO:110, b is an integer of 15 to 2214, where both a and b correspond to the positions of nucleotide residues shown in SEQ ID NO:110, and where b is greater than or equal to a +14.

Features of Protein Encoded by Gene No: 101

Preferred polypeptides of the invention comprise the following amino acid sequence:

| | |
|---|---|
| MNSFSVIASIVVLLPFPGLSVSACLPSHSHQCKTFILLFLPSSEKTLXXXPPSHSS | (SEQ ID NO:520) |
| TLGGQGGQIMRSGDRXHXG, | |
| VVFFXXFFEMESHSVAQAGVQWRNLGSLQALPPGFMPFSCLSLPGSWDYRRP | (SEQ ID NO:521) |
| PPSPANLXCIFSRDGGHHVSQXGLDLLTS, | |
| IVVLLPFPGLSVSACLPSHSHQCKTFIL, and/or | (SEQ ID NO:522) |
| PGFMPFSCLSLPGSWDYRRPPPSPAN. | (SEQ ID NO:523) |

Polynucleotides encoding these polypeptides are also provided.

This gene is expressed primarily in adipose tissue.

Therefore, polynucleotides and polypeptides of the invention are useful as reagents for differential identification of the tissue(s) or cell type(s) present in a biological sample and for diagnosis of diseases and conditions which include, but are not limited to, obesity and other metabolic disorders. Similarly, polypeptides and antibodies directed to these polypeptides are useful in providing immunological probes for differential identification of the tissue(s) or cell type(s). For a number of disorders of the above tissues or cells, particularly of the endocrine system, expression of this gene at significantly higher or lower levels may be routinely detected in certain tissues or cell types (e.g., adipose, metabolic, neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 239 as residues: Arg-28 to Asn-33.

The tissue distribution in adipose tissue indicates that polynucleotides and polypeptides corresponding to this gene are useful for the diagnosis and/or treatment of obesity and other metabolic and endocrine conditions or disorders. Furthermore, the protein product of this gene may show utility in ameliorating conditions which occur secondary to aberrant fatty-acid metabolism (e.g., aberrant myelin sheath development), either directly or indirectly. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:111 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1439 of SEQ ID NO:111, b is an integer of 15 to 1453, where both a and b correspond to the positions of nucleotide residues shown in SEQ ID NO:111, and where b is greater than or equal to a +14.

Features of Protein Encoded by Gene No: 102

Preferred polypeptides of the invention comprise the following amino acid sequence:

| | |
|---|---|
| YRFKNPKCRLFSVPCR, | (SEQ ID NO:524) |
| TQNRELLAWKPKGRDDICTSHNTTHIQKMPGE | (SEQ ID NO:525) |
| ANSCCPRGAKSYHIDCWPPALFPRCVAYLFLNKPATLRKKYYCKPYHTQLHP | |
| AWHREKSAFWIFERVSQS KQSLTSLVYSVNELLVLSNLAQWALG, | |
| AWKPKGTDDICTSHNTTHIQKMP, | (SEQ ID NO:526) |
| CPRGAKSYHIDCWPPALFPRCVAYL, | (SEQ ID NO:527), |
| SYHIDCWPPALFPRCVAYLFLNKPAT, and/or | (SEQ ID NO:528) |
| RKKYYCKPYHTQLHPAWHREKSAFWIFET. | (SEQ ID NO:529) |

Polynucleotides encoding these polypeptides are also provided.

This gene is expressed primarily in dendritic cells and activated monocytes.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly inflammation, immune defects, mutiple myeloma, or immuodeficiecies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 240 as residues: Thr-27 to Arg-33.

The tissue distribution in dendritic cells and monocytes indicates that polynucleotides and polypeptides corresponding to this gene are useful for the diagnosis and/or treatment of inflammatory and immune disorders such as cancers, particularly of dendritic cells and monocytes, but also of hematopoietic progenitors. Similarly, polynucleotides and polypeptides corresponding to this gene are useful for the treatment and/or diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency, etc. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:112 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1538 of SEQ ID NO:112, b is an integer of 15 to 1552, where both a and b correspond to the positions of nucleotide residues shown in SEQ ID NO:112, and where b is greater than or equal to a +14.

Features of Protein Encoded by Gene No: 103

When tested against K562 leukemia cell lines, supernatants removed from cells containing this gene activated the ISRE assay. Thus, it is likely that this gene activates leukemia cells through the Jak-STAT signal transduction pathway. The interferon-sensitive response element is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the ISRE element, can be used to indicate proteins involved in the proliferation and differentiation of cells.

The gene encoding the disclosed cDNA is thought to reside on chromosome 5. Accordingly, polynucleotides related to this invention are useful as a marker in linkage analysis for chromosome 5.

This gene is expressed primarily in placenta, adipose tissue and fibroblasts.

Therefore, polynucleotides and polypeptides of the invention are useful as reagents for differential identification of the tissue(s) or cell type(s) present in a biological sample and for diagnosis of diseases and conditions which include, but are not limited to, disorders of the skin, developing organs and metabolic disorders. Similarly, polypeptides and antibodies directed to these polypeptides are useful in providing immunological probes for differential identification of the tissue(s) or cell type(s). For a number of disorders of the above tissues or cells, particularly of the epidermal system, metabolic system and embryogenesis, expression of this gene at significantly higher or lower levels may be routinely detected in certain tissues or cell types (e.g., epidermal, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution indicates that polynucleotides and polypeptides corresponding to this gene are useful for the treatment and/or diagnosis of disorders of the epidermal system, metabolic system and embryogenesis. Furthermore, the tissue distribution indicates that polynucleotides and polypeptides corresponding to this gene are useful for the diagnosis and/or treatment of disorders of the placenta. Specific expression within the placenta indicates that this gene product may play a role in the proper establishment and maintenance of placental function. Alternately, this gene product may be produced by the placenta and then transported to the embryo, where it may play a crucial role in the development and/or survival of the developing embryo or fetus. Expression of this gene product in a vascular-rich tissue such as the placenta also indicates that this gene product may be produced more generally in endothelial cells or within the circulation. In such instances, it may play more generalized roles in vascular function, such as in angiogenesis. It may also be produced in the vasculature and have effects on other cells within the circulation, such as hematopoietic cells. It may serve to promote the proliferation, survival, activation, and/or differentiation of hematopoietic cells, as well as other cells throughout the body.

Many polynucleotide sequences, such as EST sequences, are publicly available and accessible through sequence databases. Some of these sequences are related to SEQ ID NO:113 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1475 of SEQ ID NO:113, b is an integer of 15 to 1489, where both a and b correspond to the positions of nucleotide residues shown in SEQ ID NO:113, and where b is greater than or equal to a +14.

Features of Protein Encoded by Gene No: 104

5 Preferred polypeptides of the invention comprise the following amino acid sequence: ICLDSCSQVSVTSLWSFLRVHSLVQTLW (SEQ ID NO:530). Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the immune system, including neutropenia, cancer, inflammatory diseases and allergies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 242 as residues: Ala-35 to Asp-44.

The tissue distribution in neutrophils indicates that polynucleotides and polypeptides corresponding to this gene are useful for the treatment and/or diagnosis of diseases of the immune system. Representative uses are described in the “Immune Activity” and “Infectious Disease” sections below, in Example 11, 13, 14, 16, 18, 19, 20, and 27, and elsewhere herein. Briefly, the expression primarily in neutrophils, indicates this protein may be useful as a growth factor for the differentiation or proliferation of neutrophils for the treatment of neutropenia following chemotherapy or may be useful in the treatment of immune dysfunction or anti-inflamatory by inhibiting infiltration of neutrophils to the site of injury or distress. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:114 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 593 of SEQ ID NO:114, b is an integer of 15 to 607, where both a and b correspond to the positions of nucleotide residues shown in SEQ ID NO:114, and where b is greater than or equal to a +14.

Features of Protein Encoded by Gene No: 105

This gene is expressed primarily in osteoarthritic cells, and stromal cells.

Therefore, polynucleotides and polypeptides of the invention are useful as reagents for differential identification of the tissue(s) or cell type(s) present in a biological sample and for diagnosis of diseases and conditions which include, but are not limited to, skeletal, immune, and hematopoietic diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, skeletal, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in stromal cells indicates that polynucleotides and polypeptides corresponding to this gene are useful for the diagnosis and/or treatment of immune disorders. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Representative uses are described in the “Immune Activity” and “Infectious Disease” sections below, in Example 11, 13, 14, 16, 18, 19, 20, and 27, and elsewhere herein. Briefly, the protein can be used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues.

In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO: 115 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1484 of SEQ ID NO:115, b is an integer of 15 to 1498, where both a and b correspond to the positions of nucleotide residues shown in SEQ ID NO:115, and where b is greater than or equal to a +14.

Features of Protein Encoded by Gene No: 106

Preferred polypeptides of the invention comprise the following amino acid sequence:

| | |
|---|---|
| HYCCDFGTSLLGFYVPFHYYVHMVNIILTTIDFYHYKFCCSQNANKHCFKHF | (SEQ ID NO:531) |
| QIMTTVPYLNINKENLRFKNIFK, | |
| TSLLGFYVPFHYYVHMVNIILTTIDFY, and/or | (SEQ ID NO:532) |
| FQIMTTVPYLNINKENLRFKNI. | (SEQ ID NO:533) |

Polynucleotides encoding these polypeptides are also provided.

The gene encoding the disclosed cDNA is believed to reside on chromosome 5. Accordingly, polynucleotides related to this invention are useful as a marker in linkage analysis for chromosome 5.

This gene is expressed primarily in spleen, breast, placenta, ovarian cancer, and, to a lesser extent, in B-cell lymphoma, pancreas tumor, osteoclastoma, thyroid, bone marrow, fetal liver, and stromal cells.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders characterized by immune cell activation and proliferation, particularly of the reproductive system. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, reproductive, metabolic, skeletal, endocrine, hepatic, placental, ovarian, and cancerous and wounded tissues) or bodily fluids (e.g., lymph, serum, bile, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 244 as residues: Ser-21 to Ser-27.

The tissue distribution in spleen and reproductive tissues indicates that the product of this gene is useful for modifying or detecting the proliferation or activation of cells in the hematopoietic system. Representative uses are described in the “Immune Activity” and “Infectious Disease” sections below, in Example 11, 13, 14, 16, 18, 19, 20, and 27, and elsewhere herein. Briefly, the secreted protein can be used to determine biological activity, to raise antibodies, as tissue markers, to isolate cognate ligands or receptors, to identify agents that modulate their interactions and as nutritional supplements. It may also have a very wide range of biological acitivities.

Typical of these are cytokine, cell proliferation/differentiation modulating activity or induction of other cytokines; immunostimulating/immunosuppressant activities (e.g., for treating human immunodeficiency virus infection, cancer, autoimmune diseases and allergy); regulation of hematopoiesis (e.g., for treating anaemia or as adjunct to chemotherapy); stimulation or growth of bone, cartilage, tendons, ligaments and/or nerves (e.g., for treating wounds, stimulation of follicle stimulating hormone (for control of fertility); chemotactic and chemokinetic activities (e.g., for treating infections, tumors); hemostatic or thrombolytic activity (e.g., for treating haemophilia, cardiac infarction etc.); anti-inflammatory activity (e.g., for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:116 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1783 of SEQ ID NO:116, b is an integer of 15 to 1797, where both a and b correspond to the positions of nucleotide residues shown in SEQ ID NO:116, and where b is greater than or equal to a +14.

Features of Protein Encoded by Gene No: 107

Preferred polypeptides of the invention comprise the following amino acid sequence:

| | |
|---|---|
| ISESMSLVRSLQFYRGKNRAERTVISSSSHSCHLIDLEFQPRSDGEVSISFLEKG | (SEQ ID NO:534) |
| VELRWGMGLEDLIGLGLGVSTRRSTVRRKEPTKAGMHTACSEEMEPENREN, | |
| DGSRSVAQARVQWHHRGSLPPLPPRFKQFPLRHLRVGGITGACRHTQIIFVVL | (SEQ ID NO:535) |
| VQMGFHHVGQAGLELLTSGDPPALASQSAGITGVSHSTRPKLLSWLPSDNLL | |
| GMALYSIQWALLANSLYFQVPSPLSMLCAFLPLQVPSA, | |
| RGKNRAERTVISSSSHSCHLIDLEFQP, | (SEQ ID NO:536) |
| LGLGVSTRRSTVRRKEPTKAGMHTACSEEMEP, | (SEQ ID NO:537) |
| GDPPALASQSAGITGVSHSTRPKL, and/or | (SEQ ID NO:538) |
| ALYSIQWALLANSLYFQVPSPLSML. | (SEQ ID NO :539) |

Polynucleotides encoding these polypeptides are also provided.

This gene is expressed primarily in bone marrow, and, to a lesser extent, in dura mater.

Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, or neural diseases and/or disorders, particularly bone marrow related diseases such as multiple myeloma, immunodeficiencies, and hematopoietic disorders. Similarly, polypeptides and antibodies directed to these polypeptides are useful in providing immunological probes for differential identification of the tissue(s) or cell type(s). For a number of disorders of the above tissues or cells, particularly of the bone marrow, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 245 as residues: Gln-46 to Asn-56.

The tissue distribution in bone marrow indicates that polynucleotides and polypeptides corresponding to this gene are useful for the diagnosis and/or treatment of central nervous system disorders and hematopoietic system developmental disorders. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the treatment and/or diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:117 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 938 of SEQ ID NO:117, b is an integer of 15 to 952, where both a and b correspond to the positions of nucleotide residues shown in SEQ ID NO:117, and where b is greater than or equal to a +14.

Features of Protein Encoded by Gene No: 108

Preferred polypeptides of the invention comprise the following amino acid sequence:

(SEQ ID NO:540)

DRILLFYSRDGQTTSKGPNPACCLFLLKKFYWNTA, and/or

(SEQ ID NO:541)

DGQTTSKGPNPACCLFLLKKF.

Polynucleotides encoding these polypeptides are also provided.

This gene is expressed primarily in early stage human brain tissue.

Therefore, polynucleotides and polypeptides of the invention are useful as reagents for differential identification of the tissue(s) or cell type(s) present in a biological sample and for diagnosis of diseases and conditions which include, but are not limited to, neural diseases and/or disorders, particularly developmental disorders of the brain. Similarly, polypeptides and antibodies directed to these polypeptides are useful in providing immunological probes for differential identification of the tissue(s) or cell type(s). For a number of disorders of the above tissues or cells, particularly of the early stage human brain, expression of this gene at significantly higher or lower levels may be routinely detected in certain tissues and cell types (e.g., neural, developmental, and cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 246 as residues: Asn-16 to Gln-21.

The tissue distribution in early stage brain tissue indicates that polynucleotides and polypeptides corresponding to this gene are useful for the diagnosis and/or treatment of brain developmental disorders. Representative uses are described in the “Regeneration” and “Hyperproliferative Disorders” sections below, in Example 11, 15, and 18, and elsewhere herein. Briefly, polynucleotides and polypeptides corresponding to this gene are useful for the detection/treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival.

Moreover, the gene or gene product may also play a role in the treatment and/or detection of developmental disorders associated with the developing embryo, sexually-linked disorders, or disorders of the cardiovascular system. Moreover, the expression within embryonic tissue indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO: 118 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1171 of SEQ ID NO:118, b is an integer of 15 to 1185, where both a and b correspond to the positions of nucleotide residues shown in SEQ ID NO:118, and where b is greater than or equal to a +14.

Features of Protein Encoded by Gene No: 109

The translation product of this gene was shown to have homology to the HP1-BP74 protein from Mus musculus (See Genbank Accession No. gn∥PID|e256809; all references available through this accession are hereby incorporated herein by reference, for example, EMBO J. 15 (23), 6701-6715 (1996)) which is thought to be important in chromatin structure and function. Based on the sequence similarity, the translation product of this gene is expected to share biological activities with DNA binding proteins. Such activities are known in the art, some of which are described elsewhere herein.

Preferred polypeptides of the invention comprise the following amino acid sequence:

| | |
|---|---|
| DPRVRRTLDLGITLYLFLYIFLSL, | (SEQ ID NO:542) |
| PALGECCLDAFLFLLGKQLKKSGEKPLLGGSLMEYAILSAIAAMNEPKTCSTT | (SEQ ID NO:543) |
| ALKKYVLENHPGTNSNYQMHLLKKTLQKCEKNGWMEQISGKGFSGRFQLCF | |
| PYYPSPGVLFPKKEPDDSRDEDEDEDESSEEDSEDEEPPPKRRLQKKTPAKSP | |
| GKAASVKQRGSKPAPKVSAAQRGKARPLPKKAPPKAKTPAKKTRPSSTVIKK | |
| PSGGSSKKPATSARKEVKLPGLGLSTMKKSFRVKK, | |
| DFEFHHDTLFSYKIYFFTLKDFFMVDLPLPGNFTSFLALVAGFFEEPPLGFLMT | (SEQ ID NO:544) |
| VDEGLVFLAGVLALGGAFLGKGLAFPRWAAETLGAGLDPLCFTDAAFPGDL | |
| AGVFFCNLLLGGGSSSSESSSDDSSSSSSSSLESSGSFFGNRTPGLG, | |
| CLDAFLFLLGKQLKKSGEKPLLGGSLME, | (SEQ ID NO:545) |
| YQMHLLKKTLQKCEKNGWMEQISGKGFSGT, | (SEQ ID NO:546) |
| KTPAKSPGKAASVKQRGSKPAPKVSAAQ, | (SEQ ID NO:547) |
| SSKKPATSARKEVKLPGKGKSTMKKSFR, | (SEQ ID NO:548) |
| VDEGLVFLAGVLALGGAFLGKGL, and/or | (SEQ ID NO:549) |
| GLDPLCFRDAADPGDLAGVFFCNLL. | (SEQ ID NO:550) |

Polynucleotides encoding these polypeptides are also provided.

This gene is expressed primarily in bone marrow stromal cells, and, to a lesser extent, in human osteoblasts and T cells (helper I).

Therefore, polynucleotides and polypeptides of the invention are useful as reagents for differential identification of the tissue(s) or cell type(s) present in a biological sample and for diagnosis of diseases and conditions which include, but are not limited to, connective tissues, haemopoietic, or immune diseases and/or disorders. Similarly, polypeptides and antibodies directed to these polypeptides are useful in providing immunological probes for differential identification of the tissue(s) or cell type(s). For a number of disorders of the above tissues or cells, particularly of the skeletal and immune systems, expression of this gene at significantly higher or lower levels may be routinely detected in certain tissues and cell types (e.g., connective, hematopoietic, immune, skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 247 as residues: Glu-18 to Cys-38.

The tissue distribution in bone marrow stromal cells and T-cells suggest that polynucleotides and polypeptides corresponding to this gene are useful for the diagnosis and/or treatment of defects of stromal development, and immune system disorders. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc.

In addition, this gene product may have commercial utility in the expansion of stem cells and committed progenitors of various blood lineages, and in the differentiation and/or proliferation of various cell types. Moreover, the expression of this gene product in osteoblasts would suggest a role in the detection and treatment of disorders and conditions afflicting the skeletal system, in particular osteoporosis, bone cancer, connective tissue disorders (e.g. arthritis, trauma, tendonitis, chrondomalacia and inflammation). The protein is also useful in the diagnosis or treatment of various autoimmune disorders (i.e., rheumatoid arthritis, lupus, scleroderma, and dermatomyositis), dwarfism, spinal deformation, joint abnormalities, and chondrodysplasias (i.e. spondyloepiphyseal dysplasia congenita, familial osteoarthritis, Atelosteogenesis type II, metaphyseal chondrodysplasia type Schmid, etc.). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:119 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1084 of SEQ ID NO:119, b is an integer of 15 to 1098, where both a and b correspond to the positions of nucleotide residues shown in SEQ ID NO:119, and where b is greater than or equal to a +14.

Features of Protein Encoded by Gene No: 110

This gene is expressed primarily in rhabdomyosarcoma, CD34 positive cells, breast lymph nodes, neutrophils and endothelial cells.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, developmental, proliferative, and vascular disorders, particularly fibroids or atherosclerosis. Similarly, polypeptides and antibodies directed to these polypeptides are useful in providing immunological probes for differential identification of the tissue(s) or cell type(s). For a number of disorders of the above tissues or cells, particularly of the immune or hematopoietic systems, expression of this gene at significantly higher or lower levels may be routinely detected in certain tissues or cell types (e.g., immune, hematopoietic, developmental, vascular, endothelial, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils and lymph nodes indicates that polynucleotides and polypeptides corresponding to this gene are useful for the diagnosis, treatment and/or intervention of disorders in the immune or hematopoietic systems. Similarly, the secreted protein can also be used to determine biological activity, to raise antibodies, as tissue markers, to isolate cognate ligands or receptors, to identify agents that modulate their interactions, and as nutritional supplements. It may also have a very wide range of biological acitivities. Typical of these are cytokine, cell proliferation/differentiation modulating activity or induction of other cytokines; immunostimulating/immunosuppressant activities (e.g., for treating human immunodeficiency virus infection, cancer, autoimmune diseases and allergy); regulation of hematopoiesis (e.g., for treating anaemia or as adjunct to chemotherapy); stimulation or growth of bone, cartilage, tendons, ligaments and/or nerves (e.g., for treating wounds, stimulation of follicle stimulating hormone (for control of fertility); chemotactic and chemokinetic activities (e.g., for treating infections, tumors); hemostatic or thrombolytic activity (e.g., for treating haemophilia, cardiac infarction etc.); anti-inflammatory activity (e.g., for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. The protein may also show utility in the treatment or prevention of a variety of vascular disorders, particularly embolism, thrombis, aneurysms, stroke, or athersclerosis. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:120 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 791 of SEQ ID NO:120, b is an integer of 15 to 805, where both a and b correspond to the positions of nucleotide residues shown in SEQ ID NO:120, and where b is greater than or equal to a +14.

Features of Protein Encoded by Gene No: 111

Preferred polypeptides of the invention comprise the following amino acid sequence:

| | |
|---|---|
| TMLFYLSSQPDWQLDFFRVSFNGPVFFIIIFNDRAGFRMQALVSQAACRRSRY | (SEQ ID NO:551) |
| KLSVVY, and/or | |
| DRAGFRMQALVSQAACRRSRYKL. | (SEQ ID NO:552) |

Polynucleotides encoding these polypeptides are also provided. More preferrably, polypeptides of the invention comprise the following amino acid sequence:

| | |
|---|---|
| MAMGFPGYDLSADDIAGKFQFSRGMRRSYDAGFKLMVVEYAESTNNCQAA | (SEQ ID NO:553) |
| KQFGVLEKNVRDWRKVKPQLQNAHAMRRAFRGPXNGRFALVDQRVAEYV | |
| RYMQAKGDPITREAMQLKALEIAQEMNIPEKGFKASLGWCRRMMRRYDLSL | |
| RHKVPVPQHLPEDLTEKLVTYQRSVLALRRAHDYEYAXMGNADETPICLEVP | |
| SRVTVDNQGEKPVLVKTPGREKLKITAMLGVLADGRKLPPYIILRGTYIPPGK | |
| FPSGMEIRCHRYGWMTEDLMQDWLEVVWRRRTGAVPKQRGMLILNGFRGH | |
| ATDSVKNSMESMNTDMVIXPGGLTSQLQVLDVVVYKPLNDSVRAQYSNWL | |
| LAGNLALSPTGNAKKPPLGLFLEWVMVAWNSISSESIVQGFKKCHISSNLEEE | |
| DDVLWEIESELPGGGEPPKDCDTESMAESN | |

and fragment of such sequence described elsewhere herein. Polynucleotides encoding such polypeptides and polypeptide fragments are also provided.

The gene encoding the disclosed cDNA is believed to reside on chromosome 1. Accordingly, polynucleotides related to this invention are useful as a marker in linkage analysis for chromosome 1.

This gene is expressed primarily in human cerebellum, and to a lesser extent in colon carcinoma cells, activated T-cells, fetal spleen, and placental tissues.

Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, or neural disorders, particularly neurodegenerative disorders. Similarly, polypeptides and antibodies directed to these polypeptides are useful in providing immunological probes for differential identification of the tissue(s) or cell type(s). For a number of disorders of the above tissues or cells, particularly of the immune or central nervous systems, expression of this gene at significantly higher or lower levels may be routinely detected in certain tissues or cell types (e.g., neural, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in human cerebellum indicates that polynucleotides and polypeptides corresponding to this gene are useful for the diagnosis and/or treatment of diseases in the central nervous system and immune disorders. Moreover, polynucleotides and polypeptides corresponding to this gene are useful for the detection and/or treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:121 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 3421 of SEQ ID NO:121, b is an integer of 15 to 3435, where both a and b correspond to the positions of nucleotide residues shown in SEQ ID NO:121, and where b is greater than or equal to a +14.

Features of Protein Encoded by Gene No: 112

The translation product of this gene was shown to have homology to the human T-Star protein (See Genbank Accession No. gi|3273832). Based on the sequence similarity, the translation product of this gene is expected to share biological activities with Sma68 proteins. Such activities are known in the art, some of which are described elsewhere herein. For example, see Proc. Natl. Acad. Sci. U.S.A. 96, 2710-2715 (1999), which is hereby incorporated herein by reference.

Preferred polypeptides of the invention comprise the following amino acid sequence: GQEEWTNSRHKAPSARTAKGVYRDQPYGRY (SEQ ID NO:554). Polynucleotides encoding these polypeptides are also provided.

The gene encoding the disclosed cDNA is thought to reside on chromosome 8. Accordingly, polynucleotides related to this invention are useful as a marker in linkage analysis for chromosome 8.

This gene is expressed primarily in testes, fetal brain, fetal liver, and, to a lesser extent, in retina.

Therefore, polynucleotides and polypeptides of the invention are useful as reagents for differential identification of the tissue(s) or cell type(s) present in a biological sample and for diagnosis of diseases and conditions which include, but are not limited to, brain, developmental, immune, and liver diseases and/or diseases. Similarly, polypeptides and antibodies directed to these polypeptides are useful in providing immunological probes for differential identification of the tissue(s) or cell type(s). For a number of disorders of the above tissues or cells, particularly of the liver and brain expression of this gene at significantly higher or lower levels may be routinely detected in certain tissues or cell types (e.g., immune, hematopoietic, visual, neural, reproductive, hepatic, and cancerous and wounded tissues) or bodily fluids (e.g., lymph, bile, serum, plasma, urine, amniotic fluid, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in brain and liver tissues indicates that polynucleotides and polypeptides corresponding to this gene are useful for the treatment of neural, hepatic, or metabolic diseases. Representative uses are described in the “Regeneration”, “Infectious Disease”, and “Hyperproliferative Disorders” sections below, in Example 11, 15, and 18, and elsewhere herein. Briefly, the tissue distribution indicates that polynucleotides and polypeptides corresponding to this gene are useful for the diagnosis and/or treatment of disorders of the brain and nervous system. Such involvement may impact many processes, such as learning and cognition. It may also be useful in the treatment of such neurodegenerative disorders as schizophrenia; ALS; or Alzheimer's. Moreover, indicates that polynucleotides and polypeptides corresponding to this gene are useful for the detection and treatment of liver disorders and cancers (e.g., hepatoblastoma, jaundice, hepatitis, liver metabolic diseases and conditions that are attributable to the differentiation of hepatocyte progenitor cells). Additionally, the tissue distribution indicates that polynucleotides and polypeptides corresponding to this gene are useful for the treatment and diagnosis of conditions concerning proper testicular function (e.g., endocrine function, sperm maturation), as well as cancer. Therefore, this gene product is useful in the treatment of male infertility and/or impotence. This gene product is also useful in assays designed to identify binding agents as such agents (antagonists) -are useful as male contraceptive agents. Similarly, the protein is believed to by useful in the treatment and/or diagnosis of testicular cancer. The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:122 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1006 of SEQ ID NO:122, b is an integer of 15 to 1020, where both a and b correspond to the positions of nucleotide residues shown in SEQ ID NO:122, and where b is greater than or equal to a +14.

Features of Protein Encoded by Gene No: 113

Preferred polypeptides of the invention comprise the following amino acid sequence: ILAISLAQNFTPSWKGGERECSDLYL (SEQ ID NO:555). Polynucleotides encoding these polypeptides are also provided.

This gene is expressed primarily in apoptotic T-cells, and, to a lesser extent, in the frontal cortex of the brain.

Therefore, polynucleotides and polypeptides of the invention are useful as reagents for differential identification of the tissue(s) or cell type(s) present in a biological sample and for diagnosis of diseases and conditions which include, but are not limited to, immune or neural disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 251 as residues: Arg-19 to Gly-36, Val-44 to Leu-59.

The tissue distribution in apoptotic T-cells indicates that polynucleotides and polypeptides corresponding to this gene are useful for the treatment and diagnosis of immune disorders. Representative uses are described in the “Immune Activity”, “Regeneration”, and “Infectious Disease” sections below, in Example 11, 13, 14, 15, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Expression of this gene product in T cells also strongly indicates a role for this protein in immune function and immune surveillance. Alternatively, polynucleotides and polypeptides corresponding to this gene are useful for the detection/treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered bahaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:123 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1364 of SEQ ID NO:123, b is an integer of 15 to 1378, where both a and b correspond to the positions of nucleotide residues shown in SEQ ID NO:123, and where b is greater than or equal to a +14.

Features of Protein Encoded by Gene No: 114

When tested against HELA cell lines, supernatants removed from cells containing this gene activated the GAS (gamma activating sequence) promoter element. Thus, it is likely that this gene activates fibroblast cells, and to a lesser extent. other tissues and cell-types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence: LQTYLSPYKLF (SEQ ID NO:556). Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, immune or hematopoietic diseases and/or disorders, particularly inflammatory conditions or immunodeficiencies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils, combined with the detected GAS biological activity; indicates that polynucleotides and polypeptides corresponding to this gene are useful for the diagnosis and treatment of a malfunctioning immune system response to foreign antigens. Representative uses are described in the “Immune Activity” and “Infectious Disease” sections below, in Example 11, 13, 14, 16, 18, 19, 20, and 27, and elsewhere herein. Briefly, this gene product may be involved in the regulation of cytokine production, antigen presentation, or other processes that may also suggest a usefulness in the treatment of cancer (e.g., by boosting immune responses).

Since the gene is expressed in cells of lymphoid origin, the gene or protein, as well as, antibodies directed against the protein may show utility as a tumor marker and/or immunotherapy targets for the above listed tissues. Expression of this gene product in neutrophils also strongly indicates a role for this protein in immune function and immune surveillance. Furthermore, the protein may also be used to determine biological activity, raise antibodies, as tissue markers, to isolate cognate ligands or receptors, to identify agents that modulate their interactions, in addition to its use as a nutritional supplement. The protein is useful in the modulation of the immune response to aberrant proteins, as may be present in rapidly proliferating cells and tissues (i.e., melanoma, etc.).

Many polynucleotide sequences, such as EST sequences, are publicly available and accessible through sequence databases. Some of these sequences are related to SEQ ID NO:124 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1132 of SEQ ID NO:124, b is an integer of 15 to 1146, where both a and b correspond to the positions of nucleotide residues shown in SEQ ID NO:124, and where b is greater than or equal to a +14.

Features of Protein Encoded by Gene No: 115

Preferred polypeptides of the invention comprise the following amino acid sequence:

| | |
|---|---|
| LAAGILNSSLPALYHSVEEISQ, | (SEQ ID NO:557) |
| XYRMNTKFLESYKMSTTLSRRHQNVSLCKDMKTPAGTDTKIAFLE, | (SEQ ID NO:558) |
| SYKMSTTLSRRHQNVSLCKDM, | (SEQ ID NO:559) |
| ICIESLMLHYIALVFEMAFMFPLVYHEMGSDSIRFHLCQVDSCLPSMMRFFFSFPFL, | (SEQ ID NO:560) |
| YIALVFEMAFMFPLVYHEMGS, and/or | (SEQ ID NO:561) |
| SDSIRFHLCQVDSCLPSMMRF. | (SEQ ID NO:562) |

Polynucleotides encoding these polypeptides are also provided.

This gene is expressed primarily in melanocytes, merkel cells, synovial cells, ulcerative colitis, and, to a lesser extent, in fetal spleen, bone marrow, jurkat cells, adrenal gland tumor tissue, and rejected kidney tissue from a failed transplantation.

Therefore, polynucleotides and polypeptides of the invention are useful as reagents for differential identification of the tissue(s) or cell type(s) present in a biological sample and for diagnosis of diseases and conditions which include, but are not limited to, integumentary, skeletal, or gastrointestinal diseases and/or disorders, particularly tumors, including melanoma, lymphoma, and adrenal gland tumors. Similarly, polypeptides and antibodies directed to these polypeptides are useful in providing immunological probes for differential identification of the tissue(s) or cell type(s). For a number of disorders of the above tissues or cells, particularly of the integumentary system, expression of this gene at significantly higher or lower levels may be routinely detected in certain tissues and cell types (e.g., integumentary, skeletal, gastrointestinal, immune, hematopoietic. renal, endocrine, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in melanocytes indicates that polynucleotides and polypeptides corresponding to this gene are useful for detecting and/or treating tumors, particularly those involving melanocytes, lymphocytes and the adrenal gland. Representative uses are described in the “Chemotaxis” and “Binding Activity” sections below, in Examples 11, 12, 13, 14, 15, 16, 18, 19, and 20, and elsewhere herein. Briefly, the secreted protein can also be used to determine biological activity, to raise antibodies, as tissue markers, to isolate cognate ligands or receptors, to identify agents that modulate their interactions and as nutritional supplements.

The protein may also have a very wide range of biological acitivities. Typical of these are cytokine, cell proliferation/differentiation modulating activity or induction of other cytokines; immunostimulating/immunosuppressant activities (e.g., for treating human immunodeficiency virus infection, cancer, autoimmune diseases and allergy); regulation of hematopoiesis (e.g., for treating anaemia or as adjunct to chemotherapy); stimulation or growth of bone, cartilage, tendons, ligaments and/or nerves (e.g., for treating wounds, stimulation of follicle stimulating hormone (for control of fertility); chemotactic and chemokinetic activities (e.g., for treating infections, tumors); hemostatic or thrombolytic activity (e.g., for treating haemophilia, cardiac infarction etc.); anti-inflammatory activity (e.g., for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:125 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1661 of SEQ ID NO:125, b is an integer of 15 to 1675, where both a and b correspond to the positions of nucleotide residues shown in SEQ ID NO:125, and where b is greater than or equal to a +14.

Features of Protein Encoded by Gene No: 116

When tested against fibroblast cell lines, supernatants removed from cells containing this gene activated the EGR1 (early growth response gene 1) promoter element. Thus, it is likely that this gene activates fibroblast cells, and to a lesser extent, other tissues and cell types, through the EGR1 signal transduction pathway. EGR1 is a separate signal transduction pathway from Jak-STAT, genes containing the EGR1 promoter are induced in various tissues and cell types upon activation, leading the cells to undergo differentiation and proliferation.

Preferred polypeptides of the invention comprise the following amino acid sequence:

| | |
|---|---|
| GGVSVQDGSLREETDVGEGGRPRGGQSEGARVTRRPSPPDSNASAFDLDLDF | (SEQ ID NO:563) |
| SPFCIWCYRLETPAEVVFSPAPLRLSGPGLAPVVFVSTLPSLQPSSFCGWDLPARPRGLSGFR, | |
| FTNKSCSKMSSTHLYKGSDVLCYARSSESMSLSCGDVANAGRLTPRLHLARS | (SEQ ID NO:564) |
| ASQGPPTLPRVPPRGSRPPTAGESPAPRTXSLENHKNIDHLSSNSHGKFRIYGQNDIKI, | |
| QDVIYTFVQRFRRPMLCTILRKYEPVVRGRRKRWQAHPSSAFGKKRLPRPPH | (SEQ ID NO:565) |
| PAQGAPQREQASHSWREPGPQNTFPRKP, | |
| REETDVGEGGRPRGGQSEGARV, | (SEQ ID NO:566) |
| GPGLAPVVFVSTLPSLQPSSFCGWDLP, | (SEQ ID NO:567) |
| MSSTHLYKGSDVLCYARSSESMSL, | (SEQ ID NO:568) |
| SQGPPTLPRVPPRGSRPPTAGESPAPRT, | (SEQ ID NO:569) |
| RFRRPMLCTILRKYEPVVRGRRKRW, and/or | (SEQ ID NO:570) |
| RLPRPPHPAQGAPQREQASHSWRE. | (SEQ ID NO:571) |

Polynucleotides encoding these polypeptides are also provided.

This gene is expressed primarily in endometrial stromal cells, CD34+, human umbilical vein endothelial cells, hematopoietic cells, and in spleen tissue.

Therefore, polynucleotides and polypeptides of the invention are useful as reagents for differential identification of the tissue(s) or cell type(s) present in a biological sample and for diagnosis of diseases and conditions which include, but are not limited to, reproductive, hematopoietic, integumentary, and immune disorders, particularly multiple myeloma, immunodeficiencies, leukemias, and vascular conditions. Similarly, polypeptides and antibodies directed to these polypeptides are useful in providing immunological probes for differential identification of the tissue(s) or cell type(s). For a number of disorders of the above tissues or cells, particularly of the hematopoietic, immune, and vascular systems, expression of this gene at significantly higher or lower levels may be routinely detected in certain tissues and cell types (e.g., immune, reproductive, hematopoietic, integumentary, endothelial, and cancerous and wounded tissues) or bodily fluids (e.g., lymph, serum, plasma, amniotic fluid, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in spleen and hematopoietic cells, combined with the detected EGR1 biological activity, indicates that polynucleotides and polypeptides corresponding to this gene are useful for the treatment and/or detection of vascular, immune and/or hematopoietic disorders including arthritis, ischemia, auto-immune diseases, host-graft rejection, AIDS, leukemia and microbial infection. Representative uses are described in the “Hyperproliferative Disorders” and “Regeneration” sections below and elsewhere herein. Briefly, polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, a utility for treating or preventing vascular or integumentary disorders may be anticipated for this gene based upon its expression within endothelial tissues in addition to its EGR1 activity. The protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:126 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1050 of SEQ ID NO:126, b is an integer of 15 to 1064, where both a and b correspond to the positions of nucleotide residues shown in SEQ ID NO:126, and where b is greater than or equal to a +14.

Features of Protein Encoded by Gene No: 117

When tested against Jurket cell lines, supernatants removed from cells containing this gene activated the GAS (gamma activating sequence) promoter element. Thus, it is likely that this gene activates T-cells, and to a lesser extent, other cells and tissue cell-types, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells Preferred polypeptides of the invention comprise the following amino acid sequence:

| | |
|---|---|
| RGMRGRWLVSSGAAFPIPLNGFCESREFFPDSGSVLLHWRPNXVLIEIKVFGSRSQSLISSKNLKTSLTFIYGKVEEVLNN, | (SEQ ID NO:572) |
| LKLSSADSQAIMNIFSADCMPRLHIALQTEMIPNRAPQGGAAANLWHEAQYRRLPFSRAPEXTDAHQASAQRGAAQLPREQ, | (SEQ ID NO:573) |
| PIPLNGFCESREFFPDSGSVLLHWRPNX, and/or | (SEQ ID NO:574) |
| NIFSADCMPRLHIALQTEMIPNRAPQGGA. | (SEQ ID NO: 575) |

Polynucleotides encoding these polypeptides are also provided.

This gene is expressed primarily in neutrophils.

Therefore, polynucleotides and polypeptides of the invention are useful as reagents for differential identification of the tissue(s) or cell type(s) present in a biological sample and for diagnosis of diseases and conditions which include, but are not limited to, diseases and/or disorders of the immune system, including neutropenia, cancer, inflammatory diseases and allergies. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in neutrophils, combined with the detected GAS biological activity, indicates that polynucleotides and polypeptides corresponding to this gene are useful for the treatment and/or diagnosis of diseases of the immune system. Representative uses are described in the “Immune Activity” and “Infectious Disease” sections below, in Example 11, 13, 14, 16, 18, 19, 20, and 27, and elsewhere herein. Briefly, the protein may be useful as a growth factor for the differentiation or proliferation of neutrophils for the treatment of neutropenia following chemotherapy, or may be useful in the treatment of immune dysfunction or as an anti-inflammatory agent by inhibiting infiltration of neutrophils to the site of injury or distress. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:127 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1593 of SEQ ID NO:127, b is an integer of 15 to 1607, where both a and b correspond to the positions of nucleotide residues shown in SEQ ID NO:127, and where b is greater than or equal to a +14.

Features of Protein Encoded by Gene No: 118

Contact of cells with supernatant expressing the product of this gene has been shown to increase the permeability of the plasma membrane of renal mesangial cells to calcium. Thus it is likely that the product of this gene is involved in a signal transduction pathway that is initiated when the product binds a receptor on the surface of the plasma membrane of both mesangial cells and other cell types, in addition to other cell-lines or tissue cell types. Thus, polynucleotides and polypeptides have uses which include, but are not limited to, activating mesangial cells. Binding of a ligand to a receptor is known to alter intracellular levels of small molecules, such as calcium, potassium and sodium, as well as alter pH and membrane potential. Alterations in small molecule concentration can be measured to identify supernatants which bind to receptors of a particular cell. In addition, when tested against fibroblast cell lines, supernatants removed from cells containing this gene activated the EGR1 (early growth response gene 1) promoter element. Thus, it is likely that this gene activates fibroblast cells, and to a lesser extent other tissues and cell types, through the EGRI signal transduction pathway. EGR1 is a separate signal transduction pathway from Jak-STAT, genes containing the EGR 1 promoter are induced in various tissues and cell types upon activation, leading the cells to undergo differentiation and proliferation.

The translation product of this gene was shown to have homology to a conserved Caenorhabditis elegans protein, F45G2.10, which is thought to be important in developmental and cellular processes (See Genbank Accession No. gn∥PID|e1346724).

Preferred polypeptides of the invention comprise the following amino acid sequence: TFRLVSAHLKTRKLINPEAAERRWRDWDSRQGWLSVK (SEQ ID NO:576), and/or KTRKLINPEAAERRWRDWDSR (SEQ ID NO:577). Polynucleotides encoding these polypeptides are also provided.

This gene is expressed primarily in bone marrow cell lines, and, to a lesser extent, in human endometrial stromal cells, human adult small intestine and human pancreas tumor.

Therefore, polynucleotides and polypeptides of the invention are useful as reagents for differential identification of the tissue(s) or cell type(s) present in a biological sample and for diagnosis of diseases and conditions which include, but are not limited to, haemopoietic and gastrointestinal tract diseases and/or disorders and stromatosis, in addition to endothelial, mucosal, or epithelial cell diorders. Similarly, polypeptides and antibodies directed to these polypeptides are useful in providing immunological probes for differential identification of the tissue(s) or cell type(s). For a number of disorders of the above tissues or cells, particularly of the immune and digestive systems, expression of this gene at significantly higher or lower levels may be routinely detected in certain tissues and cell types (e.g., haemopoietic, immune, reproductive, gastrointestinal, endocrine, developmental, and cancerous and wounded tissues) or bodily fluids (e.g., lymph, serum, plasma, urine, bile, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 256 as residues: Gly-25 to Arg-31, Ile-47 to Glu-57, Glu-120 to Arg-138.

The tissue distribution in bone marrow cells, combined with the detected calcium flux and EGR1 biological activity indicates that polynucleotides and polypeptides corresponding to this gene are useful for immune and gastrointestinal tract disorders, and stromatosis, particularly tumors and proliferative disorders. Representative uses are described in the “Immune Activity” and “Infectious Disease” sections below, in Example 11, 13, 14, 16, 18, 19, 20, and 27, and elsewhere herein. Briefly, polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:128 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1023 of SEQ ID NO:128, b is an integer of 15 to 1037, where both a and b correspond to the positions of nucleotide residues shown in SEQ ID NO:128, and where b is greater than or equal to a +14.

Features of Protein Encoded by Gene No: 119

Preferred polypeptides of the invention comprise the following amino acid sequence:

| | |
|---|---|
| WNYTVNNLYLFSFSIVSMKFMHVLSINIFFGRARWLTPVIPALLEAEAGGSLGQEFKTSLGKDGETPSLLKIQKLAGHGGRRL, | (SEQ ID NO:578) |
| DQPGKHGETLSLLKMQKLTWCGGMPFVIPSYSRSPRPENRLNLGDRGCTELLHSSLGNRVRLSKKKEVYMMELYSK, | (SEQ ID NO:579) |
| VIPALLEAEAGGSLGQEFKTSLGKDGET, | (SEQ ID NO:580) |
| NRLNLGDRGCTELLHSSLGNRVRLSKKKE, and/or | (SEQ ID NO:581) |
| HEIFGQVF. | (SEQ ID NO:582) |

Polynucleotides encoding these polypeptides are also provided.

This gene is expressed primarily in human fetal brain, fetal/liver spleen, and brain stem tissues, and to a lesser extent in human bone marrow.

Therefore, polynucleotides and polypeptides of the invention are useful as reagents for differential identification of the tissue(s) or cell type(s) present in a biological sample and for diagnosis of diseases and conditions which include, but are not limited to, neurological, developmental, and immunological diseases and/or disorders. Similarly, polypeptides and antibodies directed to these polypeptides are useful in providing immunological probes for differential identification of the tissue(s) or cell type(s). For a number of disorders of the above tissues or cells, particularly of the central nervous and immune systems, expression of this gene at significantly higher or lower levels may be routinely detected in certain tissues and cell types (e.g., neural, developmental, immune, and cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in fetal brain tissue indicates that polynucleotides and polypeptides corresponding to this gene are useful for the diagnosis and/or treatment of disorders relating to central nervous system (CNS) and immune system. In addition, polynucleotides and polypeptides corresponding to this gene are useful for the detection/treatment of neurodegenerative disease states, behavioural disorders, or inflammatory conditions. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Moreover, the expression within fetal tissue and other cellular sources marked by proliferating cells indicates this protein may play a role in the regulation of cellular division, and may show utility in the diagnosis, treatment, and/or prevention of developmental diseases and disorders, cancer, and other proliferative conditions. Representative uses are described in the “Hyperproliferative Disorders” and “Regeneration” sections below and elsewhere herein.

Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type =specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases.

The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:129 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1132 of SEQ ID NO:129, b is an integer of 15 to 1146, where both a and b correspond to the positions of nucleotide residues shown in SEQ ID NO:129, and where b is greater than or equal to a +14.

Features of Protein Encoded by Gene No: 120

Preferred polypeptides of the invention comprise the following amino acid sequence: HASEHLAALPYNVKIGK (SEQ ID NO:583). Polynucleotides encoding these polypeptides are also provided.

The gene encoding the disclosed cDNA is believed to reside on chromosome 5. Accordingly, polynucleotides related to this invention are useful as a marker in linkage analysis for chromosome 5.

This gene is expressed primarily in fetal brain tissue, fetal liver/spleen tissue, and osteoclastoma, and to a lesser extent in T cells/helper I.

Therefore, polynucleotides and polypeptides of the invention are useful as reagents for differential identification of the tissue(s) or cell type(s) present in a biological sample and for diagnosis of diseases and conditions which include, but are not limited to, developmental, neural, immune, or haemopoietic diseases and/or disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues and cell types (e.g., developmental, neural, skeletal, immune, haemopoietic disorders, and cancerous and wounded tissues) or bodily fluids (e.g., lymph, serum, plasma, amniotic fluid, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 258 as residues: Ile-31 to Glu-36, Leu-59 to Glu-73, Ser-109 to Ser-121, Ser-175 to Gln-182, Lys-258 to Lys-264.

The tissue distribution in fetal brain tissue indicates that polynucleotides and polypeptides corresponding to this gene are useful for the detection, treatment, and/or prevention of neurodegenerative disease states, behavioral disorders, or inflammatory conditions. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates it plays a role in normal neural function. Potentially, this gene product is involved in synapse formation, neurotransmission, learning, cognition, homeostasis, or neuronal differentiation or survival.

Moreover, expression of this gene product indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types.

Moreover, the expression within fetal tissue and other cellular sources marked by proliferating cells (i.e., osteoclastoma, etc.) indicates this protein may play a role in the regulation of cellular division, and may show utility in the diagnosis, treatment, and/or prevention of developmental diseases and disorders, cancer, and other proliferative conditions. Representative uses are described in the “Hyperproliferative Disorders” and “Regeneration” sections below and elsewhere herein. Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation.

Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:130 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1158 of SEQ ID NO:130, b is an integer of 15 to 1172, where both a and b correspond to the positions of nucleotide residues shown in SEQ ID NO:130, and where b is greater than or equal to a +14.

Features of Protein Encoded by Gene No: 121

Preferred polypeptides of the invention comprise the following amino acid sequence:

| | |
|---|---|
| LVCILLVHWIPPLGAWGLSLMLFLILEQRCGKGKWRNALLSVSFSVPQLQMQKVSLDSTPLNVNHDKMDIWKLTPKL, | (SEQ ID NO:584) |
| IMIKWIFGNLLLSCDLGCISTSGLPQYQGLRLLNFEYSLGFMLRSLWSRSAIQCFFS, | (SEQ ID NO:585) |
| LLLSCDLGCISTSGLPQYQGL, and/or | (SEQ ID NO:586) |
| LRLLNFEYSLGFMLRSLWSRS. | (SEQ ID NO:587) |

Polynucleotides encoding these polypeptides are also provided. The gene encoding the disclosed cDNA is believed to reside on chromosome 11. Accordingly, polynucleotides related to this invention are useful as a marker in linkage analysis for chromosome 11.

This gene is expressed primarily in fetal liver/spleen tissue, infant brain, prostate carcinoma, and keratinocytes, and to a lesser extent in human gall bladder tissue.

Therefore, polynucleotides and polypeptides of the invention are useful as reagents for differential identification of the tissue(s) or cell type(s) present in a biological sample and for diagnosis of diseases and conditions which include, but are not limited to, metabolic, developmental, immune, and gastrointestinal diseases and/or disorders, particularly those relating to the gall bladder. Similarly, polypeptides and antibodies directed to these polypeptides are useful in providing immunological probes for differential identification of the tissue(s) or cell type(s). For a number of disorders of the above tissues or cells, particularly of the gastrointestinal tract system, expression of this gene at significantly higher or lower levels may be routinely detected in certain tissues and cell types (e.g., metabolic, developmental, integumentary, reproductive, gastrointestinal, and cancerous and wounded tissues) or bodily fluids (e.g., lymph, serum, bile, plasma, seminal fluid, amniotic fluid, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 259 as residues: Ser-18 to Gly-26.

The tissue distribution in fetal brain tissue indicates that polynucleotides and polypeptides corresponding to this gene are useful for the detection, treatment, and/or prevention of neurodegenerative disease states, behavioral disorders, or inflammatory conditions. Representative uses are described in the “Regeneration” and “Hyperproliferative Disorders” sections below, in Example 11, 15, and 18, and elsewhere herein. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates it plays a role in normal neural function. Potentially, this gene product is involved in synapse formation, neurotransmission, learning, cognition, homeostasis, or neuronal differentiation or survival.

Moreover, expression of this gene product indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g., by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types.

Moreover, the expression within fetal tissue and other cellular sources marked by proliferating cells (i.e., prostate carcinoma, etc.) indicates this protein may play a role in the regulation of cellular division, and may show utility in the diagnosis, treatment, and/or prevention of developmental diseases and disorders, cancer, and other proliferative conditions. Representative uses are described in the “Hyperproliferative Disorders” and “Regeneration” sections below and elsewhere herein. Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. The tissue distribution in gall bladder tissue indicates that polynucleotides and polypeptides corresponding to this gene are useful for the diagnosis and treatment of gall bladder disorders, or related metabolic conditions, such as gall stones. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:131 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 649 of SEQ ID NO:131, b is an integer of 15 to 663, where both a and b correspond to the positions of nucleotide residues shown in SEQ ID NO:131, and where b is greater than or equal to a +14.

Features of Protein Encoded by Gene No: 122

Preferred polypeptides of the invention comprise the following amino acid sequence:

| | |
|---|---|
| ASPHLFIEKWGRAFILRKLLLVPVISKRIINIMAHQVKPPIFCAMIMCNLFCSGYEHLLFTLMRFFSFEQIFDEVVFH, | (SEQ ID NO:588) |
| KLLLVPVISKRIINIMAHQVKPPIF, and/or | (SEQ ID NO:589) |
| PEQKRLH. | (SEQ ID NO:590) |

Polynucleotides encoding these polypeptides are also provided.

The gene encoding the disclosed cDNA is believed to reside on chromosome 4. Accordingly, polynucleotides related to this invention are useful as a marker in linkage analysis for chromosome 4.

This gene is expressed primarily in glioblastoma, infant brain, uterus, and gall bladder, and to a lesser extent in placental tissue.

Therefore, polynucleotides and polypeptides of the invention are useful as reagents for differential identification of the tissue(s) or cell type(s) present in a biological sample and for diagnosis of diseases and conditions which include, but are not limited to, neural and developmental diseases and/or disorders, particularly glioblastoma multiform. Similarly, polypeptides and antibodies directed to these polypeptides are useful in providing immunological probes for differential identification of the tissue(s) or cell type(s). For a number of disorders of the above tissues or cells, particularly of the central nervous system (CNS), expression of this gene at significantly higher or lower levels may be routinely detected in certain tissues and cell types (e.g., neural, developmental, reproductive, metabolic, and cancerous and wounded tissues) or bodily fluids (e.g., lymph, serum, plasma, urine, amniotic fluid, bile, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 260 as residues: Ser-40 to Gly-45, Leu-73 to Arg-80.

The tissue distribution in glioblastoma indicates that polynucleotides and polypeptides corresponding to this gene are useful for the diagnosis and treatment of neural cell disorders. Representative uses are described in the “Regeneration” and “Hyperproliferative Disorders” sections below, in Example 11, 15, and 18, and elsewhere herein. Briefly, polynucleotides and polypeptides corresponding to this gene are useful for the detection/treatment of neurodegenerative disease states, behavioural disorders, or inflammatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:132 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 762 of SEQ ID NO:132, b is an integer of 15 to 776, where both a and b correspond to the positions of nucleotide residues shown in SEQ ID NO:132, and where b is greater than or equal to a +14.

Features of Protein Encoded by Gene No: 123

The translation product of this gene was shown to have homology to several highly conserved integral membrane proteins (See, for example, Genomics 31 (3), 295-300 (1996), Biochim. Biophys. Acta, Gene Struct. Expr. 1306 (2-3), 137-141 (1996), which are hereby incorporated herein by reference). Based on the sequence similarity, the translation product of this gene is expected to share biological activities with membrane proteins and receptors. Such activities are known in the art, some of which are described elsewhere herein.

When tested against U937 and Jurket cell lines, supernatants removed from cells containing this gene activated the GAS (gamma activating sequence) promoter element. Thus, it is likely that this gene activates myeloid cells, including their progenitors, and to a lesser extent, other tissues and cell types, through the Jak-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

Preferred polypeptides of the invention comprise the following amino acid sequence:

| | |
|---|---|
| FAVIRFESIIHEFDPWFNYRSTHHLASHGFYEFLNWFDERAWYPLGRIVGGTV | (SEQ ID NO:591) |
| YPGLMITAGLIHWILNT | |
| LNITVHIRDVCVFLAPTFSGLTSISTFLLTRELWNQGAGLLAACFIAIVPGYISR | |
| SVAGSFDNEGIAIFA | |
| LQFTYYLWVKSVKTGSVFWTMCCCLSYFYMVSAWGGYVFIINLIPLHVFVLL | |
| LMQRYSKRVYIAYSTFYI | |
| VGLILSMQIPFVGFQPIRTSEHMAAAGVFALLQAYAFLQYLRDRLTKQEFQTL | |
| FFLGVSLAAGAVFLSVI | |
| YLTYTGYIAPWSGRFYSLWDTGYAKIHIPIIASVSEHQPTTWVSFFFDLHILVC | |
| TFPAGLWFCIKNINDE RXFGKXGF, | |
| EFDPWFNYRSTHHLASHGFYEFLNWFD, | (SEQ ID NO:592) |
| TRELWNQGAGLLAACFIAIVPGY, | (SEQ ID NO:593) |
| TYYLWVKSVKTGSVFWTMCCCL, | (SEQ ID NO:594) |
| GVFALLQAYAFLQYLRDRLTKQEFQ, and/or | (SEQ ID NO:595) |
| YSLWDTGYAKIHIPIIASVSEHQPTTW. | (SEQ ID NO:596) |

Polynucleotides encoding these polypeptides are also provided.

This gene is expressed primarily in human colon carcinoma (HCC) cell line, and to a lesser extent in human eosinophils.

Therefore, polynucleotides and polypeptides of the invention are useful as reagents for differential identification of the tissue(s) or cell type(s) present in a biological sample and for diagnosis of diseases and conditions which include, but are not limited to, gastrointestinal or immune diseases and/or disorders, particularly colon carcinoma and leukemia. Similarly, polypeptides and antibodies directed to these polypeptides are useful in providing immunological probes for differential identification of the tissue(s) or cell type(s). For a number of disorders of the above tissues or cells, particularly of the excretory and immune system, expression of this gene at significantly higher or lower levels may be routinely detected in certain tissues and cell types (e.g., gastrointestinal, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

Preferred epitopes include those comprising a sequence shown in SEQ ID NO: 261 as residues: Glu-49 to Ser-54.

The tissue distribution in human colon carcinoma cell lines, combined with the detected GAS biological activity, indicates that this protein may play a role in the regulation of cellular division, and may show utility in the diagnosis, treatment, and/or prevention of developmental diseases and disorders, cancer, and other proliferative conditions. Representative uses are described in the “Hyperproliferative Disorders” and “Regeneration” sections below and elsewhere herein.

Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases.

The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:133 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1529 of SEQ ID NO:133, b is an integer of 15 to 1543, where both a and b correspond to the positions of nucleotide residues shown in SEQ ID NO:133, and where b is greater than or equal to a +14.

Features of Protein Encoded by Gene No: 124

This gene shares homology with elongation factor 1-Alpha (giardia intestinalis), and the human eukaryotic release factor 3b (See Genbank Accession No. gi|4099482; all references available through this accession number are hereby incorporated herein, by reference; for example, FEBS Lett. 440 (3), 387-392 (1998)); FEBS Lett. 1999 Jan 22;443(1):41-7; J Biol Chem. 1998 Aug 28;273(35):22254-9; and Genes Dev. 1998 Jun 1;12(11):1665-77) which promotes the GTP-dependent binding of aminoacyl tRNA to the A-site of ribosomes during protein biosynthesis.

Preferred polypeptides of the invention comprise the following amino acid sequence:

| | |
|---|---|
| MGHMLYLLGNINKRTMHKYXQESKKAGKASFAYAWVLDETGEERERGVT | (SEQ ID NO:597) |
| MDVGMTKFETTTKVITLMDAPGHKDFIPNMITGAAQADVAVLVVDASRGEF | |
| EAGFETGGQTREHGLLVRSLGVTQLAVAVNKMDQVNWQQERFQEITGKLG | |
| HFLKQAGFKESDVGFIPTSGLSGENLITRSQSSELTKWYKGLCLLEQIDSFKPP | |
| QRSIDKPFRLCVSDVFKDQGSGFCITGKIEAGYIQTGDRLLAMPPNETCTVKGI | |
| TLHDEPVDWAAAGDHVSLTLVGMDIIKINVGCIFCGPKVPIKACTRFRARILIF | |
| NIEIPITKGFPVLLHYQTVSEPAVIKRLISVLNKSTGEVTKKKPKFLTKGQNAL | |
| VELQTQRPIALELYKDFKELGRFMLRYGGSTIAAGVVTEIKE, | |
| LYLLGNINKRTMHKYXQESKK, | (SEQ ID NO:598) |
| LDETGEERERGVTMDVGMTKFET, | (SEQ ID NO:599) |
| GHKDFIPNMITGAAQADVAVLV, | (SEQ ID NO:600) |
| GFETGGQTREHGLLVRSLGVTQL, | (SEQ ID NO:601) |
| WQQERFQEITGKLGHFLKQAGFK, | (SEQ ID NO:602) |
| TSGLSGENLITRSQSSELTKWY, | (SEQ ID NO:603) |
| PQRSIDKPFRLCVSDVFKDQGSG, | (SEQ ID NO:604) |
| LISVLNKSTGEVTKKKPKFLTK, | (SEQ ID NO:605) |
| QRPIALELYKDFKELGRFMLRYGGS, and/or | (SEQ ID NO:606) |
| QKGPPIEDAIASSDVLETASKSANPPHTIQASEEQSSTPAPVKKSGKLRQQIDVKAELEKRQGGKQLLNL VVIGHVDAGKSTL. | (SEQ ID NO:607) |

Polynucleotides encoding these polypeptides are also provided.

The gene encoding the disclosed cDNA is thought to reside on chromosome 6. Accordingly, polynucleotides related to this invention are useful as a marker in linkage analysis for chromosome 6.

This gene is expressed primarily in colon tissue from a patient having ulcerative colitis, brain tissue, lung tissue, testes and endometrial tumor.

Therefore, polynucleotides and polypeptides of the invention are useful as reagents for differential identification of the tissue(s) or cell type(s) present in a biological sample and for diagnosis of diseases and conditions which include, but are not limited to, ulcerative colitis, and testes and endometrial tumors. Similarly, polypeptides and antibodies directed to these polypeptides are useful in providing immunological probes for differential identification of the tissue(s) or cell type(s). For a number of disorders of the above tissues or cells, particularly of the immune system and reproductive system, expression of this gene at significantly higher or lower levels may be routinely detected in certain tissues or cell types (e.g., reproductive, developmental, immune, cancerous and wounded tissues) or bodily fluids (e.g., serum, seminal fluid, amniotic fluid, pulmonary surfactant or sputum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in ulcerative colitis, testes and endometrial tumors indicates that polynucleotides and polypeptides corresponding to this gene are useful for diagnosing or treatment of a variety of reproductive or gastrointestinal disorders. Representative uses are described in the “Infectious Disease”, “Chemotaxis”, and “Binding Activity” sections below, in Examples 11, 12, 13, 14, 15, 16, 18, 19, and 20, and elsewhere herein. the tissue distribution indicates that polynucleotides and polypeptides corresponding to this gene are useful for the treatment and diagnosis of conditions concerning proper testicular function (e.g., endocrine function, sperm maturation), as well as cancer. Therefore, this gene product is useful in the treatment of male infertility and/or impotence. This gene product is also useful in assays designed to identify binding agents as such agents (antagonists) are useful as male contraceptive agents. Similarly, the protein is believed to by useful in the treatment and/or diagnosis of testicular cancer. The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications.

Moreover, the expression within cellular sources marked by proliferating cells, combined with the homology to the elongation release factors indicates this protein may play a role in the regulation of cellular division, and may show utility in the diagnosis, treatment, and/or prevention of developmental diseases and disorders, cancer, and other proliferative conditions.

Representative uses are described in the “Hyperproliferative Disorders” and “Regeneration” sections below and elsewhere herein. Briefly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Because of potential roles in proliferation and differentiation, this gene product may have applications in the adult for tissue regeneration and the treatment of cancers. It may also act as a morphogen to control cell and tissue type specification. Therefore, the polynucleotides and polypeptides of the present invention are useful in treating, detecting, and/or preventing said disorders and conditions, in addition to other types of degenerative conditions. Thus this protein may modulate apoptosis or tissue differentiation and is useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:134 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2143 of SEQ ID NO:134, b is an integer of 15 to 2157, where both a and b correspond to the positions of nucleotide residues shown in SEQ ID NO:134, and where b is greater than or equal to a +14.

Features of Protein Encoded by Gene No: 125

Preferred polypeptides of the invention comprise the following amino acid sequence:

| | |
|---|---|
| NGFFSFSMYIILCQTFFSVAALRWTGDSIGFINLSFSHLFIPQTFVEGHQALGRG | (SEQ ID NO:608) |
| KWFYKLVLSGIKEIYNLYYLIVATSHMWFSNKISITSPTTFSSLVRSRPRETVPFIVFSAFYKLR, | |
| IILCQTFFSVAALRWTGDSIG, | (SEQ ID NO:609) |
| GFINLSFSHLFIPQTFVEGHQ, | (SEQ ID NO:610) |
| QALGRGKWFYKLVLSGIKEI, and/or | (SEQ ID NO:611) |
| IYNLYYLIVATSHMWFSNKIS. | (SEQ ID NO:612) |

Polynucleotides encoding these polypeptides are also provided.

This gene is expressed primarily in skin, and to a lesser extent in uterine cells and tissues.

Therefore, polynucleotides and polypeptides of the invention are useful as reagents for differential identification of the tissue(s) or cell type(s) present in a biological sample and for diagnosis of diseases and conditions which include, but are not limited to, integumentary and reproductive disorders and/or diseases. Similarly, polypeptides and antibodies directed to these polypeptides are useful in providing immunological probes for differential identification of the tissue(s) or cell type(s). For a number of disorders of the above tissues or cells, particularly of the integumentary system, expression of this gene at significantly higher or lower levels may be routinely detected in certain tissues and cell types (e.g., integumentary, melanocyte, reproductive, and cancerous and wounded tissues) or bodily fluids (e.g., lymph, serum, plasma, urine, amniotic fluid, synovial fluid and spinal fluid) or another tissue or cell sample taken from an individual having such a disorder, relative to the standard gene expression level, i.e., the expression level in healthy tissue or bodily fluid from an individual not having the disorder.

The tissue distribution in skin indicates that polynucleotides and polypeptides corresponding to this gene are useful for the diagnosis and/or treatment of diseases relating to integumentary conditions. Representative uses are described in the “Biological Activity”, “Hyperproliferative Disorders”, “Infectious Disease”, and “Regeneration” sections below, in Example 11, 19, and 20, and elsewhere herein. Briefly, polynucleotides and polypeptides corresponding to this gene are useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. In addition, such disorders may predispose (i.e., increase an individuals susceptibility) to viral and bacterial infections of the skin (i.e. cold sores, warts, chickenpox, molluscum contagiosum, herpes zoster, boils, cellulitis, erysipelas, impetigo, tinea, althletes foot, and ringworm).

Moreover, the protein product of this gene may also be useful for the treatment or diagnosis of various connective tissue disorders such as arthritis, trauma, tendonitis, chrondomalacia and inflammation, autoimmune disorders such as rheumatoid arthritis, lupus, scleroderma, and dermatomyositis as well as dwarfism, spinal deformation, and specific joint abnormalities as well as chondrodysplasias (ie. spondyloepiphyseal dysplasia congenita, familial osteoarthritis, Atelosteogenesis type II, metaphyseal chondrodysplasia type Schmid). Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:135 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 406 of SEQ ID NO:135, b is an integer of 15 to 420, where both a and b correspond to the positions of nucleotide residues shown in SEQ ID NO:135, and where b is greater than or equal to a +14.

| | | | | | | | | | 5′ NT | | | | | |
|---|---|---|---|---|---|---|---|---|---|---|---|---|---|---|
| | | | | NT | | | | | of | AA | First | Last | | |
| | | ATCC | | SEQ | | 5′ NT | 3′ NT | 5′ NT | First | SEQ | AA | AA | First | Last |
| | | Deposit | | ID | Total | of | of | of | AA of | ID | of | of | AA of | AA |
| Gene | cDNA | Nr and | | NO: | NT | Clone | Clone | Start | Signal | NO: | Sig | Sig | Secreted | of |
| No. | Clone ID | Date | Vector | X | Seq. | Seq. | Seq. | Codon | Pep | Y | Pep | Pep | Portion | ORF |
| 1 | HUSIG64 | 209423 | pSport1 | 11 | 1010 | 1 | 1010 | 9 | 9 | 139 | 1 | 21 | 22 | 334 |
| | | 10/30/97 | | | | | | | | | | | | |
| 2 | HATCI78 | 209368 | Uni-ZAP XR | 12 | 1559 | 1 | 1559 | 283 | 283 | 140 | 1 | 20 | 21 | 42 |
| | | 10/16/97 | | | | | | | | | | | | |
| 3 | HSIDR70 | 209368 | Uni-ZAP XR | 13 | 1589 | 1 | 1589 | 110 | 110 | 141 | 1 | 17 | 18 | 86 |
| | | 10/16/97 | | | | | | | | | | | | |
| 4 | HFADD53 | 209368 | Uni-ZAP XR | 14 | 1255 | 1 | 1255 | 183 | 183 | 142 | 1 | 22 | 23 | 121 |
| | | 10/16/97 | | | | | | | | | | | | |
| 5 | HPMGT5I | 209423 | Uni-ZAP XR | 15 | 1191 | 1 | 1191 | 152 | 152 | 143 | 1 | 29 | 30 | 275 |
| | | 10/30/97 | | | | | | | | | | | | |
| 6 | HFVAB79 | 209368 | Uni-ZAP XR | 16 | 1186 | 1 | 1186 | 139 | 139 | 144 | 1 | 15 | 16 | 194 |
| | | 10/16/97 | | | | | | | | | | | | |
| 7 | HDTBP5I | 209407 | pCMVSport | 17 | 1182 | 1 | 1182 | 93 | 93 | 145 | 1 | 25 | 26 | 182 |
| | | 10/23/97 | 2.0 | | | | | | | | | | | |
| 8 | HLHFR19 | 209407 | Uni-ZAP XR | 18 | 1171 | 1 | 1171 | 24 | 24 | 146 | 1 | 30 | 31 | 121 |
| | | 10/23/97 | | | | | | | | | | | | |
| 9 | HMEET96 | 209407 | Lambda ZAP | 19 | 1337 | 73 | 1200 | 121 | 121 | 147 | 1 | 30 | 31 | 266 |
| | | 10/23/97 | II | | | | | | | | | | | |
| 10 | HTXCV12 | 209423 | Uni-ZAP XR | 20 | 1162 | 1 | 1162 | 183 | 183 | 148 | 1 | 27 | 28 | 91 |
| | | 10/30/97 | | | | | | | | | | | | |
| 11 | HCEFB70 | 209423 | Uni-ZAP XR | 21 | 1837 | 1 | 1837 | 223 | 223 | 149 | 1 | 24 | 25 | 108 |
| | | 10/30/97 | | | | | | | | | | | | |
| 12 | HDTAV25 | 209423 | pCMVSport | 22 | 1054 | 1 | 1054 | 100 | 100 | 150 | 1 | 38 | 39 | 87 |
| | | 10/30/97 | 2.0 | | | | | | | | | | | |
| 13 | HSATA21 | 209368 | Uni-ZAP XR | 23 | 1066 | 1 | 1060 | 49 | 49 | 151 | 1 | 25 | 26 | 73 |
| | | 10/16/97 | | | | | | | | | | | | |
| 14 | HKIXIO3 | 209368 | pBluescript | 24 | 928 | 1 | 928 | 61 | 61 | 152 | 1 | 24 | 25 | 71 |
| | | 10/16/97 | | | | | | | | | | | | |
| 15 | HDTDC56 | 209407 | pCMVSport | 25 | 966 | 1 | 966 | 210 | 210 | 153 | 1 | 24 | 25 | 151 |
| | | 10/23/97 | 2.0 | | | | | | | | | | | |
| 16 | HLTBF35 | 209407 | Uni-ZAP XR | 26 | 1146 | 1 | 1132 | 136 | 136 | 154 | 1 | 16 | 17 | 60 |
| | | 10/23/97 | | | | | | | | | | | | |
| 17 | HEPAB80 | 209423 | Uni-ZAP XR | 27 | 802 | 1 | 802 | 67 | 67 | 155 | 1 | 28 | 29 | 122 |
| | | 10/30/97 | | | | | | | | | | | | |
| 18 | HFOXB13 | 209423 | pSport1 | 28 | 1169 | 1 | 1169 | 36 | 36 | 156 | 1 | 21 | 22 | 54 |
| | | 10/30/97 | | | | | | | | | | | | |
| 19 | HTOAK16 | 209368 | Uni-ZAP XR | 29 | 1466 | 1 | 1466 | 87 | 87 | 157 | 1 | 18 | 19 | 110 |
| | | 10/16/97 | | | | | | | | | | | | |
| 20 | HBXDC63 | 209368 | ZAP Express | 30 | 1226 | 1 | 1226 | 165 | 165 | 158 | 1 | 30 | 31 | 47 |
| | | 10/16/97 | | | | | | | | | | | | |
| 21 | HASAU43 | 209407 | Uni-ZAP XR | 31 | 1094 | 1 | 1094 | 33 | 33 | 159 | 1 | 17 | 18 | 81 |
| | | 10/23/97 | | | | | | | | | | | | |
| 22 | HAGEA31 | 209423 | Uni-ZAP XR | 32 | 1037 | 1 | 1037 | 151 | 151 | 160 | 1 | 25 | 26 | 155 |
| | | 10/30/97 | | | | | | | | | | | | |
| 23 | HEQAF19 | 209423 | pCMVSport | 33 | 1376 | 1 | 1376 | 84 | 84 | 161 | 1 | 23 | 24 | 294 |
| | | 10/30/97 | 3.0 | | | | | | | | | | | |
| 24 | HTXHB33 | 209368 | Uni-ZAP XR | 34 | 1220 | 1 | 1220 | 243 | 243 | 162 | 1 | 17 | 18 | 59 |
| | | 10/16/97 | | | | | | | | | | | | |
| 25 | HMWFT65 | 209368 | Uni-Zap XR | 35 | 1346 | 1 | 1346 | 72 | 72 | 163 | 1 | 28 | 29 | 121 |
| | | 10/16/97 | | | | | | | | | | | | |
| 26 | HNGAZ68 | 209368 | Uni-ZAP XR | 36 | 1026 | 1 | 1026 | 238 | 238 | 164 | 1 | 18 | 19 | 72 |
| | | 10/16/97 | | | | | | | | | | | | |
| 27 | HTWFH07 | 209407 | pSport1 | 37 | 832 | 1 | 832 | 14 | 14 | 165 | 1 | 25 | 26 | 122 |
| | | 10/23/97 | | | | | | | | | | | | |
| 28 | HMQDF12 | 209407 | Uni-ZAP XR | 38 | 706 | 1 | 627 | 63 | 63 | 166 | 1 | 27 | 28 | 142 |
| | | 10/23/97 | | | | | | | | | | | | |
| 29 | HFABH95 | 209407 | Uni-ZAP XR | 39 | 1347 | 1 | 1347 | 199 | 199 | 167 | 1 | 21 | 22 | 116 |
| | | 10/23/97 | | | | | | | | | | | | |
| 30 | HNGDD48 | 209423 | Uni-ZAP XR | 40 | 1467 | 1 | 1467 | 85 | 85 | 168 | 1 | 20 | 21 | 58 |
| | | 10/30/97 | | | | | | | | | | | | |
| 31 | HPMBY46 | 209423 | Uni-ZAP XR | 41 | 914 | 1 | 914 | 63 | 63 | 169 | 1 | 21 | 22 | 125 |
| | | 10/30/97 | | | | | | | | | | | | |
| 32 | HRKPA09 | 209423 | pBluescript | 42 | 1131 | 1 | 1131 | 101 | 101 | 170 | 1 | 33 | 34 | 86 |
| | | 10/30/97 | | | | | | | | | | | | |
| 33 | HAGAQ26 | 209368 | Uni-ZAP XR | 43 | 1333 | 157 | 1333 | 251 | 251 | 171 | 1 | 20 | 21 | 62 |
| | | 10/16/97 | | | | | | | | | | | | |
| 34 | HCWFL55 | 209368 | ZAP Express | 44 | 1004 | 1 | 1004 | 40 | 40 | 172 | 1 | 19 | 20 | 47 |
| | | 10/16/97 | | | | | | | | | | | | |
| 35 | HKAAE44 | 209368 | pCMVSport | 45 | 1494 | 1 | 1494 | 113 | 113 | 173 | 1 | 39 | 40 | 136 |
| | | 10/16/97 | 2.0 | | | | | | | | | | | |
| 36 | HNGEU90 | 209407 | Uni-ZAP XR | 46 | 1166 | 1 | 1166 | 17 | 17 | 174 | 1 | 20 | 21 | 88 |
| | | 10/23/97 | | | | | | | | | | | | |
| 37 | HCFCC07 | 209407 | pSport1 | 47 | 1536 | 1 | 1536 | 94 | 94 | 175 | 1 | 47 | 48 | 57 |
| | | 10/23/97 | | | | | | | | | | | | |
| 38 | HLWBI63 | 209407 | pCMVSport | 48 | 1038 | 1 | 1038 | 149 | 149 | 176 | 1 | 30 | 31 | 63 |
| | | 10/23/97 | 3.0 | | | | | | | | | | | |
| 39 | HDUAC77 | 209423 | pSport1 | 49 | 1176 | 1 | 1176 | 193 | 193 | 177 | 1 | 19 | 20 | 60 |
| | | 10/30/97 | | | | | | | | | | | | |
| 40 | HFOYV27 | 209423 | pSport1 | 50 | 731 | 1 | 731 | 171 | 171 | 178 | 1 | 18 | 19 | 103 |
| | | 10/30/97 | | | | | | | | | | | | |
| 41 | HGBHI35 | 209423 | Uni-ZAP XR | 51 | 1437 | 71 | 1276 | 87 | 87 | 179 | 1 | 16 | 17 | 292 |
| | | 10/30/97 | | | | | | | | | | | | |
| 42 | HRDEU27 | 209423 | Uni-ZAP XR | 52 | 1369 | 1 | 1369 | 285 | 285 | 180 | 1 | 18 | 19 | 45 |
| | | 10/30/97 | | | | | | | | | | | | |
| 43 | HNGJE50 | 209368 | Uni-ZAP XR | 53 | 1037 | 1 | 1037 | 77 | 77 | 181 | 1 | 36 | 37 | 46 |
| | | 10/16/97 | | | | | | | | | | | | |
| 44 | HNHDU48 | 209368 | Uni-ZAP XR | 54 | 1373 | 1 | 1373 | 99 | 99 | 182 | 1 | 20 | 21 | 54 |
| | | 10/16/97 | | | | | | | | | | | | |
| 45 | HFXJU68 | 209423 | Lambda ZAP | 55 | 1347 | 1 | 1347 | 148 | 148 | 183 | 1 | 25 | 26 | 66 |
| | | 10/30/97 | II | | | | | | | | | | | |
| 46 | HMMAH60 | 209368 | pSport1 | 56 | 822 | 1 | 822 | 142 | 142 | 184 | 1 | 15 | 16 | 50 |
| | | 10/16/97 | | | | | | | | | | | | |
| 47 | HNGFR31 | 209407 | Uni-ZAP XR | 57 | 536 | 1 | 536 | 108 | 108 | 185 | 1 | 23 | 24 | 90 |
| | | 10/23/97 | | | | | | | | | | | | |
| 48 | HFPDB26 | 209423 | Uni-ZAP XR | 58 | 1262 | 50 | 1192 | 65 | 65 | 186 | 1 | 29 | 30 | 54 |
| | | 10/30/97 | | | | | | | | | | | | |
| 49 | HFRAW86 | 209423 | Uni-ZAP XR | 59 | 1269 | 1 | 1269 | 162 | 162 | 187 | 1 | 16 | 17 | 63 |
| | | 10/30/97 | | | | | | | | | | | | |
| 50 | FITEDX90 | 209368 | Uni-ZAP XR | 60 | 1829 | 1 | 1829 | 63 | 63 | 188 | 1 | 17 | 18 | 112 |
| | | 10/16/97 | | | | | | | | | | | | |
| 51 | HTXGG45 | 209407 | Uni-ZAP XR | 61 | 1112 | 1 | 1112 | 52 | 52 | 189 | 1 | 19 | 20 | 59 |
| | | 10/23/97 | | | | | | | | | | | | |
| 52 | HTXJI95 | 209407 | Uni-ZAP XR | 62 | 1674 | 1 | 1674 | 164 | 164 | 190 | 1 | 23 | 24 | 63 |
| | | 10/23/97 | | | | | | | | | | | | |
| 53 | HLYBD32 | 209407 | pSport1 | 63 | 1045 | 35 | 1045 | 98 | 98 | 191 | 1 | 23 | 24 | 70 |
| | | 10/23/97 | | | | | | | | | | | | |
| 54 | HOUDK26 | 209423 | Uni-ZAP XR | 64 | 1051 | 1 | 1051 | 214 | 214 | 192 | 1 | 30 | 31 | 174 |
| | | 10/30/97 | | | | | | | | | | | | |
| 55 | HROAJ03 | 209423 | Uni-ZAP XR | 65 | 1182 | 1 | 1182 | 19 | 19 | 193 | 1 | 20 | 21 | 192 |
| | | 10/30/97 | | | | | | | | | | | | |
| 56 | HTXAJ12 | 209423 | Uni-ZAP XR | 66 | 675 | 1 | 675 | 91 | 91 | 194 | 1 | 18 | 19 | 111 |
| | | 10/30/97 | | | | | | | | | | | | |
| 57 | HKAEL80 | 209423 | pCMVSport | 67 | 1105 | 1 | 1105 | 398 | 398 | 195 | 1 | 17 | 18 | 79 |
| | | 10/30/97 | 2.0 | | | | | | | | | | | |
| 58 | HNHFL04 | 209423 | Uni-ZAP XR | 68 | 1279 | 1 | 1279 | 162 | 162 | 196 | 1 | 16 | 17 | 87 |
| | | 10/30/97 | | | | | | | | | | | | |
| 59 | HPCAM01 | 209368 | Uni-ZAP XR | 69 | 1638 | 1 | 1638 | 311 | 311 | 197 | 1 | 24 | 25 | 41 |
| | | 10/16/97 | | | | | | | | | | | | |
| 60 | HJACA79 | 209368 | pBluescript | 70 | 887 | 1 | 887 | 84 | 84 | 198 | 1 | 28 | 29 | 68 |
| | | 10/16/97 | SK- | | | | | | | | | | | |
| 61 | HMADK33 | 209368 | Uni-ZAP XR | 71 | 864 | 1 | 864 | 161 | 161 | 199 | 1 | 24 | 25 | 152 |
| | | 10/16/97 | | | | | | | | | | | | |
| 62 | HMSFI26 | 209368 | Uni-ZAP XR | 72 | 1217 | 1 | 1217 | 120 | 120 | 200 | 1 | 34 | 35 | 62 |
| | | 10/16/97 | | | | | | | | | | | | |
| 63 | HMSJR08 | 209368 | Uni-ZAP XR | 73 | 1717 | 1 | 1717 | 165 | 165 | 201 | 1 | 28 | 29 | 63 |
| | | 10/16/97 | | | | | | | | | | | | |
| 64 | HMWIO93 | 209368 | Uni-Zap XR | 74 | 1276 | 1 | 1276 | 72 | 72 | 202 | 1 | 18 | 19 | 42 |
| | | 10/16/97 | | | | | | | | | | | | |
| 65 | HNGAK47 | 209368 | Uni-ZAP XR | 75 | 1144 | 1 | 1144 | 89 | 89 | 203 | 1 | 23 | 24 | 40 |
| | | 10/16/97 | | | | | | | | | | | | |
| 66 | HNGAL31 | 209368 | Uni-ZAP XR | 76 | 918 | 1 | 918 | 34 | 34 | 204 | 1 | 20 | 21 | 43 |
| | | 10/16/97 | | | | | | | | | | | | |
| 67 | HNGIZ06 | 209368 | Uni-ZAP XR | 77 | 1065 | 1 | 1065 | 108 | 108 | 205 | 1 | 16 | 17 | 41 |
| | | 10/16/97 | | | | | | | | | | | | |
| 68 | HNHBI75 | 209368 | Uni-ZAP XR | 78 | 1126 | 1 | 1126 | 12 | 12 | 206 | 1 | 15 | 16 | 41 |
| | | 10/16/97 | | | | | | | | | | | | |
| 69 | HOFNT24 | 209368 | pCMVSport | 79 | 984 | 1 | 984 | 63 | 63 | 207 | 1 | 22 | 23 | 112 |
| | | 10/16/97 | 2.0 | | | | | | | | | | | |
| 70 | HSAXI95 | 209368 | Uni-ZAP XR | 80 | 1247 | 1 | 1247 | 147 | 147 | 208 | 1 | 19 | 20 | 44 |
| | | 10/16/97 | | | | | | | | | | | | |
| 71 | HCMTB45 | 209368 | Uni-ZAP XR | 81 | 958 | 1 | 958 | 215 | 215 | 209 | 1 | 20 | 21 | 123 |
| | | 10/16/97 | | | | | | | | | | | | |
| 71 | HCMTB45 | 209368 | Uni-ZAP XR | 136 | 946 | 1 | 946 | 209 | 209 | 264 | 1 | 27 | 28 | 70 |
| | | 10/16/97 | | | | | | | | | | | | |
| 72 | HE9CP41 | 209368 | Uni-ZAP XR | 82 | 1392 | 1 | 1392 | 132 | 132 | 210 | 1 | 21 | 22 | 41 |
| | | 10/16/97 | | | | | | | | | | | | |
| 73 | HHENV10 | 209368 | pCMVSport | 83 | 1155 | 1 | 1155 | 143 | 143 | 211 | 1 | 27 | 28 | 50 |
| | | 10/16/97 | 3.0 | | | | | | | | | | | |
| 74 | HSKDD72 | 209407 | Uni-ZAP XR | 84 | 1373 | 1 | 1373 | 94 | 94 | 212 | 1 | 23 | 24 | 64 |
| | | 10/23/97 | | | | | | | | | | | | |
| 75 | HAGDO20 | 209407 | Uni-ZAP XR | 85 | 1258 | 184 | 1258 | 218 | 218 | 213 | 1 | 20 | 21 | 76 |
| | | 10/23/97 | | | | | | | | | | | | |
| 76 | HCFBH15 | 209407 | pSport1 | 86 | 1318 | 1 | 1318 | 156 | 156 | 214 | 1 | 22 | 23 | 44 |
| | | 10/23/97 | | | | | | | | | | | | |
| 77 | HSYBX48 | 209423 | pCMVSport | 87 | 978 | 38 | 961 | 246 | 246 | 215 | 1 | 34 | 35 | 65 |
| | | 10/30/97 | 3.0 | | | | | | | | | | | |
| 78 | HATDQ62 | 209423 | Uni-ZAP XR | 88 | 1863 | 323 | 1863 | 412 | 412 | 216 | 1 | 25 | 26 | 61 |
| | | 10/30/97 | | | | | | | | | | | | |
| 79 | HMEJE13 | 209423 | Lambda ZAP | 89 | 2086 | 1 | 1131 | 147 | 147 | 217 | 1 | 26 | 27 | 55 |
| | | 10/30/97 | II | | | | | | | | | | | |
| 80 | HNAAF65 | 209423 | pSport1 | 90 | 891 | 1 | 891 | 140 | 140 | 218 | 1 | 21 | 22 | 212 |
| | | 10/30/97 | | | | | | | | | | | | |
| 81 | HNFHY30 | 209423 | Uni-ZAP XR | 91 | 1974 | 1 | 1974 | 134 | 134 | 219 | 1 | 30 | 31 | 40 |
| | | 10/30/97 | | | | | | | | | | | | |
| 82 | HNFIR81 | 209423 | pBluescript | 92 | 1423 | 1 | 1423 | 19 | 19 | 220 | 1 | 20 | 21 | 59 |
| | | 10/30/97 | | | | | | | | | | | | |
| 83 | HNTBI57 | 209423 | pCMVSport | 93 | 1365 | 134 | 1365 | 210 | 210 | 221 | 1 | 26 | 27 | 58 |
| | | 10/30/97 | 3.0 | | | | | | | | | | | |
| 84 | HSAYR13 | 209423 | Uni-ZAP XR | 94 | 756 | 1 | 756 | 171 | 171 | 222 | 1 | 19 | 20 | 45 |
| | | 10/30/97 | | | | | | | | | | | | |
| 85 | HTOHV49 | 209407 | Uni-ZAP XR | 95 | 938 | 1 | 729 | 62 | 62 | 223 | 1 | 19 | 20 | 61 |
| | | 10/23/97 | | | | | | | | | | | | |
| 86 | HSFAG37 | 209368 | Uni-ZAP XR | 96 | 928 | 1 | 928 | 264 | 264 | 224 | 1 | 18 | 19 | 51 |
| | | 10/16/97 | | | | | | | | | | | | |
| 87 | HTXBU52 | 209407 | Uni-ZAP XR | 97 | 1715 | 557 | 1715 | 574 | 574 | 225 | 1 | 34 | 35 | 50 |
| | | 10/23/97 | | | | | | | | | | | | |
| 88 | HLHFP18 | 209407 | Uni-ZAP XR | 98 | 678 | 1 | 678 | 25 | 25 | 226 | 1 | 24 | 25 | 46 |
| | | 10/23/97 | | | | | | | | | | | | |
| 89 | HFXBW09 | 209423 | Lambda ZAP | 99 | 1541 | 1 | 1541 | 159 | 159 | 227 | 1 | 29 | 30 | 51 |
| | | 10/30/97 | II | | | | | | | | | | | |
| 90 | HNGEM62 | 209423 | Uni-ZAP XR | 100 | 881 | 1 | 881 | 78 | 78 | 228 | 1 | 21 | 22 | 65 |
| | | 10/30/97 | | | | | | | | | | | | |
| 91 | HNGJF92 | 209423 | Uni-ZAP XR | 101 | 947 | 1 | 947 | 40 | 40 | 229 | 1 | 31 | 32 | 46 |
| | | 10/30/97 | | | | | | | | | | | | |
| 92 | HMEED18 | 209368 | Lambda ZAP | 102 | 1369 | 28 | 1369 | 34 | 34 | 230 | 1 | 34 | 35 | 221 |
| | | 10/16/97 | II | | | | | | | | | | | |
| 93 | HMIAM45 | 209368 | Uni-ZAP XR | 103 | 1231 | 1 | 1231 | 68 | 68 | 231 | 1 | 37 | 38 | 48 |
| | | 10/16/97 | | | | | | | | | | | | |
| 94 | HSAVK10 | 209368 | Uni-ZAP XR | 104 | 1242 | 1 | 1242 | 131 | 131 | 232 | 1 | 32 | 33 | 40 |
| | | 10/16/97 | | | | | | | | | | | | |
| 95 | HSDHC81 | 209368 | Uni-ZAP XR | 105 | 1151 | 1 | 1151 | 184 | 184 | 233 | 1 | 22 | 23 | 52 |
| | | 10/16/97 | | | | | | | | | | | | |
| 96 | HSLCT04 | 209368 | Uni-ZAP XR | 106 | 1628 | 1 | 1628 | 159 | 159 | 234 | 1 | 36 | 37 | 49 |
| | | 10/16/97 | | | | | | | | | | | | |
| 97 | HMDAB56 | 209368 | Uni-ZAP XR | 107 | 1465 | 1 | 1465 | 273 | 273 | 235 | 1 | 33 | 34 | 44 |
| | | 10/16/97 | | | | | | | | | | | | |
| 98 | HUDBZ89 | 209407 | ZAP Express | 108 | 1265 | 1 | 1265 | 197 | 197 | 236 | 1 | 17 | 18 | 54 |
| | | 10/23/97 | | | | | | | | | | | | |
| 99 | HLYCT47 | 209407 | pSport1 | 109 | 1006 | 1 | 1006 | 47 | 47 | 237 | 1 | 22 | 23 | 68 |
| | | 10/23/97 | | | | | | | | | | | | |
| 100 | HOSDJ25 | 209423 | Uni-ZAP XR | 110 | 2214 | 985 | 2214 | 1076 | 1076 | 238 | 1 | 18 | 19 | 40 |
| | | 10/30/97 | | | | | | | | | | | | |
| 100 | HOSDJ25 | 209423 | Uni-ZAP XR | 137 | 1258 | 1 | 1258 | 146 | 146 | 265 | 1 | 18 | 19 | 40 |
| | | 10/30/97 | | | | | | | | | | | | |
| 101 | HADAO89 | 209423 | pSport1 | 111 | 1453 | 1 | 1453 | 244 | 244 | 239 | 1 | 22 | 23 | 44 |
| | | 10/30/97 | | | | | | | | | | | | |
| 102 | HMSGB14 | 209423 | Uni-ZAP XR | 112 | 1552 | 1 | 1552 | 138 | 138 | 240 | 1 | 18 | 19 | 77 |
| | | 10/30/97 | | | | | | | | | | | | |
| 103 | HPMGD01 | 209423 | Uni-ZAP XR | 113 | 1489 | 140 | 1489 | 157 | 157 | 241 | 1 | 36 | 37 | 52 |
| | | 10/30/97 | | | | | | | | | | | | |
| 104 | HNHFU32 | 209407 | Uni-ZAP XR | 114 | 607 | 1 | 607 | 175 | 175 | 242 | 1 | 30 | 31 | 52 |
| | | 10/23/97 | | | | | | | | | | | | |
| 105 | HMIAL40 | 209368 | Uni-ZAP XR | 115 | 1498 | 1 | 1498 | 235 | 235 | 243 | 1 | 19 | 20 | 42 |
| | | 10/16/97 | | | | | | | | | | | | |
| 106 | HAMFY69 | 209407 | pCMVSport | 116 | 1797 | 314 | 1797 | 359 | 359 | 244 | 1 | 17 | 18 | 48 |
| | | 10/23/97 | 3.0 | | | | | | | | | | | |
| 107 | HBMCT17 | 209407 | pBluescript | 117 | 952 | 1 | 952 | 160 | 160 | 245 | 1 | 25 | 26 | 74 |
| | | 10/23/97 | | | | | | | | | | | | |
| 108 | HEBFI91 | 209407 | Uni-ZAP XR | 118 | 1185 | 1 | 1185 | 132 | 132 | 246 | 1 | 20 | 21 | 43 |
| | | 10/23/97 | | | | | | | | | | | | |
| 109 | HHEAH86 | 209407 | pCMVSport | 119 | 1098 | 1 | 1098 | 75 | 75 | 247 | 1 | 16 | 17 | 64 |
| | | 10/23/97 | 3.0 | | | | | | | | | | | |
| 110 | HRDFD27 | 209423 | Uni-ZAP XR | 120 | 805 | 1 | 805 | 82 | 82 | 248 | 1 | 36 | 37 | 83 |
| | | 10/30/97 | | | | | | | | | | | | |
| 111 | HTPCS72 | 209423 | Uni-ZAP XR | 121 | 3435 | 2141 | 3431 | 2365 | 2365 | 249 | 1 | 29 | 30 | 71 |
| | | 10/30/97 | | | | | | | | | | | | |
| 111 | HTPCS72 | 209423 | Uni-ZAP XR | 138 | 1598 | 306 | 1598 | 530 | 530 | 266 | 1 | 29 | 30 | 71 |
| | | 10/30/97 | | | | | | | | | | | | |
| 112 | HFFAL36 | 209368 | Lambda ZAP | 122 | 1020 | 1 | 1020 | 68 | 68 | 250 | 1 | 35 | 36 | 56 |
| | | 10/16/97 | II | | | | | | | | | | | |
| 113 | HFXBT12 | 209368 | Lambda ZAP | 123 | 1378 | 1 | 1378 | 79 | 79 | 251 | 1 | 18 | 19 | 66 |
| | | 10/16/97 | II | | | | | | | | | | | |
| 114 | HNGJF70 | 209368 | Uni-ZAP XR | 124 | 1146 | 1 | 1146 | 94 | 94 | 252 | 1 | 16 | 17 | 45 |
| | | 10/16/97 | | | | | | | | | | | | |
| 115 | HATEE46 | 209407 | Uni-ZAP XR | 125 | 1675 | 136 | 863 | 241 | 241 | 253 | 1 | 21 | 22 | 53 |
| | | 10/23/97 | | | | | | | | | | | | |
| 116 | HJMBN89 | 209407 | pCMVSport | 126 | 1064 | 306 | 1064 | 348 | 348 | 254 | 1 | 13 | 14 | 56 |
| | | 10/23/97 | 3.0 | | | | | | | | | | | |
| 117 | HNHEK61 | 209407 | Uni-ZAP XR | 127 | 1607 | 1 | 1607 | 45 | 45 | 255 | 1 | 24 | 25 | 41 |
| | | 10/23/97 | | | | | | | | | | | | |
| 118 | HEQAO65 | 209407 | pCMVSport | 128 | 1037 | 5 | 1037 | 152 | 152 | 256 | 1 | 27 | 28 | 160 |
| | | 10/23/97 | 3.0 | | | | | | | | | | | |
| 119 | HFCDV54 | 209407 | Uni-ZAP XR | 129 | 1146 | 1 | 1146 | 27 | 27 | 257 | 1 | 29 | 30 | 50 |
| | | 10/23/97 | | | | | | | | | | | | |
| 120 | HHEAD14 | 209407 | pCMVSport | 130 | 1172 | 1 | 1172 | 53 | 53 | 258 | 1 | 18 | 19 | 278 |
| | | 10/23/97 | 3.0 | | | | | | | | | | | |
| 121 | HGBHE57 | 209407 | Uni-ZAP XR | 131 | 663 | 1 | 663 | 14 | 14 | 259 | 1 | 19 | 20 | 68 |
| | | 10/23/97 | | | | | | | | | | | | |
| 122 | HGLAF75 | 209407 | Uni-ZAP XR | 132 | 776 | 1 | 776 | 231 | 231 | 260 | 1 | 28 | 29 | 121 |
| | | 10/23/97 | | | | | | | | | | | | |
| 123 | HHEMQ28 | 209407 | pCMVSport | 133 | 1543 | 286 | 1543 | 442 | 442 | 261 | 1 | 31 | 32 | 58 |
| | | 10/23/97 | 3.0 | | | | | | | | | | | |
| 124 | HMWEC56 | 209368 | Uni-Zap XR | 134 | 2157 | 1013 | 2146 | 1067 | 1067 | 262 | 1 | 17 | 18 | 67 |
| | | 10/16/97 | | | | | | | | | | | | |
| 125 | HERAR44 | 209407 | Uni-ZAP XR | 135 | 420 | 1 | 420 | 60 | 60 | 263 | 1 | 40 | 41 | 45 |
| | | 10/23/97 | | | | | | | | | | | | |

Table 1 summarizes the information corresponding to each “Gene No.” described above. The nucleotide sequence identified as “NT SEQ ID NO:X” was assembled from partially homologous (“overlapping”) sequences obtained from the “cDNA clone ID” identified in Table 1 and, in some cases, from additional related DNA clones. The overlapping sequences were assembled into a single contiguous sequence of high redundancy (usually three to five overlapping sequences at each nucleotide position), resulting in a final sequence identified as SEQ ID NO:X.

The cDNA Clone ID was deposited on the date and given the corresponding deposit number listed in “ATCC Deposit No:Z and Date.” Some of the deposits contain multiple different clones corresponding to the same gene. “Vector” refers to the type of vector contained in the cDNA Clone ID. “Total NT Seq.” refers to the total number of nucleotides in the contig identified by “Gene No.” The deposited clone may contain all or most of these sequences, reflected by the nucleotide position indicated as “5′ NT of Clone Seq.” and the “3′ NT of Clone Seq.” of SEQ ID NO:X. The nucleotide position of SEQ ID NO:X of the putative start codon (methionine) is identified as “5′ NT of Start Codon.” Similarly, the nucleotide position of SEQ ID NO:X of the predicted signal sequence is identified as “5′ NT of First AA of Signal Pep.” The translated amino acid sequence, beginning with the methionine, is identified as “AA SEQ ID NO:Y,” although other reading frames can also be easily translated using known molecular biology techniques. The polypeptides produced by these alternative open reading frames are specifically contemplated by the present invention.

The first and last amino acid position of SEQ ID NO:Y of the predicted signal peptide is identified as “First AA of Sig Pep” and “Last AA of Sig Pep.” The predicted first amino acid position of SEQ ID NO:Y of the secreted portion is identified as “Predicted First AA of Secreted Portion.” Finally, the amino acid position of SEQ ID NO:Y of the last amino acid in the open reading frame is identified as “Last AA of ORF.”

SEQ ID NO:X and the translated SEQ ID NO:Y are sufficiently accurate and otherwise suitable for a variety of uses well known in the art and described further below. For instance, SEQ ID NO:X is useful for designing nucleic acid hybridization probes that will detect nucleic acid sequences contained in SEQ ID NO:X or the cDNA contained in the deposited clone. These probes will also hybridize to nucleic acid molecules in biological samples, thereby enabling a variety of forensic and diagnostic methods of the invention. Similarly, polypeptides identified from SEQ ID NO:Y may be used to generate antibodies which bind specifically to the secreted proteins encoded by the cDNA clones identified in Table 1.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:X and the predicted translated amino acid sequence identified as SEQ ID NO:Y, but also a sample of plasmid DNA containing a human cDNA of the invention deposited with the ATCC, as set forth in Table 1. The nucleotide sequence of each deposited clone can readily be determined by sequencing the deposited clone in accordance with known methods. The predicted amino acid sequence can then be verified from such deposits. Moreover, the amino acid sequence of the protein encoded by a particular clone can also be directly determined by peptide sequencing or by expressing the protein in a suitable host cell containing the deposited human cDNA, collecting the protein, and determining its sequence.

The present invention also relates to the genes corresponding to SEQ ID NO:X, SEQ ID NO:Y, or the deposited clone. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologs. Species homologs may be isolated and identified by making suitable probes or primers from the sequences provided herein and screening a suitable nucleic acid source for the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith and Johnson, Gene 67:31-40 (1988). Polypeptides of the invention also can be purified from natural or recombinant sources using antibodies of the invention raised against the secreted protein in methods which are well known in the art.

Signal Sequences

Methods for predicting whether a protein has a signal sequence, as well as the cleavage point for that sequence, are available. For instance, the method of McGeoch, Virus Res. 3:271-286 (1985), uses the information from a short N-terminal charged region and a subsequent uncharged region of the complete (uncleaved) protein. The method of von Heinje, Nucleic Acids Res. 14:4683-4690 (1986) uses the information from the residues surrounding the cleavage site, typically residues −13 to +2, where +1 indicates the amino terminus of the secreted protein. The accuracy of predicting the cleavage points of known mammalian secretory proteins for each of these methods is in the range of 75-80%. (von Heinje, supra.) However, the two methods do not always produce the same predicted cleavage point(s) for a given protein.

In the present case, the deduced amino acid sequence of the secreted polypeptide was analyzed by a computer program called SignalP (Henrik Nielsen et al., Protein Engineering 10:1-6 (1997)), which predicts the cellular location of a protein based on the amino acid sequence. As part of this computational prediction of localization, the methods of McGeoch and von Heinje are incorporated. The analysis of the amino acid sequences of the secreted proteins described herein by this program provided the results shown in Table 1.

As one of ordinary skill would appreciate, however, cleavage sites sometimes vary from organism to organism and cannot be predicted with absolute certainty. Accordingly, the present invention provides secreted polypeptides having a sequence shown in SEQ ID NO:Y which have an N-terminus beginning within 5 residues (i.e., +or −5 residues) of the predicted cleavage point. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. These polypeptides, and the polynucleotides encoding such polypeptides, are contemplated by the present invention.

Polynucleotide and Polypeptide Variants

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in Table 1, the ORF (open reading frame), or any fragement specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identiy are: Matrix=Unitary, k-tuple=4, Mismatch Penalty=l, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignement of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequnce are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in Table 1 or to the amino acid sequence encoded by deposited DNA clone can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is becuase the FASTDB program does not account for N- and C-terminal truncations of the subject sequence when calculating global percent identity. For subject sequences truncated at the N- and C-termini, relative to the the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N— and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequnce are manually corrected for. No other manual corrections are to made for the purposes of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).) A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5, 5-10, 5-25, 5-50, 10-50 or 50-150, conservative amino acid substitutions are preferable.

Polynucleotide and Polypeptide Fragments

In the present invention, a “polynucleotide fragment” refers to a short polynucleotide having a nucleic acid sequence contained in the deposited clone or shown in SEQ ID NO:X. The short nucleotide fragments are preferably at least about 15 nt, and more preferably at least about 20 nt, still more preferably at least about 30 nt, and even more preferably, at least about 40-nt in length. A fragment “at least 20 nt in length,” for example, is intended to include 20 or more contiguous bases from the cDNA sequence contained in the deposited clone or the nucleotide sequence shown in SEQ ID NO:X. These nucleotide fragments are useful as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:X or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:Y or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-60, can be deleted from the amino terminus of either the secreted polypeptide or the mature form. Similarly, any number of amino acids, ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:Y falling within conserved domains are specifically contemplated by the present invention. Moreover, polynucleotide fragments encoding these domains are also contemplated.

Other preferred fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Epitopes & Antibodies

In the present invention, “epitopes” refer to polypeptide fragments having antigenic or immunogenic activity in an animal, especially in a human. A preferred embodiment of the present invention relates to a polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al., Proc. Natl. Acad. Sci. USA 81:3998-4002 (1983).)

Fragments which function as epitopes may be produced by any conventional means. (See, e.g., Houghten, R. A., Proc. Natl. Acad. Sci. USA 82:5131-5135 (1985) further described in U.S. Pat. No. 4,631,211.) In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).) Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985).) A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.) As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimeric, single chain, and humanized antibodies.

Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides are familiar and routine techniques in the art.

Moreover, polypeptides of the present invention, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).) Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the polypeptides of the present invention can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the polynucleotides or the polypeptides of the present invention.

Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in SEQ ID NO:X. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:X will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

In addition to the foregoing, a polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

The polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, polynucleotides of the present invention can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (1251, 121I), carbon (14C), sulfur (35S), tritium (3H), indium (112In), and technetium (99mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder.

Moreover, polypeptides of the present invention can be used to treat disease. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to a polypeptide of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

Biological Activities

The polynucleotides and polypeptides of the present invention can be used in assays to test for one or more biological activities. If these polynucleotides and polypeptides do exhibit activity in a particular assay, it is likely that these molecules may be involved in the diseases associated with the biological activity. Thus, the polynucleotides and polypeptides could be used to treat the associated disease.

Immune Activity

A polypeptide or polynucleotide of the present invention may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, a polynucleotide or polypeptide of the present invention can be used as a marker or detector of a particular immune system disease or disorder.

A polynucleotide or polypeptide of the present invention may be useful in treating or detecting deficiencies or disorders of hematopoietic cells. A polypeptide or polynucleotide of the present invention could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a polypeptide or polynucleotide of the present invention could also be used to modulate hemostatic (the stopping of bleeding) or thrombolytic activity (clot formation). For example, by increasing hemostatic or thrombolytic activity, a polynucleotide or polypeptide of the present invention could be used to treat blood coagulation disorders (e.g., afibrinogenemia, factor deficiencies), blood platelet disorders (e.g. thrombocytopenia), or wounds resulting from trauma, surgery, or other causes. Alternatively, a polynucleotide or polypeptide of the present invention that can decrease hemostatic or thrombolytic activity could be used to inhibit or dissolve clotting. These molecules could be important in the treatment of heart attacks (infarction), strokes, or scarring.

A polynucleotide or polypeptide of the present invention may also be useful in treating or detecting autoimmune disorders. Many autoimmune disorders result from inappropriate recognition of self as foreign material by irmune cells. This inappropriate recognition results in an immune response leading to the destruction of the host tissue. Therefore, the administration of a polypeptide or polynucleotide of the present invention that inhibits an immune response, particularly the proliferation, differentiation, or chemotaxis of T-cells, may be an effective therapy in preventing autoimmune disorders.

Examples of autoimmune disorders that can be treated or detected by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by a polypeptide or polynucleotide of the present invention. Moreover, these molecules can be used to treat anaphylaxis, hypersensitivity to an antigenic molecule, or blood group incompatibility.

A polynucleotide or polypeptide of the present invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a polypeptide or polynucleotide of the present invention that inhibits an immune response, particularly the proliferation, differentiation, or chemotaxis of T-cells, may be an effective therapy in preventing organ rejection or GVHD.

Similarly, a polypeptide or polynucleotide of the present invention may also be used to modulate inflammation. For example, the polypeptide or polynucleotide may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

A polypeptide or polynucleotide can be used to treat or detect hyperproliferative disorders, including neoplasms. A polypeptide or polynucleotide of the present invention may inhibit the proliferation of the disorder through direct or indirect interactions. Alternatively, a polypeptide or polynucleotide of the present invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by a polynucleotide or polypeptide of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by a polynucleotide or polypeptide of the present invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

A polypeptide or polynucleotide of the present invention can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, the polypeptide or polynucleotide of the present invention may also directly inhibit the infectious agent, without necessarily eliciting an immune response.

Viruses are one example of an infectious agent that can cause disease or symptoms that can be treated or detected by a polynucleotide or polypeptide of the present invention. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. A polypeptide or polynucleotide of the present invention can be used to treat or detect any of these symptoms or diseases.

Similarly, bacterial or fungal agents that can cause disease or symptoms and that can be treated or detected by a polynucleotide or polypeptide of the present invention include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosorniasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

Regeneration

A polynucleotide or polypeptide of the present invention can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, bums, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, a polynucleotide or polypeptide of the present invention may increase regeneration of tissues difficult to heal. For example, increased tendon/ligament regeneration would quicken recovery time after damage. A polynucleotide or polypeptide of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a polynucleotide or polypeptide of the present invention to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the polynucleotide or polypeptide of the present invention.

Chemotaxis

A polynucleotide or polypeptide of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide of the present invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that a polynucleotide or polypeptide of the present invention may inhibit chemotactic activity. These molecules could also be used to treat disorders. Thus, a polynucleotide or polypeptide of the present invention could be used as an inhibitor of chemotaxis.

Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (b) determining if a biological activity of the polypeptide has been altered.

Other Activities

A polypeptide or polynucleotide of the present invention may also increase or decrease the differentiation or proliferation of embryonic stem cells, besides, as discussed above, hematopoietic lineage.

A polypeptide or polynucleotide of the present invention may also be used to modulate mammalian characteristics, such as body height, weight, hair color, eye color, skin, percentage of adipose tissue, pigmentation, size, and shape (e.g., cosmetic surgery). Similarly, a polypeptide or polynucleotide of the present invention may be used to modulate mammalian metabolism affecting catabolism, anabolism, processing, utilization, and storage of energy.

A polypeptide or polynucleotide of the present invention may be used to change a mamrnmal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

A polypeptide or polynucleotide of the present invention may also be used as a food additive or preservative, such as to increase or decrease storage capabilities, fat content, lipid, protein, carbohydrate, vitamins, minerals, cofactors or other nutritional components.

Other Preferred Embodiments

Other preferred embodiments of the claimed invention include an isolated nucleic acid molecule comprising a nucleotide sequence which is at least 95% identical to a sequence of at least about 50 contiguous nucleotides in the nucleotide sequence of SEQ ID NO:X wherein X is any integer as defined in Table 1.

Also preferred is a nucleic acid molecule wherein said sequence of contiguous nucleotides is included in the nucleotide sequence of SEQ ID NO:X in the range of positions beginning with the nucleotide at about the position of the 5′ Nucleotide of the Clone Sequence and ending with the nucleotide at about the position of the 3′ Nucleotide of the Clone Sequence as defined for SEQ ID NO:X in Table 1.

Also preferred is a nucleic acid molecule wherein said sequence of contiguous nucleotides is included in the nucleotide sequence of SEQ ID NO:X in the range of positions beginning with the nucleotide at about the position of the 5′ Nucleotide of the Start Codon and ending with the nucleotide at about the position of the 3′ Nucleotide of the Clone Sequence as defined for SEQ ID NO:X in Table 1.

Similarly preferred is a nucleic acid molecule wherein said sequence of contiguous nucleotides is included in the nucleotide sequence of SEQ ID NO:X in the range of positions beginning with the nucleotide at about the position of the 5′ Nucleotide of the First Amino Acid of the Signal Peptide and ending with the nucleotide at about the position of the 3′ Nucleotide of the Clone Sequence as defined for SEQ ID NO:X in Table 1.

Also preferred is an isolated nucleic acid molecule comprising a nucleotide sequence which is at least 95% identical to a sequence of at least about 150 contiguous nucleotides in the nucleotide sequence of SEQ ID NO:X.

Further preferred is an isolated nucleic acid molecule comprising a nucleotide sequence which is at least 95% identical to a sequence of at least about 500 contiguous nucleotides in the nucleotide sequence of SEQ ID NO:X.

A further preferred embodiment is a nucleic acid molecule comprising a nucleotide sequence which is at least 95% identical to the nucleotide sequence of SEQ ID NO:X beginning with the nucleotide at about the position of the 5′ Nucleotide of the First Amino Acid of the Signal Peptide and ending with the nucleotide at about the position of the 3′ Nucleotide of the Clone Sequence as defined for SEQ ID NO:X in Table 1.

A further preferred embodiment is an isolated nucleic acid molecule comprising a nucleotide sequence which is at least 95% identical to the complete nucleotide sequence of SEQ ID NO:X.

Also preferred is an isolated nucleic acid molecule which hybridizes under stringent hybridization conditions to a nucleic acid molecule, wherein said nucleic acid molecule which hybridizes does not hybridize under stringent hybridization conditions to a nucleic acid molecule having a nucleotide sequence consisting of only A residues or of only T residues.

Also preferred is a composition of matter comprising a DNA molecule which comprises a human cDNA clone identified by a cDNA Clone Identifier in Table 1, which DNA molecule is contained in the material deposited with the American Type Culture Collection and given the ATCC Deposit Number shown in Table 1 for said cDNA Clone Identifier.

Also preferred is an isolated nucleic acid molecule comprising a nucleotide sequence which is at least 95% identical to a sequence of at least 50 contiguous nucleotides in the nucleotide sequence of a human cDNA clone identified by a cDNA Clone Identifier in Table 1, which DNA molecule is contained in the deposit given the ATCC Deposit Number shown in Table 1.

Also preferred is an isolated nucleic acid molecule, wherein said sequence of at least 50 contiguous nucleotides is included in the nucleotide sequence of the complete open reading frame sequence encoded by said human cDNA clone.

Also preferred is an isolated nucleic acid molecule comprising a nucleotide sequence which is at least 95% identical to sequence of at least 150 contiguous nucleotides in the nucleotide sequence encoded by said human cDNA clone.

A further preferred embodiment is an isolated nucleic acid molecule comprising a nucleotide sequence which is at least 95% identical to sequence of at least 500 contiguous nucleotides in the nucleotide sequence encoded by said human cDNA clone.

A further preferred embodiment is an isolated nucleic acid molecule comprising a nucleotide sequence which is at least 95% identical to the complete nucleotide sequence encoded by said human cDNA clone.

A further preferred embodiment is a method for detecting in a biological sample a nucleic acid molecule comprising a nucleotide sequence which is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1; which method comprises a step of comparing a nucleotide sequence of at least one nucleic acid molecule in said sample with a sequence selected from said group and determining whether the sequence of said nucleic acid molecule in said sample is at least 95% identical to said selected sequence.

Also preferred is the above method wherein said step of comparing sequences comprises determining the extent of nucleic acid hybridization between nucleic acid molecules in said sample and a nucleic acid molecule comprising said sequence selected from said group. Similarly, also preferred is the above method wherein said step of comparing sequences is performed by comparing the nucleotide sequence determined from a nucleic acid molecule in said sample with said sequence selected from said group. The nucleic acid molecules can comprise DNA molecules or RNA molecules.

A further preferred embodiment is a method for identifying the species, tissue or cell type of a biological sample which method comprises a step of detecting nucleic acid molecules in said sample, if any, comprising a nucleotide sequence that is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1.

The method for identifying the species, tissue or cell type of a biological sample can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

Also preferred is a method for diagnosing in a subject a pathological condition associated with abnormal structure or expression of a gene encoding a secreted protein identified in Table 1, which method comprises a step of detecting in a biological sample obtained from said subject nucleic acid molecules, if any, comprising a nucleotide sequence that is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1.

The method for diagnosing a pathological condition can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

Also preferred is a composition of matter comprising isolated nucleic acid molecules wherein the nucleotide sequences of said nucleic acid molecules comprise a panel of at least two nucleotide sequences, wherein at least one sequence in said panel is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1. The nucleic acid molecules can comprise DNA molecules or RNA molecules.

Also preferred is an isolated polypeptide comprising an amino acid sequence at least 90% identical to a sequence of at least about 10 contiguous amino acids in the amino acid sequence of SEQ ID NO:Y wherein Y is any integer as defined in Table 1.

Also preferred is a polypeptide, wherein said sequence of contiguous amino acids is included in the amino acid sequence of SEQ ID NO:Y in the range of positions beginning with the residue at about the position of the First Amino Acid of the Secreted Portion and ending with the residue at about the Last Amino Acid of the Open Reading Frame as set forth for SEQ ID NO:Y in Table 1.

Also preferred is an isolated polypeptide comprising an amino acid sequence at least 95% identical to a sequence of at least about 30 contiguous amino acids in the amino acid sequence of SEQ ID NO:Y.

Further preferred is an isolated polypeptide comprising an amino acid sequence at least 95% identical to a sequence of at least about 100 contiguous amino acids in the amino acid sequence of SEQ ID NO:Y.

Further preferred is an isolated polypeptide comprising an amino acid sequence at least 95% identical to the complete amino acid sequence of SEQ ID NO:Y.

Further preferred is an isolated polypeptide comprising an amino acid sequence at least 90% identical to a sequence of at least about 10 contiguous amino acids in the complete amino acid sequence of a secreted protein encoded by a human cDNA clone identified by a cDNA Clone Identifier in Table 1 and contained in the deposit with the ATCC Deposit Number shown for said cDNA clone in Table 1.

Also preferred is a polypeptide wherein said sequence of contiguous amino acids is included in the amino acid sequence of a secreted portion of the secreted protein encoded by a human cDNA clone identified by a cDNA Clone Identifier in Table 1 and contained in the deposit with the ATCC Deposit Number shown for said cDNA clone in Table 1.

Also preferred is an isolated polypeptide comprising an amino acid sequence at least 95% identical to a sequence of at least about 30 contiguous amino acids in the amino acid sequence of the secreted portion of the protein encoded by a human cDNA clone identified by a cDNA Clone Identifier in Table 1 and contained in the deposit with the ATCC Deposit Number shown for said cDNA clone in Table 1.

Also preferred is an isolated polypeptide comprising an amino acid sequence at least 95% identical to a sequence of at least about 100 contiguous amino acids in the amino acid sequence of the secreted portion of the protein encoded by a human cDNA clone identified by a cDNA Clone Identifier in Table 1 and contained in the deposit with the ATCC Deposit Number shown for said cDNA clone in Table 1.

Also preferred is an isolated polypeptide comprising an amino acid sequence at least 95% identical to the amino acid sequence of the secreted portion of the protein encoded by a human cDNA clone identified by a cDNA Clone Identifier in Table 1 and contained in the deposit with the ATCC Deposit Number shown for said cDNA clone in Table 1.

Further preferred is an isolated antibody which binds specifically to a polypeptide comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC Deposit Number shown for said cDNA clone in Table Further preferred is a method for detecting in a biological sample a polypeptide comprising an amino acid sequence which is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC Deposit Number shown for said cDNA clone in Table 1; which method comprises a step of comparing an amino acid sequence of at least one polypeptide molecule in said sample with a sequence selected from said group and determining whether the sequence of said polypeptide molecule in said sample is at least 90% identical to said sequence of at least 10 contiguous amino acids.

Also preferred is the above method wherein said step of comparing an amino acid sequence of at least one polypeptide molecule in said sample with a sequence selected from said group comprises determining the extent of specific binding of polypeptides in said sample to an antibody which binds specifically to a polypeptide comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC Deposit Number shown for said cDNA clone in Table 1.

Also preferred is the above method wherein said step of comparing sequences is performed by comparing the amino acid sequence determined from a polypeptide molecule in said sample with said sequence selected from said group.

Also preferred is a method for identifying the species, tissue or cell type of a biological sample which method comprises a step of detecting polypeptide molecules in said sample, if any, comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

Also preferred is the above method for identifying the species, tissue or cell type of a biological sample, which method comprises a step of detecting polypeptide molecules comprising an amino acid sequence in a panel of at least two amino acid sequences, wherein at least one sequence in said panel is at least 90% identical to a sequence of at least 10 contiguous amino acids in a sequence selected from the above group.

Also preferred is a method for diagnosing in a subject a pathological condition associated with abnormal structure or expression of a gene encoding a secreted protein identified in Table 1, which method comprises a step of detecting in a biological sample obtained from said subject polypeptide molecules comprising an amino acid sequence in a panel of at least two amino acid sequences, wherein at least one sequence in said panel is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

In any of these methods, the step of detecting said polypeptide molecules includes using an antibody.

Also preferred is an isolated nucleic acid molecule comprising a nucleotide sequence which is at least 95% identical to a nucleotide sequence encoding a polypeptide wherein said polypeptide comprises an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

Also preferred is an isolated nucleic acid molecule, wherein said nucleotide sequence encoding a polypeptide has been optimized for expression of said polypeptide in a prokaryotic host.

Also preferred is an isolated nucleic acid molecule, wherein said polypeptide comprises an amino acid sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

Further preferred is a method of making a recombinant vector comprising inserting any of the above isolated nucleic acid molecule into a vector. Also preferred is the recombinant vector produced by this method. Also preferred is a method of making a recombinant host cell comprising introducing the vector into a host cell, as well as the recombinant host cell produced by this method.

Also preferred is a method of making an isolated polypeptide comprising culturing this recombinant host cell under conditions such that said polypeptide is expressed and recovering said polypeptide. Also preferred is this method of making an isolated polypeptide, wherein said recombinant host cell is a eukaryotic cell and said polypeptide is a secreted portion of a human secreted protein comprising an amino acid sequence selected from the group consisting of: an amino acid sequence of SEQ ID NO:Y beginning with the residue at the position of the First Amino Acid of the Secreted Portion of SEQ ID NO:Y wherein Y is an integer set forth in Table 1 and said position of the First Amino Acid of the Secreted Portion of SEQ ID NO:Y is defined in Table 1; and an amino acid sequence of a secreted portion of a protein encoded by a human cDNA clone identified by a cDNA Clone Identifier in Table 1 and contained in the deposit with the ATCC Deposit Number shown for said cDNA clone in Table 1. The isolated polypeptide produced by this method is also preferred. Also preferred is a method of treatment of an individual in need of an increased level of a secreted protein activity, which method comprises administering to such an individual a pharmaceutical composition comprising an amount of an isolated polypeptide, polynucleotide, or antibody of the claimed invention effective to increase the level of said protein activity in said individual.

Having generally described the invention, the same will be more readily understood by reference to the following examples, which are provided by way of illustration and are not intended as limiting.

### EXAMPLES

### Example 1

### Isolation of a Selected cDNA Clone from the Deposited Sample

Each cDNA clone in a cited ATCC deposit is contained in a plasmid vector. Table 1 identifies the vectors used to construct the cDNA library from which each clone was isolated. In many cases, the vector used to construct the library is a phage vector from which a plasmid has been excised. The table immediately below correlates the related plasmid for each phage vector used in constructing the cDNA library. For example, where a particular clone is identified in Table 1 as being isolated in the vector “Lambda Zap,” the corresponding deposited clone is in “pBluescript.”

| Vector Used to Construct Library | |
|---|---|
| Plasmid | Corresponding Deposited |
| Lambda Zap | pBluescript (pBS) |
| Uni-Zap XR | pBluescript (pBS) |
| Zap Express | pBK |
| lafmid BA | plafmid BA |
| pSport1 | pSport1 |
| pCMVSport 2.0 | pCMVSport 2.0 |
| pCMVSport 3.0 | pCMVSport 3.0 |
| pCR ® 2.1 | pCR ® 2.1 |

Vectors Lambda Zap (U.S. Pat. Nos. 5,128,256 and 5,286,636), Uni-Zap

XR (U.S. Pat. Nos. 5,128, 256 and 5,286,636), Zap Express (U.S. Pat. Nos. 5,128,256 and 5,286,636), pBluescript (pBS) (Short, J. M. et al., Nucleic Acids Res. 16:7583-7600 (1988); Alting-Mees, M. A. and Short, J. M., Nucleic Acids Res. 17:9494 (1989)) and pBK (Alting-Mees, M. A. et al., Strategies 5:58-61 (1992)) are commercially available from Stratagene Cloning Systems, Inc., 11011 N. Torrey Pines Road, La Jolla, Calif., 92037. pBS contains an ampicillin resistance gene and pBK contains a neomycin resistance gene. Both can be transformed into E. coli strain XL-1 Blue, also available from Stratagene. pBS comes in 4 forms SK+, SK−, KS+and KS. The S and K refers to the orientation of the polylinker to the T7 and T3 primer sequences which flank the polylinker region (“S” is for SacI and “K” is for KpnI which are the first sites on each respective end of the linker). “+” or “−” refer to the orientation of the fl origin of replication (“ori”), such that in one orientation, single stranded rescue initiated from the f1 ori generates sense strand DNA and in the other, antisense.

Vectors pSport1, pCMVSport 2.0 and pCMVSport 3.0, were obtained from Life Technologies, Inc., P. O. Box 6009, Gaithersburg, Md. 20897. All Sport vectors contain an ampicillin resistance gene and may be transformed into E. coli strain DH10B, also available from Life Technologies. (See, for instance, Gruber, C. E., et al., Focus 15:59 (1993).) Vector lafmid BA (Bento Soares, Columbia University, NY) contains an ampicillin resistance gene and can be transformed into E. coli strain XL-1 Blue. Vector pCR®2.1, which is available from Invitrogen, 1600 Faraday Avenue, Carlsbad, Calif. 92008, contains an ampicillin resistance gene and may be transformed into E. coli strain DH10B, available from Life Technologies. (See, for instance, Clark, J. M., Nuc. Acids Res. 16:9677-9686 (1988) and Mead, D. et al., Bio/Technology 9: (1991).) Preferably, a polynucleotide of the present invention does not comprise the phage vector sequences identified for the particular clone in Table 1, as well as the corresponding plasmid vector sequences designated above.

The deposited material in the sample assigned the ATCC Deposit Number cited in Table 1 for any given cDNA clone also may contain one or more additional plasmids, each comprising a cDNA clone different from that given clone. Thus, deposits sharing the same ATCC Deposit Number contain at least a plasmid for each cDNA clone identified in Table 1. Typically, each ATCC deposit sample cited in Table 1 comprises a mixture of approximately equal amounts (by weight) of about 50 plasmid DNAs, each containing a different cDNA clone; but such a deposit sample may include plasmids for more or less than 50 cDNA clones, up to about 500 cDNA clones.

Two approaches can be used to isolate a particular clone from the deposited sample of plasmid DNAs cited for that clone in Table 1. First, a plasmid is directly isolated by screening the clones using a polynucleotide probe corresponding to SEQ ID NO:X.

Particularly, a specific polynucleotide with 30-40 nucleotides is synthesized using an Applied Biosystems DNA synthesizer according to the sequence reported. The oligonucleotide is labeled, for instance, with ³²P-γ-ATP using T4 polynucleotide kinase and purified according to routine methods. (E.g., Maniatis et al., Molecular Cloning: A Laboratory Manual, Cold Spring Harbor Press, Cold Spring, NY (1982).) The plasmid mixture is transformed into a suitable host, as indicated above (such as XL-1 Blue (Stratagene)) using techniques known to those of skill in the art, such as those provided by the vector supplier or in related publications or patents cited above. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:X (i.e., within the region of SEQ ID NO:X bounded by the 5′ NT and the 3′ NT of the clone defined in Table 1) are synthesized and used to amplify the desired cDNA using the deposited cDNA plasmid as a template. The polymerase chain reaction is carried out under routine conditions, for instance, in 25 μl of reaction mixture with 0.5 ug of the above cDNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of a gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).) Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene.

### Example 2

### Isolation of Genomic Clones Corresponding to a Polynucleotide

A human genomic P1 library (Genomic Systems, Inc.) is screened by PCR using primers selected for the cDNA sequence corresponding to SEQ ID NO:X., according to the method described in Example 1. (See also, Sambrook.)

### Example 3

### Tissue Distribution of Polypeptide

Tissue distribution of mRNA expression of polynucleotides of the present invention is determined using protocols for Northern blot analysis, described by, among others, Sambrook et al. For example, a cDNA probe produced by the method described in Example 1 is labeled with P³² using the rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70° C. overnight, and the films developed according to standard procedures.

### Example 4

### Chromosomal Mapping of the Polynucleotides

An oligonucleotide primer set is designed according to the sequence at the 5′ end of SEQ ID NO:X. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5 minute cycle at 70° C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of a Polypeptide

A polynucleotide encoding a polypeptide of the present invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000× g). The cell pellet is solubilized in the chaotropic agent 6 Molar Guanidine HCl by stirring for 3-4 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

In addition to the above expression vector, the present invention further includes an expression vector comprising phage operator and promoter elements operatively linked to a polynucleotide of the present invention, called pHE4a. (ATCC Accession Number 209645, deposited on Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgamo sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of a Polypeptide from an Inclusion Body

The following alternative method can be used to purify a polypeptide expressed in E coli when it is present in the form of inclusion bodies. Unless otherwise specified, all of the following steps are conducted at 4-10C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000× g centrifugation for 15 min., the pellet is discarded and the polypeptide containing supernatant is incubated at 4° C. overnight to allow further GuHCl extraction.

Following high speed centrifugation (30,000× g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Commassie blue stained 16% SDS-PAGE gel when 5 μg of purified protein is loaded. The purified protein can also be tested for endotoxin/LPS contamination, and typically the LPS content is less than 0.1 ng/ml according to LAL assays.

### Example 7

### Cloning and Expression of a Polypeptide in a Baculovirus Expression System

In this example, the plasmid shuttle vector pA2 is used to insert a polynucleotide into a baculovirus to express a polypeptide. This expression vector contains the strong polyhedrin promoter of the Autographa californica nuclear polyhedrosis virus (AcMNPV) followed by convenient restriction sites such as BamHI, XbaI and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the cDNA sequence contained in the deposited clone, including the AUG initiation codon and the naturally associated leader sequence identified in Table 1, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five μg of a plasmid containing the polynucleotide is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One μg of BaculoGold™ virus DNA and 5 μg of the plasmid are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 8

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transfection with a selectable marker such as dhfr, gpt, neomycin; hygromycin allows the identification and isolation of the transfected cells.

The transfected gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (199Q); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10: 169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of the gene of interest. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

A polynucleotide of the present invention is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 9

### Protein Fusions

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and a polynucleotide of the present invention, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc Region:

| | |
|---|---|
| GGGATCCGGAGCCCAAATCTTCTGACAAAACTCACACATGCCCACCGTGC | (SEQ ID NO:1) |
| CCAGCACCTGAATTCGAGGGTGCACCGTCAGTCTTCCTCTTCCCCCCAAAA | |
| CCCAAGGACACCCTCATGATCTCCCGGACTCCTGAGGTCACATGCGTGGT | |
| GGTGGACGTAAGCCACGAAGACCCTGAGGTCAAGTTCAACTGGTACGTGG | |
| ACGGCGTGGAGGTGCATAATGCCAAGACAAAGCCGCGGGAGGAGCAGTA | |
| CAACAGCACGTACCGTGTGGTCAGCGTCCTCACCGTCCTGCACCAGGACT | |
| GGCTGAATGGCAAGGAGTACAAGTGCAAGGTCTCCAACAAAGCCCTCCCA | |
| ACCCCCATCGAGAAAACCATCTCCAAAGCCAAAGGGCAGCCCCGAGAAC | |
| CACAGGTGTACACCCTGCCCCCATCCCGGGATGAGCTGACCAAGAACCAG | |
| GTCAGCCTGACCTGCCTGGTCAAAGGCTTCTATCCAAGCGACATCGCCGT | |
| GGAGTGGGAGAGCAATGGGCAGCCGGAGAACAACTACAAGACCACGCCT | |
| CCCGTGCTGGACTCCGACGGCTCCTTCTTCCTCTACAGCAAGCTCACCGTG | |
| GACAAGAGCAGGTGGCAGCAGGGGAACGTCTTCTCATGCTCCGTGATGCA | |
| TGAGGCTCTGCACAACCACTACACGCAGAAGAGCCTCTCCCTGTCTCCGG | |
| GTAAATGAGTGCGACGGCCGCGACTCTAGAGGAT | |

### Example 10

### Production of an Antibody from a Polypeptide

The antibodies of the present invention can be prepared by a variety of methods. (See, Current Protocols, Chapter 2.) For example, cells expressing a polypeptide of the present invention is administered to an animal to induce the production of sera containing polyclonal antibodies. In a preferred method, a preparation of the secreted protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a secreted polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 11

### Production of Secreted Protein for High-throughput Screening Assays

The following protocol produces a supernatant containing a polypeptide to be tested. This supernatant can then be used in the Screening Assays described in Examples 13-20.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 mil DMEM(Dulbecco's Modified Eagle Medium)(with 4.5 G/L glucose and L-glutamine (12-604F Biowhittaker))/10% heat inactivated FBS(14-503F Biowhittaker)/1× Penstrep(17-602E Biowhittaker). Let the cells grow overnight.

The next day, mix together in a sterile solution basin: 300 ul Lipofectamine (18324-012 Gibco/BRL) and 5 ml Optimem 1 (31985070 Gibco/BRL)/96-well plate. With a small volume multi-channel pipetter, aliquot approximately 2 ug of an expression vector containing a polynucleotide insert, produced by the methods described in Examples 8 or 9, into an appropriately labeled 96-well round bottom plate. With a multi-channel pipetter, add 50 ul of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a 12-channel pipetter with tips on every other channel, adds the 200 ul of DNA/Lipofectamine/Optimem I complex to the odd wells first, then to the even wells, to each row on the 24-well plates. Incubate at 37° C. for 6 hours.

While cells are incubating, prepare appropriate media, either 1%BSA in DMEM with 1× penstrep, or CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄-H₂O; 71.02 mg/L of Na₂HPO₄; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/mil of L-Proline; 26.25 mg/ml of L-Serine; 101.05 mg/mil of L-Threonine; 19.22 mg/ml of L-Tryptophan; 91.79 mg/ml of L-Tryrosine-2Na-2H₂O; 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of 1-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; and 0.680 mg/L of Vitamin B12; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; and 10 mg/L of Methyl-B-Cyclodextrin complexed with Retinal) with 2 mm glutamine and 1× penstrep. (BSA (81-068-3 Bayer) 100 gm dissolved in 1L DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37° C. for 45 or 72 hours depending on the media used: 1%BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 13-20.

It is specifically understood that when activity is obtained in any of the assays described below using a supernatant, the activity originates from either the polypeptide directly (e.g., as a secreted protein) or by the polypeptide inducing expression of other proteins, which are then secreted into the supernatant. Thus, the invention further provides a method of identifying the protein in the supernatant characterized by an activity in a particular assay.

### Example 12

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proximal region encoding Trp-Ser-Xxx-Trp-Ser (SEQ ID NO:2)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | | JAKs | | | | |
|---|---|---|---|---|---|---|
| Ligand | tyk2 | Jak1 | Jak2 | Jak3 | STATS | GAS(elements) or ISRE |
| IFN family | | | | | | |
| IFN-a/B | + | + | − | − | 1,2,3 | ISRE |
| IFN-g | | + | + | − | 1 | GAS (IRF1>Lys6>IFP) |
| Il-10 | + | ? | ? | − | 1,3 | |
| gp130 family | | | | | | |
| IL-6 (Pleiotrophic) | + | + | + | ? | 1,3 | GAS (IRF1>Lys6>IFP) |
| Il-11(Pleiotrophic) | ? | + | ? | ? | 1,3 | |
| OnM(Pleiotrophic) | ? | + | + | ? | 1,3 | |
| LIF(Pleiotrophic) | ? | + | + | ? | 1,3 | |
| CNTF(Pleiotrophic) | −/+ | + | + | ? | 1,3 | |
| G-CSF(Pleiotrophic) | ? | + | ? | ? | 1,3 | |
| IL-12(Pleiotrophic) | + | − | + | + | 1,3 | |
| g-C family | | | | | | |
| IL-2 (lymphocytes) | − | + | − | + | 1,3,5 | GAS |
| IL-4 (lymph/myeloid) | − | + | − | + | 6 | GAS (IRF1 = IFP >>Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1>IFP>>Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1,3,5 | |
| EPO | ? | − | + | − | 5 | GAS(B-CAS>IRF1=IFP>>Ly6) |
| Receptor Tyrosine Kinases | | | | | | |
| EGF | ? | + | + | − | 1,3 | GAS (IRF1) |
| PDGF | ? | + | + | − | 1,3 | |
| CSF-1 | ? | + | + | − | 1,3 | GAS (not IRF1) |

To construct a synthetic GAS containing promoter element, which is used in the Biological Assays described in Examples 13-14, a PCR based strategy is employed to generate a GAS—SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

| | |
|---|---|
| 5′:GCGCCTCGAGATTTCCCCGAAATCTAGATTTCCCCGAAATGATTTCCCC | (SEQ ID NO:3) |
| GAAATGATTTCCCCGAAATATCTGCCATCTCAATTAG:3′ | |

The downstream primer is complementary to the SV40 promoter and is flanked with a Hind III site: 5′:GCGGCAAGCTTTTTGCAAAGCCTAGGC:3′ (SEQ ID NO:4)

PCR amplification is performed using the SV40 promoter template present in the B-gal:promoter plasmid obtained from Clontech. The resulting PCR fragment is digested with XhoI/Hind III and subcloned into BLSK2-. (Stratagene.) Sequencing with forward and reverse primers confirms that the insert contains the following sequence:

| | |
|---|---|
| 5′:CTCGAGATTTCCCCGAAATCTAGATTTCCCCGAAATGATTTCCCCGAAA | (SEQ ID NO:5) |
| TGATTTCCCCGAAATATCTGCCATCTCAATTAGTCAGCAACCATAGTCCCG | |
| CCCCTAACTCCGCCCATCCCGCCCCTAACTCCGCCCAGTTCCGCCCATTCT | |
| CCGCCCCATGGCTGACTAATTTTTTTTATTTATGCAGAGGCCGAGGCCGCC | |
| TCGGCCTCTGAGCTATTCCAGAAGTAGTGAGGAGGCTTTTTTGGAGGCCT | |
| AGGCTTTTGCAAAAAGCTT:3′ | |

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS—SV40 promoter element is subcloned into the pSEAP-Promoter vector obtained from Clontech using HindIII and XhoI, effectively replacing the SV40 promoter with the amplified GAS:SV40 promoter element, to create the GAS—SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 13-14.

Other constructs can be made using the above description and replacing GAS with a different promoter sequence. For example, construction of reporter molecules containing NFK-B and EGR promoter sequences are described in Examples 15 and 16. However, many other promoters can be substituted using the protocols described in these Examples. For instance, SRE, IL-2, NFAT, or Osteocalcin promoters can be substituted, alone or in combination (e.g., GAS/NF-KB/EGR, GAS/NF-KB, Il-2/NFAT, or NF-KB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 13

### High-throughput Screening Assay for T-cell Activity

The following protocol is used to assess T-cell activity by identifying factors, such as growth factors and cytokines, that may proliferate or differentiate T-cells. T-cell activity is assessed using the GAS/SEAP/Neo construct produced in Example 12. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th 1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI+10% serum with 1%Pen-Strep. Combine 2.5 mls of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing a polypeptide as produced by the protocol described in Example 11.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100, 000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using sellophene covers) and stored at −20° C. until SEAP assays are performed according to Example 17. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

The above protocol may be used in the generation of both transient, as well as, stable transfected cells, which would be apparent to those of skill in the art.

### Example 14

### High-throughput Screening Assay Identifying Myeloid Activity

The following protocol is used to assess myeloid activity by identifying factors, such as growth factors and cytokines, that may proliferate or differentiate myeloid cells. Myeloid cell activity is assessed using the GAS/SEAP/Neo construct produced in Example 12. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 12, a DEAE-Dextran method (Kharbanda et. al., 1994, Cell Growth & Differentiation, 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasrnid DNA, 140 mM NaCl, 5 mM KCl, 375 uM Na₂HPO₄.7H₂O, 1 mM MgCl₂, and 675 uM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 11. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 17.

### Example 15

### High-throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells can be assessed.

The EGR/SEAP reporter construct can be assembled by the following protocol. The EGR-1 promoter sequence (−633 to +1)(Sakamoto K et al., Oncogene 6:867-871 (1991)) can be PCR amplified from human genomic DNA using the following primers:

| | |
|---|---|
| 5′GCGCTCGAGGGATGACAGCGATAGAACCCCGG-3′ | (SEQ ID NO:6) |
| 5′GCGAAGCTTCGCGACTCCCCGGATCCGCCTC-3′ | (SEQ ID NO:7) |

Using the GAS:SEAP/Neo vector produced in Example 12, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 11. EGR-SEAP/PC 12 stable cells are obtained by growing the cells in 300 ug/ml G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 11, 37° C. for 48 to 72 hr. As a positive control, a growth factor known to activate PC12 cells through EGR can be used, such as 50 ng/ul of Neuronal Growth Factor (NGF). Over fifty-fold induction of SEAP is typically seen in the positive control wells. SEAP assay the supernatant according to Example 17.

### Example 16

### High-throughput Screening Assay for T-cell Activity

NF-κB (Nuclear Factor κB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-κB regulates the expression of genes involved in immune cell activation, control of apoptosis (NF-κB appears to shield cells from apoptosis), B and T-cell development, anti-viral and antimicrobial responses, and multiple stress responses.

In non-stimulated conditions, NF-κB is retained in the cytoplasm with I-κB (Inhibitor KB). However, upon stimulation, I-κcB is phosphorylated and degraded, causing NF-κB to shuttle to the nucleus, thereby activating transcription of target genes. Target genes activated by NF-κB include IL-2, IL-6, GM-CSF, ICAM-1 and class 1 MHC.

Due to its central role and ability to respond to a range of stimuli, reporter constructs utilizing the NF-κB promoter element are used to screen the supernatants produced in Example 11. Activators or inhibitors of NF-kB would be useful in treating diseases. For example, inhibitors of NF-κB could be used to treat those diseases related to the acute or chronic activation of NF-kB, such as rheumatoid arthritis.

To construct a vector containing the NF-κB promoter element, a PCR based strategy is employed. The upstream primer contains four tandem copies of the NF-κB binding site (GGGGACTTTCCC) (SEQ ID NO:8), 18 bp of sequence complementary to the 5′ end of the SV40 early promoter sequence, and is flanked with an XhoI site:

| | |
|---|---|
| 5′:GCGGCCTCGAGGGGACTTTCCCGGGGACTTTCCGGGGACTTTCCGGGAC | (SEQ ID NO:9) |
| TTTCCATCCTGCCATCTCAATTAG:3′ | |

The downstream primer is complementary to the 3′ end of the SV40 promoter and is flanked with a Hind III site:

| | |
|---|---|
| 5′:GCGGCAAGCTTTTTGCAAAGCCTAGGC:3′ | (SEQ ID NO:4) |

PCR amplification is performed using the SV40 promoter template present in the pB-gal:promoter plasmid obtained from Clontech. The resulting PCR fragment is digested with XhoI and Hind III and subcloned into BLSK2-. (Stratagene) Sequencing with the T7 and T3 primers confirms the insert contains the following sequence:

| | |
|---|---|
| 5′:CTCGAGGGGACTTTCCCGGGGACTTTCCGGGGACTTTCCGGGACTTTCC | (SEQ ID NO:10) |
| ATCTGCCATCTCAATTAGTCAGCAACCATAGTCCCGCCCCTAACTCCGCCC | |
| ATCCCGCCCCTAACTCCGCCCAGTTCCGCCCATTCTCCGCCCCATGGCTGA | |
| CTAATTTTTTTTATTTATGCAGAGGCCGAGGCCGCCTCGGCCTCTGAGCTA | |
| TTCCAGAAGTAGTGAGGAGGCTTTTTTGGAGGCCTAGGCTTTTGCAAAAA | |
| GCTT:3′ | |

Next, replace the SV40 minimal promoter element present in the pSEAP2-promoter plasmid (Clontech) with this NF-κB/SV40 fragment using XhoI and HindIII. However, this vector does not contain a neomycin resistance gene, and therefore, is not preferred for mammalian expression systems.

In order to generate stable mammalian cell lines, the NF-κB/SV40/SEAP cassette is removed from the above NF-κB/SEAP vector using restriction enzymes SalI and NotI, and inserted into a vector containing neomycin resistance. Particularly, the NF-κB/SV40/SEAP cassette was inserted into pGFP-1 (Clontech), replacing the GFP gene, after restricting pGFP-1 with SalI and NotI.

Once NF-κB/SV40/SEAP/Neo vector is created, stable Jurkat T-cells are created and maintained according to the protocol described in Example 13. Similarly, the method for assaying supernatants with these stable Jurkat T-cells is also described in Example 13. As a positive control, exogenous TNF alpha (0.1,1, 10 ng) is added to wells H9, H10, and H11, with a 5-10 fold activation typically observed.

### Example 17

### Assay for SEAP Activity

As a reporter molecule for the assays described in Examples 13-16, SEAP activity is assayed using the Tropix Phospho-light Kit (Cat. BP400) according to the following general procedure. The Tropix Phospho-light Kit supplies the Dilution, Assay, and Reaction Buffers used below.

Prime a dispenser with the 2.5× Dilution Buffer and dispense 15 μl of 2.5× dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 18

### High-throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-4 (Molecular Probes, Inc.; catalog no. F-14202), used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 ul of buffer after the final wash.

A stock solution of 1 mg/mil fluo-4 is made in 10% pluronic acid DMSO. To load the cells with fluo-4, 50 ul of 12 ug/ml fluo-4 is added to each well. The plate is incubated at 37° C. in a CO₂ incubator for 60 min. The plate is washed four times in the Biotek washer with HBSS leaving 100 ul of buffer.

For non-adherent cells, the cells are spun down from culture media. Cells are re-suspended to 2−5×10⁶ cells/ml with HBSS in a 50-ml conical tube. 4 ul of 1 mg/ml fluo-4 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 ul/well. The plate is centrifuged at 1000 rpm for 5 min. The plate is then washed once in Denley CellWash with 200 ul, followed by an aspiration step to 100 ul final volume.

For a non-cell based assay, each well contains a fluorescent molecule, such as fluo-4. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event which has resulted in an increase in the intracellular Ca⁺⁺ concentration.

### Example 19

### High-throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, the identification of novel human secreted proteins capable of activating tyrosine kinase signal transduction pathways are of interest. Therefore, the following protocol is designed to identify those novel human secreted proteins capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 ul of the supernatant produced in Example 11, the medium was removed and 100 ml of extraction buffer ((20 mM HEPES pH 7.5, 0.15 M NaCl, 1% Triton X-100, 0.1% SDS, 2 mM Na3VO4, 2 mM Na4P2O7 and a cocktail of protease inhibitors (# 1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000× g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 ul ATP/Mg₂₊ (5 mM ATP/50 mM MgCl₂), then 10 ul of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 ul of Sodium Vanadate (1 mM), and then 5 ul of water. Mix the components gently and preincubate the reaction mix at 30° C. for 2 min. Initial the reaction by adding 10 ul of the control enzyme or the filtered supernatant.

The tyrosine kinase assay reaction is then terminated by adding 10 ul of 120 mm EDTA and place the reactions on ice.

Tyrosine kinase activity is determined by transferring 50 ul aliquot of reaction mixture to a microtiter plate (MTP) module and incubating at 37° C. for 20 min. This allows the streptavadin coated 96 well plate to associate with the biotinylated peptide. Wash the MTP module with 300 ul/well of PBS four times. Next add 75 ul of anti-phospotyrosine antibody conjugated to horse radish peroxidase(anti-P-Tyr-POD(0.5 u/ml)) to each well and incubate at 37° C. for one hour. Wash the well as above.

Next add 100 ul of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 20

### High-throughput Screening Assay Identifying Phosphorylation Activity

As a potential alternative and/or compliment to the assay of protein tyrosine kinase activity described in Example 19, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 ug/ml) for 2 hr at room temp, (RT). The plates are then rinsed with PBS and blocked with 3% BSA/PBS for 1 hr at RT. The protein G plates are then treated with 2 commercial monoclonal antibodies (100 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4° C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 ul of the supernatants obtained in Example 11 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation.

### Example 21

### Method of Determining Alterations in a Gene Corresponding to a Polynucleotide

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:X. Suggested PCR conditions consist of 35 cycles at 95° C. for 30 seconds; 60-120 seconds at 52-58° C.; and 60-120 seconds at 70° C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products is cloned into T-tailed vectors as described in Holton, T. A. and Graham, M. W., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 22

### Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/mil. The antibodies are either monoclonal or polyclonal and are produced by the method described in Example 10. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 23

### Formulating a Polypeptide

The secreted polypeptide composition will be formulated and dosed in a fashion consistent with good medical practice, taking into account the clinical condition of the individual patient (especially the side effects of treatment with the secreted polypeptide alone), the site of delivery, the method of administration, the scheduling of administration, and other factors known to practitioners. The “effective amount” for purposes herein is thus determined by such considerations.

As a general proposition, the total pharmaceutically effective amount of secreted polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the secreted polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the secreted protein of the invention are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The secreted polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(−)-3-hydroxybutyric acid (EP 133,988). Sustained-release compositions also include liposomally entrapped polypeptides. Liposomes containing the secreted polypeptide are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, the secreted polypeptide is -20 formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the polypeptide uniformly and intimately with liquid carriers or finely divided solid carriers or both. Then, if necessary, the product is shaped into the desired formulation. Preferably the carrier is a parenteral carrier, more preferably a solution that is isotonic with-the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The secreted polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any polypeptide to be used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

Polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous polypeptide solution, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

### Example 24

### Method of Treating Decreased Levels of the Polypeptide

It will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a pharmaceutical composition comprising an amount of the polypeptide to increase the activity level of the polypeptide in such an individual.

For example, a patient with decreased levels of a polypeptide receives a daily dose 0.1-100 ug/kg of the polypeptide for six consecutive days. Preferably, the polypeptide is in the secreted form. The exact details of the dosing scheme, based on administration and formulation, are provided in Example 23.

### Example 25

### Method of Treating Increased Levels of the Polypeptide

Antisense technology is used to inhibit production of a polypeptide of the present invention. This technology is one example of a method of decreasing levels of a polypeptide, preferably a secreted form, due to a variety of etiologies, such as cancer.

For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 23.

### Example 26

### Method of Treatment Using Gene Therapy

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoR1 and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB 101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 27

### Method of Treatment Using Gene Therapy—in vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85(l):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 28

### Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

### Example 29

### Knock-out Animals

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:X or a polynucleotide fragment of the cDNA sequence included in ATCC Deposit No:Z, which is hybridizable to SEQ ID NO:X;
(b) a polynucleotide encoding a polypeptide fragment of SEQ ID NO:Y or a polypeptide fragment encoded by the cDNA sequence included in ATCC Deposit No:Z, which is hybridizable to SEQ ID NO:X;
(c) a polynucleotide encoding a polypeptide domain of SEQ ID NO:Y or a polypeptide domain encoded by the cDNA sequence included in ATCC Deposit No:Z, which is hybridizable to SEQ ID NO:X;
(d) a polynucleotide encoding a polypeptide epitope of SEQ ID NO:Y or a polypeptide epitope encoded by the cDNA sequence included in ATCC Deposit No:Z, which is hybridizable to SEQ ID NO:X;
(e) a polynucleotide encoding a polypeptide of SEQ ID NO:Y or the cDNA sequence included in ATCC Deposit No:Z, which is hybridizable to SEQ ID NO:X, having biological activity;
(f) a polynucleotide which is a variant of SEQ ID NO:X;
(g) a polynucleotide which is an allelic variant of SEQ ID NO:X;
(h) a polynucleotide which encodes a species homologue of the SEQ ID NO:Y;
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a secreted protein.

3. The isolated nucleic acid molecule of claim 1, wherein the polynucleotide fragment comprises a nucleotide sequence encoding the sequence identified as SEQ ID NO:Y or the polypeptide encoded by the cDNA sequence included in ATCC Deposit No:Z, which is hybridizable to SEQ ID NO:X.

4. The isolated nucleic acid molecule of claim 1, wherein the polynucleotide fragment comprises the entire nucleotide sequence of SEQ ID NO:X or the cDNA sequence included in ATCC Deposit No:Z, which is hybridizable to SEQ ID NO:X.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 8.

10. The recombinant host cell of claim 9 comprising vector sequences.

11. An isolated polypeptide comprising an amino acid sequence at least 95% identical to a sequence selected from the group consisting of:
(a) a polypeptide fragment of SEQ ID NO:Y or the encoded sequence included in ATCC Deposit No:Z;
(b) a polypeptide fragment of SEQ ID NO:Y or the encoded sequence included in ATCC Deposit No:Z, having biological activity;
(c) a polypeptide domain of SEQ ID NO:Y or the encoded sequence included in ATCC Deposit No:Z;
(d) a polypeptide epitope of SEQ ID NO:Y or the encoded sequence included in ATCC Deposit No:Z;
(e) a secreted form of SEQ ID NO:Y or the encoded sequence included in ATCC Deposit No:Z;
(f) a full length protein of SEQ ID NO:Y or the encoded sequence included in ATCC Deposit No:Z;
(g) a variant of SEQ ID NO:Y;
(h) an allelic variant of SEQ ID NO:Y; or
(i) a species homologue of the SEQ ID NO:Y.

12. The isolated polypeptide of claim 11, wherein the secreted form or the full length protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition, comprising administering to a mammalian subject a therapeutically effective amount of the polypeptide of claim 11 or the polynucleotide of claim 1.

18. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

19. A method of diagnosing a pathological condition or a susceptibility to a pathological condition in a subject comprising:
(a) determining the presence or amount of expression of the polypeptide of claim 11 in a biological sample; and
(b) diagnosing a pathological condition or a susceptibility to a pathological condition based on the presence or amount of expression of the polypeptide.

20. A method for identifying a binding partner to the polypeptide of claim 11 comprising:
(a) contacting the polypeptide of claim 11 with a binding partner; and
(b) determining whether the binding partner effects an activity of the polypeptide.

21. The gene corresponding to the cDNA sequence of SEQ ID NO:Y.

22. A method of identifying an activity in a biological assay, wherein the method comprises:
(a) expressing SEQ ID NO:X in a cell;
(b) isolating the supernatant;
(c) detecting an activity in a biological assay; and
(d) identifying the protein in the supernatant having the activity.

23. The product produced by the method of claim 20.


SEQUENCE LISTING (612 sequences)

SEQ ID NO: 1 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 2 - protein, 5 aa, Homo sapiens
  Site (3): Xaa equals any of the twenty naturally ocurring L-amino acids
WSXWS

SEQ ID NO: 3 - DNA, 86 bp, Homo sapiens
gcgcctcgagatttccccgaaatctagatttccccgaaatgatttccccgaaatgatttccccgaaatatctgccatctcaattag

SEQ ID NO: 4 - DNA, 27 bp, Homo sapiens
gcggcaagctttttgcaaagcctaggc

SEQ ID NO: 5 - DNA, 271 bp, Homo sapiens
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 6 - DNA, 32 bp, Homo sapiens
gcgctcgagggatgacagcgatagaaccccgg

SEQ ID NO: 7 - DNA, 31 bp, Homo sapiens
gcgaagcttcgcgactccccggatccgcctc

SEQ ID NO: 8 - DNA, 12 bp, Homo sapiens
ggggactttccc

SEQ ID NO: 9 - DNA, 73 bp, Homo sapiens
gcggcctcgaggggactttcccggggactttccggggactttccgggactttccatcctgccatctcaattag

SEQ ID NO: 10 - DNA, 256 bp, Homo sapiens
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 11 - DNA, 1010 bp, Homo sapiens
gcgtccgtatgttccagtgtgggttattgcagcagctttgtactatcctaatggctactggggttcctgctgatatcctgactgagaccataaatactgtatcagaagttattcgaggctgccaagtaaaccaagactactttgcatctgtaaatgcaccttcaaacccaccaagaccggcaattgtagtacttctcatgtccatggttaatgaaaggcagccatttgttttgcgctgtgctgttctctattgtttccagtgtttcttgtataaaaaccaaaaaggacaaggagaaatcgtgtcaacacttttaccttctaccattgatgcaacaggtaattcagtttcagctggccagttattatgtggaggtttgttttctactgattcactttcaaactggtgtgctgctgtggcccttgcccatgcgttgcaagaaaatgccacccagaaagaacagttgctcagggttcaacttgctacaagtattggcaaccctccagtttctttacttcaacagtgcaccaatattctttcacagggaagcaaaatacaaacaagagttggattattaatgttgctttgtacctggctaagcaattgtcccattgcagtaacgcattttcttcacaattcagccaatgttccattccttacaggacaaattgcagaaaatcttggagaagaagagcagttggtccaaggcttatgtgcccttttgttgggcatttcgatttatttcaatgataactcacttgagagctacatgaaagagaagctaaaacaactgattgagaagaggattggcaaagagaatttcatagagaaactaggatttattagcaaacatgagttgtattccagagcatctcagaaaccccagccaaactttcccagtccagaatacatgatatttgatcatgagtttacgaagctggtaaaagaacttgaaggtgttataactaaggctatttataagtccagtgaagaagataaaaaaaaaaaaaaa

SEQ ID NO: 12 - DNA, 1559 bp, Homo sapiens
  SITE (1547): n equals a,t,g, or c
ccggctactaggaactagtggatcccccgggctgccaggtttttattctttatatataaaatgtctcccagataccaaaagatcaaataaaaatcccacccctcctactgcgttcctaggttggctgtatacttatttgaggaatgtttcacagcccttgtttcagtgcagtattttctggtttatggggctggaatcaaagggaccgctattccctctgccccaacacacagacttacgaatcagtgctccctatagcaccatactgcaggattggtgtgcatgactgtggcatccatcagacatattctggtagagatctggttaccgatagccttagccatgggcacccgaggcctcactcaaattgtagctgtcattcaatccagaagccaatgggcactctcttgaagtctgcttggctcttctgttctcttttttagtaggtctgatttattaatatgatcaatacaggttaacttgtgaagtcctatatacaacactcttcctcctctagacaactttgctattgctacttagaaatgatgtaaatcattggaggtgtgataaatttacaaatatttagtttttaacccatagcaagggagatattgggctagagtgtaaaacacagagcatttaatatacataggaaatttaggagtaaaattttgatgctaattttccaaaatcatttctactatttctcgtcattataagattgctcacatattttgaatggcatcccacagtagtcacaggcaaattagaataacatatgctttacaaaacaaaaaggcatatcagaaaatgagtcaaagtaaaaaaaatggcaggtaccatccattctccagtaaataatgaatatggagtgtatttaattacaaatttccaaatatatattcgcataataaagacaaggaaaatgaaacaaaatcaaatgtgttttgttgaaataagcgcatgagcttgaaggatcmagtactgatggctccaggtgttttccatgttagattccctttgaaagtactgatgccatcagcacttgtcagctggtacccaatgatgtaaattttgagcttgaatacttttcatrggarrgcatcacattcctaaaggagacaaatgaccactaaaaactaaccattaatatagattacttatggagggttttatcttgactactaattaagggagtaattgtatctctaaactttatatcagattgccagtggatttgccagattattaatgcttttttagaatacatggggggtggggttataatcacataacacagtttttcatattttttaaaaggggttggaccaagacataagtttcattgacaatcttaatgttttcactgtagacaatatgttagaataactgcagtgagtcacacagcatataacttcttaatggtaaattcagaaccagtcttccagataagcaagtataacattttagttaaaaaaaaaaaaaaaaaaaaaaaaaacaaaaaaaaaaaaaaaaactcgagggggggtcccgtacccaatctcctnanatgcatcgta

SEQ ID NO: 13 - DNA, 1589 bp, Homo sapiens
ggcagagcttttgctctgagaacaatgctaccggtagtttccagtgtgtttgctttgcctttttatttgaattttagaatttattattttaaaattttatcttatttaaatgttattcatttttcttctactaattttgagtatcacagcttcgtactccttgacttgcattctctcaggagctggggagccaagctcggtctccgcttcggtggtttcaggtcccgggttctgtctggcggctctgcttctaatgcggactggcggttttgcagcaacgcttttgccagtagcgcccactgagcggtttttcagttgctgcaccgttcttagcgcccaacggaacgtttcccgtacgcggagtccataagttgtctgtgtcgtcactttctcggttctggcamcgctagtatctcctaggaacttcctaaamccgttttacggtgcagaacggtaattgcaccgagcctctgtgccaactagtacttccgtaataccgttagccggctcttttcctgtcacaacacattaaaacgtgttaaaacacattaaacggattaaacacattaracggattaaaacacattaaaacgtcacaacacatttaaacagagtcggtccattcytccagacagtatgtttcgggatggaagcacccytcctaagaatccgcagtttctttstagcagcaccgttatggcgcytaaacggatatttgtttggcaataccagcgctatccgctaggtgccggcgcttgctgagtctcagcgccgccagattcttgctagcgaggtccagagcagtgcagagacctagcggacagttttccggtggcagcaatgctcatttcccggacacagatggtttttggsccctgaagaactctttaatccctgcgttttgtagcgctcgttctctgtaaggtttgcttartatccgtttcatttcgacttcttttctcgctgtttttctgtcggaattacgatttgttttgtttctatgtgtyctctagaattttaccggttttcatttgtctactaattttcgtgcattcgttactattgagtttcataatatctgggtgccmtctgcccackagctccggagagcataaatacccaggctgatggtagtgctaaaggttctgcttcatctcgtcaatcagcgcaaacgcttatgtcctcctgctctgaggcttaattaagcgatggagcaacattttcttggctatgtgaagcctgcttgagattccgcagaggtggtgcctgagctccagcctcggcctattttgaattcagaagactgtgatattactgtgtgagccaccactttgggaggccagcatgggcagatcacttgaggtcaggagttggagaccagcctggcccaagtggtgaaaccccatctctactaaaaatacaaaaattagccggtgatgatgcatgcctgcaatcccagctactcgggaggctgaggcaggagaatctcttgagctcggaggcagaggttgcagtgagctcagatcgtgccaccgtcctccagcctgggcagactgagactccatcaaaaaaaaaaaaaaaaaactcga

SEQ ID NO: 14 - DNA, 1255 bp, Homo sapiens
gctgaaatgtctcatttagctaaatggaaacattcaccagacacttctagctagctctcccacatcactttgagccacacaggcacttccatgtttgggactgaattgcctgggaatccgtatacccaaaatctgagccctcattcttacccttgggtgatgcctctccatagccctcagcaatgctctccactcgctggatggggctacatctcgtgcagattttgtggcgctgttggaccagttcggcaaccattacatccaggaagctatctacggctttgaggagtcctgttctatctggtacccaaacaagcaggtccagcggcgactctggctggagtatgaagacatcagtaaaggcaactccccatcagatgagtctgaggagcgggaaagagaccccaagtgctgacattcccagaatacatcaccagcttgtcagactccggcaccaagcgcatggcggctggagtccgcatggagtgccagagcaagggacgatgcccctcgtcctgccccctgtgtcatgtgacatccagccctgacacccctgctgagccggttctgctggaggtgaccaaagcagcccccatctatgaactagtgaccaacaaccagacccagaggctcttgcaggaggctaccatgagctctctctggtgctcagggactggagatgtcatcgaggactggtgtcgatgtgactccactgcttttggagctgatggactccccacctgtgcgcctctcccacagcctgtgtatggttctctttctctctttcagcattactctggaaacagataatgagcgtctactatttgcaaagaaataggctgggtactgggaaacaaagaaaaatatgacacaatacctgcctttagagagctttcaatctaccaagggaggcagcacagatggttttagtagagaaagggtgtgacaagtgttaacaagaggcatttaaagaaagtgtgaagagggaatggtcaagtctgaactgacgccatggtgacggctctgtggaggaggtggcgcttgaacttctccttgaaggtggagaaagtaggacagcataactagaagacaatggggagaaaagtgccattgtaatggaagaagttagagatctacactgagagcttaactagaccaaaaacataaccactcattgaacacttactgggtgtccaggcattgtatggagcagttagatactgctcatttaatagacaaaaaaaaaaaaaaaaaa

SEQ ID NO: 15 - DNA, 1191 bp, Homo sapiens
gaattcggcacgagcggaggccgaagagttgagtccgttgctaagcaacgaacttcacagacagcgatccccaggtgtttcatttggtttatcagtgtttaatttgatgaatgccatcatgggaagtggcatccttggcttagcttatgttatggctaataccggtgtctttggatttagcttcttgctgctgacagttgctctcctggcttcttactcagtccatcttctgcttagtatgtgtattcagacagctgtaacatcttatgaagatcttggactctttgcatttggattacctggaaagttggtggtggcaggcaccataataattcagaatattggagctatgtcatcttatcttttaattattaaaacagagcttcctgctgctattgcagaatttttgactggagactatagtagatattggtatcttgatggacaaacactactaataatcatatgtgttggcattgtgttccctcttgcacttcttcccaaaataggctttcttggctacacaagtagtttatcattttyctttatgatgttctttgctcttgtggtaataattaaaaaatggtccatcccttgtcctctgacattaaattatgtagagaaaggcttccagatttcaaaygttacagatgattgtaagccaaagctctttcatttctccaaagagagtgcttatgccttaccaaccatggctttttcatttctctgccatacctcaatattgcccatatactgtgaacttcaaagtccttcaaagaaaagaatgcagaatgttaccaatacagcaattgctttaagttttctcatttattttatatctgcactctttgggtacctcactttttatgggtctcattctgtcgcacaggttggcgtgcagtggtgtgatctcagctcattgcaacctctgcctcccggactcaagcaatcctcccacctcagcctccagagtagctgagactacagaaaaggaagagataccatggagatgtgcacccagaggaaaggccacgcaaggacacagcaagaaggcaactgtttacaagccaagggaagaggcctcaggagaaccaaacgtgtccacaccttgatcttgcacttcccaacctccagaactgtgagcaaataaatgatgttgtttaatcaaaaaaaaaaaaatcaagggggccg

SEQ ID NO: 16 - DNA, 1186 bp, Homo sapiens
gaattcggcacgagattgaatgttccagataatccctttcccagtcctgcctgacatctgggtagggggtttgtccctggaattctgggacactggctggggtttgaggagagaagccagtacctacctggctgcaggatgaagctggccagtggcttcttggttttgtggctcagccttgggggtggcctggctcagagcgacacgagccctgacacggaggagtcctattcagactggggccttcggcacctccggggaagctttgaatccgtcaatagctacttcgattcttttctggagctgctgggagggaagaatggagtctgtcagtacaggtgccgatatggaaaggcaccaatgcccagacctggctacaagccccaagagcccaatggctgcggctcctatttcctgggtctcaaggtaccagaaagtatggacttgggcattccagcaatgacaaagtgctgcaaccagctggatgtctgttatgacacttgcggtgccaacaaatatcgctgtgatgcaaaattccgatggtgtctccamtcgatctgctctgaccttaagcggagtctgggctttgtctccaaagtggaagcctgtgattccctggttgacactgtgttcaacaccgtgtggaccttgggctgccgcccctttatgaatagtcagcgggcagcttgcatctgtgcagaggaggagaaggaagagttatgaggaagaagtgattccttcctggttttgagtgacaccacagctgtcagccttcaagatgtcaagtcttcgartcagcgtgactcattcgttcttccaacagtttggacaccacaaagcaggagaaagggaacatttttctacagctggaaagtgagtcctatcctttgaggaaatttgaaaaaagacatggagtggtttgaaagctactcttcatttaagactgctctccccaaccaagacacatttgcctggaaattcagttcttagcttaaagactaaaatgcaagcaaaccctgcaattcctggacctgatagttatattcatgagtgaaattgtggggagtccagccatttgggaggcaatgactttctgctggcccatgtttcagttgccagtaagcttctcacatttaataaagtgtactttttagaacatttggaaaaaaaaaaaaaaaaaactcga

SEQ ID NO: 17 - DNA, 1182 bp, Homo sapiens
cccacgcgtccggtaaaatataaagaaactgaaccagtgtgtcttttcaccatagatataagagttcggaccgcccagcacacaaggtcagcatgctgctcctctgtcacgctctcgctatagctgttgtccagatcgttatcttctcagaaagctgggcatttgccaagaacatcaacttctataatgtgaggcctcctctcgaccctacaccatttccaaatagcttcaagtgctttacttgtgaaaacgcaggggataattataactgcaatcgatgggcagaagacaaatggtgtccacaaaatacacagtactgtttgacagttcatcacttcaccagccacggaagaagcacatccatcaccaaaaagtgtgcctccagaagtgaatgtcattttgtcggttgccaccacagccgagattctgaacatacggagtgtaggtcttgctgtgaaggaatgatctgcaatgtagaattacccaccaatcacactaatgcagtgtttgccgtaatgcacgctcagagaacatctggcagcagtgcccccacactctaccttaccagtgcttgcctgggtctttgtgcttccattgctgtgatgccaccattcctaggagaggcagagaccagcctctaaagcacaagccaaaaactgtgtgaacggtgaactttggagtgaagatcaatcttgcacttggtgaagagtgcacattggacctcaaggcgaaagccagtggtttgcttggataaaatgttcccgcatgaggccacaggactgaggatgggaatttggcagggcctgagaagatggtctgacttccaggcttcctggtcaaagagagctacgtttgggcagttctgcagagaggatcctggcaactagtcccacctgactaggcctttagctgaaaggatttcttgacctccttgactgcctcagaggctgccaggtcaaaccctcttgtttatgtgattagctcagagcatctctatgaaatctaacccttcccctcatgagaaagcagttttccccaccaacagcatagtcaatgagaaaggcaactgtacgaagaaaacttccagtggaactaatatgaaatctatttgcaaattatggggggaaataaagcttttaaattatacaatgtaaaaaaaaaaaaaaaaaaaaaaaaaaaaaa

SEQ ID NO: 18 - DNA, 1171 bp, Homo sapiens
gcttcagacctttgtgatacaccatgctgcgtgggacgatgacggcgtggagaggaatgaggcctgaggtcacactggcttgcctcctcctagccacagcaggctgctttgctgacttgaacgaggtccctcaggtcaccgtccagcctgcgtccaccgtccagaagcccggaggcactgtgatcttgggctgcgtggtggaacctccaaggatgaatgtaacctggcgcctgaatggaaaggagctgaatggctcggatgatgctctgggtgtcctcatcacccacgggaccctcgtcatcactgcccttaacaaccacactgtgggacggtaccagtgtgtggcccggatgcctgcgggggctgtggccacgtgccagccactgtgacactagccagtgagtctgctcctttgcctccctgccatggtgcggtccctcctcatctctcccaccctgaagcccccaccattcatgctgcctcttgttactcttagcataaaatgggccttaactgcagaaatgtcaaatcagaacagtagctgcctagtaatgcccagtgatgggggacccttgtgcccttggaaaacctcactccaagtagaggctgtatctggagtgagtgtctacagagaggggaattggtcagtgcatggcagaacttgacatggcagaactgttccgtgggccccagagcagggcctgccgtgccttcctacttggtcatcctgtggctgtaggtgcactgtcccaactgctcacacactttccagtcccctccctccattctcaccacagttacagtcggagcagggagagacaggacaccaagagacatgggcagggccacgtgtagtctggacagagcccacccagcccagacttggcctcctgtctctctgtgaggggcaagcatagtccgaaggcctggattataaatattagaacataatgaaaaggaaactgtgtggcaaagggataaagggatacagagaaaagaaacaaggaagagatgaggtgacagtttggaccaaggaaaatgccaagagaagacttcacactacctttttttttttctggttttgccattcttttattcatgttgttacactcagtataggatagtttattaaaatcattatgtctgtagaaaaaaaaaaaaaaaaaaaaaa

SEQ ID NO: 19 - DNA, 1337 bp, Homo sapiens
  SITE (22): n equals a,t,g, or c
cggggcttcggcgccaggccangcgctagtcggtctggtaaggatttacaaaaggtgcaggtatgagcaggtctgaagactaacattttgtgaagttgtaaaacagaaaacctgttagaaatgtggtggtttcagcaaggcctcagtttccttccttcagcccttgtaatttggacatctgctgctttcatattttcatacattactgcagtaacactccaccatatagacccggctttaccttatatcagtgacactggtacagtagctccagaaaaatgcttatttggggcaatgctaaatattgcggcagttttatgcattgctaccatttatgttcgttataagcaagttcatgctctgagtcctgaagagaacgttatcatcaaattaaacaaggctggccttgtacttggaatactgagttgtttaggactttctattgtggcaaacttccagaaaacaaccctttttgctgcacatgtaagtggagctgtgcttacctttggtatgggctcattatatatgtttgttcagaccatcctttcctaccaaatgcagcccaaaatccatggcaaacaagtcttctggatcagactgttgttggttatctggtgtggagtaagtgcacttagcatgctgacttgctcatcagttttgcacagtggcaattttgggactgatttagaacagaaactccattggaaccccgaggacaaaggttatgtgcttcacatgatcactactgcagcagaatggtctatgtcattttccttctttggttttttcctgacttacattcgtgattttcagaaaatttctttacgggtggaagccaatttacatggattaaccctctatgacactgcaccttgccctattaacaatgaacgaacacggctactttccagagatatttgatgaaaggataaaatatttctgtaatgattatgattctcagggattggggaaaggttcacagaagttgcttattcttctctgaaattttcaaccacttaatcaaggctgacagtaacactgatgaatgctgataatcaggaaacatgaaagaagccatttgatagattattctaaaggatatcatcaagaagactattaaaaacacctatgcctatacttttttatctcagaaaataaagtcraaagactatgawawmawagttttttataccttatttaagagaaacaacctgacgtgcaccawtcagtctgcacatcccaacccttcacattttataaattattgtagatcatgttttgttaggagcccttttatggagaggacattttcccatgncttaagtaatccagccttt

SEQ ID NO: 20 - DNA, 1162 bp, Homo sapiens
ggcacgaggcgccccggactcttctcagttgagagtgcggttcctgggcaggtttccacaccagttcctttccgcgtccttcggccctggctctggctgcctggcggaggtggggtagcatttgtcatttgcacactgctggctttatctttggggctgcaccccgaggcaacaaatgcaggatgctctgtcacccacatgtccaccaccatctggtttgccttttggctactttgactttctccttaaatgcttcctgtgctgagcaaacattccacagccagcagagcaatggagagttcatggccactcttcccagtatcagcaagcaatttggggtgatcgtttggaagcctcagaggaaagatgtcatcaggcttcctgtggctttgtccttcagcatggggctcggcttgctttcacctgccttaggaagatttctggcttctgagctctgatatggggagaagataagggctgggatctttgagtctgcccctagctgggtatgtgcgtccggtgtgcgggccttggagtttttggtaatgactcacttgtgctctttctgggatctgtctccctcccacatgaccccgtggggtccctgaatgactgttttagagtacccatgtgggttccctgagtcacagcaggggatgtttaataaggaggttagcactgagcttggggacgtgctgtcacaccagcaggacgctgcaggaaggagcaggctacttcctttcttgacgtgcaaataactcgtataggctaatcaacaggcttataagttaaaagggctaccgctcggccccttggggattccatcccctcctctgtaacttggagatgtttgtttctgctgcagactcagagggttgcgatgaagagtggtgggactgagttgagaagcttatcccttcgctgggtgggaggtttctaattgccctgttctttgggggatccttaagtccagcttccaggtgggggcagcgataggaccaagttctcctagtagtctctgggaagccacttgagggaagctgccggtcatgcccatgcacccattggtcttctgccagcaggccctgtaggtcgtgccatgttccatgtccttctgggttcttgggggagaaggaagctgttgaaaaaaaaaaaaaaaaaa

SEQ ID NO: 21 - DNA, 1837 bp, Homo sapiens
aggaagaaatgaataatgtgagcattaggggcggcagtgggattactcagtgttgagagaaacaacacggaaagcccagagcagaggagggagaaggccccattctctcagctttaatctactgcacctcggagggcagagctctaaccagacaccctggataagagtccgctggctcctggaagctccctggtagacccccagatctctctttgggtgctgatggccattctgctggcctgcttcacagccgtcttggccttcatctgcctccagttctggtgtgtccgttgccatgagccgcgatggtcttacagggctggccacatggaggaggccaatgggttggtgagatggccagaggaggccccggatcttggtcagagggaggaagacctgcaggggctccccctggtggaaatgccacgcaagaactccagagatggagctgaactggatcccgaagccaaccaggatgcccctgatgcgggtgccttacagagggggggtggtgacccacccgctatactgcctcattgtggggaatgagagtggggaggagagcgcccgcatcatgtagcctaaagctttccacacagtaggtcgtttgtacaaatgtgtctatagactacccatttctctcccatcaaacgtcactgctattgtaggtcacctgggttggatgaatgccccatgacaaagcttctcaagctggaaaatgtatcccccagggtatgccaaggcacaaacraggcatctcagcagcagcrtccactcggaggacacagttaagaattgaaagcattgttttaacattaaaacaaacatggatatgttataaagtagaaagcaaaaatttgcatgacttaaataaaacacagcacttgaaggaaacacttgagtwacaagtaggctctcagargtacacttttgggcaaaggtagatgtccagtttcctctgcccttgaggggattatttggaaataatttgggattatttggaaataatttgtgagaacccttgctctggagtgttttccaacttttaggcaatcacataccaccgytytctattttttaaaaccatggattatcttcactattattaataatactttcctttaaattgaytcattttttaagcgtaaacttattttcaaagacagccttatattactccataagtgaaaaaccagcaccctcytgctgcaaacagaagggaagagacataagaataaacatcatgaaaacaagataatattaaataatctgacttagcttctattgcctgccagtgattctgagcttaatgcctgcttgcttgtctttgataaaaagggagatccagtgttggagaggtaataaagacatattagcaccaatatttgtctttctccttgatatagcataagatttgaaagagaatcgaaaagggaagtgctgtttttactttgcgagtcagtgttagttaaatgccatgtctgtgaaccacctcaaatgctgtcagtcagcttcccttgatttgagaaaattgtcttgaccagactaggcaggctgttctggagaagacgcctgtactctccaagcatcatccaagaccttcccagtaccctcttatatctatagagcacaaaatcccagaatcacaggacgacactgcagtgaatcaactaagaaacagcaggagctgagaagccaagacgagaagccccacatccctattcccttgcctacctcatgcattccctgctcggcacccagacttttgcccccattcctgctacttgtgaacaaataaagattcatatactcacaaaaaaaaaaaaaaaaaactcga

SEQ ID NO: 22 - DNA, 1054 bp, Homo sapiens
cacgcgtccgcggcacgctgggaaagaatcctccagttttagctcctacttcaacagcatttccttatctatacagtaacccaagtgggatgtctccttatgcttctcagggttttccatttcttcctccatatcctccacaagaagcaaacaggagtatcacttctttatctgttgctgacactgtttcttcttcaacaacaagtcataccacagccaagcctgccgctccttcatttggtgtcctttcaaatctgccattacccattcccacagtggatgcttcaataccgacaagccaaaatggttttgggtacaagatgccagatgtccctgatgcatttccagaactctcagaactaagtgtgtcacaactcacagatatgaatgaacaagaggaggtattactagaacagtttctgactttgcctcaactaaaacaaattattaccgacaaagatgacttagtaaaaagtattgaggaactagcaagaaaaaatctccttttggagcccagcttggaagccaaaagacaaactgttttagataagtatgaattacttacacagatgaagtccactttcgaaaagaagatgcaaaggcagcatgaacttagtgagagctgtagtgcaagtgcccttcaggcaagattgaaagtagctgcacatgaagctgaggaagaatctgataatattgcagaagacttcttggagggaaagatggaaatagatgattttctcagtagcttcatggaaaagagaacaatttgccactgtagaagagccaaggaagagaaacttcagcaggcgatagcaatgcacagccaatttcatgctccactataggtaaattgtatttcaagtttgagtctcaaggtgattgcatcagtgttctttaaatagacatgttgttaacggtgcctgttcatcagccttaagcataattctgtcattatagttactgtgctatgtaacatagaatgctttgtattatataataagcataatataaacatataactagtagaaaaaaaaaaaaaaaaaaaaaaaaaaaa

SEQ ID NO: 23 - DNA, 1066 bp, Homo sapiens
gaattcggcacgagaaaaagcaaaagtaaaataaatattaaggaagaaatgtcaagagtagtctctctcttctttttcattctcttttcctttttcttctttgctttctccttatcgtcttctctttcttttgtacactatgagaaactagtgcaagtgaaagaatgtttggattcatttctcaaaaaaataaaaattaaggaatacaagactagacaatgttatcatttaataaggtgggagaacaatggggccaagttgcaaagttgaacacaggtggaagcatcagcaaagcagagccgtagaagaagtcaagcaactttgtccaaggcagggttttaagctcacattaggcaggtgggggctaagcagagctcctcagagggtcagatgcatttgtagataccttactgctctactgtctcctgttccagaaaatacctatgcatataacattcataaaactttaatatacgtacaaaactagtatcttgactgtagtgctagtttgattcttatgttttgcaattcatattttgtaattgaaaatatgaaagagtgtgtgtatagaagaaagaagagagaaggaaggaaggaaggaagaagaaagagaaaggagagaaagaaaaagagggaaaattgagattaaggttcaagtgattgagtttggcgtctcctcccaagggattttacattggctttggttcaactctgatcttaaattgcaactgcctataaaatgtattttataaattggggcaggggggtataaaatgttattttagcttgtagaattctagtggattggagtgtagttacagatcgaaagaactatagatgggagataatgagaagtggaattaagccatgcaaagtaaaaatctaagaggcagctctatgaaatatgactctactgaacaggtttaggagacagcagctggtgaggcaaactcctgctaggaagaattttctaattctaagctactttcaaatttgatagggctgaaaaatatctctctgaatcaaatgcagacattaaaaaaaaaaaaaaaaaaaaaggccgctc

SEQ ID NO: 24 - DNA, 928 bp, Homo sapiens
ggcacttcatctaaagtagtaactcagaaagtgcacttatcttctgttgaatttcctttcatgtcagcctctctaaaaaaccacctgacccactgctttttgctattgctgcttaaagagcttgtttcccccaccatgattagctttgtgcctacactaaggcactcctacagattcttcaacctcttctcatgtgatgcagaaagtacaaaggagagccctggccgaactgtccagtttagtaaaacacccagaggagtaactatgtttatttagggtgtttcagagcttgctgctctgcctagtctattgtaagagctctgaaattattagagcttttcctaaaaatatccacacttggatgtgactcagtcatagaaaaacaactgacccaagaaacagagagtctgagttttggttcttggtttgttttgttcttattgtgttttcattttgttgcatgaaacatgaaacctccggtcttgggatagatttgagtcctgactcagccagtgataagttgtttgaccttgggtgagttactaaacttctctggggctaaatttcttcatctaagaagtggattagatacagcaacatattatagttcgaagcttctggctggacgttgtggctcacgactgtaatcccagcattttgggaggctgaggtgggaggatctcttgaacccagaagttcaagactagcctgggcaacatggcaaaaccctgtctctactaaacatacaaaaattagtcaggcatgatggagcatgcctgtaatcccagctactcaggaagctgaggtgggaggatcgattgagcccaggagtttgagattgcagtgaaccatgattgaaccactatactccaacctgggcaacagagcaagaccctgtctcaaaaaaaaaaaaaaaaaaaaaaaaaaa

SEQ ID NO: 25 - DNA, 966 bp, Homo sapiens
ccacgcgtccggtatttttgtcaatgacgccactgaaagggatcaagtccgtgattttacctcaggttttcctctgtgcctacatggcagcgttcaacagcatcaatggaaacagaagttacacttgtaagccactagaaagatcattactaatggcgggagccgttgcttcttcaactttcttaggagtaatccctcagtttgtccagatgaagtatggcctgactggcccttggattaaaagactcttacctgtgatcttcctcgtgcaagccagtggaatgaatgtctacatgtcccgaagtcttgaatccattaaggggattgcggtcatggacaaggaaggcaatgtcctgggtcattccagaattgctgggacaaaggctgttagagaaacgctagcatccagaatagtgctgtttgggacctcagctctgattcctgaagtcttcacctacttttttaaaaggacccagtatttcaggaaaaacccagggtcattgtggattttgaaactgtcttgtactgtcctggcaatgggactgatggtgccattttcttttagtatatttccacagattggacagatacagtactgtagtcttgaagagaaaattcagtctccaacagaagaaacagaaatcttttatcacagaggggtgtaggcgtgagttttaggtgaatttatgtggttcctgcttgaaaaccttcccctctccaggttcggtttagagaactttgccacaggtcttctggggtttttaaggctggctggagaagacagtgggagggtgcccccgtctgacacccctggggttgctgaggggacggttggagtggggatcggcctgcgaaaggatactgtgaaatcactaattaactaataaacctgtctcaagttgaggatttgaaaaaaaaaaaaaaaaaaaaaaaaaaaaaaaaaaaaaaaaaaaaaaaaaaaaaaaa

SEQ ID NO: 26 - DNA, 1146 bp, Homo sapiens
gctgacctcataatccgcccgcctcggcctcccatagtgctgggattacaggcgtgagcaccgcgtccggctgtcagaatttcttttaatgctacacatatataagcaaataatgtttttaagaagctaaccttgatgttaagagtggcaggtgttctccagtttttacctctttcatatgggaccaaagtagctagtttatggaacacatatgaaaatgtggttatgccaccaagttttactactacacttgtcttaccacttttaagtcatgaattctataattattcatacccctttgcctgtgatcagaagtaacttttaaaattatcacttgactttggatggaactagtataaaggcaggaattttgtctttcagtggagatttattctgttcaaggttgaagtggacacatcattatcttgggatggtacttctttatttaaatagtcctttaaagttctttgagtggcagagaacgtttttggtttcagtctgagaaggctaccaaatggtttaagttcatttcatagttaggagaaaaagattttgagtagtctaatgtcgtcagaaaggattaaaacgttgtatgtaccaagaaggcagaatgamgaaggatcacgttcacaaatgctgtatgtttaacaaaatacgtttagatggtaatatccaatagtcttatcaagtgctacaatctttttaaacagggaatggccaaatccagtgctattgaaacagcctgaagaatgcaatcttaatttgcctgtatgggacccaagggttagtgtattattttttcccctaccaattcacactgtgcaataacaagtaaaaatcatctgcataaactccagggagacttccagccttttacttatgaatggtcatgtccgtattacactttcttttagataacctcaactataattgtcctcaactataattgatgtgagaagcataattatgtaccctgtaaaccacatttaattgtacatagtttttaatacagattttactaacattttaatttattctatatagaactaccttgtaagtcaaagttgtgtgggcatttgtgctttaaaaaaaataaaaggatactaaaaaaaaaaaaaaaaaaaactcgagactagttctctctct

SEQ ID NO: 27 - DNA, 802 bp, Homo sapiens
  SITE (337): n equals a,t,g, or c
ggtgggtgaccagagagtcctgtctatcctaggaggagaacattcagcccaaatcccagccccatcatgcacagatcagagccatttctgaaaatgtcgctgctgattctgcttttcctgggattggcagaagcctgtactcctcgtgaagtcaacttgctgaaagggatcataggtctcatgagcagactgtcaccggatgagatcctaggcttgctgagcctccaagtactgcatgaagaaacaagtggctgcaaggaggaagttaaacccttctcaggcaccaccccatccaggaaaccactccccaagagggaagaacacgtggaayttcctngaaatgcgsctacatggtgrtngacctacctcttcgtatcctacaacaaaggggactggttcactttttcctcccaagtgttactgccaytactgtaacttggaactggacatcagggatgatccctgctgttctttctagtgagcctgctccatctcagcttagccttcacaaggcctccatctcccaggcattctaacctctgaagaaagctctctgtcccctggactgcctgtgtggagggtaatgaactgggtcctttaaggaatggcacctgggtgcccagaggcatggccagaaggtgtctgtgggggccatgccttagggggatgcacccagggcggctgagagagcaactgcaggagtttcccctaaaatctctcctccagatcgttctcgaactttccccactacttccataataaaatgtatacttgttgaaaaaaaaaaaaaaaaaaactcgag

SEQ ID NO: 28 - DNA, 1169 bp, Homo sapiens
ggaatgtgagtgcaaatttgaatttccatgtacacatgtccccgtgtgcacatatctgtctgtatgtgctagtgtttctatgtaatgtgactagatgtaaatgtgttagggcattcacaacctgggacacagagaaagtgaaatattttatggcacactggagtaaactgaagagggttaggggtactagagttgagtgaaaaggaatttcttacattttcctcatattatacaattatgggaagaaaattaaaatgcagaattttaggggagttattaaatattgaatttgtgtacaactttcaaatgaaatcttttcagttttttatgacacacttgagctcacttctagaaacatgtcttagtctgttttgtgctgctctaacagaatacttgagactgggtaatttttaacaagcagagatttctttcttacagttctggaggctaggaagtccaaggttgaggggcatgcatctagcaagggcctccttcctgcgtcatcccatagtgaagggcagaaaggcaagagaacacgcttttgcatgagagagagaaagagagagaagagaagggaagggaagagaaagaagagaagagaagagaagagaagagaagaaaagggagccaaactcatctatttatcaggaacccttcttatgatagaaacccactcccatgaaaacaggattaatctgtttatgaggacagagtcctcattacctcatcasttcttaaaggtctcacttctcagtactgctgcattggggattaggtttccaacacatgaacttcggaggacacattcaagccgtagcattctyccttgactcccmaaatccatgtccttctcatgtctaaaatagattaatyccatcccaatwgccccaaagtcttgactcgttccagcaccaactcaaaattccaaagtccagagtcccatttgaatcagacaggagagactccaggtgcaattcatcctgaggcaaatttctctccagatgttagcctatgaaaatagcaaattactttcttccaaaatacaatggtgggacaggcataggatagacattcccattccaaaagggagaaataagcaagaagaaaggggtaactggtcccaagtaagtccaaaatccaacagaaaaaaaaaaaaaaagggcggccgc

SEQ ID NO: 29 - DNA, 1466 bp, Homo sapiens
ggcacgagtgcctcaaagactattatttgggaggatctagtgcaaatgttagtaatgtggatattgtgtagtgtcccaggatattaatgtttttagcctcttggcttttattctgtattgttgccccaaaagatgatgctcacttatctttcatccagtgtaaggatatctggaaagacaacagaaagtatagctgttttcatttcaaaagtgatcagctgcttgagctagcaagcaaggcttgcactagcttccaggcgcagtcacgcagtttcacagcaggcgcggttccctcggagcacccagagctgccctgcggtagtcagcagttgtgctgtggctgcactgccaggctgggtggcargtggatcggagccagcagatgtggctcaggaagtgccttcttggcctctccttaatctctttcagastctgtgggcccttgattgcactgtgggttgtttcagactccagtattaggagactgaaccccttggtggttttttggtgtgtgtgtgctgagmtgggttgaggacatgttaagcaggtggggtgcytcccctgggtttgctccgggtggtacctgtggtgtggggtggttctgagtagttctggccccactgctggagtatctgcccaytcagtttgtgagatggcagggcttcatcctggtctggtgcctcattttcttctttagcagtgggcttagaaccaatgcagattcccaagttaagtattttttctgtagcttaattattacaggcttctggtacctaagccctttcttactttctgttctgaggggaagagaagataatgttgtttctccgccccccccggagtggccccaggaccttgcatggcatttgcagcatttgcagcgtgcttgggtttgctttactagggtgaaagtgttgcaccccccagcacccacaaaggcacctctgctcaccctccggtgaggttctgactggccctgggacatcacstgctccaggatcctatgtggctcatcccaggagagatgtgggagggaaggggaaaaaaggcttacatttgctgagtggaattcatgtagatctgagttccgcattgattcctaagctgcagagcccttatgccttggctgttttgtgaatgttagtcggtcttaacctttttcaccgagttagcattggctgtctcaggaggctcacagctcctgctcctcctccaggggagtgcgccctcctcctctgtcggtagctgtcaggtgcccctttcctctgcagcagactgtcctgggtccttgcctggccttccccttacacgtgagcctgcagcttcattcacagcccctgtgtagaaagataggcactaaaagcagctgactggcagccctagaaacatgaagggtttcatttatagtttcagtccttttccttctttcgagccttaatttaaaaaaaaaaaaaaaaaaactcgta

SEQ ID NO: 30 - DNA, 1226 bp, Homo sapiens
ggcagtgcccgcatggactaaaataagtattagattctgccttcaagcttgctaccagatgcgactgtaaaattgaagtacatcatctcacttgccactataaagcctgctaccagatgcagcttaattatcacttgccactcactcactgatagggttttgatatgagtctgcaagcaattgatttattgtggtccctgtgcactcaaacctctctgctaacattaatctgtatttgcagccactcccaagccctatcatcatcacctcagctccacctcagatcatcaagcattagattctcatagggacatgcaacctagatccctaacatgtgcagtttacaatagggtttgcactcctatgataatctaatgttgccactcatctgacaggaggtggagctcaggcagtaatgcgagtgatggagaatggctgtaaagacagatgaagctgtgtttcctcacttgctactcacctcctgctctgctgcctggttcctaacagggtgttgagaaccctgggattatattgtataggatgtcaccattatgtgtccattatgtatcagtattatgtatccactgtatagactgatatttattttttaccacctcagaactggtttcggtttttagtagatatggttacatagatagggcactttgttgagatactaggaaatgtgttttgaggacttattctttttcagtggtgaaatgaattcttttggactgttacttcccctgagaaaaattaggaattttatttgcctaaagcatgagagaactatcaaaaccatgagaatttrtcagagatttaggagaggtacaatgtcaagggatacaakacaggcawtttgtgtaattttaaccttgagacagttagaaatcctggaaacctcctgagacactgagcagtccttttgtcagccattacagaggaacaaaattagatatccagatgtgccaaggaagaggagccttggtacatacttaaggatttagtctggcccctgagattctaaaccctactcgtgataattagcccacctttgaatcatcataatcttttattagattatggtgaaatgcccataatgtagttagatggcaacataaaaagtaaatactttattgagtgagataacatcatatggaggcttaaagtatttcttctatatatcatatacaatatctggcacaaactaaaaaaaaaaactcgag

SEQ ID NO: 31 - DNA, 1094 bp, Homo sapiens
ggcagagccccagggtgagaagtttgggagacatgttccattttggtctgtgggatctgcatttcttccttatagttatggctcatagagatgactgctctttcaaaggtggttgtggtctattagagcgtttccaatgtccccatacttctttctcgagtgctagtcaaaagcgacttgcagatggtatggaatgtctttgtgagatagaaagaacacagactaggatcagaaaaatctgcctcccaaccctccatggccatcttctggctgtgtgactttaccgccctaactctttaaaatagccccacacctacctgccaggattgtcttcagaattacataaaataacacataccaaagtctctagaatagagcatgtcacataatagactcaacaaaggtcagcatctcttctttctttttgggatgactaaagtagcctaaataaataagtattccgcaatgcatgacttgataacataaacttttgtaatactctattacctccaagcataaaacaattattgagacaaaagaaatacaatattttctaagttccaagacaaaaaaaaaacttaccaataaagaaaggagctgtctgttccacaaaaccaacagccacagttttttccttcttaaatgtcagactttgaaaaggttgacatttctgttcaccagcatatacacgtgggccctctcttactgatgaccacagagtcctggggaccttcctgtgctcccagcccagctctcctctccggccacactgctgcctccttcacgcatacactgggtggtgtgctgggctgccctccttaccacaagttttattcctcagcacacacttcagatcacagaaaggagacaaataaggtggaggaaggaagatgggttgatgtaactaggtcacttggtaatttcaatttcagacgtaaatttttctgtgtttcagaattattgatctgtggaatatttcttgattcttcttggaaattacaaattaattcaaatgattgtaaagttctctgaaaggctctacagttttcttaagagaataaaaaaacctgaaaataaaaaaaaaaaaaaaaaaa

SEQ ID NO: 32 - DNA, 1037 bp, Homo sapiens
  SITE (6): n equals a,t,g, or c
ggcggncccatgaaagactgcgagtacagccagatcagcacccacagctcctcccccatggagtcgccccacaagaagaagaaaatcgcggcccggaggaaatgggaggtgttcccgggaagaaacaagttcttctgtaacgggaggatcatgatggcccggcagacgggcgtcttctacctgacgctcgtcctcatcctggtcactagcggactcttcttcgccttcgactgtccgtacctggcggtgaaaatcacccctgccatccctgcagtcgctggcatcctgttcttctttgtgatggggaccctgctccgcaccagcttcagcgaccccggagtcctcccacgagccacacctgatgaagccgccgatctggaaaggcaaataggtaacactgaaagtctgcccatggcctctggtcacttcccgcctgggcccagctacagtggggaaggcaggccgagggctktgcaggaggagctgascgctgggaaggaaggaggccagaagtcagcgttccttagctcgctgggtgggcaggatgagctgaagaagaggtgtgatataaggctggagggacaggtatcctggaggcaggactgcaggcccacttgagcaaagcatcagtgtgagctgtgcttctgatgtttctttgaaacccaagtgtttgattccactctactaagcagcctccaccccgagagattacagttgtaggttgctgcccttactcccttttgcaatccttagagcatgaccagtttccatgatataaatctaggaaagttacatcttaggcagctttcttgtttatccaggccaggattgagaatttcctttatttaggtataataaacatgtatctgtgatatgtattgagatgaatagctttatttttccttagatattaaaacctatactaaagtttattacaacccattttgaagatattaaaacagatcctaatcccttacacaataaacttttacagtttttttttttaaaaaaaaaaaaaaaaaaaaaaaaaactcgag

SEQ ID NO: 33 - DNA, 1376 bp, Homo sapiens
ggccacccttcccccacccagagactgggcagctgtgtctggtggactctgcgccccgccccctgcagccgtacttgcgcctcatgcggttggacaagcccattggaacctggcttctgtatttaccatgtacctggagcattggtttggcagctgaaccaggttgttttccagattggtacatgctctccctctttggcactggagctattctgatgcgtggagcaggctgtactattaatgacatgtgggaccaggactatgataaaaaggttacaagaacagccaatcgtccaatagccgctggagacatttcaacttttcagtcctttgtttttcttgggggacagctaaccctggcactgggtgttcttctgtgtctaaattactacagtatagctctgggagcaggatccttacttcttgtcatcacctacccactaatgaaaagaatttcatactggcctcaactagccttgggcttgacatttaattggggagcgttacttggatggtctgctatcaagggttcctgtgatccatctgtttgcctgcctctttatttttctggagttatgtggacactaatatatgacactatttatgcccatcaggacaaaagagatgatgttttgattggtcttaagtcaacggctctgcggttcggagaaaataccaagccgtggctcagcggcttcagtgttgcaatgctgggggcactgagcytagtgggtgtgaacagtggacagactgctccytactacgctgccctgggtgctgtaggagcccatctgactcaccagatttacactctagacatccacagacctgaggattgttggaataaatttatctccaaccgaacactgggactaatagtttttttagggattgtccttgggaatttgtggaaagaaaagaagacagacaaaacaaagaagggtatagagaataaaatagaaaattaatgaatgaaatttatctaggaatttttaaaacattttttacaaaatataattagatttgaatacaaaatctgatacaatatgttaaagaattaagaacctgaagatgaagatttagagcatatttacctggattttacttatttgctagcaaaattcccccttgtcacagaaaccagggactcttcaggatttgagatggccttgagtattttagttgatacattcttctgcccattataattctcacctgaagttatggggattgcacgggttttggcactttagaaaaagcctgatgtgggtcttacataaatgaatgtctgtataagaaaatggactcttttttttagggaaaaataaaagcaactatgggaaaaaaaaaaaaaaaaaaaaaaaaagggcggccgc

SEQ ID NO: 34 - DNA, 1220 bp, Homo sapiens
  SITE (803): n equals a,t,g, or c
ggagaagtgcctaacatcccatgtgtgctaaggtggcagatcctgagctctccagctgccctcactgtggcctcacggcacaaccagggccagagtcaggaaacatcagtcactcgcttagggaagggagtccgagaaccctcttcgtggactcaacttcccaggcttctgtccctgctgcagaatgcccaggccacagagaagggacccccttttcaggagcttccacgtcacaggcttttatggggcctttcttgcttgtgtttctgtttcccatcctgagggtgtgtggaataatacgagagcctacccaggactggagcgtgttattagaaagagcacgcttgactgctcccgggcagccacctgccctttttcccttggaaagtggacctatggccactgcacaaaacacttcctaataccaggctgctggtgactacattttgtggtttctttgttagttgatatttatagttcagatctcatcactctgccaagtgcaataaacattttgttttaactggaatataacttttattattgttattatttttcttttaaatgtcttaaaagtgaccagcttgtgcaagtggagtcagaaaccctaaatagtctaaatgccaagtcagtgcgctctgtagatcagtgtacgattagaagacaggctctcatttattactgcttgtatttatataatggaagttaggtaacataagcccctctgcaggccagcttagatgttgatgatgcttaaatggtaaaatctgtgagaatgttcaacaatgccctgtatttgtacngcctaaaactgaataatacatatgtgacatgaataaataggtcagtgatttttatgacatattaatttcctttacctaagttcctgggaaatatagctgaacagttacagagaatgaaaacaaacactggcatcttacaaacacctgctttgattatttagtctaattaggttaaatttaactatttccattttaacaaattgaaaacaattcttatctgttcctcggtcatgcactttatcagtgttggatgcagatgtgtttaatatgacacagttggtgctgttttctcagctctgggtgggaaataaaaaggaattgagaacatatttttagagtcttctggggactttgtatttctgggcctatagctatgaaattttaaggaggaggaaaaaaaaaaaaaaaaaactcgta

SEQ ID NO: 35 - DNA, 1346 bp, Homo sapiens
  SITE (537): n equals a,t,g, or c
ggcagaggcttgtgaagggtaaagtttaaacccctctgcttagcccctgcctccagcctctgccaggagtaatgtgctcccatagtactctgatccacttgtatttggtgcttccttttttttttcttttccttccttcctcctttcctttcccttcctyttcctsttcctccattcttccctccctccgtcttcctccattcttccctccctccctattcctccattcttccctccctccctccctctcacatcctttaggactcagcatcacctcctctaggcagtctttcctggaytaccaccayttatgcacaaaacacctaagcaytaccttatgtggcctcatttatcactgcttaaatattttttawacacgtgctgtgatgtggcacatgcaggtgtcattcttgakgatccactggttattgccttgaggggatgacaactgcccggtagggtwacctggtgtgactgcacctaaaacagcaataccaaagggcccattgccagttctgtcactgaccagctggggcntctgagtatatcccttaaccactttggaccttaatttggcatctgtcaaatgagatggtggaacttgaggaactctaaggcccctactgtgcaggtcttattaatgattacaacagcagcagcagccagtgtttactgaggacttacaaagcaccaagcactttgcctatcctaatccttacatcaactctacgaagttagtatggttactatccctattttacagatgaggagactaaggctaagagaggttatatgacttgaccacaaggtcataataaagaaacagatttgaatccaggcattctgactttactgttcttagccacataatgggcacasnttygacacacrgttttgtgtactgtttggtggtcactcacagactccatcccagactctgcatgaaccatccctgttctacatttttaaggctcaaactggagtctgggtgaaacctggggacagaagactgctatagtcacaattattagagggaaatgggtgaggaccagtggccagctctgttcatgaacctttgacaattctcacagagagtcttgctttggacagagacnacttacgttgctgttttcagttaccctctttaggaggggagagtaggcctgagtcatgcttcagacacagattaaaatcagatttggtaccaggtgcagtggttcacgcctgtaatcccagcactttgggaggctgagttaggagtatcacttgaggccagaagtttgagagcagcctgggcgacatagtgagacatcctctctctttaaaaaaaaaaaaaaaaaactcga

SEQ ID NO: 36 - DNA, 1026 bp, Homo sapiens
ggcacgagcctctgtcaccaaacagtatgaagtatctaaacaatgttttaaaacaaattatttgtagccttcactgttggctgtctccatggcaggtgagctgcgaccttttgtcagcagcctagctgagatggctgctgatgcctgcaggtataagtgactgtcaattttccttactcatttatcttgctgtctcatgtttaaactagaagaagttgttcatctcaagtgtcctcaatgtcaatctatcatgtttgcctaattttgctcttgtacatcacatctcacagtcaccagaacatgagtagctgtctccaggtgcctctgtctctgttatcttgcccactgaagggagagcacttaagccaatttgcaggagaccacagtttgccagaggtcagagacagaaatcaccactgcattttgtttaaagaatcacatcagaaaagaaaataaaggacagggagggaaaagaagggaaacctaataaacacgccggtcctaaagtttgattccaagatttatgacagaatcaggcaaaactaaattaaaataatatctgtgaaaactggacaacctgaacataagttgatttttccagagaccaaagaacaaatcattgcacaaacacataccttttcaaactgaaaatgattccagagttaacttcatggacctaaatatgaatattaacatctcacaaatactatttgtaattttatccttgagcagtatagtggagaggtgacttctaagcaaattattagtaggaagtaaggttaagtgggcatattccatttttacatcctttcttctccatttatttacttcctatatgtttaggggcttccaccacccttatcctctgaaataacactagagcttttgccatttcctccacccaaagcttctcagatgttggaccagcaaggcgatcaagtttgttgtttgtttgtttatttgtttgtttgtttgtttgagactgggtctcactgtcacccaggttggagtcagagtggctgcgta

SEQ ID NO: 37 - DNA, 832 bp, Homo sapiens
gctataaccaaccatgctagcaaacttcacactgttcattctgactctaatatcttttctgctgttagtctgttctccgtgtaaacatctcaagatgatgcagctccatggcaaaggatctcaagatctcagcaccaaggtccatataaaacctttgcaaactgttatctccttccttatgttatttgccatttactttctgtgtataatcacatcaacttggaatcctaggacacagcagagcaatcttgtattcctgctttaccaaactcttgcaatcatgtatccttcattccactcattcatcctgattatgagaagtaggaagctaaaacagacctctctttcagttttgtgtcaggtgacgtgctgggtgaaataacagaaaccctcaactccatagattcacaaggggagcatggtgtgtcttttagcagaaaagaaactgatggtgtctagaacgttttatatttctgtcagtttgttgtagtgtatgtatttgagtaatttcaaaacagattcctaggatagtcttttatatatatataatatatataaaattcatatatatataaaatacgtatgggtgtatatgtgtgcatgtgtgtgaataataacattgaccataaattatgaagcctagtatatttcatatatataagtatgtgtattttatgatagctaattgtatgatatttcatttgaagaatttatctctctttgtaattaagaaattacagcatttatcagaaaatcattgctgttttccattgtaatttgtaccacatacatgtacttaactatcaaaaaaaaaaaaaaaaaaagggcggcc

SEQ ID NO: 38 - DNA, 706 bp, Homo sapiens
ggcagaggtgacaagccccgccaagacagacctgcaagtcttcgtctcaagggacctccctcatgccaggcccctgcctctcacagcagcaccctttcctctcattgtccctgttccctttttgcctgtggatctgtttggccagggtccctggggtcaggaatatttgcaagactcagccagctccttcccagcccagcctcttggggctgggactttctcaccctgcggcaggcacaacagatgctgggacccagtctctgcccaggtcacagcacaagtgcacatcagcactatggggcctatgtcctgcccagagacctctgctccttcctgctcacatccacagttcagggcacggcgcccctcaagaactccagagtcacctgtctcatcggctcccagcaagtgcctctttgtctatgatgtcccccttctctgaggcctggacccacccatctttgtccctggggcctgctcccagccactgaggcccgctctggccaggggagaaggagctgccgtgcgtcttccctgtgccccgtctccctgcttggttctcccctcccttccctggccggctgccatggccaggagctaagtgcctttttgtgtgcaaccacttaccctttctctgaaaaacctgttctcaggaaggatctgataaactcatttactctyaaaaaaaaaaaaaaaaaaaaaaaaa

SEQ ID NO: 39 - DNA, 1347 bp, Homo sapiens
  SITE (83): n equals a,t,g, or c
gggcagccctcaggccctccggcagcctggccgggcccgagtggccatggcagcactggtgtggctgctggcgggagcacatngtcaagcctcaacaagtggatcttcacagtgcacggctttgggcggcccctgctgctgtcggccctgcacatgctggtggcagccctggcatgccaccggggggcacggcgccccatgccaggcggcactcgctgccgagtcctactgctcagtctcacctttggcacgtccatggcctgcggcaacgtgggcctaaggctgtgcccctggacctggcacaactggttactaccaccacacctctgttcancctggccctgtcggcgctgctgctgggccgccgccaccacccacttcagttggccgccatgggtccgctctgcctgggggccgcctgcagcctggctggagagttccggacaccccctaccggctgtggcttcctgctcgcagccacctgcctccgcggactcaagtcggttcagcaaagtgccctgctgcaggaggagaggctggacgcggtgaccctgctttacgccacctcgctgcccagcttctgcctgctggcgggtgcagccctggtgctggaggctggcgttgccccaccgcccactgctggcgactctcgcctctgggcctgcatcctgctcagctgcctcctgtctgttctctataacctggccagcttctccctgctggccctcacctctgccctcaccgtccacgtcctgggcaacctcaccgtggtgggcaacctcatcctgtcccggctgttgtttggcagccgcctcagtgccctcagctacgtgggcatcgcactcactctttcaggaatgttcctttaccacaactgcgarttcgtggcctcctgggctgcccgtcgggggctgtggcggagggaccagcccagcaagggtctttgagacctgggggatctcaggagccacctgggatggccctggcctgaatccagcctccgctgtggccatagaaggaatggagaacagggctgggcatggtsgctcacgcctataatcccagcacttccagagtccgaggtgggtggatcacctgaggccaggagttcgagaccagcctggctagcatggcaaaacctcatctctactaaaaatagaaaaattagctgggcatggtggcgcgtgcctatagtcccagctacatgggaggctgaggtgggaggatcacttgagccctggagatcgaggctgcagtaagccaagatcgcatgctactgcactccagcctgggagacagagcgagacgctgtctcaattaaaaaaaaaaaaaaaaaccggcacgtag

SEQ ID NO: 40 - DNA, 1467 bp, Homo sapiens
gaattcggcacgagcagagcaagactccaactcaaaaaaaataaaaagaaagaaagaaacatccctggcaccctgctctccatcatgtctgtgtgtttaccgctgcacctccctttcctgatgctagcaaaggtagccaccagcttttgcagatggcagctcacactatttgtgtccactttttacaaagatgccctggttcatactgtcaatgacagaaatcaggaagcagaattggaagcactcaaaaagtcctgttgacagtgcacatcccctcatgccctggaatatcacacagagagctttgcagtgagctggaaagtcctccagactcaaggccgggcctgggsccacaacagactttgacagccctgcagtyccgggctcaccagggaarggagccaggagttcagcccccgcagcggcccctgctcacagcacaggcaaaagccacagccacacaggatggggcctccctacttcagcccaattgtgatttcctgccttgcaaatcatctctcttgaagctcttcagctcctcttcatttcttacaccagcaccagcagctggccttcttcttgagccaattcccagaaagcaatargctgagtgtcactttttcagggttgctgagagctgcccatggggggaggcgggacaatagcagcagcrgggttacccagcaamscaactccctcccaagcaccagccctgtggcaggtggacttcaaagttacctccctgaggaaagcacaaatcatgtgctggacctttgacatcctccccgagggtcacacactgacaattacactctgggtataggatccaccccgctgggactggctaggcaccctgcagaatcccaagagggccctgccgacagctaggacccgccaagcccattagacctcctagcaccttccactgtccctgtttcctttggtttttgttgtgttttttgttttgtagagataagtatcacttggtcgcccaggctggagtgcagtggtacaatcatggtacactgcaacctcaaactcctgggctcaagtgatcctccacctcggcctcccaaaatgctgagattacaggcaccatgcctgggtcatatttttttttttttgagatgatgtttcactctcgttgcccaggctggagtacaatggcgcgatctcggctcactgcaacctctgcctcccgggttcaagcgattcgcctgcctcagcctcccaagtagctgggattacaggcacccaccaccatgcctggttattttttgtatttttagtagagacatagtttcaccatgttggctgggctggtcttgaactcctgacctcaggtgatctacccaccctttagcccccaaagtgctgggattacaggtgtgagccactgtgcccagccaatttaaaaaaatttttttaagagatagggtctcactatgttgcccaggctggtctcga

SEQ ID NO: 41 - DNA, 914 bp, Homo sapiens
ggcacgagtgaaaatctactctatcagctgctgtggttgccaccattctcaggaccctcgccatgaaagcccttatgctgctcaccctgtctgttctgctctgctgggtctcagctgacattcgctgtcactcctgctacaaggtccctgtgctgggctgtgtggaccggcagtcctgccgcctggagccaggacagcaatgcctgacaacacatgcataccttggtaagatgtgggttttctccaatctgcgctgtggcacaccagaagagccctgtcaggaggccttcaaccaaaccaaccgtaagctgggtctgacatataacaccacctgctgcaacaaggacaactgcaacagcgcaggaccccggcccactccagccctgggccttgtcttccttacctccttggctggccttggcctctggctgctgcactgagactcattccattggctgcccctcctcccacctgccttggcctgagcctctctccctgtgtctctgtatcccctggctttacagaatcgtctctccctagctcccatttctttaattaaacactgttccgagtggtctcctcatccatccttcccacctcacacccttcactctcctttttctgggtcccttcccacttccttccaggacctccattggctcctagaagggctccccactttgcttcctatactctgctgtcccctacttgaggagggattgggatctgggcctgaaatggggcttctgtgttgtccccagtgaaggctcccacaaggacctgatgacctcactgtacagagctgactccccaaatccaggctcccatatgtaccccatcccccatactcacctctttccattttgagtaataaatgtctgagtctgaaaaaaaaaaaaaaaaaaaaaaaaaaa

SEQ ID NO: 42 - DNA, 1131 bp, Homo sapiens
ggcacgaggattcttcttttatatactgattacaagactgacacctatcaagtatgatgtgaatctgattctgacagctgtcactggaagcgtcggtggaatgttcttggtagctgtgtggtggcgatttggaatcctctcgatctgcatgctctgtgttggactagtgctggggttcctcatctcgtcagtgactttctttactccactgggaaacctaaagatttttcatgatgatggtgtattctgggtcactttctcttgcatagctatcctcattccagtagttttcatgggctgcctaagaatactgaacatactgacttgtggragtcattgggctcctattcggtggttttaagccattgacagttactggtccacaagcctttcctacatcactttgaacgtactcaagagagcgctcaacaaggratttccacagagctttcacaaatgtgccttttcaaactaatgacttcattatcctggcagtatggggcatgctggctgtaagtggaattacgttacagattcgaagagagagaggacgaccgttcttccctccccacccatacaagttatggaagcaagagagagagcgccgagtgacaaacattctggaccctagctaccacattcctccattgagagagaggctctatggccgattaacccagattaaagggctcttccagaaggagcagccagctggagagagaacgcctttgcttctgtagatgcccaggggcttggtcagtgtgcctcagctttggagttcatgcctggagtggttcaacagtctctggtgcaagtctaataagagatcaggcatatatatctgttctttgcataatattatggtgcccttattgatatatggtaagggtgtactaggggattaggatgattgtaagagaatgagaaagatgaccaaaaggttggtggtagggaggctttttcttatttccaaatacttgagaaattaccttttggtttacaaatctatgatcaacttattccattaaatagatacattaaaaaaattaaaaactgaattcttctgtcagaaaaaaaaaaagaaaaaaaaaaaaaaaaaaaaaaaaaaaaaaaaaaaaaaaaaaaaaaaaa

SEQ ID NO: 43 - DNA, 1333 bp, Homo sapiens
  SITE (411): n equals a,t,g, or c
cctggaacacttcaacaaccagtatccagccgcagaggtggtgaactttggcacctggttcctcttcagcttccccatatccctcatcatgctggtggtcagctggttctggatgcactggctgttcctgggctgcaattttaaagagacctgctctctgagcaagaagaagaagaccaaaagggaacagttgtcagagaagagsmtccaagaagaatatgaaaaactgggagacattagctacccagaaatggtgactggwtttttcttcatcctgatgaccgtactgtggtttamccgggagcctggctttgtccctggctgggattctttctttgaaaagaaaggctaccgtactgatgccacagtctctgtcttccttggcttcctcctcttcctcattccagcganagaagccctgctttgggaaaaagaatgatggagagaaccaggagcactcactkgggaccgagcccatcatcacgtggaaggacttccagaagaccatgccctgggagattgtcattctggttgggggaggctatgctctggcttctggtagcaagagctctggcctctctacatggattgggaaccagatgttgtccctgagcagcctcccaccgtgggctgtcaccctgctggcatgcatcctcgtgtccattgtcactgagtttgtgagcaacccagcaaccatcaccatcttcctgcccatcctgtgcagcctgtctgaaacgctgcacattaaccccctctacaccctgatcccagtcaccatgtgcatctcctttgcagtgatgctgcctgtgggcaatccccctaatgccatcgtcttcagctatgggcactgccagatcaaagatatggtgaaagctggcctgggagtcaacgttattggactggtgatagtaatggtggccatcaacacctggggagttagcctcttccacctggacacttacccagcatgggcgagggtcagcaacatcactgatcaagcctaacgccaagtgtacaaactggcccaaccacaggagctgccagtatccagcagtatctggaccacaggcaaagaaaaccactaggaccaccaggagcacacaaccccagacccacgccggagggcatccctccaccagaagattccgccacctcaagtgaactgcaggaatcctccaacaaccacaaacacatgcttcgctgttagtgtcttcttcctgccctcagcaccacagctcaagaaaacctaaagtttcaatacaanccataggctcacaaaaaaaaaaaaaaaaaaaaaaaaaaaaaaaaaaaaaaaaaanaaaaaaaaaaaaaa

SEQ ID NO: 44 - DNA, 1004 bp, Homo sapiens
gcttcactggagttctaagttttctcccctctgttttgaatgagtcagctctgcttctcactactgctttcttccacatgccacggaggggttgccagcctcttgacctcagaccttagctctcagtcccatcgtttctccatctgcactaatgtgaatcactctaagtattctagtctctgatgtgttttgaaggcagaagcagtcagagggcactgctcaccaggctgggctgggcaggcagatcacacggaagccctgccctgtcacagttgttaatactgcagggagatggtggggagacactatggggaacttgaggagtcatggttcacaatgtacttctaaaccactgtgagtttttttgctcttgtctttggaatataatactttattgctgggggataatgagtatttactttaaaaaacagatgcatttctaagtccctctgttttgtcttgacttccagctccccaacatactcacattccactacttattctctattttaactttactgcttcttttacttttttttagttttacttttattttttatttttttgagacagartcttgctctgtcacacaggctggagtgcaatgacgcgattttggctcactgcaagctccgcctcccaggttcatgtcattctcctgcctcagcctcccaagtagctgggactacaggtgcccgccaccacgccctgctaattttttgtatttttagccgargcaggtggatcacctgargtcagcagtttgaaaccagcctggccaacatggcgaaaccccatctctactaaaaatacaaaattagcagggcgtggtggtgcactcctgtaatcctagctacttgggaggctgagacaggagaatcacttgaacccaggagccagaagtcgcagtgagccgtgatcatgccattgcaccccagcctgggcaaaaagagcgaaactccatctcaaaaaataaaaacaaaaaaaaaaaaaaaaaactcgag

SEQ ID NO: 45 - DNA, 1494 bp, Homo sapiens
gtcgacccacgcgtccggcggcggcaggcgcgggcgagggccacggggagaggagacgcagccccgcgggtggcacgctcggccgggccccggcccgcgctcaacgggcgcgatgctcttctcgctccgggagctggtgcagtggctaggcttcgccaccttcgagatcttcgtgcacctgctggccctgttggtgttctctgtgctgctggcactgcgtgtggatggcctggtcccgggcctctcctggtggaacgtgttcgtgcctttcttcgccgctgacgggctcagcacctacttcaccaccatcgtgtccgtgcgcctcttccaggatggagagaagcggctggcggtgctccgcyttttctgggtacttacggtcctgagtctcaagttcgtcttcgagatgctgttgtgccagaagctggcggagcagactcgggagctctggttcggcctcattacgtccccgctcttcattctcctgcagctgctcatgatccgcgcctgtcgggtcaactagcctcaccgaggtgccggagagggagcgctggacaactagaatgttgacctcgagccgaggccctacttgcagcgcaccggaggagaggctctctagtctgaaggcaccgccggcttgcgccgagctgagtgccgggtttccctattccaatcctgtttgaaatggtttcttcagcagggcttaaaagagcagccttcatcctgaaaatgtatttccttttgtttaatgctttgagtagataatcctgaattgaggtcatgaggaggccccccaggccagacagtcctgaacccctctgacacttggaaactgaatataagtaaaatgtccaggtggactctgagtatttcctgtggatcctgggaaagtactgttgcacaaaggctgcaaagctggactcaggaatgtcctccaaccagcagcgctgacctaagagctccctgtgccgtctatccagaccagacttcggtagatgcctttgttagatctatcacatgtaaacgagcttgtatctccttccctgtgccacgagagagattggctttttattccagtctaggcagagacagaagaatgttgaataagagcacgattagagtcctgtctggttatctgttgcccaagaaaagaactctgctgtccaggcactgcttggcttactatcccagcaaagactgcagttttgtggacttttgaccaccttgggctggcactcttagcacacctgagacagatttaagcctccctaagagactgaagagaggaacaggtgtcagatactcataggcactgagatctacaaatgggaagcttgtgagtggcccatctttgttggcctacgaactttggtttgatgccagtcaggtgccacatgagaacctttgctgagatgcaaataaagtaagagaatgttttcctgaaaaaaaaaaaaaaaaaaaaaaaaaaaaaaaaaaaaaaaaaaaagggcggcc

SEQ ID NO: 46 - DNA, 1166 bp, Homo sapiens
gagacctgcccatcgaatggaattgagcttcgtcaggcgcctacttctctttacattcttcttctccacattcagcccacccccacccaccccatgcttggaaggtcttatgtcctgcctgccttctccaytaayyaagaacacggcgggctcccaaaccaaatccctaagggagataggaacgggaatatcagacactcacgtatcaccttcccctgctcaagctccactttgcagccggagtcccacctgggattcatcagatccaaactccatggactagttaggcctggaaaagaccttagaggtaggaggtctttacagcttagcaaacattctttgagtacctgctatatgttgaggtgggagacttataaacaagtaagttacactgcagtgtgaaaatgacagtgtagatgataaagctgaagcttaagcgatgctgacacatagacttctgttagctctagggctttttcctgccttaactgacagactttgacaaatcttctaggactgtgtgaggaatgaatatagatgatcgaaaggggaattcttttaagacttgtgaaagaagctacattggagaactgaccttcccatgtactgaggacggcctgtgtgtgtgtatgtgtgcacatgggcatgtgcacacatgtgtgtaggaacctgtgtacatttcttttatcattctcctgatgcctttgttagtgtttctggctccctgcctgttcttaataaaatccacagtttatcttgtagcttracctttcttgaggccatgctacatttcaattttggtataatagaaagatgttgtcaaagctgatgggggctaggtcctaagctcattgaagactggaggctgtgccttgttttcctctacatcccctaagccttttgtacagtgtgtggcacctagagggtgcttactgtcagcactgaattgcgcccagtgcccaccagggacctgcacctctcatcaggaatgactttgggattgccagctgttctatctgctcctctcagcatgaacactaagcagagtgccttgtttttactgcacttgggagccatatcctgttgcctttttggatgattcgggggaaattccttgtctgtggttccgtatgtgttgcaggctgagggttggcttgctccctcga

SEQ ID NO: 47 - DNA, 1536 bp, Homo sapiens
ccacgcgtccggggcagaaaggaaccgggttgtcttgggccgggcagggcgggtggtgactctcaaaaggaaataggatcatggcagcagatgatgacaatggtgatggaacaagtttatttgatgtcttttctgcttctcctcttaagaacaatgatgaaggctcactggacatatacgctgggttggacagtgctgtttctgacagcgcttccaaatcctgtgtaccatcaagaaattgtttggacttatatgaagagatcctgactgaagaaggaactgcaaaggaggcaacatataatgatttgcaagtagaatatggaaaatgtcaactacaaatgaaagagctgatgaaaaagtttaaagaaatacagacacagaatttcagcttaataaacgaaaaccagtctcttaagaagaatatttcagcacttatcaaaactgccagagtggaaataaaccgcaaggatgaagaaataagtaatcttcaccaaaagattgtcctgagtttccacattttcgaaataatcataaaactgcaaggacatttgatacagttaaaacaaaagatcttaaatctagatctccacatttggatgattgttcaaagactgatcaccagagctaaaagtgatgtttctaaagatgtacatcatagcacttcactgccaaatctggaaaaggaaggaaaaccacattctgataaaaggagtacttcacatttacctacatctgttgagaaacactgcactaatggtgtttggtcacgttctcattatcaggttggcgagggtagctcaaatgaggatagtagaagaggaagaaaagatattagacatagccagtttaacagaggaactgaaagagtacgaaaagacttaagtactggctgtggtgatggtgaaccaaggatattggaggctagtcaaaggctacaagggacatcctgagaaatatggtaaaggtgaaccaaagactgaaagcaaaagttcgaagtttaaaagtaactcagattctgactataaaggtgaacgcattaactcttcttgggagaaagagacccctggagaaaggtcacacagtcgagtagactctcaaagtgacaaaaaactagaaagacaaagtgaaagatcacaaaatataaataggaaagaagttaaatcacaagacaaagaagaaagaaaagttgatcaaaaacctaaatcagtagtaaaggaccaagatcactggagaagatctgaacgagcatcacttcctcattccaagaatgaaataacattttctcataattcaagtaaataccatctagaagagagaagaggatgggaagattgtaaaagagacaagagtgtaaacagtcatagttttcaagatggaagatgtccatcttctctttcaaacagtagaactcacaaaaacattgactctaaggaagttgatgccatgcatcagtgggaaaatacacctttaaaagcagaaagacatagaactgaagataagaggaaaagagaacaagaaagcaaaaaaaaaaaaaaa

SEQ ID NO: 48 - DNA, 1038 bp, Homo sapiens
ccacgcgtccgagacatttaaactagattcccagtcctctccttcaaaagcttggtctttgtttttcctatagggaaaaaagtcaaaataagttccaaaaactatcctcaaagtagtattgtgcttgtagtaaatgaaggttggatggatggatactgacaatggtggcaggcatttcaagccttttaaattagtactttttgtcgtcttgcttattaaaattttgttaattttagcaaagaccaattgttgtgataaactggtgttttttggatgcttcaagcacacgttaaccaattttttaattccccttttggttcctcccattgttctaaaataggactttcatattattaaaacctcaaaagatgatccacccaggatgaacaaagatcaccaaggggaaagaaaacattttttatctttacagaaaacatgttaagattatatatagatgtattctttacattggatattgtattagagtcctccttacaagaaatgaaatagtttttagcactcttagcattagagttcctagattggtgttgatagctacagttttaaaatgtataacctgaaaatgaaggttaattttgcattgtaagagcacatttgatctatgtaaaaagtgtccatttggtgtatttttttaaaaaagagaaagcactttcatattaagtagcatgtgtatgaatttagattttcatatttgttgtgtctgtattcagtgaagtaaattgagcatttaaatgtttgttgatggcaacattaactattaaattaaagcaccttatactctgctgcttaacttgcttgtaattgcacctttgttacctgcacattttcatatagaatattgttgtaacattgcttcatgtgggtctggatggaagattagtgggcctacaggatcatttatttatattgtttatattacaataatatattgtagatcagttgtaagttcatttctttacaaataaaagcctcttccatttgaaaaaaaaaaaaaaaaaaaaaaaaaaaaaaaaaaaa

SEQ ID NO: 49 - DNA, 1176 bp, Homo sapiens
ccacgcgtccgaggacaaaaaacaggccttttaaaattctgttcagaaccagaagatgatcaagaaagggaaatgcccactagcagcttctactgtaaggttagagctgacagagaaagaactccttaactactatttgcttcattctctacaaaggaaactagagaagtgggttgatgtaatagaaagaacatgtgtttgtggggccaggcaaacctgggtttaattctgtttcaacactgcttaacaaaatttatgggaggctattgttttggattgggctcctgcaccaggcccctccgggaccaaaccaaaatggagtcactcatactaaaactccaggtcactgaaccaaaactaagttgttttatctgaccttccaagaaatcaggagggagaaaacaaccaaatctccaaacaggccagttttaatcagcgtgataaggaagtcctctcttttttaaccctataaagaaagtaactttttgaaatgatcaatacactttgtattccttagttctgctttctttagcccctttctgcctataaagcccacttcctctgctcaacttactgaagcagtattccattttatagaatgagatgctgcccaattctggaatcactaataaaagccaattagatctttacatttgttgaaattttgtctttgacaacattactagttatattattctgggtatccttttccccatctgtaaaatggacatagcgatatccttcccatcagatttttctcattaatagaagtaatacattcaaaacactgagccaggctcacaccagtgagcaacttgttaatattactcagaagcatattatagatattgacagaaagcaatactgttcctaattaggggaagaaattctaagagagtacctaagagtttgaatttagattataacatttgctctgagtattttacattacagcctttggggggaaaagtacaaatgagatctgagaacagtggtactcatctttgaggaattatggaaaacgtaatagaacactaaacatgggaaaacatcggccttcaggttgaaaagtggaaatctcaatccctgaatttttttttttttttactaagtaacttttttgcccattggtgtcatttaaccaaaagaagaagaaattccaaaaaaaaaaaaaaa

SEQ ID NO: 50 - DNA, 731 bp, Homo sapiens
ccacgcgtccgagacgccacctgggcggacagccaggagctctccatggccaggctgcctgtgtgcatgttccctgtctggtgcccctttgcccgcctcctgcaaacctcacagggtccccacacaacagtgccctccagaagcagcccctcggaggcagaggaaggaaaatggggatggctggggctctctccatcctccttttctccttgccttcgcatggctggccttcccctccaaaacctccattcccctgctgccagcccctttgccatagcctgattttggggaggaggaaggggcgatttgagggagaaggggagaaagcttatggctgggtctggtttcttcccttcccagagggtcttactgttccagggtggccccagggcaggcaggggccacactatgcctgcgccctggtaaaggtgacccctgccatttaccagcagccctggcatgttcctgccccacaggaatagaatggagggagctccagaaactttccatcccaaaggcagtctccgtggttgaagcagactggatttttgctctgcccctgaccccttgtccctctttgagggaggggagctatgctaggactccaacctcagggactcgggtggcctgcgctagcttcttttgatactgaaaacttttaaggtgggagggtggcaagggatgtgcttaataaatcaattccaagcctcaaaaaaaaaaaaaaaaaaaaa

SEQ ID NO: 51 - DNA, 1437 bp, Homo sapiens
cgcccgacgccggaactgcgagctctcagcgggagccgagacggtgcagggccggagaagcaccttcactcccagcctgcgccccgatgctgcgcgttctgtgcctcctgcgcccctggaggccccttcgggcccgcggctgcgcttccgacggggcggccgggggctcagagatccaagtgcgcgccctggcgggtccggaccaagggatcactgagattctgatgaacagaccttctgcccgcaatgccttggggaatgtcttcgtcagtgagctgctggaaactctggcccagctgcgggaggaccggcaagtgcgtgtcctgctcttcagaagtggagtgaagggcgtgttctgtgcaggtgcagacctgaaggagcgggaacagatgagtgaagcagaggtgggggtgtttgtccagcgactccggggcctgatgaatgacatcgcagccttccctgcacccaccattgcggctatggatgggtttgccttgggcggaggcctagagcttgccctggcctgtgacctccgagtggcagcttcctcggcagtcatgggactgattgagaccacgcgagggctcctcccgggggcaggagggactcaraggctgccccgttgtctgggggtggccctggcgaaggagctcatcttcacgggccgacgactgagtggaactgaggcccacgtactggggctggtgaatcacgctgtggcccagaacgaggagggggacgccgcctaccagcgggcacgagcactggcccaggagatcctgccccaggcccccattgccgtgcggctgggcaaagtagccattgaccgaggaacggaggtggacattgcatctgggatggccattgaagggatgtgctatgcccagaatattccaacccgggaccggctagagggcatggcagccttcagggagaagcggactcccaaatttgttggcaaatgacccccattttaaccttcagcatgggagatgcatgccctgaagagcaggatccagaaggaagatttgtggccagattgccttcatcatttcacctctccagacttccatttcttcacaaggatgatgatggaaataaaatgactggcgtgatgcctggaaccaaggtgctgatcctaccacctactgctaccttccttagcttcaccctggctagaaataatcacgagggttgggtttgctttggaaaatgcctgtctctctacttgaatgataaagaattaaattagatctctctgagtcttggtatcattggctctcagcccctgacctctctcagttatcaggcactcattagagatgtcagaagattttaagatacccctagtttcttcctgtggaacaacagaggtaataaataaactctggacatcggttgaaccagtgtcaggggtcagactgcagatcccagtct

SEQ ID NO: 52 - DNA, 1369 bp, Homo sapiens
  SITE (3): n equals a,t,g, or c
agncagacgtagaacgtagtggatccccaggggtgcaggaattcggcacgagatttgatacccagtgccatattgtccctaagaaaggttgcacaaatttacactcccaccaagagtgtgggagagccatttgccacatagactcaccagtattttttttttaatttttgatccatttggaagttatttgggaacttgggtgtttttccccaaaagcaaaggcaattgcctcaacaccagttatcaaaggatccctacagatctattttccctgtagatctgaaatgctgtcttcattgtatcttttgctgatgcccccgtacaaatttacaggtgagctccatcctcctgtagcagccacctgcctactgacagtcctactcgggtgtctgataggtgtctcaagtgatggatggatatgacagtagagtccttgatttactgtcccatggcccctgctcatcttctctttcttgattgatggtgccatcatctacccagttactttgaccaaagcaatgggagtcatcctggattccttkctttytctccacttctaagccatcagtatactggcactgtgttaaagccatatttcaaaccagaccacttgtcaccattcctgtcacttctacctcttttatcccaacatcacctcttgggtagaccattgcaaactggaagtgcatttccagaatattcttgttggtatagaaccctctgtctacatagtagctagagcagttttttttttttttagatgttaaacagatacatcgctctgctaactaaaaccctttaaagtgttttccatctcaattagaatagaattcatagtcctcaccagccactgcaaggtttatataatctagcccctgcctatcttccttgcctcttctctgttacccacaacctgctttttgtttcatgatgtgtgaactcatttcaaccttagggtcctgctcttgcctcttcctttgcctagaatgcttttcccttgtcctaaatcatctgtgttatgctagtttttargtctcaactcatgtcaccccgttgscctttatctcattgtctggctttattttctctaaaacacttggcactatgtaratgttctatttatttacttattttaagggtagaatctttatctgttttgcttggtgccaattattcaacatgttgaatagtgcctggcacctagcaggcactggagcctatttctggaatttcatgttgcaccattgccctctctgtttgttctccattactaaattcctttcaagccaaccccatggcctccatgactttttcaaaaaaaaaaaaaaaaaaaaaaaaaaaaaaaaaaaaaaaaaaaaaaaaaactcga

SEQ ID NO: 53 - DNA, 1037 bp, Homo sapiens
ggcacgagctcgtgccraattcggcacgagggtcatagtccacagaggtaaaagttaacaattctgatgctcttgtatgtgcataccagaggctctagggaagaattccctctttctttcttccaccttcttgtggctgctggcattctttggcttgtggtcacatcactcctatcttgaaggccagcatcttcaaatctgtttcttcttcacatagccttctgtgtgtgcagtgccctctacctctctcttataaagacatttgtgattaaatggagggtttaggataatctcgtcaagatccttaacttaatcacaactgcaaaaacctctttcccaaataaggtaacattcacaggttccagggattaggacctattatctttggtaagtattattcagcctaccacaatagctaaaacaattctgaaaaagaagaataaagtgagagaaatcagtttatctgatttcgatacttattgtatagctatggtaaataaggctgcatggtattaaagaaaggacatatatgaatgaaacagaatagaggacccagaaatagacccacacaaaggagcccaaattatttttaaccaaggtagaagacaatttattggaggaaagacagccttttcaacaaatggtactataacaattagatatccataggcaaaaaaaaaaaaaagaatcttgatctaaggctcacaccttatataaaataatattaaactcatggccaggcacagtgactcatgcctataatcccaatacactgggaggctgaggcaagagtatcacttgaggccaggggttcaagactagcctgggcaacacagtgaaactctatctctacaaaaaaattataaactagctgggcatggtggcacatgcctgtagtcacaactactcacgaggctgagaagatcacttaagctgagttgttcaaggttctaatgagctacaatcgtgccactgcactccagcctaggtgacagacaaagaccccatctcaaaaaaaaaaaaaaaaaaaactcgta

SEQ ID NO: 54 - DNA, 1373 bp, Homo sapiens
ggcacgagagcaaagagaagtgtccaccaggcccgctgcaccaacgctgcgtctttaacagctctggggctgggcgcgtcatggctacgagaaagcgcatgaccacttccctgtttggtttggtttgtgttgtgtgtcagggcgcaggggtttctgctttcactcaagttaatttattttccttttccttggtaattgtgaaaaaacaaaataaaacctcctgtgagccttttggaacttctggaaaagtccctttgctgtgarcsgctgactctgagaagagctttgagcagggctggaaaccatttttctgcaaccttttctttcctggggtatgtctgggtgcacacaggctccccacaaggcaaaggctgtccctggatggttggcaaaatgcgccacaccagagtgggtttgtgttggcaggaggcatgaraaaaccttgctgatggcaggggaggacggcgacaccacgatgggaacaaaatcctcctccttacytctaattacaaagaggaaaaagtcactgaaaaaaaaagtttaaaatgtcttaatataagagtcatatataatccaaagctaccaaaggccaagtgtttagggggaagtttctggtggttaaccccacttcagggggatttaaagtggttgtggtgaggatttggttccaggtatgcgtcctgccaacctgggtgggtgttccctttggtggagcctcttgaaaaatgarggartggctgggtgcagtggctcatacctgtaatcccascactttgggaggccgaggcggccagatcacctgaggccaggagttcaaaaccagcctggccaacatggtgaaacttcatctytacaaatatacaaaaattagctaggcatgatggcaggctcctgtaatcccagctacttgggacgctgaascaggagaatctcttgaacccaggagtaggaggttscagtgagctgagattgtgccactgcattcccgtctgggcgacagagcaagactccatctcaaaaaaaaaaaaaaaaaaaaagagggagtggctgggtgcagtggctcatgcctgtatcccagcatattgggaggccaaggagggaggattgcttgagcccaggagttccagatcagcctgggcaatctsscaaaacccgggcagtacaaaatccataaaaattagcggggcatggtggtgcgcgcctgtagttccagctacttgggaggctaaggttggagagttacttgagcctaggaggttgaggctgcagttagccatgattgtacctctgtacgccagcctgggtgacaaagcaagagcctgtctcaaaaccaaaaccaaaacaaaaaacaaaacaaaaaaaaaaaactcgta

SEQ ID NO: 55 - DNA, 1347 bp, Homo sapiens
ggcacgagctggtctatagtgaagttactggccattccaaaggctatggatttgtggaatacatgaaaaaggactttgctgcaaaggctagactggagctattgggtagacagttgggagcatcagcactctttgcacaatggatggatgttaatctattggcttcagagctcattcattctaagtgcctttgtattgataaactccccagtgactacagggattcagaagagctgttgcaaattttttccagtgtccataaacctgtgttttgccagcttgcacaggatgaaggtagttacgttggtggctttgcagtggttgaatatagcactgcggagcagctgaagaggtccagcagcagcagacggtatgaccatcaaggcagcaaagtccaggtttccttctgtgctcctggagcgccagggcgaagtacattagcagcattgatagcggctcaacgtgtgatgcacagtaatcaaaagggcttacttccagagccaaatccagtacaaattatgaaaagtttaaacaaccctgccatgttgcaagttcttctacagccccagttatgtggacgagctgttaaaccaggtatggaccatgtatgtatactgattaattaaagaatgctagcatgaagttaattttttcacatgtgaaatatggaaaaatacattgatttgtgaaaaatatatttaaattagtataaaatatttaatttttctaggacctgttaatgaattttaacaataatctgaggtgatatattttgactatttttgacatatacaacctgcttatgtggtattttttactattaataaagctaaatatcaaaccttctgtttagctcttagttgacatgaatttatagaccaaggtaaaggttaacacactacattgttataacctatattaacaaagaattaatactctctatgtaatatttttagcagaattattttgttgaaaagtgccaagtgtttgtttcctctttgttcttcccctttttgttgtaaaattgtttcactttgtagcaaatgatgaaaacattattattttctaagtgttatgcaaatctttataatatcagtatacattaaatatctacctatttagtattctttctctagtaagagcttaccttctgtgcattctgaaatgtacaactttttatgtacaaaaatgtctgttttagcattatgaggaaatgaatgcctatacagtggtctccccttatctatgttcttgtgttccacagtttagttacctgcagtctgaaaatattaagtggaaaattccaaaaataaactacttataggttttaaaaaaaaaaaaaaaaaa

SEQ ID NO: 56 - DNA, 822 bp, Homo sapiens
ccgggttgacccacgcgtccgcggacgcgtgggcaaatattggtaatgctgggaaaagggagttcagaatgccaaaacgtttctggttttatttgtcttgggtgaggacccagaggggtgggagatggaggtgtgagcagcatggtctgttgtggttttttcttgttgtggagtagagttagatcatacatgaagctctctgggcataggtggagtagcagctgtccacaccattgctattcaaagtgtggtttgcacaccagtaatggaaaatcatctgtgcacactgtttagtttaactgatactttttttttcatagcaagatttcttaatgaaggaagtaatgtattgatttacattctgactcattgtctttatcttgtctttgatcagtttgtagactggcactggtccacactttgaataacactattcttcattctactttccatgtacccggatgccaggcaaacagggagttttacgctgggtggagaacggaacattctgctgactccttgaaagggcttatctcaccaggcatggtagctcacgactgtaatcccagctctttgggaggctgaggtgggaggattgcttgagctcaggagtttgagaccagcctgggtaacatagggataccttgtccctacttaaaaaaaaaaaaaaaaaaattagctgggtgtggttgtgcacacctgtagttccagctattccaraggctgaggcaggaggataggttgagcatgggargttgaggctgcartgtgccttgatggcgccactgcactccatcctggttgacaaaaaaaaaaaaaaaa

SEQ ID NO: 57 - DNA, 536 bp, Homo sapiens
  SITE (536): n equals a,t,g, or c
ggacgagtggggagctggaaggaggatggagtgggaagataatcttcccttggagttcagctgtcccgtgaccaaactcctctctgtccccagctggactcctctagatgctcagatgctccttctcttctttccttctctgtcacaccattcttctgttccttggctcttctgctcatctccttgtggagscawaggtttggggtttatatgagtacaggataggtgacatggtggatcaaaaggcaacattttgtgtgcaaaaacaggaatgcctgttcccattagggtcatgggttkccagggttgagggtggggcctttgctagggaaccaccctcttctacccagtattttcctgtctcctgtctgtatcaataggtacacaatatwtattaaattaatkaatgactatacattatgaaatgggaaatgcaaggtataaaggagaattgctgtccttgaaaagaaatttagtttgtttttttgttgagatggagtcttgctctaagctagagtgcagaatgtaatcaaggn

SEQ ID NO: 58 - DNA, 1262 bp, Homo sapiens
  SITE (12): n equals a,t,g, or c
atcctgcagttnttctacgcaaataaaattctgtaagcgcagagcagcagggaaatggttctgtatgagtgcattgctgagccatcatgttcccctgttttatctcactggatgcctcttctcactgcttgcctcttgggattgtaatgggaaagagggtgctgggagagcaatcaaaggcaaaaataatacatggaattgtatgattttatctaaagtaaaattctagactgctttcacttatttcatttcctccccatacaaatttgatgagcaggtacacactttctttaaaacattccaagtgtatatagattcagtgcttatttctcagctttttctttcttaagttcatctctgtcacctagcttttatttttaatactcaatttctgaggcttaggaaatacttgttaccttaagcgtttttttgttttttttttgttgttgttttgtttttttttagtgtatttgctattgatactttgctattgatacctttacttgccaagatttatttaaagttggccaagataccaagaaggtggcttgcaggggcatctttggcttctcataattttaaacagtcattgctttcaagatcatcctaaacaagaattaatagagagaaaaaaatctataaagcatgtgttgaaaatcctcaggctcatgataacttacgggataacaatagtaggtaacaccgagtgcttacaattcagtgggcacggtcaaagtgctttttgtaagttctatttaatacttgcgacaaccctgagatagtaagtattattattgttctcattttatagatgagaaagctaacacatagggaaattaggtaactttccccagataactcagtaaacagtggagctggaattcaaacatgtacggtctgacttcttgagcccatactcaataatcatgccatcctgcctttccatggaataactgatggatggttagaaatgttaaaaacaaagcaaatgcaaacatctctgcttctgctcctctactaagcccagttaacttttgattttatatgtactaagtatacatttttattaatttatgtgagctgagtgtttttattaattactttttggctattaatgtcaattattttggtgatcaatcctagttatccaaggagtcacatcactcgtaaaaattacaagaaaaatctctaagtccctctcaccctccctctaaccagattgcatcccaagcttctatagaaactctggtaccactcgtgcc

SEQ ID NO: 59 - DNA, 1269 bp, Homo sapiens
gaattcggcacgagarataacgagatgctactagtctgggtaccagctgtacgaggaagcatacagctaagtaggtcagtgattaccaatcagtgtggtgagtacatgaaacagcaggcaaggtttgggacagccctgtttgaaatttggaagtgaaaatcatggtgcacaaagccatccttgctctgcttccctggggattctctgctgatgaattactggcttccctaatgatgtktcttacagagaagtatcagaactgcagttctaccacagacatastgaatcaacaactcaggagcttgggccagaactttatgtttcaacaaaatctccagttgattctgatgtagcctaaagtttaagaaccacattgctatagagcataaattatttgagggtagtgctcacggattatttaaactgatatttctagtgtccagtgcttgacctaaagtaagaattccagacatgtttatgaaagagtgaagagggtcaaagattttgcttttgaaccttttgagtttctgtatgactcctttgggtcatctaggaagatgggattatgaagctgtcagggtccatatwacaacsaaaagtaaatgtccaggaaagacctgtgactgaaaaagtcatgctattcacccttcaagtcccactccaagtcatacctgctttgcagatctttctaacttaaaccccgkgtcctatgatcctcctgtctgcgtccattctttattatttaagtgaccatatcctaactgaatattgaccatcattactcatttttataccttcaacagctggcacactttttagaatagtgttaatgcagaacaaatctttcctcattaataaggatttgtggcatacctatgttaatgttgaattaaatcagtgttgaaattattagcaaagctattcctatcagacttagtcctgaagacaagccaggtcttggtaattttacaccttacattattttcaaacagatcagggtacagtccargagccaggttttattgggcaccttccttctcttttaacattattgtttgttctctccctgctgtctcttgaccagggaaatgtcttcaatatcatttattttgagatcttgctcgaaactgtcattggagatgttagctgttaatgaatgcaaaaagttgataagttttagctttctttttttgatatgttggctttagaaagtcaaaattctaagactaaaagaccttcaagagagaaattaataaaaaaaaactcgta

SEQ ID NO: 60 - DNA, 1829 bp, Homo sapiens
ggcacgagtggccctgatagataacggaagtgaggaaggatgacctgaaagtggtcaggattatgatgacgtcgagccttggcctttccttcctccttaacttaatcctgggtatgaaattcacctatctgattcctcaaaataaatatatacaactcttcactaccatcctcagtttcttctcaggagtcctctctctcctagagtgcaagttgtctaccagtagctgtacctgcctgaacatccataaatctgacaacgaatgtaaggaatctgagaattctatcgaagatatttcattaccagaacgcactgcaatgcctcgtagcattgtccgtgcacacactgtgaattccctaaacaaaaaagtccaaacacgtcacgtaacctgggctctgtgatttggaatctatttcttgcagtatatgctcatctttatggaaaaagctttgtgggtgtgtgctgtgtctccaaccatgttgtctctatttggaattatggttggggtttgtaaaaagatctggaagatggttttttaaaaaatcctggcctgctgaatgaatagtttctcctgcaacattgtggttaatataataaatattatcatataaataatacttcctgtattgttaagtctatacatcacaaacacttatgatatataacagttgtatagtgaaatatttcaaacatacataagaaatacaaaatgatgatcaaacacctatgtacacatcaacccaactttataaaattttaacattatttcctggagaagtttttccgttaaatttatagatataactgatgttcctctaaaaacttctccaatttcatttccatctcttgtttcctatcataaaacacttttaaggatgcgttgctgatgattctacattcatgttgacattttcactacaaatttatacatctataaaaattatatagaccagcttttcaaatgtttgaaagttggtcaatattacactgtagagatcatgttgtatttattttcataaatgctgaaataaatcttctaattattttagaattcctgaatctatctatgtaagtaaaacttggtccttcatgcctttttgtggttcatactttgtggggctaagtaatattccatcgtaagactgtaccacagttggtttatctatttcacctactgaaggatatcttggtgttccaagttggggcagttatgaataaaactgttataaacaccggtgttgcaaggttttggtgtgaacattaagctttcactttttgtggggtaaatacccaggaacatggattgccggggttatatggagaagaatgggtttagtttgtaaaaaacttccaaactgtcttccaaagtggctataccattttgcattctaccagcaatatatgagagttcctgccgtaccacatccttgtcagcatttgatgttgtcagtattgtgaattttgaccactttaatagatgtgaatgtgtcttattgtttaaatttgcaattctctagtgatttataatgtgaaaagcatcttttcatatggttacttggcatctatgaggtatctgttccattcttttgccctgtttgtaatcagccttttcatttttttggtgagtgtttaaatgttatttgtatattttggataacagtcctttatcagatatgtcttgcaaatactttctcccagcccgtggtttgtattttcattcttgacaacgtcttttgtagcacactggtttttaattttaataaagtccagcttattaattattaaaaaaaaaaaaaaaaaaaaaaaaaaaaaaaaaaaaaaaaaaaaaa

SEQ ID NO: 61 - DNA, 1112 bp, Homo sapiens
ggatagcaggggctcagtcatattttttggatgaatgaacaaaccctgaagatgctacatctgactctatatcttcattttatccttttcgtatttcctattacctctaatttctcttccctgcacccatttctatttatttcatcccagtttacctcctgctgccagattaattttcctaatgcacaggctctatcatatcatgagtttctcattgctacatatgactaatttgccaatatttttgcacatcagaatgtgtatcactttgaggctggttctgtgtttgttttagtttaggaaaagctgttcagattgtctgtaaatccgtatggggatctttgcataggattttaaagcagccacacatcttgtacaaaatgtataagattaattttctatgttaggaccatttgttttcaccaattccatagagctccaatgtgtaaaagaagacactgatctaactcttgtgttaaatatttagtaactcatttatctggaagaaagcaaaacaaaacaaaaatacaaggaataaaaatcactgggagtgcttttcattcactgaataatgagttttgcaaggagcacgtggatggtgacattatatcttttacatctttattttctgtttcttttttgactccttatcagtgaatttatcttattttatacttttactttctatttctttcttgactctttgttggtgaattggtagcaagagacttactgtctgatcagaactttgaatcttcctgcctctctttctttgaggttgacagggataaagataattaagatagcgcttgggtgtgatgacactggaagacaggctgggtcagggcctgtagtararacttcccccctctattgaatgttaatctgaaagtgaatctgaaagcagatggtcatgaactacccagggtctccattaagcccatgaagtttattttaaaactcttaaaatagattgagattcaaattgagattcatgtctattttttaaacattgtgtcttaacaaagtagatgttcagtcatacagttaggcaaatgttctaaggaaagatgtttmccatgctaagttaaaaaaaaaaaaaaaaaactcga

SEQ ID NO: 62 - DNA, 1674 bp, Homo sapiens
  SITE (734): n equals a,t,g, or c
gagaagtcctagtggctctctgctggtgctgaggcaggaagggccagaagctggtgagcttccctgaaccctggtgtgcctgggagtcagttccgaagaaggcagctgtcccagagatgtgacaggacctggctgttgtttctctctgaccctaacaggatttatgccctgcatccgcggtgtttttcactgttttattcttattattcttattctcttggcgtcacatgcgtttagtggatctggaaatcaaaggctgaaggaagcgctgacattgatcgtatctgttaatgtggatattgccagacacaggcccttcttggagcgtatacatgttaagaagggcaacacttagcccccaaaacagtaccccagtgttgcgtttacatgcagcagaaatagctggatggagagagattttgagccagggtctgccaatgccacttcagcctcarggctgagctgggcctggagaggaaagctgctgagccaccgttggagcaaatgactgaggacctctgctgcccctacttctgcccgagagctgagctgacctgattgtgctttggcatcaccacccactctggccccaacatctagccttgccaagttcaggtgctagtcatgaccatgtagagctcaccgtgtacccaaagactgtggcagcttcctggctgctggagctttccaggccccctatcttctgtttattcactcasccagcattaactgancatggactggatagggttgctgcccttgtggaatgcaccatctggararararaggaatgtggtatcgcagacgctacagttgctgccagtttagggatagaagtatcagggatgtcttcccagaagcagtgatgctccaacaacatctcaggcacttggctgtggccacatatcggtgcagaaggagaagcccatgcaaggcacccacagttgaggaagcagagggtggcaagccacgagcagtgccctcaggcacaggattccagaagcatgggcaagagcctggagggtccacatccccacattggttctggggacatttgcaactccttgtcttgagtgtcaacaacaggcagctctttgttcagggcagggcagggtatttggagatgactggtttgccctgtccaaagctgctgctcacactgttgagaggcttgacgcctggggtgggacatgggctgtgtgcatatcggcgaggctgtctggcctggcggctggacamagcctcctagtggagatgatatttggcctgtgaactgaatcaggtgaaggataggagaagaaamcascatatttggagacttagtggccagagcatgtcttggcagagaaactggaaggaggctggaatggctgcaccaggcagaaggaagactcctaaagtggggatgacgggtaacctgggtcacatgtgcgagatcttcttgtagcctgggggaggagcttacactgtcctgagagcagtggggagcctcaggagcctgcctccagagtatctaggcattagttacatgccatgttacagatgagattgcccaggcacagtggctcatgcctaaaatcccagcactttgggagaccacagtgggaagatcacttgaagccaggagttccagaccagcctgtctctacaaaaaaaaaaaaaaaaaactcgta

SEQ ID NO: 63 - DNA, 1045 bp, Homo sapiens
tcgacccacgcgtccgagatgcacgaactgattaattcatttgttctagggctctgaggagtcgtctacttaaccttttgggttgctggtcttacctatgttctcacgcctccattttctcacccactcactcagccttctccatttaccctcccaagtctttggcgaggtacactcatcctgcgtatcatcactgccatgtcctgataccccagctctgccatattgcccttcttttttgcggtatgatgaccacatagaggcccaacctcttaaacacatcaataccaatgatcacatttcaatctagacttctaagcaacggctgaaatctctccaggccaaaggagagtttgtatcaccttaccagaagcttctccggaacaattggccagaagcctagagttcagaaacccagacacatgcagtaagcaatttccagtttctctataatttagaagaggacaccatgatatgtaatgcggggtctggaggttggaatgcctccataaaacacctgccatattttttggtccaagccttagtgttataaatcaagaaggctgtaaataagacttcagcyttttgtgctggtgaagtttgtttcctttaacttatcctccaagagtaccgaggcaccgagatctaccatttgccacctcatccatttctatggcagaacaccgcctggggagaggaattcgattccccgaatcaggatgactgtgtggggcttctgcaaaggttgcatcacgagtcctatttctgagctatctgagatccccattaagaatttaaaagcaataaaataacggagatttttgactatcaacatgaatgctgtgtgggcttttacagttaatgattgcccttgagtgctgaataatctgtggcctgaaaaaagaaatgttcttatcttctaaatttggtaatcaagaacaagatagagtaatgaatgtaaaggaacactgttgcaagttgagtgtttccaaaaaaaaaaaaaaaaaaagggcggccgctctagtaggataccaagtctttacgt

SEQ ID NO: 64 - DNA, 1051 bp, Homo sapiens
ggtttttcccgggatacatctgtgttgagtcactttgcattcaacagtgcctcgccaccaaaatcatacataagaggaaaactaggactggaagaatatgctgtcttttacccaccaaatggtgttatcccttttcatggattttcaatgtatgttgcaccactttgttttctataccatgaaccttccaaattgtatcagatattccgtgagatgtatgtgcgttttttcttcagactccattccatctcttctcatccttctggtattgtgtcactctgtctgctgtttgaaactcttcttcaaacttatcttccccaactcttttatcatctacgagaaattggggctcaaccacttcgcatatcatttaagtggatggttcgagctttctctggatacttagctacagatcagctcttgcttttatgggatagaatcctaggatacaactctctggaaattcttgctgtgctggcagctgccgtgtttgctttccgagcagtgaacctgatggaggtgacatcactggctgcagctgaaaatctagctgcccacagtgaacagttctgcactgctcctctattccctgagctttacagagtccagatccctgtactgctgaactcaggcagaaagaagagtgcagtttattggactccaatctcattcaacagaacaaagaagttgaggttgcaaggaagaacctataatgatgggtcatggaatataacctagaaaagaagagaaataaaagagactgtgtttcaccatgttgcccaggctggtctcgaacttctgagctcaagcaatccaccctcctcagcctccagaagtgctgggattacaggcatgagacaccaagtccagccataaggttcttattctatatatacatgaaatgatatcacttgaaggtagactgtgataagttaaatacgtatattttttaaatcttcaaacaaccactaaaataaaagaacaaagagttacaactaaaaaaaaaaaaaaaaaaactacgtagggggggacggcgtacccaattacgccc

SEQ ID NO: 65 - DNA, 1182 bp, Homo sapiens
ggcacgagcccccagcacatggaagccctgttacagtccctcgtgatagtcttgcttgggttcaaatccttcttaagtgaagagctgggctctgaggttttgaacctactgacaaataaacagtatgagttgctttcaaagaaccttcgcaagaccagagagttgtttgttcatggcttacctggatcagggaagactatcttggctcttaggatcatggagaagatcaggaatgtgtttcactgtgaaccggctaacattctctacatctgtgaaaaccagcccctgaagaagttggtgagtttcagcaagaaaaacatctgccagccagtgacccggaaaaccttcatgaaaaacaactttgaacacatccagcacattatcattgatgacgctcagaatttccgtactgaagatggggactggtatgggaaagcaaagttcatcactcagacagcaagggatggcccaggagttctctggatctttctggactactttcagacctatcacttgagttgcagtgcctcccccctccctcagaccagtatccaagagaagagatcaacagagtggtccgcaatgcaggtccaatagctaattacctcaacaagtaatgcagaagcccgacaaaatcctccacctaacctcccccctgggtccctggtgatgctctatgaacctaaatgggctcaaggtgtcccaggcaacttagagattattgaagacttgaacttggaggagatactgatctatgtagcgaataaatgccgttttctcttgcggaatggttattctccgaaggatattgctgtgcttttcaccaaagcaagtgaagtggaaaaatataaagacaggcttctaacagcaatgaggaagagaaaactgtctcagctccatgaggagtctgatctgttactacagatcggtgatgcgtcggatgttctaaccgatcacattgtgttggacagtgtctgtcgattttcaggcctggaaagaaatatcgtgtttggaatcaatccaggagtagccccaccggctggggcctacaatcttctgctctgtttggcttctagggcaaaaagacatctgtatattctgaaggcttctgtgtgacaggaaacccaagcctaagaaacaattaagtggttctcatctctaaaaaaaaaaaaaaaaaaaaaa

SEQ ID NO: 66 - DNA, 675 bp, Homo sapiens
ggcacgagctgctcttcttcttcaacatgctcttctgggtgatttccatggtgatggtggctgtgggtgtctacgctcggctaatgaagcatgcagttctccctctgcctcaccgctgtgttcctgctgcagctggccgctgggatcctgggcttcgtcttctcagacaaggctcgagggaaagtgagtgagatcatcaacaatgccattgtgcactaccgagatgacttggatctgcagaacctcattgattttggccagaaaaaggtatgggtcagccagtggtctgggggactgtgggtaaaagtgaatgtcatcccaagagatgcctcaccctctatgcctgtggggctcttcattacctgccaggtaatggcttctgggaaggggtttggcaaaaaaagcacacgtagcagagtgctttaaatgtacttttaaagacacagaacagtatatatagtaatctactgtgttataaatggttacttacagggggtgaggaactgggcagattcttgaatattacctcttcaaaagtgacattttaggctggtccaaagggagtgagttatctcatttgattgttcacagtcagctacagatccaactccttgttctactctttccccccttctcagtgctgcacttgactagactaaaaaaaaaaaaaaaaaa

SEQ ID NO: 67 - DNA, 1105 bp, Homo sapiens
  SITE (797): n equals a,t,g, or c
gggggaaaaaaggacacgttgaattctgttgctttaaatgtatatttttttattgtgctaaaatgcacagaacataaaatttgccattagtaacactgagtacattcacagtgtcgtgcaaccatcagcactgtctagcgccagaactttttcatcaccccaaagggaaaccccgtatccatgaaggactcactccccattcgccctctccagcccttggcagccaccagaatgctttctgtctccataaattcatttttaataagtgcaattctgtgtgactttaaaataaataaacatgagcacgatgagttgcttattggaaggatatccatgcggggaggccggcgtgtggagtgcgtargcctccggacgggcaggagttgaaggggcgtggatgtgccgccctctcctccccttgctctttccttggggtcactgcctgagtatccctctttgcaaatggccccaaataatgtctcagcccccacgtctgcatcgcctcctagcttcaggaccctccaccaaaaaacattccaagcttcagactcactcctgggaaaattccaatggcctcactctcccttttgagccagccagatcccatggcctgtggcgggctgcctttgagtcctgagcacctgtgagytagggaagcaggacaggcacacccagggaaggggaagagtcgtcgtcagtcacagtaattgatatctttggaatcgtctaagagatacttagcgtgtgcctaaaacattcatttctttttttgtttgttttttgwgacgaagtctcgntctgtcgcccaggctggagtgcagtggcgtgatctcagctcaccgcaacctcctcctcccgggttcaagcgattctcctgcctcagcctcctgagtggctgggactgcaggcacacactaccacgcctggctagttttttgtatwtttagkggagacggggtttcactatgttggccaggctggtctcaaactcctgacctcgtgatctgcctacctcggcctcccaaagtggtgggattacaggcatgagccactgcacccagccaactagtcttaaaaaaaaaaaaaaaaaaaaaagggcggccgc

SEQ ID NO: 68 - DNA, 1279 bp, Homo sapiens
ggcacaggttaaacgttgtataaaatgtttcagtggggctggagacagtggcttcttggcatcagctgcacagggttggaccacagagcaggccccgcgaagtgttattccgagcagccccgcctcagctcgcagcctggactccaagtgttactgtgtccatgctgagtataattgatttgcttttcctactttctccaactttcggtttaatcacagaattgcttttcagtccagaagttcccaaagctctttcctgccctctgaaggctctgggaggtggttctcattcacatgagcccctgggratgtttgctccagttcctccaggctgtgaatcaagtactccattccccaagggcctgggggccagtaagatcctcaccctgggggctcaggctgaattcaggaggaggagtcactgaactgcattcaggaaaataaacacatcttgctcagctgagtgagcctttgaatttttctccctgggaagtcacatgtggggtggccaraggtcaccgtgtggtcagccagggatcttggctgggcaacgatgcctcccacagacccttatcaagaccacggcagtcttcactttggccatctcctctggtgtgtgagaagaagccattctctgggttgaactgaggggcttcagatgaacaattctgttccttcacccttggctaatggcccactggcatgcagacctgctagtgaattcaggagtttcccagctctaaggtgagtcctggcagctctactgggcctggccaactccatctccaggtcaggtttcccgtcacaaggcaactactagggagacccccgamccccgtccagctcccaccaggctgaattaragtccccstkgctgcccctcacccaytcccccatattcaccattccagttacttttgctgtgaagtaggccacccaaactagggagaataaaacaacaaccattttatgcagctcatggctacagtgtgggaagaatttaaacagggcatggaggagatgtctcctgtctgtctcaaaatgtctggagtctcagctaggaagactcmaagtctgggagtartttggcatctgggggttgggatcatctggagactcctttactcctgggtctttctggtccacaggctggaagggcktcagaattaggaccgccagccacagtcctgagcttggcgtctccatgtggcttggcatcctgcagcacacacagcagcctctgggctcgaggggggggcccggtacccaatcgcctgag

SEQ ID NO: 69 - DNA, 1638 bp, Homo sapiens
ggcacgagagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggtgggtcctgggttttctgcaaacatggaggaccactttctgataggacattttcctttcttctttctgttttctttcccttgtttttgcacaaagccattatgcagggaatattttttaatctgtagtattcaagatgaatcaaaatgatggctggtaatacggcaataaggtagcaaaggcaggtgctttgcagaaagaatgcttggaaacttgagtctccctagaagtgaaaagtgagcagaggcccctagaaaccctcctctgaatcctcctaattccttaaaatagatgcaaaatggtaagccgaggcatcgcgcaaaagctggtgcgatgcttcagggaaaatggaaaacccacgcaagaataatgattgattccggttccaaaaggtgtcacctacctgtttcagaaaagttagactttccatcgccttttccttccatcagttgagtggctgagagagaagtgcctcatccctgagccacacagggggcgtgggagcatcccagttatccctggaaagctagaaggggacagaggtgtccctgattaagcaggaaacagcacccttggcgtccccagcaggctccccactgtcagccacacacctgcccccatcacaccaagccgacctcagagttgttcatcttccttatgggacaaaaccggttgaccagaaaatgggcagagagagatgacctcggaagcatttccacagatggtgtcaggggtttcaagaagtcttagggcttccaggggtcccctggaagctttagaatatttatgggtttttttttcaaatatcaattatatggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatctcattgatgtgatgttacattccctttaatctgccaactgtggtcaaagttcataggtgtcgtacatttccattatttgctaaaatcatgcaatctgatgcttctcttttctcttgtacagtaagtagtttgaagtgggttttgtatataaatactgtattaaaaattaggcaattaccaaaaaaaaaaaaaaaaaaa

SEQ ID NO: 70 - DNA, 887 bp, Homo sapiens
ttttttttttttttttttattagaataaatgttgttgccaaatgaaaacatgacactgtatataactgctgaggcctattctcatgtttaaccttcctaagccagttttcttaagttggtggagatggaagactatagtaattttcctagcatgtctggcatctgctgctataaaagagacagcagtaagcatgaagactgtgtttcccatatttgtccaaatcactttgattttgcttttagagtcaagagtcttaaaaattggtgatttttccaattttttctgctaatagtatatttaaaaattagcatatgctttaggtacatgaaactttaaaaaagtaattataatgtacagttaaaaatttatagtaagtggatctcacaattcattttctaattagtaattgatagttttacccattaaaatgacaatgaaaatatttttactatgggttttcctcccatttgtttctaatcatacctttgataatattttataaaggcttataatcatagcagggaattaatttactacttaaattttatgtatatgtacagtacatttaaaaattaagtagtcagggcatggtggctcacgcttgtaatctcagcactttgagaggccaaggtgggtggatcatgaggtcaggagttcaagatcagcctagtcaacatggtgaaaccctgtcactactaaaaatacaaaaattacccgggcgtggtggtgcatggctgtaatcccagctcctcaggaggctgaggcaggagaatcacttgaacccaggaggcggaggttgcagtgagctgagatcacaccattgcactccagcctgggtgacatagtgagactctgtccccctctcaaaaaaaaaaaaaaaaaaaaa

SEQ ID NO: 71 - DNA, 864 bp, Homo sapiens
ggcacgagcggaccgggcccgcggggctgctgcggggcgatcgggccgggccgctgccgcgccatggactcccgtgtccagcctgagttccagcctcactgagtggccacccccaaagtgctgccagccgaggaagcccccagcactgaccatgtctattatggaccacagccccaccacgggcgtggtcacagtcatcgtcatcctcattgccatcgcggccctgggggcctttgatcctgggctgctggtgctacctgcggctgcagcgcatcagccagtcagaggacgaggagagcatcgtgggggatggggagaccaaggaacccttcctgctggtgcagtattcggccaarggaccgtgcgtggagagaaaggccaagctgatkamtcccaaacggsccggaartycacggstgavccaggatgcaaaggccycctggtccctgtttgcaagccggccaagarggggctgggaggggcaaaamccatacggatgcgctgctgtctgagaggaagggctgacacttgctggcatggcctctgcgggtttcgtccatcgcatgcactgatgcccggggacttggctgtcctgggcttcccctcggcctccaggtgaggctgcccattgcaggcactgggtaggcctgaccttgctggggctcatggccctgtagcgcttttgttacttgaatgtctagctgagcctgtttttgatggagctactactgtaatgcgtgaactaacaaacctgtgaactgtaaataggcccctggaagcacgtgcttaagcccttttgctgatttttaaaaatatcatctagcgcaaaaaaaaaaaaaaaaaaaaaaaaaaaaaaaaaaaaaa

SEQ ID NO: 72 - DNA, 1217 bp, Homo sapiens
tctggaacctctctcttaattcatatttcccgtaagtctgtccatctgttgtgtggaatctcagtttgtgatattggatagatgcaaataagcaaagctgttacttttcatagtttcaaatgaaaaactcaacatcactactgtataaattattttctagtctatctgtgtttatttttaaattccttttactattctatacattgcacattgctctgggggtaaaaatccartataaaccattagctcattttattgaccattcttgtattcagcaagtatcccaagtacagtggtccataccttgaattttttttcactttttaagtgagatataatttacataccataacaacttagtgggtttcagttatttcaaatacaaggttgtwcatatatcatcactgtctaattccagaacattttatttttattttttttcagcagtggggtcctgccatgttgcccaggctggtcttgaactcctgggctcaagtgatcctcctgcctcagtctcccaaagtcctgggattacaggtgtgcgccaccacacccaccctcaaaacatttttatttcctaaaaaagaaaccccacatccataggcagttccacattccgttcttcctatgatccagctcttggcagctactatagttkgtttyctgtttctgtggatttgtctattctggacatagcatgtaattggagtcatacaatatatggctttttgtgcctggcttctttcacttagcataatgtttttaagattcattcatgttgtagcattatcagcactttgtttcttttatggctaaataacactgcattgtgtgsacataccacattttgtttatccgttaatcagttgatggatatttgggttgtttccacttcggggctattatggatgatgcttctctgaatatttgtgtacaagtttttgtgtggacatttgtttttagttcacttgagtatgtacctaggatggaattactgggtcatgtggtaactgttttaatttcgtaggaactgccaaattgtttcctaaagtggctacggtattttacattcccatcagtactgtatgacagttccgtttatccacatccactccaacacttgttgttatctgttttgattatataatagctattttagtgggtatgaagtcgtatttcaaaaaaaaaaggaattcgatatcaagcttatcgatacc

SEQ ID NO: 73 - DNA, 1717 bp, Homo sapiens
  SITE (712): n equals a,t,g, or c
gctcctaggggcaggacttggcagttgctcaggagatgtttgagaggtgggcctttccggagaagtggccctttaaccctggactgtgggccatttaggagcatcacaccagttttagaatgtcagtagggacactgcgacacagacagtgacctgggagcaggatgcaggagccacatggcaagtttctgtcctggggcaggtggctctggtggtggtctcttgccgcaccagctctggttcaggctgttaacatgcctcccgcatacatccagatagagaactggtacatgatgctcctgatgggctgggagacaaaatgttgccatgtcaggagtctgtgggtgggaacataatgaaagggaccccgacgtggggacagtgtggccaggcagcatggagaagtgggtgccactggctcagcctgggcctgagctcactggggaccggagctactttgcatgttgctgagggatggggagggcagggctcttccttcacagaatgctctttctcttgcttggtaggatgttctttggcaaaaacaaggtgatgatggtggccttgggtcggagcccatctgatgaatacaaagacaacctgcaccaggtaagtctctggccctcacggggtggagctaaggcaaagccgccctctctcccactactcccatgtgacagcctcccccaccattggcaggccagaagctcanggararcanggcctggcttaaactctgggttytccyttmacytccgracccaaggaatarcasttcctgcytcccaytcyttcttgctatgacaaccaaaaastctttaaatgttgccaaatgtacccggtgagcaaaaacgtgcttagtagagaaccaytgttctaatgtgaccaagctgtcctcactcntgatttgtaggtcagcaaaaggttgaggggtgaggtggtctcctgttcaccaaccgcacaaaggaggaggtgaatgagtaagtactgctgaggaacggagggaaagaggcgggtgagaggggatttcagggagggaatgatgcaggaacttcgttccatatgtggcatcaagttggaactgctcctggaagcctgtcactgagctagtggcctgggaccacagccctctctgtcctctccagcatggtgctctgggctggctgctggccagcagccaaggtctgtgatgggggaaggcccagggccacgggacacttgggggagcctgactcatgggctggtggcgtggccaggagcatctcctcctaccacttctcttttcccttaggtggttcacgaaatacacagaaatggactacgcccgagctggtaacaaagcagctttcactgtgagcctggatccagggcccctggagcagttcccccactccatggagccacagctcaggcagctgggcctgcccaccgccctcaagagaggtatgggcagccctggagccaaaaggtcacagcctagagtccaagagcacgggcctgcaagctcatccctttctagctgagcaagttatttctacaagcttccattttctcatgagaaaatggggctaagagtgcatgcctcacagtgggggtgaaagcagacagtaacttacagccaaaatgcagcccttgaggcccatcaggggccttgcgcttgtttaaaaaaaaaaaaagggaattcgatatcaagcttatcgataccgtcgacctcgta

SEQ ID NO: 74 - DNA, 1276 bp, Homo sapiens
ggcacagtgaaaacttggtatttaaggaaaatgttgggagaagcccacagataacacacttgctctgtttaatgctaataaattgcattttttctttattgttattattgtcacatgcagatgggatgcatttatttataagttctggggataggatacttttttgcctgtactttttacattccagagtttgtgcttgacttcatcttgactaagacagatgagaatatttcctagaatttgtttttagtcttatttggatcctgtcacactgcagcattttattgtcctcagctgtctgtgatcctggaaacatacgtgtgactgaagctcccaaacacccaatctctgaagaactggaaactcccataaaagacagccacctgatccctacgcctcaagcccccagtattgcctttccactcgccaacccccctgtggctccgcaccctagagaaaagattataacgatagaggagactcatgaagaattaaaaaaacagtacatatttcagttatcatctctgaatcctcaagaacgtattgactattgtcatctgattgagaaactaggtaccagtattttacttaaatccaaaatgtcccatataataaccatatttggaagtcaaatgtagtcaatattaatatcatttgtaaagtctagaagtagaggaaaagaacatgggtaacttgtctaaggctatacattgagtcagcattggaactttgattttaaaaagctctaaagtcagtagatcatgggatttaaaattcaaatggaatctagcagttgaggtttaagaatcttctgtgctgtttgactattcattcactaatgaagctttaaagacttactatataaataaaaccacattttaatgaacttgaaaggttaataatatctagagaggagctccctcttgtttttttgtttgtttgtttccagttagaaacagtgtggaggccaggcacggtggctcgtgcctgtaatctcagcactttgggaggctgaggtgggcggatcacctggggtcaggagtttaagactaggctggccaacatggtgaagccctgtctctactaaaaatacaaaaaaatttgcgagacgtggaggcacatgcctgtgatcctagttactcgggaggctgaggycaggagaatcgcttgagcctgggaggcggaggttgcaatgagccaagatagcgccactgtactccaggctgggtgacaaagcaagactctgtctcaaaaaaaaaaaaaaaaaaaactcgta

SEQ ID NO: 75 - DNA, 1144 bp, Homo sapiens
gcacacatacgtatgcatataaggattatcatatataaatttatataacaatttttatgcatgagtgtgaataaatatatgcatatatatgtctgtatatgtaaacataatgcatatagtaatttacatatatctgtgtgtatatatgtgtgtggcacagtcacacacacacacacaaatatgtatacagatgcttcctggcttacaataggatttcatcctgataaattcatcgtaaatcaaaagtattgcaagttgaaaatgcatttcataccccagtaagttcatcatttgktcaaaagtattgtaagtcagaatacatttgacatctggataagtccattataaagtcaaaacattttaagtctaatcattgtaatttgggtaccgtctatgtagatacgtaaatcatacattaagggtgactaggtgccaggttgaatgttatgaaaatgaatttcaagtctcacaggcacattcacccattacaaatatgtaccacattcacctattacaaatatgtacacatgtatgtgttcatgttcatactacaatggcagagttgcataattgtgacagaaatcaaatggcttacaataactaaggcatttctacatagccttttaaagtaaaaagtttattcattgttggtctacataacgtggaggaatttgtagcggacaggctattacagtcagtgaattgaaaggaagggagaagttgggggagactagtagctttttgaaggtattattttagagatttatgaakttttggagaacaagggatgaggaaaaagtattgaagaatttgggagagcaggatatcaattagtttctgactttattgggaatgcagatcagagaaaggctgggatagaaaactgaaataataattatagccttcggtgaatatcagcaggactgatgggactatagggagggtagactaggtgatagagcccattgtggcagtttcggtaggacatcattggtgtatacgtatatgttatttgtgattttgtttatctttttttaataagcaaaaggaaaagtgtcctgatatgttttggctttgtgaccccatccgaatctcaccttgaattgtaacaaagttttaccatgttaaacaggctagtctcgta

SEQ ID NO: 76 - DNA, 918 bp, Homo sapiens
ctgcaggaatcggcagrgcagttttcatcatctatgatcttaagtttcctcatgctctttctcattgttaagacaataccacttattttagcttattgttacaatagtatttcatttttttctaacaacttggtattagtcaagatgggatataataacaaatgactcttgaaatcttcatgggtgccatgggaagtttcaagaacacaacttgaaagtgaattgcatgactttgttctgtgtttcattgaccactactcattcagtgagcctgaaagtaactgtgtatatcactgttagtatactatgcatgccagatacccaagactcaaacttttcctttcctttagataccacctacttagtcatcaattttggttcaacctactccactaaatagctttgacttccattcacttaccactttagattagtgctatagactcctattttacctccttcatatcaatccccttaaaactcccaatagcttccattgtttcaaccaaaagctcaaattcctttaatataaagtgttatatgaactggcaccctatatactctatatcctaatctctcatctttcatttatttctttaactcctgactcatgtaacaaaaatgtatttatctcagcacgtatgtactcccttgttatgctctcccaaatgtccctgtatttttcattgaatagcaattgccacattttattttatatgcttgtgttaccatttatatatatatttactacagactctgcttttagaaggcatacactggccaggcgcagtggctcacacctgtaatcccagcactttgggaggctgaggctggcggatcatgaggtcaggagatcgagaccatcctggctaacacggtgtaactccatctctactaaaaataaaaaaaaaactcgag

SEQ ID NO: 77 - DNA, 1065 bp, Homo sapiens
ggcacgagagagaggtggggttaatgtgatggaaagttggcattgcttagtttctggggctgccttctagatagcttctaaaccccaaagcttctctccacagtgccatgcgtctactgagtactctgcttagcttttaccccttcagcaactgctttttgctcagtttctgtgattctcacccacccgtgtggcttaggaattcacaagtgtttccagaggaagttgtgtgaaaggtggctagaggaccagaatctttttccctcgtgtcctggctgtcctgtggccctggactccacatttttatcactagcccactgggcccccaccctgttgggtgtcagtgctccccaaactgacaagtgccctgaggaggaaacgctcggatgcaggcgcagctcagttcatttcctttctatgtgggatcctcaccttctatgtctttcttggtctccaaagcctcacacagctgttccttgctgttggcatcacttagtctcgcagtccctggtgtttccatcaggaaagtcaggcaggcgttagcttcactgccaggagggaatctcctgtggcctccatggcgctgtgttttcttccagtcttttttctatgcacagacacatcatcccctttgtttccttttgtgatgctgtttctaaaaggatctttatttctagaagaaactttaggaggcaaaaacagcgcagcccccttaacagagtggctctggtggcaactttcccttgttaatttgtcctgtagcccctactttcccaagcgcttgctgtttgtggggctacaggacacagcagctgaaaggggctgtgggcatcgccagcatgtaccctcttatcccatttgctgacaaggatcctgaaggcccaagcatagaaagaagttgctatggctgccatgtgtcagcagcatagccatggccaactcagggccctgactcctacctgacccccttctgaatgacactcaaggtaagggtccccttcccactcacaggtgaggtgaaacatttcaccttgaaaagcctcttgcccccagcctcccctcgta

SEQ ID NO: 78 - DNA, 1126 bp, Homo sapiens
  SITE (1124): n equals a,t,g, or c
ggcaggactgcatgactggcaagctttggctgttgctgcctaggttgggccatgctgcagccgcccccaccactgcactctctggatcagaactggaaggtacctcgatatctttgcttatagcattggacagatgaagggctggttacctgtcaggaagtgaagagcgtggttaagagtcctctatgctaggttgtcacagtcaccagctactagactcttggctacaacatttctcaccaagagcagtgtccttggggaaaaactaaacagggatgagaaaggggttaggaataaaactytctcctagagaccaggtcagaatacataatgggtttaacttcgcaataaagtgacaaggtgcacttgaataagccaccctgatacacggaaagcactgggcacagaagtaactttcccattgaatcaggagttgatcccataaaccttactattagccaagtttacatttatgaacattttacacacactactcagttatatattaaagacaaaaattgataaaatacttatactttggtaagccatagagccaattctcttttcaacctagttgttcatttcaccagtgggcaaaaatcattatttttaaaggtttccaatttaagagcacagaccacccagctattatagagctctatagtttagccctcgaaggtgagtcccagatgcagttcagggatggtctgaacctttgaacagggcaaatccaagcactctaactcctggttccctgctctatccctcatccatgccaccttttttaagcatccatatggattagtgaatctcatttccaaatctatgcttggttagcataatctctcatccagaactcccctttgaatttgagatcctatatctaagggcccacctccattcctcccttcaggtctcacagacaccttaaagtcaatgtcttatctgtttccctttcacgctcccaaacgagtcattgttcatcttccactccttatttcagtaactggaactccatccctccagaagcacaaaacagaacctgggagtcatccttgattcttgctatttcctcacctcccatatccaacctatccccaagtcctgatgactttacctgctgnaa

SEQ ID NO: 79 - DNA, 984 bp, Homo sapiens
  SITE (232): n equals a,t,g, or c
tcgacccacgcgtccgcgcgtccgtcggtcggtgcgtccgggccgccggcttcgmcctcgccatggcaccctggctgccgctgctgtckctgctggggctgctcctgggcrccgctcccgccccgccccgccgagcagccgacgctcaggcccgggaggcggcgtacccggagctgctggggcccgcccgcttcgcgctggagatgtacaaccgcggccgggcagccgggangcgggcgacgctgggggccgtgcgcggtcgcgtccgccgggcgggcgaggggtcgctgtactccctgagggcgaccctggaggagcctccctgcaacgannccacggtgtgccagctccctgtgtcyaagagaccytgctctgcagctttgaagtcttggacgagctaggaaaacacatgctactgaggcgggactgcggcccagtggataccaaggtcacagatgacaaaaacgagacattgagttcagtccttccactgttgaacaaggaacccctgccccaggacttttctgtgaaaatggcctcaatcttcaaggagttcgttaccacctataatcggacgtatgagtcgaaggaggaaacccagtggcgcatgtctgtcttttccaacaacatgatgcgagcacagaagatccaggcactggaccgtggcacagctcagtatggggtcaccaagttcagtgaccttacagaggaggarttccataccatctacctgaatcccctcctaagagagtaccatggcaagaacatgcgcctagacaagtctgctggcgactctgccccatccgartgggactggargaraaagggggscgtcaccaaagtcaagaaccaggcatgtktggctcctgctgggctttctcartcactggtaacgtggagggccagtggttcctgaaacaggggcctgntctscctctccgancargarctcttggactgtgacaaggtggacaaggctgcctg

SEQ ID NO: 80 - DNA, 1247 bp, Homo sapiens
gcgagttcatctaactatgtgctccacgagggatctctccttgagacttgacagttgatttgcagacagaagtatgccctcatggaactctcacctccaggggttccccttagacctcacatccattaggagggggtgtggaaaggatgcccacgtggccacttttacaactgctgtcctgctcatttccttccctactttgtgaaacgttcactttctgctccaaagatgaagtgtcacgttggaaggcgggatgctttgtgccccttccagcaagctaacttccaaataaattctctawttttatatcagaccttgttcttgttaattagactttacatgaagtgagcaactaagcttttctgttacaagacttcatgcccacagatacattcaagtcccaggtggaaggatgatctggattaggcaaggtgctgaccatgggaacaggagacagtcaagggaatctgaggaagcacatgtttgtgtccaatatagtcccctccttgtgccactcagatgtgctcatgccctccaactgtggctggttttataagcagatgccttgtgtagttgcacggggtcttgtgcttgaggggctttttgcttggattaatgttctgcacttgcatttttatttttcagacagcggatactcctccactgaagagggaatagttctgcagtaattccctaggggtttgctttctcctctcctacgggcttgatggagacaggcacagagtcctgtgatgcccacgtkgcatgcctctagcagctttgaattctgctggatcatctggcacaatgggccgagcaggttgggccagaccctgtatctgctgtagagatattatatgttctggattccacttgaaaccaggagtctttgcattcattctgtaacccatctggttttggcagaggctaggtaggttaactgaacagggatttaaaaaggactttattaacacatcagagcaatatcctctactgtggtatttttggactccaaaatccctagaggctcaataaacgccgtgcttctcttttagtgataggaagtatataggaagccatttaccttaaacaggatgtttctgctcggcatggtggttcacgcctgtaataccagcactttgggagaccaacgcaggcagatcactggaggccaggagtttgagactagcctgagcaacatggtgaaaccccatctctacttaaaaaaaaaaaaaaaaaactcgtag

SEQ ID NO: 81 - DNA, 958 bp, Homo sapiens
gaattcggcacgagtgagattgcatccagacagagttttaaaagtttcccggttgagtttaatgtacagttgaagttgagacatgaatctctgcatgtaggggaaattttgtgtctggttagtcaagaaactatggaaaccaattcttgatattttgaaccattcacgaagatagtttgagtcatgagcatgctgttgtctagagtgggcggggatgactcattggagtggatgcgctgctctgtacttgatttttttgagtctgaaattagctttccaggctggggcagggaggggagcacaggtgggatcagtactgcccccaagcggtggagctgtggtggtggatcaaatactgctgccgcctgtctgcacaaacatatttctctcttccagcccttcagaagtgtattggaatatgtcgwtaacaataatgatggtagtgaagatgatgatgatgtgggtaattctggctaccttattgggtccaagctccccacaattcgttgcacaaagcactctacatacattctctttagtcctgatcaaaccacctttcagagtaggatttagtgtcctattttaaagatgaaggagctcgggctcagagagagatcgtttagacacacacacaactttggaatgaaacatttacagccgggcgcggtggcgcgtgcctgtagtcccagctacttgggaggctgaggctggaggatcgcttgagtccaggagttctgggctgtagtgcgctatgccgatcgggtgtccgcactaagtttggcatcaatatggtgacctcccgggagtggaggaccaccaggttgcctaaggaggggtgaaccggtccaggtyggaatgaaacatttacaaaaattgacatttccttatgcatagatatttcactaggtccttaaaacccacgtgaatctgtgattaaaaaaaaaaaaaaaaaactcga

SEQ ID NO: 82 - DNA, 1392 bp, Homo sapiens
attcggcagagcagaaaaccagactgcacttgctttataaaacagagctttatttttccttcataataagcagagttgcagtgttgctggtattgattcactggcgtggtggtatcaggacagatgtctctatgattaatttttggcctgtcactcatgtttgcatatggctgttgtggctccaagcattggaagcaagaggacagggaagcaacattgactgtaccaggaactccaaaacagtcttcacatcttaatggttggacaatgccaaatggtcactcttttctggaagttgactggggacaagatagtggtaaggattagatttggccagaaagtttctgccacagtgagctttcctgtctaaatccttattttaactgttgtcacttaatattcacactttggaaggacatctactgttggttacaattatgaaaccaacttgaatactttttagttgaacatttcagtagtcttaattatgtttaaataggtttcacaatttactgtttttagtttagtttccggctccccccaacccccaacttttgytagagagttactctcttaacttttgctagaaagtagcaaagttctctactctacatgttcagggctggctgtagaatttcgttttttaaggaaacaggaagacagaactaattatgcaagtcttcatttagctttttaaaaaaacagctttattgagttagaattgacatgcagtaaatggtacatatttaaagcgtacaatttgttaagttttgacataagtatacattgtgaaaacatcagtcaccacaatcaggatacttattttaaaaaacaactttatttaggattagtatactgataatgtgtccattgtaagtgtacattttcagttttgacaaatgtatagatttttgtaactaccaccaccagtcaagatgaaaacgtttctagcactccagaaagttcccttgtgtcccttcttggtcagttattcccaccatgctctcaggcaaccacagttctgcttctatcactatataagtgacagaatttttctacagaatttcacatagatggaatcatacaatatgtactgttctgtctggcttcttgaggtaagccaaatgtcttttaagagtcatgcatgtttttgcatttattagtagtttattctttttttgttggtgagtagcattcattgtatggatatattccagtctgttttattcattcactttttggacatttgggttgttatcaattttgggctcttttgaattaatccctccctccttccctccttcccyccctccctccttccctccctccctccctctctccctccctccttccttccctccctccctccctcccttttttttttcggcacga

SEQ ID NO: 83 - DNA, 1155 bp, Homo sapiens
ccgggtcgacccacgcgtccggtgagtgcactctaggatgttcacatgatgacaaaatcacctaacaatgtagacgcttcagaacatataccctttgttaatcgatgcatcactgtatatatgtgtgtatatacacacatatatgtacatatatttaatacatttgtgtatgtgtgtgtatatatatatatatatacttctcattatttatactctagacccagagcctcctagctggtctccaaaattggactctcatctctctttgagacagccttcaaatgatcgtttttaaagtgctaattaactcctcttctcaaaatgcttcaatggcccactaatctctaccgaatcaaggaattcagccatactgtcccaagatatctttccttggccagttggagcctcatttcagctgctctgggtttatcccctgtctcttctttcccacttccaagcctgtgctcagcccacctcctcttctggggatgccccacaccccactctgccatatctgccaaacctttcatctccccgtgaagctcttgacaccaaatacagtttactttagaaaatgtattttttccactttctcaactaaacttttccttgtgtgatctgcttttccgctgccaaggcacatcgtttttaattctctacagcactgctcatatcttgcccagtattatagcttctacatattggtcttgcttcttatttttgagcacaaaaactaagccactccactttctcttaccagtgaatccagcttaaaaaaactgtgagcaacctatcagtattttgttgacatgaactctatagaaaccttagtccctggatcttcgactctgcctcccctgacatttatctgctcccacaaagcacgcaggtgtgggaagagaagtggctgttttttgaggtcacatttcagccctgattcatcctaatgtcttcaccctttttatcctttggscactgtgtycctagagatgtgaattcaattccgcaccattctctcctttacaatgatgccaatattctcaggcttttaagactaaattttaaattacgagaaaatttgatcttcaaacttaagttggacctagaaagaacaatctcatgaactcaaaaaaaaaaaaaaaaaaaaaaaaaaaa

SEQ ID NO: 84 - DNA, 1373 bp, Homo sapiens
  SITE (877): n equals a,t,g, or c
gatgtgctgctgcctctgctgtacctcctggtccggaagcacatcaacagagcgggtatcgggaacacgtttcagggaggtgccaactgcatcatgttcgtcctctgcacccgcgccgtccgaactcggctcttctctctctgttgctgctgctgctcttctcagcctcccaccaagagcccggctggcactcccaaggctcccgcgccttccaagccaggagaatctcaggaatcccaagggaccccaggggaacttccaagcacctggagcttttgtcctttctagttctgtggcataggtgctgccttcctgggggtaggcamttctgtgagtccctgstgcagggcagaagtgcatgtctgctgcaccagaagcccccgctattgatgctctcggctccattgggagagcagctgccaactcagcttcttctacctcctagatcctcaggcagcaagttccamcggtaccagcgtcctggcccacgggtgggcgttcatctgcataagggtagcagtgagattcgggaagccggtggcccacagctgtggccacagtgcccccacccagtggaccttgatgtcctgaggaccacacagcactgtttacaatctgagggacccacatctgtgcacctatcctctgtgtgattaaataagctgtcaggggccctcagagatgtgtccccatccacacatcatggagcagattcccggctggtcagggtgttaagattctctggagaacccggctttccttggagactttgctcaacagaggaaccakttgccattcaccaggttgaaggtyccctgargttgcatggtgaccaccaggggcaagcacagcccggcgcactgctcagggacagcantctccccgggsctcytggcntctccytccatgggctgctaacccttgccgtagcttgggtttgagtgtcagcacaggccagaatccacactcctgtccccactgtcatcacttcagacacaggttccctccttccaattccaaagctggaacaagggcaggaagaggcgtctcagggaacccccgaagaccccactgtctctccagctcctttcttagaatcctcctcttcttggggagaccaaaggggatgagtagggaggggttctggcttatttacttcttagtttagttttttgtaagtaaagtggccggagagatgagtaatccaaatacgattcctcaggccgggcacagtggctcacacctgtaaccctagcactttgggaggccaaggcaggcagatcacctgaggtcaggagtttgagaccagcctggccaacatggtgaaaccctgtttctactaaatatacaaaaaaaaaaaaaaaaaactcgta

SEQ ID NO: 85 - DNA, 1258 bp, Homo sapiens
tgcactgtgggtgtccacccacaccttgtgttcctcatggcctaccccagcctttcttctccactgggtcccactgttccctggagacagagggctagcatgctgtcatttatctgaaggttgtggctgacccattctcctgggatttcccaggccacctctcctttccctttccctcacttaacccagacttgctcagctgaggctattgtccctgatgttggctttacttgtaggaggtttagtggctgctctggcctgccatggaattttggctgcaattttggcagtgtgtggagaactggtatcaggaaagggaaccaggagtagtgatgaagatgatggtggggatggggacagaggacataggggactgtccctcttgaactctgcctttgggcacatgggagatggggacaggaaagatgataacagtggaaccctgtgaagaataggagaatgggaattctaaaataccagttcccaaacaaaccatctctccattggaaaaggagctgttgcaaaccgtgttcatttaagcttctgatgaaaagcaatgtatgtttataagcttgcaagtaacagacaagatgttccaaggccgatgaggaaaatatcgcttccctgtctctgcctctaccttgctgtgcctctctggctttagcagcattggatgttgaattggtttggttcatttctctgaagtcggactgtcaggaggagccagaggttggctttctgggatgtgaaggggacagtgtcagcctaagcctctggagatgcttaggatgctgaattttaaccccttctttcactgcatggcccttcaaacagggattggcaggctccgcgagttacagagtgggtgagtcttggagagaaccaacgactaaggtagcttagctctctgctttgttcttgggagccaagagaatgcaagatcctggtgggtgagcagtggtatgaagaggggacactggtgggtgaacagtggcatggaagtcactgatgcagcctctggccctcagtctccctatcggcaaagtggggggctcttcctatctctaaagagtttcatccctgacaggtggatgggtgaaagatccagagagtgggggtgaggggtgggccctgaagactttttgtctgtagctcttgcatattgagtgtataaacccggcttctggaccaacccaagatgaataaactggggcagaaaattaaaaaaaaaaaaaaaaaaaaaaaaaaaactcgtagg

SEQ ID NO: 86 - DNA, 1318 bp, Homo sapiens
cccacgcgtccgaacagctcttaattggtgctttcagtttttaatggacttcctgatgttaagagagtgagagagcagtggtttacttaatcaccggggcccttttcatagtttcgtcttgtgtgctgtgctttttacccagctctaggagggagatgtttgtgggtaccagggttttgttagtccctctccccttttttagtataagtggcatgctggcaatagacaaataccttcataaaaagctgcttctaaatgaaataattactacctccacttgggcattatgataattatcaatagaatattgaatcagcttggtttttcaggaaaaaaaaatccaaacaaaccttccttaaacaaggaaggttttatttcaggtctccaattagagtaaacaagcatttggggcttttcctcttgttttctgtgttaggattgtgaatactagtctgctatagaaggttgctagttactttctgtaatgtaggcagtttctggggattggagacacagatgggagtaaagggtctggattcttctctttcccacctgcctgtttggctttccctcagcctcccaccctgtgcccatgtgccccttctctgatctctgtgctcagatggcagggaccataggagttcatgtgtccagaggtaacactacccgagtctgtgatactcagaagcttccagttccttattaaaacgggaaattgaacagtaaattgttactgcctaccagatttcacagcaggagactgtcattacacctttcatttgactcagagagagtgtagccatcagatgcccagattttgccattctttagtcacttggaaaatgcacccattgtcagcattttgagttattaggattcctcaaatgaaaaaaaggcaaacaatatattttgatggattgaaatccacactgttgtgctgaaaataggggaaggaaaaaaggaagccaagattttggcagaaaactgaacactcttaagaataactcctcagccgggcgcggtggctcgcgcctgtaatcccagcactttgggaggccgaagtgggcggatcacgaggtcaggagatcgagaccatcctggctaatgtggtgaaaccccgtctctactaaaaatacaaaaaattagccgggcgtggtggcgggcgcctgtagtcccagctactcgggaggctgaggcaggagaatggcgtgaacccgggaggcggagcttgcagtgagctgagatagcgccactgcactccagcctgggtgacagagcgagactccgtctcaaaaaaaaaaaaaaaaaaaaaaaaaaa

SEQ ID NO: 87 - DNA, 978 bp, Homo sapiens
  SITE (977): n equals a,t,g, or c
ggcacgaggcgcgccaaggcgtcagtcgaggagtcaaggcagcaatgaatcgtgtcttgtgtgccccggcggccggggccgtccgggcgctgaggctcataggctgggcttcccgaagccttcatccgttgcccggttcccgggatcgggcccaccctgccgccgaggaagaggacgaccctgaccgccccattgagttttcctccagcaaagccaaccctcaccgctggtcggtgggccataccatgggaaagggacatcagcggccctggtggaaggtgctgcccctcagctgcttcctcgtggcgctgatcatctggtgctamctgagggaggagagcgaggcggaccagtggttgagacaggtgtggggagaggtgccagagcccagtgatcgttctgaggagcctgagactccagctgcctacagagcgagaacttgacggggtgcccgctggggctggcaggaagggagccgacagccgcccttcggatttgatgtcacgtttgcccgtractgtcctggctatgcgtgcgtcctcagcactgaaggacttggctggtggatggggcacttggctatgctgattcgcgtgaaggcggagcagaatctcagcagatcggaaactgctcctcgcctggctcttgatgtccaaggattccatcggcaagacttctcagatccttggggaaggtttcagttgcactgtatgctgttggatttgccaagtctttgtataacataatcatgtttccaaagcacttctggtgacacttgtcatccagtgttagtttgcaggtaatttgctttctgagatagaatatctggcagaagtgtgaaactgtattgcatgctgcggcctgtgcaaggaacacttccacatgtgagttttacacaacaacaaatgaaaataaattttaattttataatatgggaaaaaaaaaaaaaaagggcggccggtaacccattgcgcccna

SEQ ID NO: 88 - DNA, 1863 bp, Homo sapiens
  SITE (82): n equals a,t,g, or c
tgggggttgggatgcagaggcatgaccccctttcttcccctgccccttcctgacatgagatgctctatgatagaggtagctngggcgtggagtgaaacagaatgtgggtttncggggaggaagtgttggggtcacatgctgcttctcctgctttcttacataggtgcttcagcgaagagtcctgcgtctccatccctgaagtggagggctacgtggtcgtccttcagcctgacgccccccagatcctgctgagtggcactgctcattttgcccgcccagctgtggactttgagggaaccaacggcgtccctttgttccctgatcttcaaatcacctgctccatttctcaccaggtggaggccaaaaaggatgagagttggcagggcacagtgacagacacacgcatgtcggatgagattgtgcacaacctggatggctgtgaaatttctctggtgggggatgacctggatcccgagcgggaaagcctgctcctggacacaacctctctgcagcagcgggggctggagctcaccaacacatctgcctacctcactattgctggggtggagagcatcactgtgtatgaagagatcctgaggcaggctcgttatcggctgcgacacggagctgccctctacaccaggaagttccggctttcctgctcggaaatgaatggccgttactccagcaatgaattcatcgtggaggtcaatgtcctgcacagcatgaaccgggttgcccaccccagccacgtgctcagctyccagcagttcctgcaccgtggtcaccagcccccgcctgagatggctggacacagcctagccagctcccacagaaactccagtacgtaagcctggtggggctgggcagggaggggcaggtggcaggtgagtgtgttgggacaggtatcctccccctccacctctgggagaggacaaggtaggtggagcaatgggttctgttccctgtggtacctgccttagttgacagctatggaccaatccctctctccctttggatcattcactttcccttgtgaagaacatgagccttgtgtcattgtagggatgcttatgcctgcartcataaagtcagtgtgggtttaggcgtgtttgttatacctgatgatgcctccttccccagccccaagcaatatcaaattagccctggacctaggatcytctgtggtgaaactatcaagtatttactaagcacytactatgtactagcactgtcctaagatgatctaaatgatttataggatagatcagtccctatccccaaggagagaattggtactattacatgtaaaattatgtgctacgtatgcagtcacaagaatatttctaaaaaatatatgcaaatatgcaaagcgttacattcagatggagtgctgcccttcaaagctgcccttcaaagcaacctggccgtggtcccaaaggttcccggtcatggtcctttacgaatggtgcaattttattttcaacttttgagttgcgtaattgtagaaaaaccagaaaatatcaaatatttcttttaaagaagaaaaatcaggccaggcacagtggctcatgcctgtaatcccaacacactaggctgagtcaggaggctcacttgagtccaggagtttgagaccagcctgggcaacacagtgagactctgtctttacaaaaaataaaaaaaattagctgggcatggtggcaggtgcctgtagtcccagctacttgggaggctgaggtaggaggatctcttgggcctgtgaggccaaggctgcagtgagccatgatcacaccactgcactccagtctgggagacagagcaaggctctgtctcaaaggaaaaaaaaaaaaaaaaaaactcgag

SEQ ID NO: 89 - DNA, 2086 bp, Homo sapiens
cgaggaatggagccggtagctgctgcggcgagtsccgcggctcctccgtagacccgcggagcaccttcgtgttgagtaacctggcggaggtggtggagcgtgtgctcaccttcctgcccgccaaggcgttgctgcgggtggcctgaatgttcgcatcttaccacatacagttctttacatggctgattcagaaactttcattagtctggaagagtgtcgtggccataagagagcaaggaaaagaactagtatggaaacagcacttgcccttgagaagctattccccaaacaatgccaagtccttgggattgtgaccccaggaattgtagtgaytccaatgggatcaggtagcaatcgacctcaggaaatagaaattggagaatctggttttgctttattattccctcaaattgaaggaataaaaatacaaccctttcattttattaaggatccaaagaatttaacattagaaagacatcaactcactgaagtaggtcttttagataaccctgaacttcgtgtggtccttgtctttggttataattgctgtaaggtgggagccagtaattatctgcagcaagtagtcagcactttcagtgatatgaatatcatcttggctggaggccaggtggacaacctgtcatcactgacttctgaaaagaaccctctggatattgatgcctcgggtgtggttggactgtcatttagtggacaccgaatccagagtgccactgtgctcctcaacgaggacgtcagtgatgagaagactgctgaggctgcgatgcagcgcctcaaagcggccaacattccagagcataacaccattggcttcatgtttgcatgcgttggcaggggctttcagtattacagagccaaggggaatgttgaggctgatgcatttagaaagttttttcctagtgttcccttattcggcttctttggaaatggagaaattggatgtgatcggatagtcactgggaactttatattgaggaaatgtaatgaggtaaaagatgatgatctgtttcatagctatacaacaataatggcactcatacatctggggtcatctaaataataattaaagtggctttcataatatgtaacttttgggttctgcctttttcagaaaatggaaacttgggccatgtgtatttcaaacaaaaataactttagatatatcttttttgtagcyttgattgatgctctaagatcacatgagggtagtatttaatatattagatgaaggacaactttggacataacactgactaggagttgagagcttttgcatcaggcagaagcaaactgattatagttgtgttgcaccagatcatgtagctgctgtgtaacatgaccttaaatagtcttcctgcataggaagagcaaaagggtattcatcaataggatatagatttaagacattccctgactaccccttgcgttgttaggtgatgtcttttagcagaatcatgaagaccttttttctcccttaataaaggagaaaaatatactgatggctggagaaatttttctctgcctttcagttttatgaattttttcagaagtaacaatattattattgactttttacttatttgataaaaattaaagaactatttttgttttggtcagataaattgacaagactaatcagtattttattataagtaaaagatttttcttctttccttaaaaatatttttttttcacctaggtctaaatagctaactaactggtagaccagagtattacatcatcttattttggttttataccaataaaacatagcgtggaactcattcaggtaatgttttgcatttcattgcttttggatgaacaaaggaagtaaactaatcctttataaatgaaaacccagaatagttggtatgtcagctagtcattcctgtcatattcccagtagaatgattttcaagtttgaatttctgtwcaaatatctaaataagagatgtgcagagagcaccaattttccttcaatatccattctttacttttcacataatgatagaacctttgatttttcaagtgggtatgcctcctagaataaagactacatttcccgagt

SEQ ID NO: 90 - DNA, 891 bp, Homo sapiens
gaaattatgcatgagctgtgattgcaagtaatttttaaacgtctgcatgtggacaaagtaaatacataaaaatgaaaatggctgtgtaagagagtaggatgaacaataataattgttttccccactgttgtgaattatgatgttatcttttaacttcacatggatggtttgggtttctctagttcttaaaagtcaaagagccaagttagccctccattccctccatcttcatcaagaggtcaggctcagaatgtcacggagagagtcgcctggcaggcctcttcgttgtggggtgagagggaacatgggtgccagaaccccagtgccaaccgctgactatccttctccctacaggacattgccgagaatggctgcgcccccaccccagaagagcagctgccamagactgcaccgtccccactggtggaggccaaggaccccaagctccgagaagaccggcggccaatcacagtccactttggacaggtgcgcccacctcgtccacatgttgttaagagaccaaagagcaacatcgcagtggaaggccggaggacgtctgtgccgagccctgagcaaaacaccattgcaacaccagctacactccacatcctacagaaaagcattacccattttgcggccaagttcccgacgagaggctggacctcttcatcacattgacttacgccgttgcttttccagactgggcagaggggctgacttcgcagtgtgtgccaaagarccggtgtctgataatcccattttcctgcttatcacctgaactgtgtcagtatcacttttagttttgttggttggttggtttgttgtttgtttaatatgccctgttttctacttctgttggaaaatatttggggttgaaataaaccagtggga

SEQ ID NO: 91 - DNA, 1974 bp, Homo sapiens
  SITE (654): n equals a,t,g, or c
aaaattgctaattaatattagtatttggttgctttatcttaaaacaatacctacctctgttaataggaaaattagaaattatatctattgttcctaaagatacaatttgtttctctaactccaagggaagtgaatgaatatgtttcagactatacttgtctgtgtattatttgtttttgtaaggtggttttttcttttgcttcaaattgagagcatacaaacaaaattccactgtataagcagtcaattttggtaataataacacatgcctagagcactcagattaaattattacacatactaataaaattattcacagtagtactccttggtaatgttggtattagggaacagggatagaacccatcttcaaagtcaaccctattacttcaagcctttgcaggtcctgctgtttccaaggatacaatccaatgcttaatttacaggaatccgaaatcatttgctgtgttaaaaacttgagattagcccttgctgcccccttccaggttggcctttggaagcgaaaggtctttaagcttaagtaaactggagaatatttctgagtaagcaggctccaggtggagggtacactccaagataccttcacttgccaaaacaacactccctgcaaggttatcagcgaaaaggcagtanaaagagccctcctgctgccagaaggtcttgttttgcaaggcttctcagcctgcctgcagccattgggtgaggagaggaaaggacacctcacctggcagagcagcacaaaggtcaggcatgtttccctcctgacctggaagctgggtcctgctccttcacatgaactgtccagggcttggtcatatacccaggtcccctcagtcctgcccagartgactccactggtctccccagaaaacaccaacagcagararaaatgaartgtgtctgttttctggtttcatattgcarctcctcttgctcttcytaggacagaaataacatctgcmatgccararamctgtgaagtaataaacaacctttcccccagctcttgacaagcattaccttgcttkgaggtgcaatcagttytgacaagtttatggytttgtgtcyttcaacccaatggacattaccytctgggggagtttaatgacccytytcagcttcacactcacttgaaggaaagagctagaagtcagtaatgatgtgttatgacttgcatggcttcaggtagttcagaacctgcaacctgtagttcagattacaggagaagaactcagacgataaattggcttccaaagaaggccaaatagaaaaaaaaaaggttcttcagaccaatagctgagtgacctgaaatttaaatgcaaaaaatgcattaaaacccacaaaaccacttcctttaaaattcttgataccaaacatgttacagcattaagtgaaccttaatctaagcaaaattcagagaagagcattcacacctttgatagaagagccagccagccagggtgctggggctcagctcttgagaaaaatgttgatgtaaaagccttggtggcctggttcagcccctgagcctccctggtaagcacatggcaggctcagaacatccataactgaatgctctgagccctgtaaaaatgcagttacaatgggaaatcaactcttaagtatgctatgggatgctgtgataaaatttgagtttctctaccttctgtttgcactttggttcatttagaaaagagtccataattgtgtgcttcaagagtgcaattccatggctgtaacagaatagagtcctctttcttctggcctttgcctctttggggatattccaactctgtggaaggtgatcatttgcgattatgatcagttatttatatttgactgtaaatgaaaacttcagagtcagtttcaaaaaacaagagatggacataaggacatgtgcttatgagtaggggacaaataactagagacaaaaaaaaaaaaaaaaaactcgta

SEQ ID NO: 92 - DNA, 1423 bp, Homo sapiens
ggcacgagatactttcttatggaacttgtatggtttcgttttttacatttaaaccttcttccccgtggtgtgtgttgtggaatctgtgtttgtgtgaggaggggcatggtgctctcagaacccacctcctgtggccagagagccctgtcctgtgagggtggttgtcacagtggcagggttcaattcagaagaccttgagggcaggctgatgtttcctgaatgggcccctggttgttgcttgtccctgactctccatttccccatctgagtggatttggacctaatagggcactggagctggttcgaatcctgactggactacttggcaactttatgtctgggagcaagttacttaacctccccaagcctgtgtctgtgaaatgcgggtaaatgaatgtagatgtttggcagcagctactccttgttgagctctcacagtgaactctcctgcctctgccctccttccccgcctcccctggtgcctagcgtcaggtctagccacttcctcctgggcccctctcccttttctgtggctggctgcctgcccgcctggcgctggacctttcatgtaacgggaatcagcatgtatattctggtctggtctgtttctacacttaattttgtttccagtagtatttccctgtaccggcagagttcacaaacacatttgaagaggctttttctcaggattcttaaccttcccaaaggaagtcccatggatgggtttctagaagtctataaaatgctctgaaattgtatttttcctgtggaaagcataactttcatctgcttgttcgtgctcaaaaaagatcatgaatgaatgattgcatgattttatgccattgtgcttatactaaaggatatgtagcccatctcttgagctgktaaactgttttgactactttaaatcgtgcagctgtgagcatctctgtaaatttagtgtacacatgtatcccctggagtggcattgcctcggcagtgagcacttatggttttataactctcttcacagactcaaatgactccagaaagctacacttcctgttgtgagtatatgatatccatttccctacatagccactaacatcaggtttttacaattttatttatttcttgctactttaagaaatttttgtggtgaaatacatataatagaagttgactatctgaatcatttttaagtatacattcagtagtgttaagtatgtcgccattgttgtacaaccaatctccagaactttttcatcttgcaaaacaaactctgtacccattaaataacattaaacattccattccctccagcctcagcaaccccattctactttctgtttctgtgagtttgactattccaagcacttcatatcagttaaatcatgaagtatttgtctgtctgtgactggcttatttctctgagcacagtgtc

SEQ ID NO: 93 - DNA, 1365 bp, Homo sapiens
ggcagagctaacccgagtgaagccacttccgggcttcccgggcgccttccgcagtcctcttccgggtgatggcggccgggtgccccggatgtagccctggcgcaagatctcttcttttttccacctcgccttccgcggattcccagcttgagaaacacctctttgccccgtcatgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctgagcctgctgggggcccagtttggtgggcccttctttcctggactttgtggaggaggcaccaagtgtctcaggcagcgaggaaattggaggccatttttcagtcaatttccctttcccaataaaagcctttagttgtgtaaaaaaaaaaaaaaaaaaaagggcggccgc

SEQ ID NO: 94 - DNA, 756 bp, Homo sapiens
agcacgagggtgggaatgtgaagagggcagcccaggccctgtgtttcgggggtgtgcctctccccgcactcccgtttctgggaatgctgttccttctaccttcagggcctgcccgccctgcggtgtagccgcactcctcccgggtgtcatttcttcagagtctttccttcatgccctttttcctcctcacgttccccctcgtgctttacccacatctgtcccgtggttcggatccagttctcccgtgcgttatgggtatccacgtgtttggtcttagccatcactccaggaaagtggctcctccctgaggacagggctctgtctctgatgctgctggcctccctccagtgctgccctcctccttttggggcttggtggatgcaagtgctgacacacaaaggcaggcaggcaggcctcgggccaggggtgtcctccaggcccctctgatggcagggaccagcgagacctggggaaactgcacagtctgtgcctgtctgtgctctcaggagactgagggaattagtgggtgggagagattttatgttggattaaaaactgcacctccaggccgggcatggtgtctcatgcctgtaatcccagcactttgggaggccaaggtgggaggatcacttgagccctggagtttgaggcttccaggaagctatgattacaccactgctctccagcctgggcaaacagagtggcaccctgtctctaaaaacaaacaaacaaacaaacaaaaaaaaaaaaaaaaaa

SEQ ID NO: 95 - DNA, 938 bp, Homo sapiens
  SITE (479): n equals a,t,g, or c
ggcacgagtggaggttcagggtcagggtccaggggcgaaggtggacgctggagaagggcagatggacagggtcaggttcagatcttggctcttgtatccttgctgtgtggctctgggccaagaacttggcctctctgcgcctcagtggctcattacagaaaatgggatgccagcacttgccttagtgggttgttttgagccaactgcaggctcagggagtagctggcatgatgtgttcctaccctgaagggcagaaaaaggggaaggaggccaccagatcccacaggtgggtccccaggtccctccccgggatgggcagcakcctggctgctccccacagcaacccctggctggccccattggcgctgctggaaatcccccamccagttctttgtgaatggaaaaggaaactgattgcgctagaagaggtctccgaatgccggccgggggtggggggcggggggggsttcctctcccantgcaggaggggccacctcagcttcctttctggggccccataccccctttttcctatctcccctctgasttgaggaggaggctgtggcccccgcccactgtttggcctctggaaccactgggtgacagtgtacatcggggcgattaagggccacctggcctctggacctccctcgctgtggctgctgcctgggaccccccaccccatgcctgactcgaaccccaacctcagctctacacacaggggcaccactgtaggaggagagagaaacctctctctagggaaccacctggaagggggccctgccctgtcttcatctgggtaccccatgtaatctaggaaactgtctttaacttgccgagggcctccatgtctgagttcctaatgttttttattttgctttttcaattaataaagctcatggcagaaactatttaaaaaaaaaaaaaaaaaactcgta

SEQ ID NO: 96 - DNA, 928 bp, Homo sapiens
ctgcaggaattcggcacgaggcagggtgggtgcatcaagggggttgtgcggtggctgacaagaggttgtaatgtggggaagggcaggagcgggctggtctttacccactgccatccccgtcctcttattaaatagagattcctagtattgctgttataaaaaatgtcctgctaaacatagttctctcaaaaatatttttctgagattctctgacaattaaatctggatcccagccccaatattcacctgcaattacattatggatgaaattaaatgtgcactttctatggtgtacatttatttttcaaacctcaggaagccatattgagctcttaatctcaggtcaagtgtcttcttatatcccctcccttgatttttgtactcataaggttgtatccagagagaagtttgaggaataattcattacctgggaattaaagtaaaccttgagcttgggagtcctaacatataaccatctctaaaattctgcaactgtagatttttaatcatctaattttagggacttcaaaatatttttctgactttacctacattcgaattaagttaaaatagcactgataatggatagtaggatccaaacagaaacattttaaatgaatctagttaagtattgagccgggcacagtggctcacacctgtaatcccagcactttgggaggccgaggcgggcagatcacctgaggtcgggagttcaagactagcctgaagaaaccccatctctactaaaaataaaaaattagctgggcgtggtggcacatgcctgtaatcacagctactcgggaggctgaggcaggagaatcacttgaacccgggaggcagaggtttcagtgagccgagatcacaccattgcagtccaccctgggtaacaagagtgaaatccgtctcaaaaaaaaaaaaaaaactcgta

SEQ ID NO: 97 - DNA, 1715 bp, Homo sapiens
  SITE (17): n equals a,t,g, or c
cacggcctatggtttangttaaaacccttaattntttttncggtaatcacttttaacactgctaatggaaattatgttttcatgtgtttcacatgaaaaagtatatatatacaaggttaatatagtgggtaaaatactttacatagtcacacatttacaaatttttcaagaggttagccactaagactttaataattttacaagggaaaaagcctttttttttytttgatatacagttttttcttcttagttctgcattagaaatggcatctgttttaggtctcaaaatataactcggctgtttcactgtatatgtacattgttttctgtaggaataggrtaatgatatataggatcatgatattccttctatccatgtgccaaatgggtgtaatgtttatttactgatgctttatgttaccaaaacatacagtaaaaaagtagaaatttatgaaatacytttgataaaaagtttattttgtgcttaccaaaaggaatgctttcacaatagtgtatcagttcttttgttttgttaaagttggaatttattctgttgccagcatttaagtagtcatggcaagtcctgtttttaagaccttttggagactggagctttctgttccattaagtcttttgtttatactacaaattgtcacctcacttagttcagatgaaatctgttactctacaaggaaggtgttcatcattaggaggcagctttactaagctggtgctttgcatggtagcaagtgctgccctttatcagcaccctgggtcatagtgtaggctagrgttaaggcactggcagacttagggatgctggacagacctgtagttcgttttaagtcatgttcacaggaatttctacaataataaacccatcatctccataggtcagatcgaagtgcattccaatgctaaatagaagttatgagtgggtttaacaattttagatgattcagcttttgttccattactgttgaactatatgaactattccattactgcagagatttaagtatctgttttaataagctctttttgttatttaaaggctgcccatgggtttctgcctagtggtaaagctgattgttaccctcctttgaaatcccttctagttctgagatgctttgagggtaactggattcgattttgggatatcttttctcacattcagacttacacttaatggtgttagaaatcaacaaaactcctttttaaaaagaaaagatattaagcctgcctacttctacaatgcattctgttacctatttgaacagtatgtttgtaactatggcaatgaagtcagtagataggaaaccagttattccttctacctttaaaaattttgagaacttgccaaccagggattaaagctattatcttgaacagagtccctaaagctagtctagtttttgccacatctgcaatgattattgtttaatttcaaaagaatcctcaggctctacaatctaggggtggtaaatgtgtttccactatacttgggaaaaggtcagtaggatgtgcatcctagggaagataaaatcgtatatggtaaaggcatttgagttaattttgcattatatctaggaaccatattatttaaaatttgaatcctattaatgctgagagatcctaagagctagtatgttgtaaaacctgccacctgaataaaatgaaaaaaaaaaaaaaaaaaaaaaaaaaaaaaactcgta

SEQ ID NO: 98 - DNA, 678 bp, Homo sapiens
acattttctatgtagtaaacatgtatgtgtatgtgtgtgggtgtgtgtctaattactttgttggacagattcctgtggtttggaactgctggggccaagtttatacaaaaatctacatttttgagtaaactccccatgactctagtaagtttccacagtatttgagtattctttatttcctaaagctgtacaaacatcagataatcaatatttgtaaggatcgataatataatttataatgaaattgctttatcagctattaatgttaactacatcatcttcatatagccttataactcatttgtgctattccattttcctctgttccttttattttcacttcccttgtaatgttagtctctttgtactgatttctgaagagttcatttatgattaaacatgttaacattttgtctagaattgcaaatatgttttttctcattcattttactatggtgttttattttttggttatacagaagttgtatattgaaatataatcttgttctgttttatgactttggagttttgtggttttttaaaacatgtttacattggtatcatttatttgtattgccttctatttcagcagttagtttgtcatttctttttccattaatcatcattctggtgttaatgaatgcattaaatatttaaagtaaaaaaaaaaaaaaaaaa

SEQ ID NO: 99 - DNA, 1541 bp, Homo sapiens
ggcacgagtcgcatcaaagtcagaaccagcgccctccgtccttgctctcagcccagtgccaggcttccttgttaggagtattcttctcatgaaattctactgatttcttacattgtagcccagatatgatttgctgagggatatctgagagaaagcctatgtgtcctttccataaagcgtaccttgactgcttttttcaaatctctctcttgctactgatctttctaacttaccttgatattgggaagtgtggtctttggagccatgaatggagaattagggaattagggaaacatgagaggtggtggaattaattcattgtatcttgattatggtggcgactatgcaggaatatgtgatgtcaaaatcattgacttgttcacctaatgtgtgtccatcttaatgcttgtaaattataccttaacaacgttaactaaagaagtaattaaactcccacattggacaaggagggatctagagcactgaatctgcacagattctctgagctcagtctaggaagagaagtggggctgaactatacagaagatgttgaaattagaaacgtactttccaggaataaattaaaaattgagtttttgtccacctacaataatcaggcactgtgccagktcctaggactgtaaagaaaactatctctgatataagcttataatctaacagagaagacaaaaagtagaaagcttaaattcagtgagtaagtgctatgatagaggcaggagtgggttgcaaaggagaagcatcaaatacagatgaatggcacaaaaggctttctgtaggacattcatatagagcctgagaaatcaggagaactcctccaggatcactagctgtattagttcattctcatgctgctaataaagacaaacccaagactggggtwacttataaaggaaagargtttagttgactcacagttcarcagggtggggaggcctcaggcagcttacaatcatggaggaaggggaagcaaacgcgtcctccttcacatgaaggcaggaaagagaagtgcccaagcaaaaggggggaaaagtcccttataaaaccatcagatctcatgagaacagcatgaaggtaaccacctccatgattaaattacctcccaccaggtccctcccactacacctggagattataggaactacaattcaagatagatttgggtggggacacagtcaaaccatatcactagccaaagcagcaggtgtgtgtgtgtgtgtgtgtgtgtgtgtgtgtgtgtgtgtgtgtgtgtgtgtattcagtatcttgaactagaccactgctcttgaagatctcttgaccatgtagaagccaggataacacatttagctggaagctaattgtgaagctgggaaattatgcatgtatagtgatgttgggcacaaactcatgttatgttcaggtaacaagcagagcattttgggttcctttctttaggagttgtctaattttacttcatttgttatttctgattatattggttaccagctcgtgccgaattcgatatcaagcttatcgatacc

SEQ ID NO: 100 - DNA, 881 bp, Homo sapiens
ggcagagcccagctgtttcttacacagtgacatcccaggtgccctggggtttgggactcctggcaggagagaagagaatgaaccaacccattctgagaagtcaggcattgttatggccctggagatgggtggtgaaggcaaaaccatgtgtgtgtgtgtctatggatgcctggattcctgaccgcagtcagcattgcccatcaattccaggtcaaaagaaggaaagggctgggtcccatggtcaccaagcccttgccgytctgctcttcttgtgaaagtgaccttcagagtttcatcagctgatagcagacagccagattgccctctagtaaagcctagtttaagatcagtttcaggaattttctgggtgtttttatctttgcagtgaagccataaaagccctcatagcagctgggcargctgcaaagttgatgcttccgagtggctcattagagtgtaatggcattcctgagagtagatgttgctgcaagcagcaggcaggaagctcatgcggcagcagcctgatggatactccgcgtcaagggggttctggtggatgcgtggcaggcaggcagcggctacactccatgggcggtgctgggttgcgaaaggggctgacagtgctgctctccggcagaggggtgggggcaggtgcatgcacattgcagatgaaaaggtgaggggtctcagtgggtgtgatgggagttagacaacagcactgtttaggaggtggtttaaagctttttctctaatttaaattgggtagtaaattctgaccctaacatgagtattgtatctgatgggattcagtttggggaaaaattcagtcttgaagtctagcaaattccagtttcggtctcaacctttgggtcatctcgtag

SEQ ID NO: 101 - DNA, 947 bp, Homo sapiens
ggcacgagcagattgtttctggcttagtagcatcatctaatggcgagcaggggcactgcagcccctggacgcaccttcctggcgatgatggtcacgtcctttttcttttgtatgagatggggatcatgggctgagcagatgccccaaaggtgtctgccatgctgtatgcaggaatgctgaggtttcatgcaaaaacgtatgttgggatcacttgacggggtgcatggaaagtatcctagcagagttttgctagtagagaatgtgccctccgctagtactctagagtcacagcccagatcctagccttcgggtggaggtgccgccctatgaacggccatcagactgtcctattgttcagagactccttttcgtcccttactacttcatctcaggcctgtgaggatctctaccaaagcagagcaagcccataaataaatagctttgtgctcaccaatgcatcagtggcgctgaaataactgtgcagtggacaccactctccctccctctgggcctttcagctgctctgtgtgtccacgctgttgtgtgccctgaaagaactttgagatttgctgaaaggtctccagctgcaatttcagaccatgtgggtaactcaaatgtctaacatttaaatatggctgaaaggggtcgactgctgggctggatggtggcagaagtagcaggctttcccttggatctctttacagggcagagatgactccttacaaatggcaccagtaatccctctgcctcctgatacccatctaacagaaaagtgcaccctgctcataagaaaaccccaggccacatttgtcttatgtgaccctggtctctggctacagctgacttgacacaaactaagacaatcagatttttttcttctggaacttgctgtgagacacagagacacagtggactggatgacaatgctgggatgtgaaggatgtgtc

SEQ ID NO: 102 - DNA, 1369 bp, Homo sapiens
cccacgcgtccgcccacgcgtccggctggcaagatggcgggaggggtgcgcccgctgmggggcctccgcgccttgtgtcgcgtgctgctcttcctctcgcagttctgcattctgtcgggcggtgaaagtactgaaatcccaccttatgtgatgaagtgtccgagcaatggtttgtgtagcaggcttcctgcagactgtatagactgcacaacaaatttctcctgtacctatgggaagcctgtcacttttgactgtgcagtgaaaccatctgttacctgtgttgatcaagacttcaaatcccaaaagaacttcatcattaacatgacttgcagattttgctggcagcttcctgaaacagattacgagtgtaccaactccaccagctgcatgacggtgtcctgtcctcggcagcgctaccctgccaactgcacggtgcgggaccacgtccactgcttgggtaaccgtacttttcccaaaatgctatattgcaattggactggaggctataagtggtctacggctctggctctaagcatcaccctcggtgggtttggagcagaccgtttctacctgggccagtggsgggaaggcctcggcaagctcttcagcttcggtggcctgggaatatggacgctgatagacgtcctgctcattggagttggctatgttggaccagcagatggctctttgtacatttagctgtggtgtgtgcttcagaaaggagcagggcttagaaaaagcccttttgtccgtagagttgatgtggtgtgagtgatatatttctatgtttttaatgtacagcatctgtactttgtttgccttgataaaggtaagataaatgaaacgctgaactatgctaatctggaatttgtttttatttgcctgaaatatatttttttctgtgaaaaaattaaaacgtacttaagccaggagaatgaattatacagtgattgaaaatccatttaattcctatgacttttgttttgtattgcccaagtcaaactacatcacttgtatctccagcccaaatgtagtctgccttgaaaagtctttcagctgtgactgcaggaagtgggagtgtttttattgttagctaattgctgtgactgcaggaagtgggagtgtttctgttgttggctaattgaagttattaggctcagcttcagtcatgtgtaagttttgcagtgtaatacatatgtagtctggtctgtatatatgaaaatttgaattaaactgcagaatgtttatgtctagttatggtttaaattttcttagtagtatataaaaggtaagagtactgaaaaattaataaaattgcaagttaaraaaaaaaaaaaaaaaaaaaaaaaaaaaaaaaaggagggggggc

SEQ ID NO: 103 - DNA, 1231 bp, Homo sapiens
ggaggaaggaagcaattctaaaaacaaattttcaaagctatttttacattaacattattttttctaaatgtgcatccattatagtagagttatcttttccttccttaagctcagaattaagtcaatttcctggtatgcaatgtggctttattttttttgttatttaaattgtttagccaaagtgagatcagcaactacttatttatatgtttgagggcactgtgagctgaaataacaaattttgtataacttatctaactaaaaatatttataatacttctcaataatgattctaatgaagatgtattgagtaatttattaccatcttttatgttttaagagttctgatagtacaagttaaaaaaaaatagcctacttctactcttttactgaaatcaaatagatggcatgtgatgattaacataaaaaagaacacattcaacataatatattgcgatagtggcagtctaatagaaaaatagcaactatttatttacttttatttattcgttttatttataatagatatgtacaaaatgtaaaattatttctaagaagatttttctttaaacaccaagcattacagtagatgttgtctgaatgtgtgtaattgagaataataatattgtataataatattaatatgattgtggctagaaggaatttatttgatcacataagtacagcaaattaagaataagatctcagcatatcctggctactacataaatgtagcaaagttattttgctaccttgaaaaaatacaagtggacaaaaaattgcctaatttcatggaaaaactataattttgccagttaatacatgagattctcaaattcacgaatttaaaatgtttaaataattatacttttgctgcctgaaaatatattggatcaaaatttcaagtaaatatcatcaggggatttggatggggaagagcaagaggggaagaaggaggagaggagatttagtctagcactatttccagctgatccccacccagcattgccaagctacaggacaccaattcaataggatgaagtcagtcaaatcctcttttaagtaatggttgaataaaataaaatgaggggaagaaaatcaagtactctaaatgattaaagcgtatgaataagtgaaaaatgtttgcaaaattgagaaggaaggagaatcgggaagtggttatttatggtgtctcaatatgtaagggtaaaaaaaaaaaaaaaaaactcga

SEQ ID NO: 104 - DNA, 1242 bp, Homo sapiens
  SITE (288): n equals a,t,g, or c
ttcgtatccactaggatggctctaatcaataacaaagtattgtcaaggatgtagaaaaattggagccctcctgccttggtgggagtgtaatatggtgccagatacaacctccatcctgaagctcatctgtatgcttcctgtttgtgtttttaaacttttactatatctttatgtcctcataagaatatgtactatcatttggtgttttaaagtgtacataaatgctgtcatcctgaacaaatcctctcgctaactgcatctttaactctatactatattttcaagatntgtccatgttgatccacgtagctccctagttccctttaactgctataagatattctgttgcgtcaatatatgacaatttatgcatgctttgttgacaggtaattggatttttagtgttttgcctttacaaaaatcactgcatcttttgcacatgtctacttgtgcatatgaactgaggtaaaattgctgggccttactgtaaatatgttgttttaattcactttgcgctgctgtaacagaataccatagactgggtgcttataaagaaaagaaatttatttctcatagttctggagaatgggaattccaagatccattcacaggttcggttgtctggggaaractttcttcacacatcctcacttggcagaacggaagggcctgggttgatgctgtgtgaamcctcttttataagggccttagtctcattcccaaggaggagctctcataacctaatcacctcttaaaggccccccactcaatactatgacattgaatttcaacatctgaattttagaggggacactgcaaacctgtcatatgtctttatctttactatcactaaattgtccaaagtgattgcaacagtgatttatatactcaacccacagagtataagaatttctcctttctagctgggcacggtggctcacgccagtagtcccagcactctgggaggccgagatgggcggatcacttgaggccaggagttcaagaccagcctggccaacacagtgaaaccccatctctgctaaaaatagaaaaagttagctagctatggtggcgcacacctgtaatcctagctatttggggggctgaggcaagagaattgcttggacctgggaggctgaggtagcagtgaactgagatcgtaccattgcactccagcctgggtgacagagcgagactctgtctcagaaaaaaaaaaaaaaaaaaaaaaaaaaaaaactcgta

SEQ ID NO: 105 - DNA, 1151 bp, Homo sapiens
gcagggacagccccacgcatgggaggtggtggccagccatcaccccagggtgaatttctctaagagcagtcctaggccagggggtgagtgtgggaaagtcagggccgtttccatcagttaacgtttcagagcgggttcccgctggaagtgctggaaatacccagatgccatctgtagcattttatgaactgtggcggatccactctctgtgtcttgagcttctgctctgtggtctgctcagtggaagcatcctgtcagtccactgtacagtggggtggagccgcagccagggtgggtgtgccgtttgattggtcacggaatgaacagggcaaaggtcactaagtagatatgatactgcaagtaggattgtggtcataggtatttttatgaatttgtgttatgtgaagttgaggatttgaatgttgtgattattatatccagataaagttctagcctggcacagtgcaggcacgtgcctgtggtcccagctgcttgaagtgggaggagagcttcagctcaggatttccaggctatagggagctgtggtcccaccattgcaccccagcctgggtgacagagtgagaccccatctcaaaaagaaaagaaaagaggctaggcgcagtggctcaggcctgtaattcaagcactttgggaggctgaggcaggcggatcacttgaagtctggagttcgggaccggcctggccaacatggtgaagcccccccatctctattaaaaatacaaaaatcatacttacccggcaggggagataccatgatcgcgaaggtggttttcccagggcaaggcttagccattgcactccggatgtgctgagcctgcgttttccccaaatgtgggaaacttgatgcgtaatttgtggtagtggagggactgtgttcacactgtcccccccgcccaaaaaaaataaaaatacaaaaatcagtcgggcgtggtggctcacgcctgtcatcccagcactttgggaagccgaggtgggtggatcacctgaggtcaggagtttgagaccagcctggccaacatggtgaaaattgggaggccgaggcgggcggatcccaaggtcaggagatcgatacatcctggctaacacagtgaaaccccgtctctactaaaaaaaaaaaaaaaaaactcgta

SEQ ID NO: 106 - DNA, 1628 bp, Homo sapiens
gaggaaatatcctctccatgaggcatacaccaagtaaatgactttgtaactttacttcatcctcttcatttacacagggcatacatgaagtaaccaatggaatcctctagggggtatttaaactcccaaaaattctgtaacggggcccttgagcccctatgcttgggtccattcccaaactgtggagtgtactttcattttcaataaatttctgcttttgttgcttcattctttccttgctttgtttgagcgttttgtccaattatttgttcaagacgccaagaacctggsacaccctccaccggtaacactcttacaattatgcagttgtgcagtgcatagcccgtgccactatatgtggcagtgttgcccacttagcaatgaggagcgcatattttcctgcattatcaccaaaacaatgttatcatcttttatttattattatttttttgagtcagggccttgccctgtcacccaggctggagtgcagtgacgcagtctcagctcactgcaacctctgcctgtcaggctgaggtgggatgatcacttgagtccaggagtttgagaccagcctgggcaacatggcaaaaccccatctctacagaaaataattagctggatgtggtgatgcatgcctgtagtcccagctaytcaggagactgagatgggaagatcacttgagcccaggarttagargctgcagtgagctatgatcatgccaccgcaatccagcttgggcaacagaatgaaaytctgtcaaaaaaaaaaaagaagagagaaaagaaaaaggaaagtaaaaattgctaaacacytccaaagtctatgtaggaaatatgtaaatggtggtccatttccaagatgaagtgcctcaagtaggcctgggtctgccagctacagaaggacagaatatcytaggcccttgcttcaatagctggagcctgcttgttggggtgcccttagttgctctcatccgaacctaagagtttagtctagaatgaaaatttactagcctgcaaaatagctcactttatctattcttttatcagcttgcctgactacctaggtcataggtcaaatacttaaaaagcccttgagcagactataattgcaatgcattatgggctgcaacaaaatgcaccgagacaaccctaaagaaaacacccaaaacccctacctggccaggcgcggtggctcatgcstgtaatcccagcactttcggaggccgaagcgggtggatcacttgtcaggagtttgagaccagcctggccaacgctggtctccataatactcagcctatgaggaaccaggagagggacctgcacactagaggataaattgcttgttgtaactgtgcggggtatgcctgcccaccagacacctgatcttgcaagactgtattaaaagtctcacttccgctattctccgtgtctctgagtccattctttgggtttggacgggtgagtttgtttctcacagtctagactctagatgtgaagaactttgatcatatcaccaaaggagatggtggtatgcaattttataagtaaaaatacactagtgtcagttttttttacaagggaaacctgatttgcatctttttaattaaaaaaaaaaaaaaaaactcgtag

SEQ ID NO: 107 - DNA, 1465 bp, Homo sapiens
ggcacgagcgagccaagtttgcaccactgcactccagcctgggcgacagagcaagactcagtctcgaaaaaaaaaaagttggaagcagaagtaaaaaacatggtaaagaatgagaactaaataaatataataattgagaggtctgcattagatgtggcagggagaacaagcaaaaagagatttcagagaagatcactggaattggcagaggccttgaagggcagagtctagcatacagaagatgtaaagccacattctgtgaaggtaagtagatgtgtttacctcttttgcactgtactggtgcattatggggtaaatrtgtattacttttcctgtattgcttagcacagagttttgcctatagcaggcaccagactgtgggcttggtagtacatgactattggtgattacagatcaaaaaggacttgaaatgatcagtttaaggtcttgatgggtattgaagactcaaaggatgatggcaccctgggagtgatccacagaaggacagattatttgaagatgttaataactaaagacaacatggatgttaaatgatgaaaaaaagttggatggaaaataaaccattggatctgcytctggagtccaagaagaatattattcttcctacctcccccttactctggctcttcctattgtagccacatgggtcagtaatgccattgaaaaacaaaattttagactaagtggggtcgcagaaattttggtctatcttaaattgatgacatcttattaaagaatytattgtataaagtgtgcttattctggcatttttttaatgaagaaaaagtgtaattcagtgcacatttatgaatttcaaagatcaataaaaatgggcaaagtatatgaacgcataatccatagaagaagatatctgacaaatgcagttcaataaatatttttttaaataaaaattagcctgtggtaagaattgaaatggagaaagaaatagaaggtagccaggttacctacagctttgtatatgacactatagagctaggactttattccatagatgagggaagtctgctaaagtgtctacattgaacccattattttgctagcattgtagttgatgtacctaaaacagacttgagccggtagagtaagtaggcagacttgtccaagtgagaaaaagatgaaactgtggtgaggataaagagagaaaaggagcagatttaagaaatattaaaacttgaaagtactaagacttgatgatgaactagatgtgttagataagagatagcatggagtctagtaaaagttctgtttttctcactcgtgtgactgcctcaataacacaaagcttgataggaaataaacatgagatagcacatggatctattacaagtttttgaaattgagcttgaaaagctacttcaaaaaataaattctaggccaggtgtgagyccatgcgcttgattaaaaaaaaaaaaaaaaactcgta

SEQ ID NO: 108 - DNA, 1265 bp, Homo sapiens
  SITE (766): n equals a,t,g, or c
ggggcagatggaaatgtctcggattttgataatgaagaagaggaacagtcagtccctcccaaagtggatgagaatgacacccgtccagatgtggagccaccactgccattgcagatccaaatagccatggacgtgatggaacgctgcatccacttgttgtcagataaaaatctgcaaatccgcctgaaggtcttggatgtgctggatctgtgtgtggttgttcttcagtcccacaaaaaccagctgcttcccttggctcatcaggcctggccctcgctcgttcaccgactcacacgggacgcccccctggcagtgcttagagccttcaagttttacgtaccctgggaagcaagtgtggtgactttcttcgcagccggttctgcaaagatgtcctgccaaagctggctggctccctagtcacccaggcccccatcagtgccagggctggaccagtttactcgcacacgctggccttcaagttgcagctggctgtcttacagggcctgggccccctctgtgagagactggacctaggtgagggtgacctgaataaagtggctgatgcctgcttgatttacctcagtgtcaaacagcccgtgaaattacaagaggctgccaggagcgtcttcctccacttgatgaaggtggacccagactccacctggttcctcctgaacgagctttactgccccgtgcagttcacacctccccaccccagcctccaccctgtgcagctgcasggggccagcgggcagcagaacccctnacacgaccaacgtgctccagctgctcaaggagctgcagtgaccctgctcccccaccacagaggccaccgatccctcccctactgccagccagaagctgggctgaccccaccccggccataggcggtggcagcggcagcagagaaggtgaattagttagccaatcgatttataaattgatcgatcacacaactgcttagaaatggattgaaggaaagtagctgactattatttatatttcataccttgtgttttcaagtgacattgtctggtggctctaagggtttaaccccttagcctaccatctctatagccccagctccctcacaggccacacacacacacacacaagaggtcagttcccctccatctgcatacacctccctgtcttcaaataatgagatggaactaatttgttttacctaacctgatctttgggaaacaaacggaaataaagacacttcttggatgaaaagtaaaaaaaaaaaaaaaaaactcgag

SEQ ID NO: 109 - DNA, 1006 bp, Homo sapiens
ccacgcgtccggcaataatgacccattgtggtttttaacttatctcatgaaaagacttaggtttgttctcagggtatttcagatgactgcctttataactggggcacatacgattactaactatagtgataggcgtttatacatttcccctttgagccatttctttatgaacagtggttcttctgctcaaagtgttctgtctcattcttatgtttctcaaatcttctttaaaaatgtaagcaaatatttttaaagaatttttatgttttccaaaattaggattttagactttagggattttgatctttggggatttcaacattcgggattatggtgttcagtgtgtattttggggggattatgatcagcatcccatacagtggaatatcatttggcaataaaaaggaattaaatattgattcatgctacaacatggtgaacctaaaaaacattatgttcagtgaaagaagccaaacctaaaaggcctacgtactgtgtggttaaatggtaaaaatggtgaatttatcacaatcaaaaccaataaacctctacaggaaaaaataaggacaaagaaaggctgttccctattaatggagactaaagagacatgacaactaaatgcagtttatgattgtggattacatccttgatcagggtgaaaatagctataaaggaaattattgggacaatttgctaaaatttgaaaatggactggatttggctggtcgcagtggcttacatctgtaatcccagaactttgggaggccaaggtgggtggattacctgaggccaggagttcaagaccagcctggccaacatggcaaaacccgtctctactaaaaatacaaaaattagatgggcgtggtggtgtgcatctgtaatctcagctactcgggaggctgaggcttgaacccgggaggcagaggttgcagtgagctgagatcacgctactgcattccagcctgggtgacagagcgagactccatctcaaaaaaaaaaaaaaaaa

SEQ ID NO: 110 - DNA, 2214 bp, Homo sapiens
gcagtcgcagcatgctttccgaggaagccggtgttgccgagattgccaaaatgctttggagtttttaactgaatctaagaaaagtccaaaatagatttgagactgtaaaaacagaaactgcagcaagggggattcagtgccaatgcatcaacaaaaaagacaaccagagttagtggaaggaaatcttcctgttttcgtgttccccacggagctcatattttatgcagatgatcagtcaacacataagcaagtgttgacactgtacaatccctatgagtttgccttaaagttcaaagttttgtgtactactccaaataagtatgttgtcgttgatgctgcaggtgcagtaaagcctcagtgttgtgtggatattgtgattcgtcatcgagatgttcgatcctgtcactatggtgtaatagacaaattccgtctccaagtttccgagcaaagccaaaggaaggctttggggaagaaaagaggttgttgctactcttctcccatcagcaaaagaacaacaaaaggaagaagaggaaaaaagattaaaggracatttaackgaaaktttattttttgagcagtcgtttcaaccaggtcttatcacaatggccatacttagaacatgagcaaggatttcaattgacttctgaagtaaatctgtcttgaaaatatgaatgtggactgccttttatctctatttcactccattaacatgcaacaaactattgaatgatttcaaataattgcaaatgtataatatatattttaaattataatttaatttgaaggactgcagaacattattttacagacagcaaggatgcttctgagtgacacctaggaaattatttgaagaaattctttttatatctayacctgttgtgtaagaaactttaaaacattkgttattttctcacctttttttctaattcactttgattgctaggggtcatgtatgcttcgaagttacaggactaaaagagcaaactgaccggcctaaaactaaaatgacatttattccctagctacaaacatcagcgttattatgttaattataccttgccctctatcattataaatggttgccatggtgtttctaaaaataagtgttttaccattaatgtgtagagggcaaacaaagcataaagtactaagggatcatgcttatcctagggtctcacagaagagaggacatatttaattaatcttgtgaattacagaacaggttgtggtccagacaccaagaatcataggggtttttttttaaaaaacctaatagaagtagggtgacctctctctttggtctaagagttctaaaggaaggtaggcatctgtttaattagttggttcaccctggctttacctctggttaatgctttgtgttaataggaaggaaaaatcactttatcttttcttccaagcccctccctgcctgacttacccagactgggattaccagataccaggtgatttatgtggagatgatttttcacctttaaactctaagccaagtgtaagaaactcttgatagctatgtctattttatatcagtcactgagacttttttttaagtttttatttattattaagacaactttgccaaaaaagtcccctaagcacaactatttacatttctttatagcctcttctgatctctaacacatatgcagttttaactgttattttcatagtaactgatcttttgtctaaggatttttacctgaaagcacaatgtattgagtctcttgaaaatcatctttcagatctttttacagaatgaacttatgcactgctactgtagtattctcaaggaatatatgtaaacacaaatgtatgcctgaggttggtttttgcagaaaacagtctctgcttctaaaaacttctatgtctagtcttccataggaaatcctcactgtttaaccatgtgaggagcctaagtcattaaacggatcatgtctgtacattgtgtaatgaatgaaaagcacataaatgtaatctactttgaactttgtaaaaatgatgtgtggaggctattcttgtttctccatctcaagtcctgtgtgtgcacgtgtgtgcaagtgcacatgtgtgtgtgtaataacacattgtaaagaacagaaattactttaaaaaataaacagaaatggagacctgaaaaaaaaaaaaaaaaaaaaaaaaaaaaaaaaaaaaaactcgagggggggtcccgtacccaat

SEQ ID NO: 111 - DNA, 1453 bp, Homo sapiens
  SITE (946): n equals a,t,g, or c
ggtcatctttctcttgctcgtacagagaggagacacccgtagaaatggagatttctactacagatgaaaatttcttttataaaagggtaacttctctgtattatcctgtgtttgctatttctgaaaataataagctgaaaatattcctctttggcataaggattatttggtgtggcatgttctgaacctccactgttggcatcctttcttgattagcagaaacctaggaacattgttgtaataatgactaaattattgtcactgtcacatttgttagtaactttttttaatataattgccattaaatgtaaaaagcagcatctaagacattcaaaatgtaatttkgatactacttttaaaaataagatgctaaattaatagataaggtgggtttcctcagtatattttcattctaaaccatccactaaagtagggctaaagaggaatttagagtaggaagacttaggttttgtattctgcctttgttcagtatcagtgtgactttggccaagttacctgacttctgaactgcattttgcttttctctaaataagtgggggtaatacctatattagaggattatgataaaaagatgtgaacatattataaaattattttataaactagaagacatttcaaagaagttaagctgccactgttagtttcacagacttgggtgtattagatgaacagcttttcagttattgcttctatagttgtcctcttgccctttcctggattatcagtttctgcctgtctacctagtcattcccatcagtgtaaaacatttataytgttatttcttccaagttcagaaaaaaccctctytygaytccccccatcccattccagcactttgggaggccaaggcgggcagatcatgaggtcaggagatcgagmccatyctggctaacatggtgacccccatctctactaaaaatacaanacaaattagccgggcttggtggtgggcgcctgtaatcccagctaccggggaggctgaggcaggagaaaggcatgaacccaggaggcagagcttgcagtgagccaagattgcgccattgcactccagcctgggcgacagagtgagactccatctcaaaaaaamgaawaaaaaaaacaacttattttaaattattttcctagaaattatgatgtcagcagaggtagctaggtggtattatggttgacttttgttatttttaagacagcttccgtatttcttaggagttttgctgaagaacatggtatggggagaacatataatattctcatacacttcttaggatgggatagatccctgtaacagaatattggttaacaagagaaaaacaagttttaagacatgtatacctcatatatacatgggagatactcgggggaagtgagtaaatctctcagaggtggcttaaataccatcatgtcctgaaaaaaaaaaaaaaagggcggccgc

SEQ ID NO: 112 - DNA, 1552 bp, Homo sapiens
  SITE (1035): n equals a,t,g, or c
ttgcctaaggcccactgtgccaaattagataatacaagaagttcatttacactgtagaccagtgacgtcaatgactgtttgctctgtgataccgtttcaaaaatccaaaatgcagacttttctctgtgccatgcaggatgcagctgtgtgtgatatggtttacagtaatatttctttctcaaagtagcaggcttgttaaggaaaagataagcaacacatctggggaaaagggcaggtggccagcaatcgatgtggtagctctttgcccctctcggacagcaggaattagcttccccaggcattttctgtatgtgagttgtattgtgggatgtacaaatatcatctgttcctttgggtttccaggccagtagctctctattttgggttcaaacatgggttctcaggccgggcgcggtggctcacgcgtgtaatcccggcactttgggaggccaaggcgggcggatcacgaggtcgggagatggagaccatcctggctaacatggtgaaaccccaactctactaaaaatacaaaaaattaggcaggcatggtggcgggtgcctgtgktcccggctactcaggaggctgaggcaggagaatggtgtggacccgggaggttggaggttgcagtaagccgagattgcaccactgcmctccagcctgggcaacagagcgagactccatctcaaaaaacaaacaaacaaacaaacaaaaacatgggttctcaaaaggcatgcccactgtctcccatggagcttgacagcccatgccattagctctcactgttaggtttctggggaaggttcttctacttgattggaaaatttccaaataaatctttccagaagatactatgcacacagctaagtggcctgtctgtggagtaacccttttgtaaacaaacagaaacctaaagcttgatgttttggggggctgcctgtcatctataggttcatttaggtgtatttaggaagaggatccatgaaaccactggtttcctgttacataataatcattaataatgatttaaaatgtgnacattgatttttttraattccraaatacaagcgtatatggtawattaagtcaaatggtatgttcagtgagcgagatggggcttggggcaaaacaatactttgcttccaaagaggatacaactctcaaggagattctttcatcttgcctttaaggtcatttaaactaattcacataatcttcagaaaactaattcacatcatctattcatgtgtaaaatcaaaaggaagactgttttcttagtctctcgttgcctaactggccatttatactactaggttgattaagggatttgcctttttctgctgatatgggaacaaaaagtcttaagcatttttaaaggcaatggaaaattcagccacatgggggaaaattgatattgtcaccattgagttgctctgtttcttggtgaagagtgaatctaatctgatttccttcttcatcagatatgcctctttaacaacaaaaaaaaaaaaaaaggaattcgatatcaagcttatcgataccgtcgacctcgta

SEQ ID NO: 113 - DNA, 1489 bp, Homo sapiens
gaattcggcacgagtgcccagctcctgctgtaattagctccacgtgtacccccttcattccctccctcccaccgagccatccctgacccaggaactttccgcagactcgccgccatctgggagtgaagcaacatggatgcagtcagccaagtccccatggaagtcgtgcttcccaagcacatcctggatatctgggttattgtcctcatcatcctggccaccattgtcatcatgacctcgttgttgctgtgcccagccactgcagtaatcatctatcgcatgcggactcatccgatccttagtggggctgtttgagagcctcccaagagggccgggtgagggatgaggacaggcatcctatccccagcctcttcctgtcttcagaaaagcagcaggagggactttggggcatggacctgagttctggttttgattctgccacgagccagctgtgtgaatttggtcaagggacctaactctctgagttccaggttccttatctttcaaatggggatggtgatccctgccctttctacctcatagggatgtgagaaccacctgacttagtggatgtgaaagctgtttgtgatcagtaaagctaccacagatataagggtgttatgctgaatcctgagaagctttcaagaaccagagaacctgattgctgatgatggccttaaaggtggtgagggagatactgggggcagagcagactttgccagtgcccctcaggtcaaaccaagccaagagcaccctgtccccattccaaggggccagcagcactttggcccaaagtattttctttaaggtgccattccttcatgttttctcagtttggagggtgatgggtagagctttccagaaccttctccattccagaatctctgcccctgtgtaatctgaaggaaggctgtgccatctttgggcactgccaagggagttggggtgatgggcttctttctgcactggagtctcacatctgttagctttgacactcaagcaatgttggaaaatgcagggtgactgagttccctgcccagctttcgggatctctggcccccatccccttgtgtgtgtccctctgcccagctcctgctgtaattagctccacgtgtacccccttcactccctcccaccagctctgcagccagcctatggcaattatattttaagaggtgttcccaggacttttgggacctactaaaacaatgatggttattttagatgtgatgatttatatttatgtagagatatttctggaccactcaagctcttcgataccaaaatcaggagcatcttgggatttattaaattatgtaagaagatagcacagatatcgggatattattgtgtgaaaatgctgcttttactttgatgtgatctcattgatgtacacaaccaagttccaataaagtgctagaatgtgaaaaaaaaaaaaaaaaaactgcgagggggggacccgtaaccctaatcgaccttaatgagtgta

SEQ ID NO: 114 - DNA, 607 bp, Homo sapiens
ggcacgagtttcaacttgagatttggaggggacagacatccaaaccgtatcattaaatttaatagttttatgcagtttttttggctctagatctgtttagactcctgcagtcaggtgtctgtaactagcctctggtcctttttgagagttcacagtttggtgcaaaccctttggatgtattatttgggaaaatgggatatctggcagcctgtgtccctgctttacattatcctttttgctgcctgccccagcctcctcattagcatccctgccaaggccagtggagaaggatggagatgcggtgacattcagctgacagttgtcacagattgataatagctaacagcacatctctcccccggctccttccctagtgcaccaattagcccagcctcatctgcacctgggactcaagttgcctaaacatatttcatttcccatagcagaagatgccatccatctagagtgagactgaaaatacaaacaattcagaagttgtgactttccatgctctgcacacagaggctaccaaatgctaagggcgcttcctccccagcaccaggcttatggttctaagctccagaaaaatatcaaataaaccctgccc

SEQ ID NO: 115 - DNA, 1498 bp, Homo sapiens
  SITE (791): n equals a,t,g, or c
gcatccccatttctgttcctggaccctcttctgtcaccccatcgacaagtaaagaaatcaagaaatccaaactgattcgaagccagtcttttaataatcaagcttttcatgcaaaatatggcaacttagagaaatgtgctagtaaaagttctacaacaggatatacaacttctgtctcagggttaggaaagacttctgtgctttcactagctgatgattcattccggactcgtaatgccagtagcgttccatcttccttttctcctaatactcccttaccgagtacttcccgtgggacaggtaactcagttgaccccaagagcagtggaagtaaagatacacaaccacggaaggctaccttaaaatccagaaaatccaatccttaaatcaactgcttgatgaaggaggcaaaacaaaggcagcaggagataatgtgattggtacacaaaagctaaagcagtggctggggctttgtttttaaattttgggttttttttttgttgttgttaatgagcagaaagagagacataatgacagctgatgttaaacttttcatatttcaaattagattccctaggaggtataatatatatttcttgagtaataatgtggttacggaattccaatgttatagtgaagtgtaatgaaaaacatctctaggaatgtgctttaaccactgctgcaaaagaracaagtctgcatttatttgtgcaggaaaccaccatttaattgttctagarttttagcatttaaaaatcgtatgaaagtctacatcagctgaattgtcctancttgataagcacttggagggggacttggaaggtgagaaagatactgcattttctcatgagtctccaagcccacttgaaaagtcacactgaaaggatgcaaatanccgtctgcgattttggcggnccctccggattgtggtgacantatgttgtacccggacattcccaaatttaaatttggcagtgraaattccacaaagatttctggtaacttkgagctataatnccttaaaaataatmgattgatgattttctttatttccagagaatttattttataaatactttgtttacattttagattgtacttgtctttatttaaatgatgaatctagtctgaatcagctgttattccagctatcatgcttaagctatgtcaacagcatttatttgtactaatgctaatatttccatcaaaatttgcctgtggaatatatacagttcttaattttaaactgtcagttcttgagatataccgtgtgaagctattgtcagcttctctatttcaaaatgcaatcagcatataactatattgatattagaagatatttgttttttaaaatatattgatgtatgataaagatgatctaatttgtgaatgcatatgtatgtgtggttactttttataatgtgaaataatgaataatgaatttactcaaaataaaacataggttaatgagaaaaaaaaaaaaaaaaaaaaactcgaa

SEQ ID NO: 116 - DNA, 1797 bp, Homo sapiens
ggaacacaaaaaaaacactgggaaaaagaaatgaaatcttagcagaagctggaactttttatacttataaaaatagtatgttcttatgtttctccttatgcatttatgtgttcacttaaaaatgtttttaaatctaaggttttctttgtttatgttcaggtaaggaactgttgtcatgatctggaaatgtttaaaacaatgtttgttagcattctgtgagcagcaaaacttatagtgataaaaatcgattgttgttaatatgatgtttaccatgtgcacatagtaatgaaaaggaacataaaagcccagcaggctcgtaccaaagtcacagcagtaatgctatgtactgcagagtctgatgagctggcctttgtgcacacttttattttcatgggattgcatcttagctgttaaaacttctagattgaaatttgacagccagggttacatattggggacttttaaagtgtctttccaaagagatttcattaaccgtttagattagaatatctttcccaattgttacagtgacatatatgctgcaatatttaacaactggagtattagccacatgggttattttttcaatctgtgttttgaatttttttattgtgtgttatttaaaatattacatatgcagctgggagaactacacctttgtgcacatagatttatatattaatttgtagaaaatattttctttatatatttccttaccatacaaggtgccttgttcatcaggaaaacttttgttttgtattttgacaagaaaggcaccttcagagtttctttttaagtatagttgacaagtgtataaatgttacacttactttcagagttctttttagatctaaagaagtcagttcaaaaatggaaatcaacaatgttaggagaaatctgaattctgttaagttagtaagtattatgtatagcatctgtttttaaccatttccattcttatccctagtgtatcagttgatcacactaagaaagcttaaagattgagcatttgaaataaatgctctttataaatgattagatttttgaagggatattgaaatcattgcgctgtgatttcatctgtgatgtgaaaaatcaatttattatccttggtgctttcccccccaccaatgcacaaataattgtgaacagcttgaaatgacttaaactgtaatccaaatgggacaatctgataagaatttcatgcattggtagttaaataacttaaattgctaaagctttagcttgaaatttatgtttagaaaaactattgatttcccatggtatgaatataactattgtaattcttcaaatgagactcttctcaccttaaatagtcatatattaattaacttataggaaataagcatactatatgttagctgttttaaaaggtaccagatgtaagagtcataaatatatgcaattaaagaagttcatagatttcacatgaatgtaaatgtgttatatggagacatgtcttgtaaacagttgaatgtatgtaagttttctgtttgtgaaaatgtagttaatgtactcactgtggaggtcataaggaagctactttttttttaaagtggaacctaattaaaatatttccagaatcaaagagacttaaatggtaaatttttaaaattttcttatctcttactttttagttttcaaagtagaaaaaatcaggaatttttttattaactagtacttacatattaaataaaatttattattggctaaaaaaaaaaaaaaaaaaaaaaaaaaaaaaaaaaaaaaaactagttctagatcgcgggcggccgc

SEQ ID NO: 117 - DNA, 952 bp, Homo sapiens
ggcacgagcctggcacatagaaggtgcccattaattggatgaacgaatgggggagttagagtcagtactagaaaaagcatgggattatacttttaagaggccaaatgaaaatttggggaatagccattttccaggaatttaaaaggagccattttttaaatgtcaataataatttattggttattattttttaagcacttattatgggtgcttattataggcatggtaaaagcacttcatccacattatttaaatctcagaatctatgagtttggtgagatcactgcagttttacagaggaaaaaacagggcagagagaacggtaatttcctcaagttctcactcttgtcacttaatagatctagaattccaacccagatctgatggtgaagtcagtataagcttcctggagaaaggagtagagttaaggtgggggatggggcttgaagacttgataggattagggttgggagtgtcaactcggagatccacagtcaggcggaaggaacccacaaaggcaggaatgcacacagcatgctcagaggagatggaacctgaaaatagagagaattagcaagtagtgcccattggatggagtaaagagccatacctagaaggttatcactgggtaaccatgataagagttttggcctggtgctgtggctcacgcctgtaatcccagcactttgggaggccaaggcaggaggatcacctgaggtcaggagttcgagaccagcctggccaacatgatgaaaccccatctgtactaaaactacgaaaattatctgggtgtggcggcaggcacctgtaatcccacctactcggaggtgacgcagggggaattgcttgaaccggggaggcagaggtggcagtgagccacgatggtgccactgcactctagcctgggcgacagagcgagatccgtctcaaaaaaaaaaaaaaaaaactcgag

SEQ ID NO: 118 - DNA, 1185 bp, Homo sapiens
ggcacgagcttatgtctatgttttgagatagaatccttctattttatagcagggatgggcaaaccacaagcaaggggccaaatccagcctgctgcctgtttttgctaaaaaagttttattggaacacagccatggccattcacttccatatcatccaatggctgcttttgtgctacaattgccaccatgcccagtggggcctgtggcacacaactgcagaagtgagtggttgtggcagaaatcacttagccttcaaagcctaaagcacttactatctgaccctttccagaaaaagtttgctgacctctgttttagagatacaaactaacatacttacagataagtgatctgaatatgaagcaaattttcaacataatctgggttgatggggaaccgtgttacatataaacaaatccagagctaatcattgccaggtgagggctgtgcagacgatcacgacactattctcttttgcatatgttttccataataaaaagtcttttgaaaaaatcaatgatttaggggtttaaatatactgtagtcactttggaaaacagtttggcagttcctcaaaagttagacatagaattaccctatgacccagcagttccctttctagatatacccccaagagaattaaaaacacacattcacacaaaaacctgcacacaaatgttcacaggagcattgccttaatagcaaaacaagcgaaacaacccaaatgcccatcaggtgacgggtggataaacacagtgcggtctgtccatacatggaatgtgactcagccacaggcakgaatgaagcgctgacacacgcagcaacacggatgaaccgtgaggtcacggagtggagtgaaagataccagtcatgaagtcacacaccgtatgatgccattgacatgaagtgttcagagcaagtaaatccttacagatggaaggcagagcggtgactgccagggactaggagtggggggccagggggtgactgctaatgggtatgggatttcatttcggggctggtggaaacgttccggagccagagagtagtgatagctgcacaactctatgtatatgctatgaatcaccaccgaatggtatatttttaaaggacgaatttatggtatgtaaattgtgtctcaataaagctgctatcttaaaattcaaaaaaaaaaaaaaaaactcgtag

SEQ ID NO: 119 - DNA, 1098 bp, Homo sapiens
tcgacccacgcgtccgcagaactctagatctgggaataaccctgtatctatttctttacatttttctttcattaatgtatttatcactattttttttttgtttttctttgcaggcctcagctgttgaagaacgctctgcagagagcagtagagaggggccagttagaacagataactggcaaaggtgcttcggggacattccaggaactccgacccacctggtgcagaggagtcttgtcttgacctgctttgggagagtgttgtcttgatgcttttctcttcctccttggaaaacagctgaagaaatcaggggagaaacccctgcttggtggaagcctgatggaatatgcaatcttgtctgccattgctgccatgaatgagccgaagacctgctctaccactgctctgaagaagtatgtcctagagaatcacccaggaaccaattctaactatcaaatgcatttgctgaaaaaaaccctgcagaaatgcgaaaagaatgggtggatggaacagatctctgggaaagggttcagtggcaccttccagctctgttttccctattatcccagcccaggagttctgtttccgaagaaagagccagatgattctagagatgaggatgaagatgaagatgagtcatcagaagaagactctgaggatgaagagccgccacctaagagaaggttgcagaagaaaaccccagccaagtccccagggaaggccgcatctgtgaagcagagagggtccaaacctgcacctaaagtctcagctgcccagcgggggaaagctaggcccttgcctaagaaagcacctcctaaggccaaaacgcctgccaagaagaccagaccctcatccacagtcatcaagaaacctagtggtggctcctcaaagaagcctgcaaccagtgcaagaaaggaagtaaaattgccgggcaagggcaaatccaccatgaagaagtctttcagagtgaaaaagtaaattttataggaaaaaagggtatcatgatgaaattcaaaatcttattttctaagcacttttgatatcaagcaagtggcttcctttttgagatattaaaaaaaaaaaaaaaagggcggccgc

SEQ ID NO: 120 - DNA, 805 bp, Homo sapiens
ggcacgaggtccctaattgtcttgtacctagccctagggtgaccagggcaggggaatcatggcgagaagcgtaagggcctgatgaagaaggtgtgctgggtgtgggctctagcccacttggttttgtgtgagaggtggctgacagcaggttgtttgctgtatgtaggagttatccagccctgcaagggcagtccctccagtgtctgcaaagcccgaagatgtctgcatccaaaatacagaataaaaagatatggttactacaagtactcagtaagactgataatctgtcatcatcatcctcatgcccttaaagcagagctaactgatgattaatatatgcttctatgttaacagtcttggactttattaatggtgggtggaagttaacttaatgtatgtatgcaaactaaaaagtggcatccttttcattaatgacccaaccattattcaagagctatgtctagttagggacttcagacttttgaaagaaatgaagaaataatgccagatacatgggctcgcacttggaatcccagctacttgggggaccgaggtgggaggaccgcttgagcccaggagttcgagaccagcctgggcaacatagcgaaaccctgcctcagttttaaaaaagaaaaaaagaagtagtgaagaaattggaaaggattctgagaagaaatatgcaaggtggaaaagagcctagaaagaaaggtgacagatgctgggatttggtcgtcagaagagatatctaggaaatagcatggcagccctcaagtactagctccacttaaaaaaaaaaaaaaaaaaa

SEQ ID NO: 121 - DNA, 3435 bp, Homo sapiens
  SITE (760): n equals a,t,g, or c
cgagcagcatcagaaagtacgawtctgctctgagggcgratgraaaatgaagawtctgacgtaaagcctccagactkgccaaacccaatgaatgctacctcccagtttcctcagcctcagcactttgcagctttggcctccgtctgcctcgggatatcacagagctgcccgagtgragtaggggtaccccttctacatggccatgggcttcccagggtatgacctctcggctgatgacatagctgggaagtttcagttcagccggggcatgcgccgcagttacgacgcagggttcaagctgatggtagtggaatatgctgagagtaccaacaactgccaggctgccaagcagtttggagtattggaaaaaaacgttcgagactggcgcaaagtgaagccacagcttcaaaacgcccacgccatgcggcgggcattccgaggccccaakaatgggaggtttgctctggtggaccagcgtgtggccgaatatgtcagatacatgcaggccaaaggggaccccatcacccgggaggcgatgcagctgaaagctctcgaaatcgcccaggaaatgaacattccagagaaagggttcaaggcaagcttgggttggtgtcgaagaatgatgagaaggtatgacctgtctctgaggcataaagtgcccgtgccccagcacctgccggaagacctgactgagaaactcgtcacttaccagcgcagtgtcctggctctgcgcagggcgcatgactatgaggtagctcanatggggaatgcagatgagacgcccatttgtttagaggtgccatcacgggtaactgttgataaccagggcgaaaagcctgtcttggtcaagacaccaggcagggaaaaactgaaaatcacagcaatgcttggtgtcttggctgatggraggaagttaccaccgtacatcattttraggggaacatatatccccccggggaagtttcccagtgggatggaaattcgctgccaccggtatgggtggatgactgaagacttgatgcaggactggttggaagtggtgtggagacggaggacaggagcagtgcccaagcagcgagggatgctgatcttgaatggcttccggggccatgccacagattccgtgaagaactccatggaaagcatgaacactgacatggtgatcatsccagggggtctgacctcacagcttcaggtgctggatgtcgtggtctacaagccactgaatgacagtgtgcgggcccagtactccaactggcttctggctgggaacctggcgctgagcccaaccgggaatgctaagaagccacccctgggcctctttctggagtgggtcatggtcgcgtggaatagcatctcaagtgagtccatcgtccaagggttcaagaagtgccatatctccagcaacttggaggaggaagacgatgtcctgtgggaaatcgagagtgagttgccaggaggaggagaaccaccaaaagattgtgacaccgaaagcatggctgagagcaactgaagggaaagggaaagcaaatggaactctgatttaaacagctggggatgaaattcctcaagatgattattcctgaaagtgtggatgcgctggatgcgcagggaacatcaggaaaaggccacggggctctgaacagccccggtccagacagcagcctgtacatccatcccaggacacagcccagcccctccccacaccatacaaggtatcagaaaagtctaggacctatcatttcatcagagacatgatcagaaaagaaactgcttctgccccatttcttgttttggagattactccatctgtccatcaaaagaaacctgtaaatatgaaagaacaaaggttatttcctggagaaaagacaatttattcaacaccaacaagggactcatcatatgggcacaactctggtgtccttctatggagaaaacctcaagtaaagttttattctgcctttgaaaatgcttccaaaagtagaccctgtccccacacaggtcaagactacagagaaggctttgtagaaatgtgtcacctatgtacacctgctacttacacatttcctcttttggaaaaatgagatacttagaataacaagaaaattaagacatactggcctggtgccagcagatggcttttctatagacaaactaggttagtgtggaagatataggttaaaataaactatgctgttttatttatcttcccaacctgattggcagctagacttttttagggtctcatttaatggccctgtttttttcattattatatttaatgatagggcaggatttcgtatgcaagctcttgtttctcaggctgcctgcagaagaagtcgctataaattatctgttgtctacatggtacaaggcccattgactcatctgatgcttgttttgttaatttctttaatatttttatcacggggcagtgggagggcttgggcttttagccacagctgttttaagacttctgatctcctgccctgcaggaataggtgggaagtcattgaatttttacactatagtaatttgcattcccacataagtttgagtgttacgaaaacattcctttaaagggatctgtgctacacaaaatatgccaggacctcacagacaaagccattgctagaaatgtcattccaatgatcagatctggaaacaggctgccataaccacttttccttcttgtagactcagctcacctgtatatttaaactgttcttggcatcttgaaacacctatttctactcaggtactcattgtcctgttactgattcacctttctgatccttttcaaccagttttcccccaaggggggaaattttacttaacctctagtatttgaacaactcaatatttgaattgttgccccatttgcttttacctgtactgtattcttggtcatctcaaatggcgtctaaacccagctactttgcattccagaagtttccattccctccaattccacctaatttttcatctgtcctagttactggctctttcttcatgtcttatttctcttgctttgggagcttaaaagattttacaagacctaattttgggttccttccttggagccatagttaccctgccaagaagagtagaaaatgggttcaactcctgtttcgctccaccaacacctctgtgagtctcatcatcagctgagcgatgatgccttacaggttgcatagcactggaactttcctagagtaacggctctgctgccagggtttctctgggctcattcttccactgacttaattatgatctatgcctaacagagccccagtacaactattttgcagaatggctgttaccctagaattactatagcacatattgagatatagttgtactccctagtagataggaactgaccccaacaataaactttgataataaagamaaaaaaaaaaaaaaaaaaaaaaa

SEQ ID NO: 122 - DNA, 1020 bp, Homo sapiens
  SITE (3): n equals a,t,g, or c
cgncacgaggtgaagttcaacccaatgcaactttccttcagtctttccgtgaaagcgcctgtgaaaaatgaggtcatatttccctttctcagtctgccccttcccgttttgctctccggttttcttctttgtcttcacagatgtttacctatgttttttctttgtttttgctgttggaagacatctaagtgatccttttcccattctctttttcactcataaatgtcctgatgtttagcaaaaggcagttctctttgctacttgagcttgtaaactgttgttaaatgagtaaccaaaaggaaagtccttgcgaagttggttaccatttcagatacaagaaccgtttatcttcccacgctgacgaattttgcgagtgagatgattatttttccttgtgtttgtaatttatttaagtaaattccttgtttgtttttcttttcagtacaccaggggtatatattttcaatatgacatgtacctttggttcagggctaagttagagtctgaaaaatgaagcctgtaggattcatggcagtgatctaattgtgattcatcttactgattgtagggcaagaagagtggactaactcaagacacaaggcaccttcagcgaggacagcaaagggcgtctacagagaccagccatatggcagatactgattgtactgtctgatgttgtgaaatagccaatctccaccagtcctgtatactgttcaaagtaatttttttctatgaacaatccctttttaaataaatcaaaatgcttaaaatctgaatggatggaacttaaaactactttgttgaaacatcaacctgggcagaaaaaaaaaaaaaaaagacatgtaaaattttgttatttccagtctgtatatgaaaaaataggtcatcaaaaggaaaaaaaataactttgattaactagtgttaaacaaaaaataggtttactaaatctcgtgccgaattcgatatcaagcttatcgataccgtcgacctcgtagggggggcccgtacccaatcgcctgt

SEQ ID NO: 123 - DNA, 1378 bp, Homo sapiens
atcttagcaatctccttggcccaaaacttcaccccatcttggaagggaggggagagagaatgttctgatctatatctgatgagggcgtgtggttgggacctgagcatcctcctggttgggctagtgatggggagagagggctgttactcacgactccctccaacagaataccagaaacaggcaggcagctcaggtgtatgtaaggatgtgaggccaagaaaccagccctcaccaagttacccctgtaaatccttgtctccccatgcacctctactttgagtcagaaatggattcattgcaggctcagttgtttgtattatgtgaatgaactgaacgtaaccaagcaccaagagagccctaaagacacagtagacctcctgtagaagggctctgatggaccttcaaacattgctyctccaactttatggtgcacacaaatcacctgtgcatgttaaaatgcagacggtgacttacagatcttgggggaggccaagtgtctgcatttccaacaagctcccagatgatggccacactgctgatctgaggaccacattttgaacagcaatccttaaaacacacaaggctgtgggaccgcactcctgagaagagacctcaytcttggaagggattytaggaggctgcattcaaaccatcccagagacagctcacaattttgttagggacctgatactaaaaattaccaaatcacaatcgtgtgctgagggcttactttgtgcttttcagatgacatttcatttgatgctctcaataaccctgaagggatttcagagatggggattatgatggagcttcagaattaaactgattaaaactggcccagagtcacacaactatgaagaggctgagttgggatttcttttaatttttattttttgaaacagggtctcactatgttgcccagactggtctcgaactcttgagcttgagcaatcctcctgcctctgcccctgagtagctgggactacagatgtgagacagcatgccgagcctgctgagttgggatttgaaccccaggtttgtctggctgtaagtcctccagtgtggaagtacgatagctacccatctcagccacggaaaacagcagagttttggttcaggcaagtagcactaagtagaaagcccaggtatgaaccagatctgcaggagcccaaagtcattgctcagaaccaccccctacccgctgagaagttcttcctgtccctggaagttctctctgagatccacccaaatgtgtcctcactaaggcgaaatctggcttctcctgtttgggcctggggagaaacataaacctagccctttgtacacaccgcctcgtgccgaattcgatatcaagcttatcgataccgtcgacctcgta

SEQ ID NO: 124 - DNA, 1146 bp, Homo sapiens
tcttttctgtaacttcatttagcactcaaaacaatatcactatttaatgttacactttgactacaaacttatctttctccttacaagctttttatgtatttaatattatcttggctatttctgtgcaaactagttaaatgttactttgaaattcttcttttctctacatcacctcagttactccagtggacagtaattgttacttactgtggtcctctgttacggttttgaatttcttcaaatgccttttgaatttaatgaaaatactacatgaaataatactggtggctacataattttcttccactttttcttaagtctctgcaatgaaacagctgacagtaaggtgtgcgtgagtggtgagatatgttaactcctatacttcactttagcttatgttgtcagggagtgatctccaaatgcaaaaatataaagtactctattcttggggaacaatgtgagcaatggaaggcttggcctttcttaggcagatccctcagattcaaaaggaacacatcttgatttaatatcaatctctatacaggggcctgagaatgggatgaggagagctgctggtctttgtactgcaatttaattcactctgattactttctatcctcccacccccactcttcctccagtctctttctgattagttatcatagtcttccccaagactctatatatgctctctgggttcctgtcaagaggaaatcctgtacacaccccctactcctctatcactgcttcctggtctgggcactctcactgtgcagcttcttctgctctggtttagccaacaatataatccatagattttctgaattccatcatagcatctggtctttgtgtgagtcctctttcattccgtgtctattacgggtatttttaaatatctttactgttgatacagtgagatttgtgggaaagaggatagaaacataagccaatctcccctcttgaaatggacatctattattaattaacactcatcaaaataaatgttcttattgttatatcttagttggataaagatgaaaagataagaccaaaatcctgctctctaatcacgatttattgtaacaataatattattgtattattggggggggcccgtacccaatcgcctcacatgcatcgtataca

SEQ ID NO: 125 - DNA, 1675 bp, Homo sapiens
cttaaagacgccaggtagagacacacagaacgtatgtattaagaatatcctctctgggctctgaaattttaggagtgattcttatccactccaagttgtaagtatttgtagaaatttgtgcaaacaaacaaaaactatcaaatgaaaagaaaatgtactcaacctaacttatagttagcagctggaattctcaactcttccctgccagcactataccacagtgtggaagaaattagtcaaatgcttgttttcctgcttctcttttcaactgttactgtgctttgtttgaaagtagttttctctctcaaagccgttgcttatatcgttaagaatgaaggtttgtgtttaaaatttattgcattgcaaagggtagtttcactgaagtcatgcaccattaaataagatgaaatatttgtatttattgtcctacttcctaagccgtaacttcttttcctctgtgaatttgcattgagtcactcatgctacactacatcgctttagtatttgagatggcatttatgtttcctctcgtttatcatgaaatggggtcagattccatcagattccacctctgtcaggtggactcttgtctgccttccatgatgagattttttttctccttcccctttctttaagagaggctgacagatctaggtgtcaatcaattggaaaccagtctctgattttttttcattagttattttctatcattagtttcactgtgtaaattagatatcaactgcacttctttaaaaaaaaatacatctccctattacctccttgaaagatttacttctgtaggcctttttcaataggctcatgactgcagacaaggaaaaaaaaagtaaaaacaaaaacagtatgtgcctgaaaatgacaaaaaaaaaatttgtaacatttaaaaaagaaacctgaatagcctttaattctttaataatacacttaaattttatgtaaatcggttttcgccacgtgtgtttgttcacattctaaatgacttaatgggattctcacggtctgtgtctttgtgtcacgtgtataaaatgggcttgtgatgtaagcgtttcatctggtcagtggttcctttgatattgtactgctgctgggagtgggctgtggaacctgccttcgggtaactgggttcctcttgggtagattggagagatgggggtgggcgtgggcaaattctcacacatgttttcttaacctatttgcagaaactttcaaaaggcatttgattaaacctcttggcagtacagtattcttgtatttgttaacgtctgtgtttaggtactggtacctttttgttttaaaatgttctaagtgttggctttaaagtgaatttatctttagtatgatagttatatgaaaattataggatttgtgtgcagagaatttttttataaagtgctttgtaaaaaaaaaaaaatgtattctagcttttgcggtacatatgtgtgataactttaatacccatgacagttaagtgcaattatttcatcactctaaaaatgctatttttgtgtcagttcctgcaggtgttttcatgtctttgcaaagtgacacattttgatgccttcttgataaagtggtagacattttgtagctttctagaaactttgtattcatacggtatcaatgaaaaataaagaaaatgaaagtgtgggtcaaaact

SEQ ID NO: 126 - DNA, 1064 bp, Homo sapiens
gcggaggggtttctgtgcaggacgggagtctcagagaggagacggatgtgggggagggaggccggccacgcggtggacagagcgagggtgccagggtgacccgaagaccgtcaccacccgacagcaacgcaagtgcctttgaccttgatttggacttttctcccttttgcatttggtgctacagacttgagacaccagcagaagttgtgttcagcccggccccgctgcgcctgtccgggccggggctggcgccggttgtgtttgtgtccaccttgccttctttgcagccaagcagtttttgtggatgggacttacctgcacgccccaggggtctttcaggattcaggatgacttttcttttacaatggtttcctctcggcagagcccgggttgtgggggatctgtgtgggttctcaacgcagatccatcctggggtctcccgggcagggatggctgacctcgagtcccctcccttcccgagaacctgctctgtcccgagggcagctaacaagggctgagccccaggtacaggttgcctcctccacggcaggaatttttaccaaaaccacaagcaaaaaacaaaacagaccaccacgaccaacaacaaagatggggggtarggttttgtaaaggttctgttaggttcatatttttatatcattttgcccataaatgcggaatttgccgtgggaatttgaagacaaatgatctatgtttttatggttttctagggaargtgttctgggggccgggctctctccagctgtgggaggcctgctccctctggggggcaccctgggcagggtgggggggccttgggaggcgcttcttgccaaatgcagacgaggggtgagcctgccagcgtttgcgacgtccccgcacgacaggctcatactttctgaggatcgtgcatagcataggacgtctgaacctttgtacaaatgtgtagatgacatcttgctacagcttttatttgtgaattaaagatgcattgatggttaaaaaaaaaaaaaaaaaaaaaaaaaaaaaaaaaaaaaaaaaaaaaaaaaaaaaagggcggccgc

SEQ ID NO: 127 - DNA, 1607 bp, Homo sapiens
ggcacgaggaaaaacttatttatcgatagacctcatttatgcaaatgtttgtaaagtatcatgttattatggtaattatttttattttcatacttatcacatcagacaagcatggtgaaataatatatattaaatacatagatagagttatcataacagagagaatctagtaaaaaaaaaagacatgaaaaacatcccattaaagaaaaagcctttatatgggacattattcacatttctgcaaatggaattttctgattaactgccatatattatattatttgaataactttaggraagrttaatttccaagctctgtccctggtagtattttttcctggctgaaattgggctttaaaacaaaacgtcagcaatgcaagctgacgattccagttacaggttcagaaacttcgagtgcaggcagttaaaagtgatttaagaaaggcaaaatcattctttcacaaatgtgtatgttaaacttgtaaattgatatatcaaaaccacaaccggaatatagcaatttgtctctctttgcctgctgtggtgtctcccgcagctgcctgccagataatatactgattgtaactttcctaatttacagagcctttaagtctaatctcctgggccagcctaaagggcttcttcatccattgaaatgccagaataattgaaattgtcttcagcagacagtcaggcaataatgaatattttctctgctgattgcatgccaaggttgcacatagcactgcaaacagaaatgatcccaaaccgagcyccccagggaggggcagccgctaacctttggcatgaggcacaataccgacggcttcccttctcacgggctcctgaartcacggacgcccaccaagcttctgcacaacgaggtgcagcacagttgccgcgtgaacaatgatgccattggcttccctgactctccagaggcagcttaaaggctgggaaaggttgtggttatggattttctgatgggggcagcgggagagatgagcagaggaggctgctgttggcattaagaggaaatataccaatagttaatcaaaagaaacccggcgttgaaggggcatgagaggacgctggcttgtcagctctggagcagcatttcccatacctttgaatggtttttgtgaaagccgggagttctttcctgactcaggatcagtgctgcttcattggcgtcctaatgrtgtgctgattgaaattaaagtgtttggtagccgaagccaatcactaatatcttcaaaaaatctaaagactagtttaaccttcatctacggaaaggttgaagaagttttgaataattaatataagcaactaatcttagattatggctacttccatgatatgcgttagaggtggaaattgggggatgagaaaggaactaccctaagacaatatggatttaagccacataggaagtgtgcagattaaacagccgggcgtggtgatgcacctctgtaatcccagcccagctactcatgaggctgaggcaggagaatcgcttgaaccctggagacggaggtttcagtaagccgagattgcatcactgcactccagcctcaaaaaaaaaaaaaaaaaaaaaaaaaactcga

SEQ ID NO: 128 - DNA, 1037 bp, Homo sapiens
agctactccgtctggccccgccttttctctgctctcctgaacctttaggcttgtctcggcccatttgaagaccaggaagttgatcaatcccgaggctgctgagagacggtggcgcgattgggacagtcgccagggatggctgagcgtgaagatgcagcgggtgtccgggctgctctcctggacgctgagcagagtcctgtggctctccggcctctctgagccgggagctgcccggcagccccggatcatggaagagaaagcgctagaggtttatgatttgattagaactatccgggacccagaaaagcccaatactttagaagaactggaagtggtctcggaaagttgtgtggaagttcaggagataaatgaagaagaatatctggttattatcaggttcacgccaacagtacctcattgctctttggcgactcttattgggctgtgcttaagagtaaaacttcagcgatgtttaccatttaaacataagttggaaatctacatttctgaaggaacccactcaacagaagaagacatcaataagcagataaatgacaaagagcgagtggcagctgcaatggaaaaccccaacttacgggaaattgtggaacagtgtgtccttgaacctgactgatagctgttttaagagccactggcctgtaattgtttgatatatttgtttaaactctttgtataatgtcagagactcatgtttaatacataggtgatttgtacctcagagcattttttaaaggattctttccaagcgagatttaattataaggtagtacctaatttgttcaatgtataacattctcaggatttgtaacacttaaatgatcagacagaataatattttctagttattatgtgtaagatgagttgctatttttctgatgctcattctgatacaactatttttcgtgtcaaatatctactgtgcccaaatgtactcaatttaaatcattactctgtaaaataaataagcagatgattcttaaaaaaaaaaaataaaaaaaaaaaaaaagggcggccgc

SEQ ID NO: 129 - DNA, 1146 bp, Homo sapiens
ggcacgagatttttggtcaggtttttatgttgtttttcagtttgaaggagtcgttatatatttttcatactgctattttgttagtagtatgctttgcatgtgcagtagtatgtcagtatgtaatagtacgtgtatgtgcagtagtattttgcttttcaaaatctcagtctttgatctgaatttaaagaatagtatttttttgtgtaagaggttatttttatcttttgctatttaaaaaatcaagtgccatttgtatatgatagtacttaagtcttcagtttgaattctaaaaatgtgtatacctatataatccacacacctgtcatcccatcatcctatcaacacccctgaaaattcccttgtgcctcttcctagtcaaccccaccactgcctcaagtaagtactcttctgatttctgttctcatagattagtttcacttgctctagaacatcatataatggggctgggcgcgatggctcacgccagtaatcccagcacttgggaggctgaggtgggcagatcacttgaggtcaggagtttgagaccagcctggcaaacatggtgaaaccctgtctctactaaaaatgcaaaaattaacctggtgtggtggcatgccttttgtaatccccagctactccaggagtccaaggcccgagaatcgcttgaacctgggagacagaggttgcactgagctgctgcactccagcctgggcaacagagtgagactgtctaagaaaaaagaagtttatatgatggaattatacagtaaataatctgtatctattttcatttagcattgtatctatgaaattcatgcatgttttatctattaatattttttttggccgggctcggtggctcacgcctgtaatcccagcacttttggaggctgaggcaggtggatcactaggtcaggagttcaagaccagcctaggcaaagatggtgaaaccccatctctactaaaaatacaaaaattagctgggcatggtggcaggcgcctgtaatcccagctactcaggaggctgaggcagagaattgcttgaacctgggaggcagaggttgcagcaagctgagatcgtgccactgcactccagcctgggcgacagagcgagactccatctcaaaaaaaaaaaaaaaaaaaaaa

SEQ ID NO: 130 - DNA, 1172 bp, Homo sapiens
ccacgcgtccgaacaccttgcagctttacctgtgaatgtcaagattggcaagatgcttatttttggtgccatatttggctgccttgacccagtggcaacactagctgcagttatgacagagaagtctccttttaccacaccaattggtcgaaaagatgaagcagatcttgcaaaatcagctttggccatggcggattcagaccacctgacgatctacaatgcatatctaggatggaagaaagcacgacaagaaggaggttatcgttctgaaatcacatactgccggaggaactttcttaatagaacatcactgttaaccctagaggatgtaaagcaggagttaataaagttggttaaggcagcaggattttcatcttccacaacttctaccagctgggaaggaaacagagcctcacagaccctctcattccaagaaattgcccttcttaaagctgtactggtggctggactgtatgacaatgtggggaagataatctatacaaagtcagtggatgttacagaaaaattggcttgcattgtggagacggcccaaggcaaagcacaagtacacccatcctcagtaaatcgagatttgcaaactcatggatggctcttataccaggagaagataaggtatgccagagtgtatttgagagaaactaccctaataaccccttttccagttttactttttggtggtgatatagaagttcagcaccgagaacgtcttctttctattgatggctggatctattttcaggcccctgtaaagatagctgtcattttcaagcagctgagagttctcattgattcagttttaagaaaaaagcttgaaaatccaaagatgtcccttgaaatgacaagattctgcagatcattacggaattgataaaaacagagaataactgaaactgaaattcatggtcaactgctttaaaaattaagatgaagatacagtcatgaaattatctgaaaatgggtcatcacattaagtatttcattacttaaaatgttggtactagccattaacttaaaggtggtgggaaaaaagcacatactttaaacatgtataattttctagttcctttttaatgatgattattctgaatgtatttgccactacatttacaataaattctttggtattatgaaaaaaaaaaaaaaaaaaaaaa

SEQ ID NO: 131 - DNA, 663 bp, Homo sapiens
ggcacgagaaaccatgaaagtcctttcttggatccactttatcttgattagtctgcattttactagttcactggatccctcctctaggggcctggggactttcactgatgctcttcctgattctagagcaaaggtgtgggaaggggaaatggaggaatgccctcctgtctgtgtcgttctctgtgccacagctacagatgcagaaggtttctctggatagcacacctctgaatgtaaatcatgataaaatggatatttggaaacttactcctaagctgtgatttagggtgtatttctacttctggactgcctcaatatcaagggctgagacttttgaattttgaatattcgttgggtttcatgttaagaagcctgtggtctaggagtgctattcagtgtttcttttcctgataaacactttgaatattttttttgtgtttttgtttccttttctgaagctgttcctccttttaaatatttttaatcacattgataaaatctatccttcaccacctctggttctactatagttgatttttattttaaatgtttaattgtatttgattaaacacttaactggattttggaataataaaactctcgtccaatttggcttttaaaaaaaaaaaaaaaaaaaaaaaaaaaaaaaaaaaaaaaaaaaaa

SEQ ID NO: 132 - DNA, 776 bp, Homo sapiens
ggcacgagctgatttctatttttaggagctacttggatttgtatgtattttttctacgtgaaaatatatgtactcttcacttttgttccagtactataattgctcatgcactctttctcccctttgagaacattcagtgaaatacaacttcatcaaagatttgctcaaaggagaagaatcgcatgagtgtgaaaagtagatgctcgtagccagaacagaaaaggttacacatgatcatggcacagaagataggaggtttgacttggtgggccataatgtttattatcctttttgaaataacagggaccagcagcagttttctcaggataaatgctctaccccacttctctatgaacaggtgtggggaggcttactttccattttcatatttatacacctctctacaaaagcaatttttaatgaaggttagtggaattgttaaaaatctgagagggaatgatgactggaggtgttttggggtttttttctgtattcattttttaatgagaaaagttttaaatgtagtacaggttagacccaactactaccttactattataggacgattctatgtttctgttaaagtattcaagtagctttctctgggggaaaaagtaccacttggacacttaaaggaattgggatttttgtctactttggataaggcagttgacttcttaagtaaaagcaatagtgtaaaatgtcattttgtttggaatgttaagtgagcaaataaaaaacatgttgaaattgtaaaaaaaaaaaaaaaaaaaaaa

SEQ ID NO: 133 - DNA, 1543 bp, Homo sapiens
  SITE (1055): n equals a,t,g, or c
cttcgccgtcatccgcttcgaaagcatcatccacgagttcgacccgtggtttaactatagatcaacacatcatcttgcatctcatgggttctatgaatttttaaattggtttgatgaaagagcatggtatccactaggaagaatagtaggtggtactgtttacccagggttgatgataaccgctggccttattcattggattttaaatacattgaacataactgttcacataagagacgtatgtgtgttccttgcaccaacttttagcggccttacatctatatctactttcctgcttacaagagaactttggaaccaaggagcaggacttttagctgcttgttttattgctattgtaccaggctacatatctcggtcagtagctggatcctttgataatgaaggcattgctatttttgcacttcagttcacatactatttatgggtaaaatctgtaaaaactgggtcagttttttggacaatgtgctgctgcttatcctatttctatatggtctctgcttggggtggttatgtatttatcatcaatcttattccactgcatgtatttgtgttgttactgatgcagagatacagcaaaagagtctacatagcatatagcactttctacattgtgggtttaatattatcaatgcagataccttttgtgggattccagccaatcagaacaagtgaacacatggcagctgcaggtgtctttgcattgctgcaagcttatgctttcttgcagtatctgagagaccgattaacaaaacaagagttccagacccttttctttttgggtgtatcactagctgcaggtgctgtgttccttagtgtcatctatttgacttatacaggttacattgcaccatggagtggcaggttttattcattgtgggatactgggtatgcaaaaatacacattccaattattgcatcagtgtctgagcatcaacctacgacttgggtgtctttcttctttgatctacatattcttgtatgtaccttcccagcaggcctttggttctgcatcaaaaatatcaacgatgaaagantatttggtaagaraggtttttaatgactactttgatatggaatagttatttttctttttgagattatttactttaaatttttgttttnctatgtttgactctatatattcaagataaattttctcctttattttgcataggtgcttaaccaagaaaaattcactgagaggctgggcatggtggcacacgcctktaatcccagcactttgggaggccgaggcgggcggatcacctgaggtcaggagttcgagaccagcctggccaacatggtgaaaccttgtctctactaaaaatacaaaaattagccgaacatggtggtgcatgcctgtaatcccagctactcaggaggctgaggcaggataattgcttgaacctgggaggcggaggttgcagtgagtcaagatcaagccactgcactccaccctgggaatcagagcgggactctgtctcaaaaaaaaaaaaaaaaaaaaaaaaaaaagggcggccgc

SEQ ID NO: 134 - DNA, 2157 bp, Homo sapiens
  SITE (309): n equals a,t,g, or c
caaaaaggaccgcccattgaagatgccattgcttcttccgatgttcttgagactgcttctaaatctgctaatccaccccacacgattcaagcatcagaagagcagagttcaaccccagcaccggtgaaaaagtctggcaagctgaggcagcaaatagatgtgaaggcggaactggagaagcggcaaggagggaagcagctactcaacttagtggtcattggtcatgttgatgctgggaaaagtactctgatgggccatatgctttatcttctgggtaatataaacaaaagaactatgcataagtatgancaggagtctaaaaaggctggcaaagcttcgtttgcatatgcatgggtcttggatgaaactggcgaagaaagggaaaggggagtaaccatggatgttggtatgacaaagtttgaaaccacaaccaaagttattacattaatggatgctccaggccataaggacttcattccaaatatgattacaggagcagcccaggcggatgtagctgttttagttgtagatgccagcaggggagagtttgaagctggatttgagactggaggacaaacacgagagcatggactcttggtccgttctctgggagtgacgcagcttgcagttgcagttaataaaatggatcaggttaattggcaacaagaaaggtttcaagagattactggaaaacttgggcactttcttaagcaagcaggttttaaggagagtgatgtaggttttattcctacaagtggtctcagtggtgaaaatctaatcacaagatctcagtcaagtgaactcacaaaatggtataaaggactatgtttattagaacaaattgattcctttaagcctccccagcgatctattgacaaaccttttagattatgtgtgtccgatgttttcaaagatcaaggatctggattttgcataactggtaaaatagaagctggttatatccaaactggtgaccgactactggcaatgcctcctaatgaaacttgtaccgtgaaaggaatcactctgcatgatgaacctgtcgactgggcggcagcaggcgatcatgttagtcttactttggttgggatggatatcatcaaaatcaatgttggctgcatattttgtggccccaaagtacccattaaagcttgcactcgtttcagagcccgaatcctcatctttaatattgaaattcctatcactaaaggatttcctgtgctgttacactaccaaactgtcagtgaacccgccgttattaaacgattgattagtgtcttaaacaaaagcacgggtgaagtcacaaagaaaaagcctaagtttttgactaaaggccagaatgcattggtagagctacagacacaaagaccaatagctcttgagctatataaagactttaaagagctggggaggttcatgctacgttacggtggttctacaatagctgctggtgttgtcactgagataaaagaatgatgggtcmgaatttctaccacgtttctggatacagtgaaatagctaacctctgtytcaagaatgcagttattaagtcaaaggaacaatgtgcaattgatatgtttttagatgagagagaaaaattaaagctaaaattagctgcaaagaagtattaataatcacctctgcaaaaattctaagttgccaactggcaaagraagtctaatgttaaaaacaactttgcctttgaamcgttaataaatggatttactttgctaagatttatggcaagtgtcaaaaatagtatctgaagatactgaatcatcatgaaatgaactctacttctggccaaagcacaatgtatttgcagttttctcttttgattcaattatactgcacatgttttaaggaaaagtaacttaattgggtttttcaggcagttgatatttgacctaagctttttttttttttttttttccagttaatgctaagaaaagatttggggaaggttataataaaagtattttgtggtgaccataagaatgtccctccccaaacaagtaaacttgtgaaagtttaatttggaattagtggaagctgttcctttgaaagccaagatattatttaagttgtaaagccagctaataaaatgccttagtttgagcataaaaaaaaaaaaaaaaaaaaaaaaaaaaactcgag

SEQ ID NO: 135 - DNA, 420 bp, Homo sapiens
ggcacgagagagagcagagctatacatagctatccaggtctaacttcacgaagaatagaatggtttcttttcattttcaatgtacatcatactttgtcagacttttttttcagttgcagctcttcgttggactggtgatagtattggctttattaatctctcattctctcacttattcattccacaaacatttgtagaaggccaccaagctctagggagaggaaaatggttttataaattagtgctttctgggataaaggaaatttataatctgtactacttaatagtagccactagccacatgtggttttcgaacaagatttccatcacctctccaaccactttctcctcattggtcagatctagaccccgagaaactgttcctttcattgttttctccgccttctacaaactgagata

SEQ ID NO: 136 - DNA, 946 bp, Homo sapiens
ggcacgagtgagattgcatccagacagagttttaaaagtttcccggttgagtttaatgtacagttgaagttgagacatgaatctctgcatgtaggggaaattttgtgtctggttagtcaagaaactatggaaaccaattcttgatattttgaaccattcacgaagatagtttgagtcatgagcatgctgttgtctagagtgggcggggatgactcattggagtggatgcgctgctctgtacttgatttttttgagtctgaaattagctttccaggctggggcagggaggggagcacaggtgggatcagtactgcccccaagcggtggagctgtggtggtggatcaatactgctgccgcctgtctgcacaaacatatttctctcttccagcccttcagaagtgtattggaatatgtcgataacaataatgatggtagtgaagatgatgatgatgtgggtaattctggctaccttattgggtccaagctccccacaattcgttgcacaaagcactctacatacattctctttagtcctgatcaaaccacctttcagagtaggatttagtgtcctattttaaagatgaaggagctcgggctcagagagagatcgtttagacacacacacaactttggaatgaaacatttacagccgggcgcggtggcgcgtgcctgtagtcccagctacttgggaggctgaggctggaggatcgcttgagtccaggagttctgggctgtagtgcgctatgccgatcgggtgtccgcactaagtttggcatcaatatggtgacctcccgggagtggaggaccaccaggttgcctaaggaggggtgaaccggtccaggtcggaatgaaacatttacaaaaattgacatttccttatgcatagatatttcactaggtccttaaaacccacgtgaatctgtgattaaaaaaaaaaaaaaaaaa

SEQ ID NO: 137 - DNA, 1258 bp, Homo sapiens
aaccctcactaaagggaacaaaagctggagctccaccgcggtggcggccgctggctgaccggcctaaaactaaaatgacatttattccctagctacaaacatcagcgttattatgttaattataccttgccctctatcattataaatggttgccatggtgtttctaaaaataagtgttttaccattaatgtgtagagggcaaacaaagcataaagtactaagggatcatgcttatcctagggtctcacagaagagaggacatatttaattaatcttgtgaattacagaacaggttgtggtccagacaccaagaatcataggggtttttttttaaaaaacctaatagaagtagggtgacctctctctttggtctaagagttctaaaggaaggtaggcatctgtttaattagttggttcaccctggctttacctctggttaatgcttgtgttaataggaaggaaaaatcactttatcttttcttccaagcccctccctgcctgacttacccagactgggattaccagataccaggtgatttatgtggagatgatttttcacctttaaactctaagccaagtgtaagaaactcttgatagctatgtctattttatatcagtcactgagacttttttttaagtttttatttattattaagacaactttgccaaaaaagtcccctaagcacaactatttacatttctttatagcctcttctgatctctaacacatatgcagttttaactgttattttcatagtaactgatcttttgtctaaggatttttacctgaaagcacaatgtattgagtctcttgaaaatcatctttcagatctttttacagaatgaacttatgcactgctactgtagtattctcaaggaatatatgtaaacacaaatgtatgcctgaggttggtttttgcagaaaacagtctctgcttctaaaaacttctatgtctagtcttccataggaaatcctcactgtttaaccatgtgaggagcctaagtcattaaacggatcatgtctgtacattgtgtaatgaatgaaaagcacataaatgtaatctactttgaactttgtaaaaatgatgtgtggaggctattcttgtttctccatctcaagtcctgtgtgtgcacgtgtgtgcaagtgcacatgtgtgtgtgtaataacacattgtaaagaacagaaattactttaaaaaataaacagaaatggagacctgaaaaaaaaaaaaaaaaaaaaaaaaaaaaaaaaaaaaaa

SEQ ID NO: 138 - DNA, 1598 bp, Homo sapiens
  SITE (1067): n equals a,t,g, or c
aggaaagaacaaaggttatttcctggagaaaagacaatttattcaacaccaacragggactcatcatatgggcacaactctggtgtccttctatggagaaaacctcaagtaaagttttattctgcctttraaaatgcttccaaaagtagaccctgtccccacacaggtcaagactacagagaaggctttgtagaaatgtgtcacctatgtacacctgctacttacacatttcctcttttggaaaaatgagatacttagaataacargaaaattaagacatactggcctggtgccagcagatggcttttctatagacaaactaggttagtgtggaagatataggttaaaataaactatgctgttttatttatcttcccaacctgattggcagctagacttttttagggtctcatttaatggccctgtttttttcattattatatttaatgatagggcaggatttcgtatgcaagctcttgtttctcaggctgcctgcagaagaagtcgctataaattatctgttgtctacatggtacaaggcccattgactcatctgatgcttgttttgttaatttctttaatatttttatcacggggcagtgggagggcttgggcttttagccacagctgttttaagacttctgatctcctgccctgcaggaataggtgggaagtcattgaatttttacactatagtaatttgcattcccacataagtttgagtgttacgaaaacattcctttaaagggatctgtgctacacaaaatatgccaggacctcacagacaaagccattgctagaaatgtcattccaatgatcagatctggaaacaggctgccataaccacttttccttcttgtagactcagctcacctgtatatttaaactgttcttggcatcttgaaacacctatttctactcaggtactcattgtcctgttactgattcacctttctgatccttttcaaccagttttcccccaaggggggaaattttacttaacctctagtatttgaacaactcaatatttgaattgttgccccatttgcttttacctgtactgtatncntggtcatctcaaatggcgtctaaacccagctactttgcattccagaagtttccattccctccaattccacctaatttttcatctgtcctagttactggctctttcttcatgtcttatttctcttgctttgggagcttaaaagattttacaagacctaattttgggttccttccttggagccatagttaccctgccaagaagagtagaaaatgggttcaactcctgtttcgctccaccaacacctctgtgagtctcatcatcagctgagcgatgatgccttacaggttgcatagcactggaactttcctagagtaacggctctgctgccagggtttctctgggctcattcttccactgacttaattatgatctatgcctaacagagccccagtacaactattttgcagaatggctgttaccctagaattactatagcacatattgagatatagttgtactccctagtagataggaactgaccccaacaataaactttgataataaaganaaaaaaaaaaaaaactcgtag

SEQ ID NO: 139 - protein, 334 aa, Homo sapiens
MFQCGLLQQLCTILMATGVPADILTETINTVSEVIRGCQVNQDYFASVNAPSNPPRPAIVVLLMSMVNERQPFVLRCAVLYCFQCFLYKNQKGQGEIVSTLLPSTIDATGNSVSAGQLLCGGLFSTDSLSNWCAAVALAHALQENATQKEQLLRVQLATSIGNPPVSLLQQCTNILSQGSKIQTRVGLLMLLCTWLSNCPIAVTHFLHNSANVPFLTGQIAENLGEEEQLVQGLCALLLGISIYFNDNSLESYMKEKLKQLIEKRIGKENFIEKLGFISKHELYSRASQKPQPNFPSPEYMIFDHEFTKLVKELEGVITKAIYKSSEEDKKKKK

SEQ ID NO: 140 - protein, 43 aa, Homo sapiens
  SITE (43): Xaa equals stop translation
MTVASIRHILVEIWLPIALAMGTRGLTQIVAVIQSRSQWALSX

SEQ ID NO: 141 - protein, 87 aa, Homo sapiens
  SITE (87): Xaa equals stop translation
MLFIFLLLILSITASYSLTCILSGAGEPSSVSASVVSGPGFCLAALLLMRTGGFAATLLPVAPTERFFSCCTVLSAQRNVSRTRSPX

SEQ ID NO: 142 - protein, 122 aa, Homo sapiens
  SITE (122): Xaa equals stop translation
MLSTRWMGLHLVQILWRCWTSSATITSRKLSTALRSPVLSGTQTSRSSGDSGWSMKTSVKATPHQMSLRSGKETPSADIPRIHHQLVRLRHQAHGGWSPHGVPEQGTMPLVLPPVSCDIQPX

SEQ ID NO: 143 - protein, 276 aa, Homo sapiens
  SITE (131): Xaa equals any of the naturally occurring L-amino acids
MANTGVFGFSFLLLTVALLASYSVHLLLSMCIQTAVTSYEDLGLFAFGLPGKLVVAGTIIIQNIGAMSSYLLIIKTELPAAIAEFLTGDYSRYWYLDGQTLLIIICVGIVFPLALLPKIGFLGYTSSLSFXFMMFFALVVIIKKWSIPCPLTLNYVEKGFQISNVTDDCKPKLFHFSKESAYALPTMAFSFLCHTSILPIYCELQSPSKKRMQNVTNTAIALSFLIYFISALFGYLTFYGSHSVAQVGVQWCDLSSLQPLPPGLKQSSHLSLQSSX

SEQ ID NO: 144 - protein, 195 aa, Homo sapiens
  SITE (138): Xaa equals any of the naturally occurring L-amino acids
MKLASGFLVLWLSLGGGLAQSDTSPDTEESYSDWGLRHLRGSFESVNSYFDSFLELLGGKNGVCQYRCRYGKAPMPRPGYKPQEPNGCGSYFLGLKVPESMDLGIPAMTKCCNQLDVCYDTCGANKYRCDAKFRWCLXSICSDLKRSLGFVSKVEACDSLVDTVFNTVWTLGCRPFMNSQRAACICAEEEKEELX

SEQ ID NO: 145 - protein, 183 aa, Homo sapiens
  SITE (183): Xaa equals stop translation
MLLLCHALAIAVVQIVIFSESWAFAKNINFYNVRPPLDPTPFPNSFKCFTCENAGDNYNCNRWAEDKWCPQNTQYCLTVHHFTSHGRSTSITKKCASRSECHFVGCHHSRDSEHTECRSCCEGMICNVELPTNHTNAVFAVMHAQRTSGSSAPTLYLTSACLGLCASIAVMPPFLGEAETSLX

SEQ ID NO: 146 - protein, 122 aa, Homo sapiens
  SITE (122): Xaa equals stop translation
MLRGTMTAWRGMRPEVTLACLLLATAGCFADLNEVPQVTVQPASTVQKPGGTVILGCVVEPPRMNVTWRLNGKELNGSDDALGVLITHGTLVITALNNHTVGRYQCVARMPAGAVATCQPLX

SEQ ID NO: 147 - protein, 267 aa, Homo sapiens
  SITE (267): Xaa equals stop translation
MWWFQQGLSFLPSALVIWTSAAFIFSYITAVTLHHIDPALPYISDTGTVAPEKCLFGAMLNIAAVLCIATIYVRYKQVHALSPEENVIIKLNKAGLVLGILSCLGLSIVANFQKTTLFAAHVSGAVLTFGMGSLYMFVQTILSYQMQPKIHGKQVFWIRLLLVIWCGVSALSMLTCSSVLHSGNFGTDLEQKLHWNPEDKGYVLHMITTAAEWSMSFSFFGFFLTYIRDFQKISLRVEANLHGLTLYDTAPCPINNERTRLLSRDIX

SEQ ID NO: 148 - protein, 92 aa, Homo sapiens
  SITE (92): Xaa equals stop translation
MLCHPHVHHHLVCLLATLTFSLNASCAEQTFHSQQSNGEFMATLPSISKQFGVIVWKPQRKDVIRLPVALSFSMGLGLLSPALGRFLASELX

SEQ ID NO: 149 - protein, 109 aa, Homo sapiens
  SITE (109): Xaa equals stop translation
MAILLACFTAVLAFICLQFWCVRCHEPRWSYRAGHMEEANGLVRWPEEAPDLGQREEDLQGLPLVEMPRKNSRDGAELDPEANQDAPDAGALQRGGGDPPAILPHCGEX

SEQ ID NO: 150 - protein, 88 aa, Homo sapiens
  SITE (88): Xaa equals stop translation
MLLRVFHFFLHILHKKQTGVSLLYLLLTLFLLQQQVIPQPSLPLLHLVSFQICHYPFPQWMLQYRQAKMVLGTRCQMSLMHFQNSQNX

SEQ ID NO: 151 - protein, 74 aa, Homo sapiens
  SITE (74): Xaa equals stop translation
MSRVVSLFFFILFSFFFFAFSLSSSLSFVHYEKLVQVKECLDSFLKKIKIKEYKTRQCYHLIRWENNGAKLQSX

SEQ ID NO: 152 - protein, 72 aa, Homo sapiens
  SITE (72): Xaa equals stop translation
MSASLKNHLTHCFLLLLLKELVSPTMISFVPTLRHSYRFFNLFSCDAESTKESPGRTVQFSKTPRGVTMFIX

SEQ ID NO: 153 - protein, 152 aa, Homo sapiens
  SITE (152): Xaa equals stop translation
MKYGLTGPWIKRLLPVIFLVQASGMNVYMSRSLESIKGIAVMDKEGNVLGHSRIAGTKAVRETLASRIVLFGTSALIPEVFTYFFKRTQYFRKNPGSLWILKLSCTVLAMGLMVPFSFSIFPQIGQIQYCSLEEKIQSPTEETEIFYHRGVX

SEQ ID NO: 154 - protein, 61 aa, Homo sapiens
  SITE (61): Xaa equals stop translation
MLRVAGVLQFLPLSYGTKVASLWNTYENVVMPPSFTTTLVLPLLSHEFYNYSYPFACDQKX

SEQ ID NO: 155 - protein, 123 aa, Homo sapiens
  SITE (89): Xaa equals any of the naturally occurring L-amino acids
MHRSEPFLKMSLLILLFLGLAEACTPREVNLLKGIIGLMSRLSPDEILGLLSLQVLHEETSGCKEEVKPFSGTTPSRKPLPKREEHVEXPXNAXTWXXTYLFVSYNKGDWFTFSSQVLLPLLX

SEQ ID NO: 156 - protein, 55 aa, Homo sapiens
  SITE (55): Xaa equals stop translation
MSPCAHICLYVLVFLCNVTRCKCVRAFTTWDTEKVKYFMAHWSKLKRVRGTRVEX

SEQ ID NO: 157 - protein, 111 aa, Homo sapiens
  SITE (93): Xaa equals any of the naturally occurring L-amino acids
MFLASWLLFCIVAPKDDAHLSFIQCKDIWKDNRKYSCFHFKSDQLLELASKACTSFQAQSRSFTAGAVPSEHPELPCGSQQLCCGCTARLGGXWIGASRCGSGSAFLASPX

SEQ ID NO: 158 - protein, 48 aa, Homo sapiens
  SITE (48): Xaa equals stop translation
MSLQAIDLLWSLCTQTSLLTLICICSHSQALSSSPQLHLRSSSIRFSX

SEQ ID NO: 159 - protein, 82 aa, Homo sapiens
  SITE (82): Xaa equals stop translation
MFHFGLWDLHFFLIVMAHRDDCSFKGGCGLLERFQCPHTSFSSASQKRLADGMECLCEIERTQTRIRKICLPTLHGHLLAVX

SEQ ID NO: 160 - protein, 156 aa, Homo sapiens
  SITE (108): Xaa equals any of the naturally occurring L-amino acids
MMARQTGVFYLTLVLILVTSGLFFAFDCPYLAVKITPAIPAVAGILFFFVMGTLLRTSFSDPGVLPRATPDEAADLERQIGNTESLPMASGHFPPGPSYSGEGRPRAXQEELXAGKEGGQKSAFLSSLGGQDELKKRCDIRLEGQVSWRQDCRPTX

SEQ ID NO: 161 - protein, 295 aa, Homo sapiens
  SITE (295): Xaa equals stop translation
MRLDKPIGTWLLYLPCTWSIGLAAEPGCFPDWYMLSLFGTGAILMRGAGCTINDMWDQDYDKKVTRTANRPIAAGDISTFQSFVFLGGQLTLALGVLLCLNYYSIALGAGSLLLVITYPLMKRISYWPQLALGLTFNWGALLGWSAIKGSCDPSVCLPLYFSGVMWTLIYDTIYAHQDKRDDVLIGLKSTALRFGENTKPWLSGFSVAMLGALSLVGVNSGQTAPYYAALGAVGAHLTHQIYTLDIHRPEDCWNKFISNRTLGLIVFLGIVLGNLWKEKKTDKTKKGIENKIENX

SEQ ID NO: 162 - protein, 60 aa, Homo sapiens
  SITE (60): Xaa equals stop translation
MGPFLLVFLFPILRVCGIIREPTQDWSVLLERARLTAPGQPPALFPLESGPMATAQNTSX

SEQ ID NO: 163 - protein, 122 aa, Homo sapiens
  SITE (30): Xaa equals any of the naturally occurring L-amino acids
MCSHSTLIHLYLVLPFFFLFLPSSFPFPSXSXSSILPSLRLPPFFPPSLFLHSSLPPSLSHPLGLSITSSRQSFLDYHHLCTKHLSXTLCGLIYHCLNIFXTRAVMWHMQVSFLXIHWLLPX

SEQ ID NO: 164 - protein, 73 aa, Homo sapiens
  SITE (73): Xaa equals stop translation
MSIYHVCLILLLYITSHSHQNMSSCLQVPLSLLSCPLKGEHLSQFAGDHSLPEVRDRNHHCILFKESHQKRKX

SEQ ID NO: 165 - protein, 123 aa, Homo sapiens
  SITE (123): Xaa equals stop translation
MLANFTLFILTLISFLLLVCSPCKHLKMMQLHGKGSQDLSTKVHIKPLQTVISFLMLFAIYFLCIITSTWNPRTQQSNLVFLLYQTLAIMYPSFHSFILIMRSRKLKQTSLSVLCQVTCWVKX

SEQ ID NO: 166 - protein, 143 aa, Homo sapiens
  SITE (143): Xaa equals stop translation
MPGPCLSQQHPFLSLSLFPFCLWICLARVPGVRNICKTQPAPSQPSLLGLGLSHPAAGTTDAGTQSLPRSQHKCTSALWGLCPAQRPLLLPAHIHSSGHGAPQELQSHLSHRLPASASLSMMSPFSEAWTHPSLSLGPAPSHX

SEQ ID NO: 167 - protein, 117 aa, Homo sapiens
  SITE (46): Xaa equals any of the naturally occurring L-amino acids
MPGGTRCRVLLLSLTFGTSMACGNVGLRLCPWTWHNWLLPPHLCSXWPCRRCCWAAATTHFSWPPWVRSAWGPPAAWLESSGHPLPAVASCSQPPASADSSRFSKVPCCRRRGWTRX

SEQ ID NO: 168 - protein, 59 aa, Homo sapiens
  SITE (59): Xaa equals stop translation
MSVCLPLHLPFLMLAKVATSFCRWQLTLFVSTFYKDALVHTVNDRNQEAELEALKKSCX

SEQ ID NO: 169 - protein, 126 aa, Homo sapiens
  SITE (126): Xaa equals stop translation
MKALMLLTLSVLLCWVSADIRCHSCYKVPVLGCVDRQSCRLEPGQQCLTTHAYLGKMWVFSNLRCGTPEEPCQEAFNQTNRKLGLTYNTTCCNKDNCNSAGPRPTPALGLVFLTSLAGLGLWLLHX

SEQ ID NO: 170 - protein, 87 aa, Homo sapiens
  SITE (87): Xaa equals stop translation
MFLVAVWWRFGILSICMLCVGLVLGFLISSVTFFTPLGNLKIFHDDGVFWVTFSCIAILIPVVFMGCLRILNILTCGSHWAPIRWFX

SEQ ID NO: 171 - protein, 63 aa, Homo sapiens
  SITE (16): Xaa equals any of the naturally occurring L-amino acids
MVTGFFFILMTVLWFXREPGFVPGWDSFFEKKGYRTDATVSVFLGFLLFLIPAXEALLWEKEX

SEQ ID NO: 172 - protein, 48 aa, Homo sapiens
  SITE (48): Xaa equals stop translation
MSQLCFSLLLSSTCHGGVASLLTSDLSSQSHRFSICTNVNHSKYSSLX

SEQ ID NO: 173 - protein, 137 aa, Homo sapiens
  SITE (84): Xaa equals any of the naturally occurring L-amino acids
MLFSLRELVQWLGFATFEIFVHLLALLVFSVLLALRVDGLVPGLSWWNVFVPFFAADGLSTYFTTIVSVRLFQDGEKRLAVLRXFWVLTVLSLKFVFEMLLCQKLAEQTRELWFGLITSPLFILLQLLMIRACRVNX

SEQ ID NO: 174 - protein, 89 aa, Homo sapiens
  SITE (40): Xaa equals any of the naturally occurring L-amino acids
MELSFVRRLLLFTFFFSTFSPPPPTPCLEGLMSCLPSPLXKNTAGSQTKSLREIGTGISDTHVSPSPAQAPLCSRSPTWDSSDPNSMDX

SEQ ID NO: 175 - protein, 58 aa, Homo sapiens
  SITE (58): Xaa equals stop translation
MTMVMEQVYLMSFLLLLLRTMMKAHWTYTLGWTVLFLTALPNPVYHQEIVWTYMKRSX

SEQ ID NO: 176 - protein, 64 aa, Homo sapiens
  SITE (64): Xaa equals stop translation
MDTDNGGRHFKPFKLVLFVVLLIKILLILAKTNCCDKLVFFGCFKHTLTNFLIPLLVPPIVLKX

SEQ ID NO: 177 - protein, 61 aa, Homo sapiens
  SITE (61): Xaa equals stop translation
MCLWGQANLGLILFQHCLTKFMGGYCFGLGSCTRPLRDQTKMESLILKLQVTEPKLSCFIX

SEQ ID NO: 178 - protein, 104 aa, Homo sapiens
  SITE (104): Xaa equals stop translation
MGMAGALSILLFSLPSHGWPSPPKPPFPCCQPLCHSLILGRRKGRFEGEGEKAYGWVWFLPFPEGLTVPGWPQGRQGPHYACALVKVTPAIYQQPWHVPAPQEX

SEQ ID NO: 179 - protein, 293 aa, Homo sapiens
  SITE (293): Xaa equals stop translation
MLRVLCLLRPWRPLRARGCASDGAAGGSEIQVRALAGPDQGITEILMNRPSARNALGNVFVSELLETLAQLREDRQVRVLLFRSGVKGVFCAGADLKEREQMSEAEVGVFVQRLRGLMNDIAAFPAPTIAAMDGFALGGGLELALACDLRVAASSAVMGLIETTRGLLPGAGGTQRLPRCLGVALAKELIFTGRRLSGTEAHVLGLVNHAVAQNEEGDAAYQRARALAQEILPQAPIAVRLGKVAIDRGTEVDIASGMAIEGMCYAQNIPTRDRLEGMAAFREKRTPKFVGKX

SEQ ID NO: 180 - protein, 46 aa, Homo sapiens
  SITE (46): Xaa equals stop translation
MLSSLYLLLMPPYKFTGELHPPVAATCLLTVLLGCLIGVSSDGWIX

SEQ ID NO: 181 - protein, 47 aa, Homo sapiens
  SITE (47): Xaa equals stop translation
MCIPEALGKNSLFLSSTFLWLLAFFGLWSHHSYLEGQHLQICFFFTX

SEQ ID NO: 182 - protein, 55 aa, Homo sapiens
  SITE (55): Xaa equals stop translation
MTTSLFGLVCVVCQGAGVSAFTQVNLFSFSLVIVKKQNKTSCEPFGTSGKVPLLX

SEQ ID NO: 183 - protein, 67 aa, Homo sapiens
  SITE (67): Xaa equals stop translation
MLIYWLQSSFILSAFVLINSPVTTGIQKSCCKFFPVSINLCFASLHRMKVVTLVALQWLNIALRSSX

SEQ ID NO: 184 - protein, 51 aa, Homo sapiens
  SITE (51): Xaa equals stop translation
MVCCGFFLLWSRVRSYMKLSGHRWSSSCPHHCYSKCGLHTSNGKSSVHTVX

SEQ ID NO: 185 - protein, 91 aa, Homo sapiens
  SITE (29): Xaa equals any of the naturally occurring L-amino acids
MLRCSFSSFLLCHTILLFLGSSAHLLVEXXVWGLYEYRIGDMVDQKATFCVQKQECLFPLGSWVXRVEGGAFAREPPSSTQYFPVSCLYQX

SEQ ID NO: 186 - protein, 55 aa, Homo sapiens
  SITE (55): Xaa equals stop translation
MSALLSHHVPLFYLTGCLFSLLASWDCNGKEGAGRAIKGKNNTWNCMILSKVKFX

SEQ ID NO: 187 - protein, 64 aa, Homo sapiens
  SITE (26): Xaa equals any of the naturally occurring L-amino acids
MVHKAILALLPWGFSADELLASLMMXLTEKYQNCSSTTDIXNQQLRSLGQNFMFQQNLQLILMX

SEQ ID NO: 188 - protein, 113 aa, Homo sapiens
  SITE (113): Xaa equals stop translation
MMTSSLGLSFLLNLILGMKFTYLIPQNKYIQLFTTILSFFSGVLSLLECKLSTSSCTCLNIHKSDNECKESENSIEDISLPERTAMPRSIVRAHTVNSLNKKVQTRHVTWALX

SEQ ID NO: 189 - protein, 60 aa, Homo sapiens
  SITE (60): Xaa equals stop translation
MLHLTLYLHFILFVFPITSNFSSLHPFLFISSQFTSCCQINFPNAQALSYHEFLIATYDX

SEQ ID NO: 190 - protein, 64 aa, Homo sapiens
  SITE (64): Xaa equals stop translation
MPCIRGVFHCFILIILILLASHAFSGSGNQRLKEALTLIVSVNVDIARHRPFLERIHVKKGNTX

SEQ ID NO: 191 - protein, 71 aa, Homo sapiens
  SITE (71): Xaa equals stop translation
MFSRLHFLTHSLSLLHLPSQVFGEVHSSCVSSLPCPDTPALPYCPSFLRYDDHIEAQPLKHINTNDHISIX

SEQ ID NO: 192 - protein, 174 aa, Homo sapiens
MYVRFFFRLHSISSHPSGIVSLCLLFETLLQTYLPQLFYHLREIGAQPLRISFKWMVRAFSGYLATDQLLLLWDRILGYNSLEILAVLAAAVFAFRAVNLMEVTSLAAAENLAAHSEQFCTAPLFPELYRVQIPVLLNSGRKKSAVYWTPISFNRTKKLRLQGRTYNDGSWNIT

SEQ ID NO: 193 - protein, 193 aa, Homo sapiens
  SITE (193): Xaa equals stop translation
MEALLQSLVIVLLGFKSFLSEELGSEVLNLLTNKQYELLSKNLRKTRELFVHGLPGSGKTILALRIMEKIRNVFHCEPANILYICENQPLKKLVSFSKKNICQPVTRKTFMKNNFEHIQHIIIDDAQNFRTEDGDWYGKAKFITQTARDGPGVLWIFLDYFQTYHLSCSASPLPQTSIQEKRSTEWSAMQVQX

SEQ ID NO: 194 - protein, 112 aa, Homo sapiens
  SITE (112): Xaa equals stop translation
MQFSLCLTAVFLLQLAAGILGFVFSDKARGKVSEIINNAIVHYRDDLDLQNLIDFGQKKVWVSQWSGGLWVKVNVIPRDASPSMPVGLFITCQVMASGKGFGKKSTRSRVLX

SEQ ID NO: 195 - protein, 80 aa, Homo sapiens
  SITE (80): Xaa equals stop translation
MCRPLLPLLFPWGHCLSIPLCKWPQIMSQPPRLHRLLASGPSTKKHSKLQTHSWENSNGLTLPFEPARSHGLWRAAFESX

SEQ ID NO: 196 - protein, 88 aa, Homo sapiens
  SITE (88): Xaa equals stop translation
MLSIIDLLFLLSPTFGLITELLFSPEVPKALSCPLKALGGGSHSHEPLGMFAPVPPGCESSTPFPKGLGASKILTLGAQAEFRRRSHX

SEQ ID NO: 197 - protein, 42 aa, Homo sapiens
  SITE (42): Xaa equals stop translation
MEDHFLIGHFPFFFLFSFPCFCTKPLCREYFLICSIQDESKX

SEQ ID NO: 198 - protein, 69 aa, Homo sapiens
  SITE (69): Xaa equals stop translation
MFNLPKPVFLSWWRWKTIVIFLACLASAAIKETAVSMKTVFPIFVQITLILLLESRVLKIGDFSNFFCX

SEQ ID NO: 199 - protein, 153 aa, Homo sapiens
  SITE (66): Xaa equals any of the naturally occurring L-amino acids
MDHSPTTGVVTVIVILIAIAALGAFDPGLLVLPAAAAHQPVRGRGEHRGGWGDQGTLPAGAVFGQXTVRGEKGQADXSQTXRKXTXXPGCKGXLVPVCKPAKXGLGGAKXIRMRCCLRGRADTCWHGLCGFRPSHALMPGDLAVLGFPSASRX

SEQ ID NO: 200 - protein, 63 aa, Homo sapiens
  SITE (63): Xaa equals stop translation
MKNSTSLLYKLFSSLSVFIFKFLLLFYTLHIALGVKIQYKPLAHFIDHSCIQQVSQVQWSIPX

SEQ ID NO: 201 - protein, 64 aa, Homo sapiens
  SITE (64): Xaa equals stop translation
MQEPHGKFLSWGRWLWWWSLAAPALVQAVNMPPAYIQIENWYMMLLMGWETKCCHVRSLWVGTX

SEQ ID NO: 202 - protein, 43 aa, Homo sapiens
  SITE (43): Xaa equals stop translation
MLINCIFSLLLLLSHADGMHLFISSGDRILFCLYFLHSRVCAX

SEQ ID NO: 203 - protein, 41 aa, Homo sapiens
  SITE (41): Xaa equals stop translation
MSVYVNIMHIVIYIYLCVYMCVAQSHTHTQICIQMLPGLQX

SEQ ID NO: 204 - protein, 44 aa, Homo sapiens
  SITE (44): Xaa equals stop translation
MILSFLMLFLIVKTIPLILAYCYNSISFFSNNLVLVKMGYNNKX

SEQ ID NO: 205 - protein, 42 aa, Homo sapiens
  SITE (42): Xaa equals stop translation
MRLLSTLLSFYPFSNCFLLSFCDSHPPVWLRNSQVFPEEVVX

SEQ ID NO: 206 - protein, 42 aa, Homo sapiens
  SITE (42): Xaa equals stop translation
MTGKLWLLLPRLGHAAAAPTTALSGSELEGTSISLLIALDRX

SEQ ID NO: 207 - protein, 113 aa, Homo sapiens
  SITE (17): Xaa equals any of the naturally occurring L-amino acids
MAPWLPLLSLLGLLLGXAPAPPRRAADAQAREAAYPELLGPARFALEMYNRGRAAGXRATLGAVRGRVRRAGEGSLYSLRATLEEPPCNXXTVCQLPVSKRPCSAALKSWTSX

SEQ ID NO: 208 - protein, 45 aa, Homo sapiens
  SITE (45): Xaa equals stop translation
MPTWPLLQLLSCSFPSLLCETFTFCSKDEVSRWKAGCFVPLPASX

SEQ ID NO: 209 - protein, 123 aa, Homo sapiens
  SITE (71): Xaa equals any of the naturally occurring L-amino acids
MTHWSGCAALYLIFLSLKLAFQAGAGRGAQVGSVLPPSGGAVVVDQILLPPVCTNIFLSSSPSEVYWNMSXTIMMVVKMMMMWVILATLLGPSSPQFVAQSTLHTFSLVLIKPPFRVGFSVLF

SEQ ID NO: 210 - protein, 42 aa, Homo sapiens
  SITE (42): Xaa equals stop translation
MINFWPVTHVCIWLLWLQALEARGQGSNIDCTRNSKTVFTSX

SEQ ID NO: 211 - protein, 51 aa, Homo sapiens
  SITE (51): Xaa equals stop translation
MYIYLIHLCMCVYIYIYILLIIYTLDPEPPSWSPKLDSHLSLRQPSNDRFX

SEQ ID NO: 212 - protein, 65 aa, Homo sapiens
  SITE (65): Xaa equals stop translation
MFVLCTRAVRTRLFSLCCCCCSSQPPTKSPAGTPKAPAPSKPGESQESQGTPGELPSTWSFCPFX

SEQ ID NO: 213 - protein, 77 aa, Homo sapiens
  SITE (77): Xaa equals stop translation
MLALLVGGLVAALACHGILAAILAVCGELVSGKGTRSSDEDDGGDGDRGHRGLSLLNSAFGHMGDGDRKDDNSGTLX

SEQ ID NO: 214 - protein, 45 aa, Homo sapiens
  SITE (45): Xaa equals stop translation
MFVGTRVLLVPLPFFSISGMLAIDKYLHKKLLLNEIITTSTWALX

SEQ ID NO: 215 - protein, 66 aa, Homo sapiens
  SITE (27): Xaa equals any of the naturally occurring L-amino acids
MGKGHQRPWWKVLPLSCFLVALIIWCXLREESEADQWLRQVWGEVPEPSDRSEEPETPAAYRARTX

SEQ ID NO: 216 - protein, 62 aa, Homo sapiens
  SITE (62): Xaa equals stop translation
MRLCTTWMAVKFLWWGMTWIPSGKACSWTQPLCSSGGWSSPTHLPTSLLLGWRASLCMKRSX

SEQ ID NO: 217 - protein, 56 aa, Homo sapiens
  SITE (56): Xaa equals stop translation
MFASYHIQFFTWLIQKLSLVWKSVVAIREQGKELVWKQHLPLRSYSPNNAKSLGLX

SEQ ID NO: 218 - protein, 213 aa, Homo sapiens
  SITE (88): Xaa equals any of the naturally occurring L-amino acids
MLSFNFTWMVWVSLVLKSQRAKLALHSLHLHQEVRLRMSRRESPGRPLRCGVRGNMGARTPVPTADYPSPYRTLPRMAAPPPQKSSCXRLHRPHWWRPRTPSSEKTGGQSQSTLDRCAHLVHMLLRDQRATSQWKAGGRLCRALSKTPLQHQLHSTSYRKALPILRPSSRREAGPLHHIDLRRCFSRLGRGADFAVCAKEPVSDNPIFLLITX

SEQ ID NO: 219 - protein, 41 aa, Homo sapiens
  SITE (41): Xaa equals stop translation
MNMFQTILVCVLFVFVRWFFLLLQIESIQTKFHCISSQFWX

SEQ ID NO: 220 - protein, 60 aa, Homo sapiens
  SITE (60): Xaa equals stop translation
MELVWFRFLHLNLLPRGVCCGICVCVRRGMVLSEPTSCGQRALSCEGGCHSGRVQFRRPX

SEQ ID NO: 221 - protein, 59 aa, Homo sapiens
  SITE (59): Xaa equals stop translation
MRRMRMKSLSPRRSWWTLWLGQGVLGAALKANTLWIAMRRRMMMMGGPANMTSWPQRMX

SEQ ID NO: 222 - protein, 46 aa, Homo sapiens
  SITE (46): Xaa equals stop translation
MPFFLLTFPLVLYPHLSRGSDPVLPCVMGIHVFGLSHHSRKVAPPX

SEQ ID NO: 223 - protein, 62 aa, Homo sapiens
  SITE (62): Xaa equals stop translation
MDRVRFRSWLLYPCCVALGQELGLSAPQWLITENGMPALALVGCFEPTAGSGSSWHDVFLPX

SEQ ID NO: 224 - protein, 52 aa, Homo sapiens
  SITE (52): Xaa equals stop translation
MKLNVHFLWCTFIFQTSGSHIELLISGQVSSYIPSLDFCTHKVVSREKFEEX

SEQ ID NO: 225 - protein, 51 aa, Homo sapiens
  SITE (51): Xaa equals stop translation
MASPVFKTFWRLELSVPLSLLFILQIVTSLSSDEICYSTRKVFIIRRQLYX

SEQ ID NO: 226 - protein, 47 aa, Homo sapiens
  SITE (47): Xaa equals stop translation
MCMCVGVCLITLLDRFLWFGTAGAKFIQKSTFLSKLPMTLVSFHSIX

SEQ ID NO: 227 - protein, 52 aa, Homo sapiens
  SITE (52): Xaa equals stop translation
MCPFHKAYLDCFFQISLLLLIFLTYLDIGKCGLWSHEWRIRELGKHERWWNX

SEQ ID NO: 228 - protein, 66 aa, Homo sapiens
  SITE (61): Xaa equals any of the naturally occurring L-amino acids
MNQPILRSQALLWPWRWVVKAKPCVCVSMDAWIPDRSQHCPSIPGQKKERAGSHGHQALAXLLFLX

SEQ ID NO: 229 - protein, 47 aa, Homo sapiens
  SITE (47): Xaa equals stop translation
MASRGTAAPGRTFLAMMVTSFFFCMRWGSWAEQMPQRCLPCCMQECX

SEQ ID NO: 230 - protein, 222 aa, Homo sapiens
  SITE (184): Xaa equals any of the naturally occurring L-amino acids
MAGGVRPLRGLRALCRVLLFLSQFCILSGGESTEIPPYVMKCPSNGLCSRLPADCIDCTTNFSCTYGKPVTFDCAVKPSVTCVDQDFKSQKNFIINMTCRFCWQLPETDYECTNSTSCMTVSCPRQRYPANCTVRDHVHCLGNRTFPKMLYCNWTGGYKWSTALALSITLGGFGADRFYLGQWXEGLGKLFSFGGLGIWTLIDVLLIGVGYVGPADGSLYIX

SEQ ID NO: 231 - protein, 49 aa, Homo sapiens
  SITE (49): Xaa equals stop translation
MCIHYSRVIFSFLKLRIKSISWYAMWLYFFCYLNCLAKVRSATTYLYVX

SEQ ID NO: 232 - protein, 41 aa, Homo sapiens
  SITE (41): Xaa equals stop translation
MLPVCVFKLLLYLYVLIRICTIIWCFKVYINAVILNKSSRX

SEQ ID NO: 233 - protein, 53 aa, Homo sapiens
  SITE (53): Xaa equals stop translation
MNCGGSTLCVLSFCSVVCSVEASCQSTVQWGGAAARVGVPFDWSRNEQGKGHX

SEQ ID NO: 234 - protein, 50 aa, Homo sapiens
  SITE (45): Xaa equals any of the naturally occurring L-amino acids
MLGSIPKLWSVLSFSINFCFCCFILSLLCLSVLSNYLFKTPRTWXTLHRX

SEQ ID NO: 235 - protein, 45 aa, Homo sapiens
  SITE (16): Xaa equals any of the naturally occurring L-amino acids
MCLPLLHCTGALWGKXVLLFLYCLAQSFAYSRHQTVGLVVHDYWX

SEQ ID NO: 236 - protein, 55 aa, Homo sapiens
  SITE (55): Xaa equals stop translation
MCWICVWLFFSPTKTSCFPWLIRPGPRSFTDSHGTPPWQCLEPSSFTYPGKQVWX

SEQ ID NO: 237 - protein, 69 aa, Homo sapiens
  SITE (69): Xaa equals stop translation
MKRLRFVLRVFQMTAFITGAHTITNYSDRRLYISPLSHFFMNSGSSAQSVLSHSYVSQIFFKNVSKYFX

SEQ ID NO: 238 - protein, 40 aa, Homo sapiens
MVAMVFLKISVLPLMCRGQTKHKVLRDHAYPRVSQKRGHI

SEQ ID NO: 239 - protein, 45 aa, Homo sapiens
  SITE (34): Xaa equals any of the naturally occurring L-amino acids
MTKLLSLSHLLVTFFNIIAIKCKKQHLRHSKCNXDTTFKNKMLNX

SEQ ID NO: 240 - protein, 78 aa, Homo sapiens
  SITE (78): Xaa equals stop translation
MQLCVIWFTVIFLSQSSRLVKEKISNTSGEKGRWPAIDVVALCPSRTAGISFPRHFLYVSCIVGCTNIICSFGFPGQX

SEQ ID NO: 241 - protein, 53 aa, Homo sapiens
  SITE (53): Xaa equals stop translation
MEVVLPKHILDIWVIVLIILATIVIMTSLLLCPATAVIIYRMRTHPILSGAVX

SEQ ID NO: 242 - protein, 53 aa, Homo sapiens
  SITE (53): Xaa equals stop translation
MYYLGKWDIWQPVSLLYIILFAACPSLLISIPAKASGEGWRCGDIQLTVVTDX

SEQ ID NO: 243 - protein, 43 aa, Homo sapiens
  SITE (43): Xaa equals stop translation
MPVAFHLPFLLILPYRVLPVGQVTQLTPRAVEVKIHNHGRLPX

SEQ ID NO: 244 - protein, 49 aa, Homo sapiens
  SITE (49): Xaa equals stop translation
MSWPLCTLLFSWDCILAVKTSRLKFDSQGYILGTFKVSFQRDFINRLDX

SEQ ID NO: 245 - protein, 75 aa, Homo sapiens
  SITE (75): Xaa equals stop translation
MSIIIYWLLFFKHLLWVLIIGMVKALHPHYLNLRIYEFGEITAVLQRKKQGRENGNFLKFSLLSLNRSRIPTQIX

SEQ ID NO: 246 - protein, 44 aa, Homo sapiens
  SITE (44): Xaa equals stop translation
MAIHFHIIQWLLLCYNCHHAQWGLWHTTAEVSGCGRNHLAFKAX

SEQ ID NO: 247 - protein, 65 aa, Homo sapiens
  SITE (65): Xaa equals stop translation
MYLSLFFFCFSLQASAVEERSAESSREGPVRTDNWQRCFGDIPGTPTHLVQRSLVLTCFGRVLSX

SEQ ID NO: 248 - protein, 84 aa, Homo sapiens
  SITE (84): Xaa equals stop translation
MKKVCWVWALAHLVLCERWLTAGCLLYVGVIQPCKGSPSSVCKARRCLHPKYRIKRYGYYKYSVRLIICHHHPHALKAELTDDX

SEQ ID NO: 249 - protein, 71 aa, Homo sapiens
MVQGPLTHLMLVLLISLIFLSRGSGRAWAFSHSCFKTSDLLPCRNRWEVIEFLHYSNLHSHISLSVTKTFL

SEQ ID NO: 250 - protein, 57 aa, Homo sapiens
  SITE (57): Xaa equals stop translation
MRSYFPFSVCPFPFCSPVFFFVFTDVYLCFFFVFAVGRHLSDPFPILFFTHKCPDVX

SEQ ID NO: 251 - protein, 67 aa, Homo sapiens
  SITE (67): Xaa equals stop translation
MRACGWDLSILLVGLVMGREGCYSRLPPTEYQKQAGSSGVCKDVRPRNQPSPSYPCKSLSPHAPLLX

SEQ ID NO: 252 - protein, 46 aa, Homo sapiens
  SITE (46): Xaa equals stop translation
MYLILSWLFLCKLVKCYFEILLFSTSPQLLQWTVIVTYCGPLLRFX

SEQ ID NO: 253 - protein, 54 aa, Homo sapiens
  SITE (54): Xaa equals stop translation
MLVFLLLFSTVTVLCLKVVFSLKAVAYIVKNEGLCLKFIALQRVVSLKSCTIKX

SEQ ID NO: 254 - protein, 57 aa, Homo sapiens
  SITE (57): Xaa equals stop translation
MTFLLQWFPLGRARVVGDLCGFSTQIHPGVSRAGMADLESPPFPRTCSVPRAANKGX

SEQ ID NO: 255 - protein, 42 aa, Homo sapiens
  SITE (42): Xaa equals stop translation
MFVKYHVIMVIIFIFILITSDKHGEIIYIKYIDRVIITERIX

SEQ ID NO: 256 - protein, 161 aa, Homo sapiens
  SITE (161): Xaa equals stop translation
MQRVSGLLSWTLSRVLWLSGLSEPGAARQPRIMEEKALEVYDLIRTIRDPEKPNTLEELEVVSESCVEVQEINEEEYLVIIRFTPTVPHCSLATLIGLCLRVKLQRCLPFKHKLEIYISEGTHSTEEDINKQINDKERVAAAMENPNLREIVEQCVLEPDX

SEQ ID NO: 257 - protein, 51 aa, Homo sapiens
  SITE (51): Xaa equals stop translation
MLFFSLKESLYIFHTAILLVVCFACAVVCQYVIVRVCAVVFCFSKSQSLIX

SEQ ID NO: 258 - protein, 279 aa, Homo sapiens
  SITE (279): Xaa equals stop translation
MLIFGAIFGCLDPVATLAAVMTEKSPFTTPIGRKDEADLAKSALAMADSDHLTIYNAYLGWKKARQEGGYRSEITYCRRNFLNRTSLLTLEDVKQELIKLVKAAGFSSSTTSTSWEGNRASQTLSFQEIALLKAVLVAGLYDNVGKIIYTKSVDVTEKLACIVETAQGKAQVHPSSVNRDLQTHGWLLYQEKIRYARVYLRETTLITPFPVLLFGGDIEVQHRERLLSIDGWIYFQAPVKIAVIFKQLRVLIDSVLRKKLENPKMSLEMTRFCRSLRNX

SEQ ID NO: 259 - protein, 69 aa, Homo sapiens
  SITE (69): Xaa equals stop translation
MKVLSWIHFILISLHFTSSLDPSSRGLGTFTDALPDSRAKVWEGEMEECPPVCVVLCATATDAEGFSGX

SEQ ID NO: 260 - protein, 122 aa, Homo sapiens
  SITE (122): Xaa equals stop translation
MIMAQKIGGLTWWAIMFIILFEITGTSSSFLRINALPHFSMNRCGEAYFPFSYLYTSLQKQFLMKVSGIVKNLRGNDDWRCFGVFFCIHFLMRKVLNVVQVRPNYYLTIIGRFYVSVKVFKX

SEQ ID NO: 261 - protein, 59 aa, Homo sapiens
  SITE (59): Xaa equals stop translation
MGKICKNWVSFLDNVLLLILFLYGLCLGWLCIYHQSYSTACICVVTDAEIQQKSLHSIX

SEQ ID NO: 262 - protein, 68 aa, Homo sapiens
  SITE (68): Xaa equals stop translation
MLVLLWLGWISSKSMLAAYFVAPKYPLKLALVSEPESSSLILKFLSLKDFLCCYTTKLSVNPPLLNDX

SEQ ID NO: 263 - protein, 46 aa, Homo sapiens
  SITE (46): Xaa equals stop translation
MVSFHFQCTSYFVRLFFQLQLFVGLVIVLALLISHSLTYSFHKHLX

SEQ ID NO: 264 - protein, 71 aa, Homo sapiens
  SITE (71): Xaa equals stop translation
MTHWSGCAALYLIFLSLKLAFQAGAGRGAQVGSVLPPSGGAVVVDQYCCRLSAQTYFSLPALQKCIGICRX

SEQ ID NO: 265 - protein, 41 aa, Homo sapiens
  SITE (41): Xaa equals stop translation
MVAMVFLKISVLPLMCRGQTKHKVLRDHAYPRVSQKRGHIX

SEQ ID NO: 266 - protein, 72 aa, Homo sapiens
  SITE (72): Xaa equals stop translation
MVQGPLTHLMLVLLISLIFLSRGSGRAWAFSHSCFKTSDLLPCRNRWEVIEFLHYSNLHSHISLSVTKTFLX

SEQ ID NO: 267 - protein, 100 aa, Homo sapiens
GRAFALRTMLPVVSSVFALPFYLNFRIYYFKILSYLNVIHFSSTNFEYHSFVLLDLHSLRSWGAKLGLRFGGFRSRVLSGGSASNADWRFCSNAFASSAH

SEQ ID NO: 268 - protein, 21 aa, Homo sapiens
LPVVSSVFALPFYLNFRIYYF

SEQ ID NO: 269 - protein, 21 aa, Homo sapiens
SFVLLDLHSLRSWGAKLGLRF

SEQ ID NO: 270 - protein, 20 aa, Homo sapiens
FGGFRSRVLSGGSASNADWR

SEQ ID NO: 271 - protein, 21 aa, Homo sapiens
FKILSYLNVIHFSSTNFEYHS

SEQ ID NO: 272 - protein, 140 aa, Homo sapiens
GAGKRPQVLTFPEYITSLSDSGTKRMAAGVRMECQSKGRCPSSCPLCHVTSSPDTPAEPVLLEVTKAAPIYELVTNNQTQRLLQEATMSSLWCSGTGDVIEDWCRCDSTAFGADGLPTCAPLPQPVYGSLSLFQHYSGNR

SEQ ID NO: 273 - protein, 20 aa, Homo sapiens
TFPEYITSLSDSGTKRMAAG

SEQ ID NO: 274 - protein, 21 aa, Homo sapiens
GVRMECQSKGRCPSSCPLCHV

SEQ ID NO: 275 - protein, 21 aa, Homo sapiens
VTSSPDTPAEPVLLEVTKAAP

SEQ ID NO: 276 - protein, 20 aa, Homo sapiens
PIYELVTNNQTQRLLQEATM

SEQ ID NO: 277 - protein, 84 aa, Homo sapiens
CLSIALSNALHSLDGATSRADFVALLDQFGNHYIQEAIYGFEESCSIWYPNKQVQRRLWLEYEDISKGNSPSDESEERERDPKC

SEQ ID NO: 278 - protein, 21 aa, Homo sapiens
MSSLWCSGTGDVIEDWCRCDS

SEQ ID NO: 279 - protein, 50 aa, Homo sapiens
NSARAEAEELSPLLSNELHRQRSPGVSFGLSVFNLMNAIMGSGILGLAYV

SEQ ID NO: 280 - protein, 21 aa, Homo sapiens
LSPLLSNELHRQRSPGVSFGL

SEQ ID NO: 281 - protein, 21 aa, Homo sapiens
LSVFNLMNAIMGSGILGLAYV

SEQ ID NO: 282 - protein, 28 aa, Homo sapiens
HLGRGFVPGILGHWLGFEERSQYLPGCR

SEQ ID NO: 283 - protein, 110 aa, Homo sapiens
FYIADHSFTARPTLRMFRISAVVATDKMTFTSGGTLFGDGCASSVAGEVMNCQTVLCILWTPFVFCPSIAVIIIPCVFTSKALEAIWKWCRVERRPHIIEVDVLGKCPAF

SEQ ID NO: 284 - protein, 25 aa, Homo sapiens
RPTLRMFRISAVVATDKMTFTSGGT

SEQ ID NO: 285 - protein, 28 aa, Homo sapiens
PSIAVIIIPCVFTSKALEAIWKWCRVER

SEQ ID NO: 286 - protein, 20 aa, Homo sapiens
TSVSFHHRYKSSDRPAHKVS

SEQ ID NO: 287 - DNA, 1187 bp, Homo sapiens
gggtcgacccacgcgtccggtaaaatataaagaaactgaaccagtgtgtcttttcaccatagatataagagttcggaccgcccagcacacaaggtcagcatgctgctcctctgtcacgctctcgctatagctgttgtccagatcgttatcttctcagaaagctgggcatttgccaagaacatcaacttctataatgtgaggcctcctctcgaccctacaccatttccaaatagcttcaagtgctttacttgtgaaaacgcaggggataattataactgcaatcgatgggcagaagacaaatggtgtccacaaaatacacagtactgtttgacagttcatcacttcaccagccacggaagaagcacatccatcaccaaaaagtgtgcctccagaagtgaatgtcattttgtcggttgccaccacagccgagattctgaacatacggagtgtaggtcttgctgtgaaggaatgatctgcaatgtagaattacccaccaatcacactaatgcagtgtttgccgtaatgcacgctcagagaacatctggcagcagtgcccccacactctacctaccagtgcttgcctgggtctttgtgcttccattgctgtgatgccaccattcctaggagaggcagagaccagcctctaaagcacaagccaaaaactgtgtgaacggtgaactttggagtgaagatcaatcttgcacttggtgaagagtgcacattggacctcaaggcgaaagccagtggtttgcttggataaaatgttcccgcatgaggccacaggactgaggatgggaatttggcagggcctgagaagatggtctgacttccaggcttcctggtcaaagagagctacgtttgggcagttctgcagagaggatcctggcaactagtcccacctgactaggcctttagctgaaaaggatttcttgacctccttgactgcctcagaggctgccaggtcaaaccctcttgtttatgtgattagctcagagcatctctatgaaatctaacccttcccctcatgagaaagcagttttccccaccaacagcatagtcaatgagaaaggcaactgtacgaagaaaacttccagtggaactaatatgaaatctatttgcaaattatggggggaaataaagcttttaaattatacaatgtaaaaaaaaaaaaaaaaaaaaaaaaaaaa

SEQ ID NO: 288 - protein, 169 aa, Homo sapiens
MLLLCHALAIAVVQIVIFSESWAFAKNINFYNVRPPLDPTPFPNSFKCFTCENAGDNYNCNRWAEDKWCPQNTQYCLTVHHFTSHGRSTSITKKCASRSECHFVGCHHSRDSEHTECRSCCEGMICNVELPTNHTNAVFAVMHAQRTSGSSAPTLYLPVLAWVFVLPLL

SEQ ID NO: 289 - protein, 21 aa, Homo sapiens
IAVVQIVIFSESWAFAKNINF

SEQ ID NO: 290 - protein, 21 aa, Homo sapiens
FYNVRPPLDPTPFPNSFKCFT

SEQ ID NO: 291 - protein, 21 aa, Homo sapiens
TCENAGDNYNCNRWAEDKWCP

SEQ ID NO: 292 - protein, 21 aa, Homo sapiens
PQNTQYCLTVHHFTSHGRSTS

SEQ ID NO: 293 - protein, 21 aa, Homo sapiens
SITKKCASRSECHFVGCHHSR

SEQ ID NO: 294 - protein, 21 aa, Homo sapiens
RDSEHTECRSCCEGMICNVEL

SEQ ID NO: 295 - protein, 115 aa, Homo sapiens
RHNDFNKLSYTECNNMNKRMAKPEKKKGSVKSSLGIFLGPNCHLISSLFLFSVSLYPFATQFPFHYVLIFIIQAFGLCLPLTERQEAKSGLGGLCPDYTWPCPCLLVSCLSLLRL

SEQ ID NO: 296 - protein, 114 aa, Homo sapiens
CEVFSWHFPWSKLSPHLFLVSFLCIPLSLCHTVSFSLCSNIYNPGLRTMLAPHRETGGQVWAGWALSRLHVALPMSLGVLSLPAPTVTVVRMEGGDWKVCEQLGQCTYSHRMTK

SEQ ID NO: 297 - protein, 23 aa, Homo sapiens
KRMAKPEKKKGSVKSSLGIFLGP

SEQ ID NO: 298 - protein, 31 aa, Homo sapiens
YNPGLRTMLAPHRETGGQVWAGWALSRLHVA

SEQ ID NO: 299 - protein, 9 aa, Homo sapiens
SCKTENLLE

SEQ ID NO: 300 - protein, 50 aa, Homo sapiens
ECGSWAGFHTSSFPRPSALALAAWRRWGSICHLHTAGFIFGAAPRGNKCR

SEQ ID NO: 301 - protein, 21 aa, Homo sapiens
TSSFPRPSALALAAWRRWGSI

SEQ ID NO: 302 - protein, 21 aa, Homo sapiens
ICHLHTAGFIFGAAPRGNKCR

SEQ ID NO: 303 - protein, 25 aa, Homo sapiens
PDTLDKSPLAPGSSLVDPQISLWVL

SEQ ID NO: 304 - protein, 251 aa, Homo sapiens [sequence omitted]

SEQ ID NO: 305 - protein, 23 aa, Homo sapiens
LPPYPPQEANRSITSLSVADTVS

SEQ ID NO: 306 - protein, 27 aa, Homo sapiens
TAKPAAPSFGVLSNLPLPIPTVDASIP

SEQ ID NO: 307 - protein, 25 aa, Homo sapiens
PDVPDAFPELSELSVSQLTDMNEQE

SEQ ID NO: 308 - protein, 29 aa, Homo sapiens
QFLTLPQLKQIITDKDDLVKSIEELARKN

SEQ ID NO: 309 - protein, 25 aa, Homo sapiens
RQTVLDKYELLTQMKSTFEKKMQRQ

SEQ ID NO: 310 - protein, 28 aa, Homo sapiens
ASALQARLKVAAHEAEEESDNIAEDFLE

SEQ ID NO: 311 - protein, 27 aa, Homo sapiens
MEKRTICHCRRAKEEKLQQAIAMHSQF

SEQ ID NO: 312 - protein, 19 aa, Homo sapiens
LLLQQHFLIYTVTQVGCLL

SEQ ID NO: 313 - protein, 16 aa, Homo sapiens
EFGTRKSKSKINIKEE

SEQ ID NO: 314 - protein, 20 aa, Homo sapiens
GTSSKVVTQKVHLSSVEFPF

SEQ ID NO: 315 - protein, 69 aa, Homo sapiens
TRPVFLSMTPLKGIKSVILPQVFLCAYMAAFNSINGNRSYTCKPLERSLLMAGAVASSTFLGVIPQFVQ

SEQ ID NO: 316 - protein, 21 aa, Homo sapiens
PLKGIKSVILPQVFLCAYMAA

SEQ ID NO: 317 - protein, 21 aa, Homo sapiens
AFNSINGNRSYTCKPLERSLL

SEQ ID NO: 318 - protein, 19 aa, Homo sapiens
PESPVYPRRRTFSPNPSPI

SEQ ID NO: 319 - protein, 11 aa, Homo sapiens
NVSANLNFHVH

SEQ ID NO: 320 - protein, 129 aa, Homo sapiens [sequence omitted]

SEQ ID NO: 321 - protein, 30 aa, Homo sapiens
VGPLLLMTTESWGPSCAPSPALLSGHTAAS

SEQ ID NO: 322 - protein, 27 aa, Homo sapiens
ETNKVEEGRWVDVTRSLGNFNFRRKFF

SEQ ID NO: 323 - protein, 10 aa, Homo sapiens
QSPRVRSLGD

SEQ ID NO: 324 - protein, 50 aa, Homo sapiens
GGPMKDCEYSQISTHSSSPMESPHKKKKIAARRKWEVFPGRNKFFCNGRI

SEQ ID NO: 325 - protein, 21 aa, Homo sapiens
SQISTHSSSPMESPHKKKKIA

SEQ ID NO: 326 - protein, 21 aa, Homo sapiens
AARRKWEVFPGRNKFFCNGRI

SEQ ID NO: 327 - protein, 27 aa, Homo sapiens
PPFPHPETGQLCLVDSAPRPLQPYLRL

SEQ ID NO: 328 - protein, 76 aa, Homo sapiens
HPMCAKVADPELSSCPHCGLTAQPGPESGNISHSLREGSPRTLFVDSTSQASVPAAECPGHREGTPFSGASTSQAF

SEQ ID NO: 329 - protein, 14 aa, Homo sapiens
TPLLSPCLQPLPGV

SEQ ID NO: 330 - protein, 11 aa, Homo sapiens
TRRSCSSQVSS

SEQ ID NO: 331 - protein, 140 aa, Homo sapiens [sequence omitted]

SEQ ID NO: 332 - protein, 146 aa, Homo sapiens [sequence omitted]

SEQ ID NO: 333 - protein, 30 aa, Homo sapiens
SLRLKGPPSCQAPASHSSTLSSHCPCSLFA

SEQ ID NO: 334 - protein, 30 aa, Homo sapiens
QQMLGPSLCPGHSTSAHQHYGAYVLPRDLC

SEQ ID NO: 335 - protein, 31 aa, Homo sapiens
DLQVFVSRDLPHARPLPLTAAPFPLIVPVPF

SEQ ID NO: 336 - protein, 39 aa, Homo sapiens
AQVHISTMGPMSCPETSAPSCSHPQFRARRPSRTPESPV

SEQ ID NO: 337 - protein, 17 aa, Homo sapiens
QAPPRQTCKSSSQGTSL

SEQ ID NO: 338 - protein, 314 aa, Homo sapiens
  SITE (27): Xaa equals any of the naturally occurring L-amino acids
AALRPSGSLAGPEWPWQHWCGCWREHXVKPQQVDLHSARLWAAPAAVGPAHAGGSPGMPPGGTAPHARRHSLPSPTAQSHLWHVHGLRQRGPKAVPLDLAQLVTTTTPLFXLALSALLLGRRHHPLQLAAMGPLCLGAACSLAGEFRTPPTGCGFLLAATCLRGLKSVQQSALLQEERLDAVTLLYATSLPSFCLLAGAALVLEAGVAPPPTAGDSRLWACILLSCLLSVLYNLASFSLLALTSALTVHVLGNLTVVGNLILSRLLFGSRLSALSYVGIALTLSGMFLYHNCEFVASWAARRGLWRRDQPSKGL

SEQ ID NO: 339 - protein, 66 aa, Homo sapiens
  SITE (28): Xaa equals any of the naturally occurring L-amino acids
GQPSGPPAAWPGPSGHGSTGVAAGGSTXSSLNKWIFTVHGFGRPLLLSALHMLVAALACHRGARRP

SEQ ID NO: 340 - protein, 21 aa, Homo sapiens
  SITE (19): Xaa equals any of the naturally occurring L-amino acids
WPGPSGHGSTGVAAGGSTXSS

SEQ ID NO: 341 - protein, 26 aa, Homo sapiens
  SITE (15): Xaa equals any of the naturally occurring L-amino acids
EWPWQHWCGCWREHXVKPQQVDLHSA

SEQ ID NO: 342 - protein, 28 aa, Homo sapiens
QQSALLQEERLDAVTLLYATSLPSFCLL

SEQ ID NO: 343 - protein, 27 aa, Homo sapiens
ACILLSCLLSVLYNLASFSLLALTSAL

SEQ ID NO: 344 - protein, 21 aa, Homo sapiens
SLNKWIFTVHGFGRPLLLSAL

SEQ ID NO: 345 - protein, 28 aa, Homo sapiens
EFGTSRARLQLKKNKKKERNIPGTLLSI

SEQ ID NO: 346 - protein, 17 aa, Homo sapiens
KSTLSAAVVATILRTLA

SEQ ID NO: 347 - protein, 100 aa, Homo sapiens
GDHSEQCLIKEMGARERRFCKARGYRDTGREAQAKAGGRRGSQWNESQCSSQRPRPAKEVRKTRPRAGVGRGPALLQLSLLQQVVLYVRPSLRLVWLKAS

SEQ ID NO: 348 - protein, 84 aa, Homo sapiens
MERGEYGGWGTYGSLDLGSQLCTVRSSGPCGSLHWGQHRSPISGPDPNPSSSRGQQSIGSKVGSPSRSQWRSWKEVGRDPEKGE

SEQ ID NO: 349 - protein, 23 aa, Homo sapiens
QAKAGGRRGSQWNESQCSSQRPR

SEQ ID NO: 350 - protein, 26 aa, Homo sapiens
VGRGPALLQLSLLQQVVLYVRPSLRL

SEQ ID NO: 351 - protein, 22 aa, Homo sapiens
YGSLDLGSQLCTVRSSGPCGSL

SEQ ID NO: 352 - protein, 20 aa, Homo sapiens
KVGSPSRSQWRSWKEVGRDP

SEQ ID NO: 353 - protein, 33 aa, Homo sapiens
ARGFFFYILITRLTPIKYDVNLILTAVTGSVGG

SEQ ID NO: 354 - protein, 214 aa, Homo sapiens
  SITE (18): Xaa equals any of the naturally occurring L-amino acids
MPQSLSSLASSSSSFQRXKPCFGKKNDGENQEHSLGTEPIITWKDFQKTMPWEIVILVGGGYALASGSKSSGLSTWIGNQMLSLSSLPPWAVTLLACILVSIVTEFVSNPATITIFLPILCSLSETLHINPLYTLIPVTMCISFAVMLPVGNPPNAIVFSYGHCQIKDMVKAGLGVNVIGLVIVMVAINTWGVSLFHLDTYPAWARVSNITDQA

SEQ ID NO: 355 - protein, 23 aa, Homo sapiens
NDGENQEHSLGTEPIITWKDFQK

SEQ ID NO: 356 - protein, 24 aa, Homo sapiens
IGNQMLSLSSLPPWAVTLLACILV

SEQ ID NO: 357 - protein, 27 aa, Homo sapiens
ATITIFLPILCSLSETLHINPLYTLIP

SEQ ID NO: 358 - protein, 26 aa, Homo sapiens
LPVGNPPNAIVFSYGHCQIKDMVKAG

SEQ ID NO: 359 - protein, 29 aa, Homo sapiens
LVIVMVAINTWGVSLFHLDTYPAWARVSN

SEQ ID NO: 360 - protein, 83 aa, Homo sapiens
  SITE (68): Xaa equals any of the naturally occurring L-amino acids
LEHFNNQYPAAEVVNFGTWFLFSFPISLIMLVVSWFWMHWLFLGCNFKETCSLSKKKKTKREQLSEKXXQEEYEKLGDISYPE

SEQ ID NO: 361 - protein, 36 aa, Homo sapiens
QELWPLYMDWEPDVVPEQPPTVGCHPAGMHPRVHCH

SEQ ID NO: 362 - protein, 37 aa, Homo sapiens
STHASGGGRRGRGPRGEETQPRGWHARPGPGPRSTGA

SEQ ID NO: 363 - protein, 133 aa, Homo sapiens
  SITE (44): Xaa equals any of the naturally occurring L-amino acids
ETCPSNGIELRQAPTSLYILLLHIQPTPTHPMLGRSYVLPAFSXNXEHGGLPNQIPKGDRNGNIRHSRITFPCSSSTLQPESHLGFIRSKLHGLVRPGKDLRGRRSLQLSKHSLSTCYMLRWETYKQVSYTAV

SEQ ID NO: 364 - protein, 106 aa, Homo sapiens
QRHQENDKRNVHRFLHTCVHMPMCTHTHTQAVLSTWEGQFSNVASFTSLKRIPLSIIYIHSSHSPRRFVKVCQLRQEKALELTEVYVSASLKLQLYHLHCHFHTAV

SEQ ID NO: 365 - protein, 24 aa, Homo sapiens
RQAPTSLYILLLHIQPTPTHPMLG

SEQ ID NO: 366 - protein, 25 aa, Homo sapiens
SHLGFIRSKLHGLVRPGKDLRGRRS

SEQ ID NO: 367 - protein, 22 aa, Homo sapiens
RNVHRFLHTCVHMPMCTHTHTQ

SEQ ID NO: 368 - protein, 25 aa, Homo sapiens
QLRQEKALELTEVYVSASLKLQLYH

SEQ ID NO: 369 - protein, 31 aa, Homo sapiens [sequence omitted]

SEQ ID NO: 370 - protein, 296 aa, Homo sapiens
LSKGNRIMAADDDNGDGTSLFDVFSASPLKNNDEGSLDIYAGLDSAVSDSASKSCVPSRNCLDLYEEILTEEGTAKEATYNDLQVEYGKCQLQMKELMKKFKEIQTQNFSLINENQSLKKNISALIKTARVEINRKDEEISNLHQKIVLSFHIFEIIIKLQGHLIQLKQKILNLDLHIWMIVQRLITRAKSDVSKDVHHSTSLPNLEKEGKPHSDKRSTSHLPTSVEKHCTNGVWSRSHYQVGEGSSNEDSRRGRKDIRHSQFNRGTERVRKDLSTGCGDGEPRILEASQRLQGTS

SEQ ID NO: 371 - protein, 27 aa, Homo sapiens
NRIMAADDDNGDGTSLFDVFSASPLKN

SEQ ID NO: 372 - protein, 23 aa, Homo sapiens
CLDLYEEILTEEGTAKEATYNDL

SEQ ID NO: 373 - protein, 26 aa, Homo sapiens
DEEISNLHQKIVLSFHIFEIIIKLQG

SEQ ID NO: 374 - protein, 22 aa, Homo sapiens
EKEGKPHSDKRSTSHLPTSVEK

SEQ ID NO: 375 - protein, 26 aa, Homo sapiens
TERVRKDLSTGCGDGEPRILEASQRL

SEQ ID NO: 376 - protein, 115 aa, Homo sapiens
KSYFRTMGGTKRGIKKLVNVCLKHPKNTSLSQQLVFAKINKILISKTTKSTNLKGLKCLPPLSVSIHPTFIYYKHNTTLRIVFGTYFDFFPYRKNKDQAFEGEDWESSLNVSDAW

SEQ ID NO: 377 - protein, 22 aa, Homo sapiens
TKRGIKKLVNVCLKHPKNTSLS

SEQ ID NO: 378 - protein, 26 aa, Homo sapiens
SIHPTFIYYKHNTTLRIVFGTYFDFF

SEQ ID NO: 379 - protein, 56 aa, Homo sapiens
TRPRRHLGGQPGALHGQAACVHVPCLVPLCPPPANLTGSPHNSALQKQPLGGRGRK

SEQ ID NO: 380 - protein, 21 aa, Homo sapiens
QPGALHGQAACVHVPCLVPLC

SEQ ID NO: 381 - protein, 21 aa, Homo sapiens
CPPPANLTGSPHNSALQKQPL

SEQ ID NO: 382 - protein, 28 aa, Homo sapiens
PDAGTASSQREPRRCRAGEAPSLPACAP

SEQ ID NO: 383 - protein, 40 aa, Homo sapiens
FLIHLEVIWELGCFSPKAKAIASTPVIKGSLQIYFPCRSE

SEQ ID NO: 384 - protein, 32 aa, Homo sapiens
HESKEKCPPGPLHQRCVFNSSGAGRVMATRKR

SEQ ID NO: 385 - protein, 27 aa, Homo sapiens
KRTLLQRLDWSYWVDSWEHQHSLHNGW

SEQ ID NO: 386 - protein, 12 aa, Homo sapiens
GPRGVGDGGVSS

SEQ ID NO: 387 - protein, 70 aa, Homo sapiens
  SITE (9): Xaa equals any of the naturally occurring L-amino acids
QRPHPQPWXPMTLMGTGIPVFAHKMLPFDPPCHLSCTHINPKPXXPQGDEQKSQGTEEWCDREGKKRRSI

SEQ ID NO: 388 - protein, 21 aa, Homo sapiens
PMTLMGTGIPVFAHKMLPFDP

SEQ ID NO: 389 - protein, 21 aa, Homo sapiens
  SITE (15): Xaa equals any of the naturally occurring L-amino acids
PPCHLSCTHINPKPXXPQGDE

SEQ ID NO: 390 - protein, 21 aa, Homo sapiens
EQKSQGTEEWCDREGKKRRSI

SEQ ID NO: 391 - protein, 70 aa, Homo sapiens
  SITE (64): Xaa equals any of the naturally occurring L-amino acids
DEWGAGRRMEWEDNLPLEFSCPVTKLLSVPSWTPLDAQMLLLFFPSLSHHSSVPWLFCSSPCGXXGLGFI

SEQ ID NO: 392 - protein, 21 aa, Homo sapiens
EWEDNLPLEFSCPVTKLLSVP

SEQ ID NO: 393 - protein, 21 aa, Homo sapiens
PSWTPLDAQMLLLFFPSLSHH

SEQ ID NO: 394 - protein, 21 aa, Homo sapiens
  SITE (15): Xaa equals any of the naturally occurring L-amino acids
HSSVPWLFCSSPCGXXGLGFI

SEQ ID NO: 395 - protein, 14 aa, Homo sapiens
ITEVRKDDLKVVRI

SEQ ID NO: 396 - protein, 15 aa, Homo sapiens
QGLSHIFWMNEQTLK

SEQ ID NO: 397 - protein, 32 aa, Homo sapiens
TLVCLGVSSEEGSCPRDVTGPGCCFSLTLTGF

SEQ ID NO: 398 - protein, 233 aa, Homo sapiens
  SITE (57): Xaa equals any of the naturally occurring L-amino acids
ADLIVLWHHHPLWPQHLALPSSGASHDHVELTVYPKTVAASWLLELSRPPIFCLFTXPALTXHGLDRVAALVECTIWXXXGMWYRRRYSCCQFRDRSIRDVFPEAVMLQQHLRHLAVATYRCRRRSPCKAPTVEEAEGGKPRAVPSGTGFQKHGQEPGGSTSPHWFWGHLQLLVLSVNNRQLFVQGRAGYLEMTGLPCPKLLLTLLRGLTPGVGHGLCAYRRGCLAWRLDXAS

SEQ ID NO: 399 - protein, 176 aa, Homo sapiens
  SITE (70): Xaa equals any of the naturally occurring L-amino acids
ILWRQAPEAPHCSQDSVSSSPRLQEDLAHVTQVTRHPHFRSLPSAWCSHSSLLPVSLPRHALATKSPNMXXSSPILHLIQFTGQISSPLGGXVQPPGQTASPICTQPMSHPRRQASQQCEQQLWTGQTSHLQIPCPALNKELPVVDTQDKELQMSPEPMWGCGPSRLLPMLLESCA

SEQ ID NO: 400 - protein, 34 aa, Homo sapiens
MLQQHLRHLAVATYRCRRRSPCKAPTVEEAEGGK

SEQ ID NO: 401 - protein, 29 aa, Homo sapiens
VTQVTRHPHFRSLPSAWCSHSSLLPVSLP

SEQ ID NO: 402 - protein, 28 aa, Homo sapiens
GQTASPICTQPMSHPRRQASQQCEQQLW

SEQ ID NO: 403 - protein, 79 aa, Homo sapiens
FITLRLGPKNMAGVLWRHSNLQTPHYISWCPLLNYRETGNCLLHVSGFLNSRLLANCSGEASGKVIQTLLWPGEISAVA

SEQ ID NO: 404 - protein, 82 aa, Homo sapiens
KIRTFLFSGHRLFSTQGQSLTVKAHTAFMLIVKNLRYFIAFKFLMGISDSSEIGLVMQPLQKPHTVILIRGIEFLSPGGVLP

SEQ ID NO: 405 - protein, 26 aa, Homo sapiens
MAGVLWRHSNLQTPHYISWCPLLNYR

SEQ ID NO: 406 - protein, 29 aa, Homo sapiens
YFIAFKFLMGISDSSEIGLVMQPLQKPHT

SEQ ID NO: 407 - protein, 8 aa, Homo sapiens
PFGLLVLP

SEQ ID NO: 408 - protein, 152 aa, Homo sapiens
GFSRDTSVLSHFAFNSASPPKSYIRGKLGLEEYAVFYPPNGVIPFHGFSMYVAPLCFLYHEPSKLYQIFREMYVRFFFRLHSISSHPSGIVSLCLLFETLLQTYLPQLFYHLREIGAQPLRISFKWMVRAFSGYLATDQLLLLWDRILGYNS

SEQ ID NO: 409 - protein, 39 aa, Homo sapiens
LCQRGWAGQPGILTDGHPLPGQAASRSHQGPVGPGFSAN

SEQ ID NO: 410 - protein, 21 aa, Homo sapiens
QPGILTDGHPLPGQAASRSHQ

SEQ ID NO: 411 - protein, 6 aa, Homo sapiens
LLRPIL

SEQ ID NO: 412 - protein, 53 aa, Homo sapiens
ARADRARGAAAGRSGRAAAAPWTPVSSLSSSLTEWPPPKCCQPRKPPALTMSI

SEQ ID NO: 413 - protein, 21 aa, Homo sapiens
AAAGRSGRAAAAPWTPVSSLS

SEQ ID NO: 414 - protein, 21 aa, Homo sapiens
SSSLTEWPPPKCCQPRKPPAL

SEQ ID NO: 415 - protein, 137 aa, Homo sapiens
EYFLEFVFSLIWILSHCSILLSSAVCDPGNIRVTEAPKHPISEELETPIKDSHLIPTPQAPSIAFPLANPPVAPHPREKIITIEETHEELKKQYIFQLSSLNPQERIDYCHLIEKLGTSILLKSKMSHIITIFGSQM

SEQ ID NO: 416 - protein, 21 aa, Homo sapiens
LIWILSHCSILLSSAVCDPGN

SEQ ID NO: 417 - protein, 21 aa, Homo sapiens
NIRVTEAPKHPISEELETPIK

SEQ ID NO: 418 - protein, 20 aa, Homo sapiens
KDSHLIPTPQAPSIAFPLAN

SEQ ID NO: 419 - protein, 21 aa, Homo sapiens
NPPVAPHPREKIITIEETHEE

SEQ ID NO: 420 - protein, 21 aa, Homo sapiens
ELKKQYIFQLSSLNPQERIDY

SEQ ID NO: 421 - protein, 6 aa, Homo sapiens
INICIY

SEQ ID NO: 422 - protein, 11 aa, Homo sapiens
  SITE (6): Xaa equals any of the naturally occurring L-amino acids
LQESAXQFSSS

SEQ ID NO: 423 - protein, 75 aa, Homo sapiens
NLHGCHGKFQEHNLKVNCMTLFCVSLTTTHSVSLKVTVYITVSILCMPDTQDSNFSFPLDTTYLVINFGSTYSTK

SEQ ID NO: 424 - protein, 30 aa, Homo sapiens
LFCVSLTTTHSVSLKVTVYITVSILCMPDT

SEQ ID NO: 425 - protein, 11 aa, Homo sapiens
LLNPKASLHSA

SEQ ID NO: 426 - protein, 20 aa, Homo sapiens
  SITE (18): Xaa equals any of the naturally occurring L-amino acids
DPRVRASVGRCVRAAGFXLA

SEQ ID NO: 427 - protein, 87 aa, Homo sapiens
  SITE (6): Xaa equals any of the naturally occurring L-amino acids
PYRGGXPYHLPESPPKRVPWQEHAPRQVCWRLCPIRXGLEEKGGRHQSQEPGMXGSCWAFSXTGNVEGQWFLKQGPXLPLRXXXLGL

SEQ ID NO: 428 - protein, 304 aa, Homo sapiens
  SITE (30): Xaa equals any of the naturally occurring L-amino acids
RPTRPRVRRSVRPGRRLRPRHGTLAAAAVXAGAAPGXRSRPAPPSSRRSGPGGGVPGAAGARPLRAGDVQPRPGSRXAGDAGGRARSRPPGGRGVAVLPEGDPGGASLQRXHGVPAPCVXETLLCSFEVLDELGKHMLLRRDCGPVDTKVTDDKNETLSSVLPLLNKEPLPQDFSVKMASIFKEFVTTYNRTYESKEETQWRMSVFSNNMMRAQKIQALDRGTAQYGVTKFSDLTEEEFHTIYLNPLLREYHGKNMRLDKSAGDSAPSEWDWXXKGXVTKVKNQACXAPAGLSQSLVTWRASGS

SEQ ID NO: 429 - protein, 85 aa, Homo sapiens
  SITE (8): Xaa equals any of the naturally occurring L-amino acids
TLAAAAVXAGAAPGXRSRPAPPSSRRSGPGGGVPGAAGARPLRAGDVQPRPGSRXAGDAGGRARSRPPGGRGVAVLPEGDPGGAS

SEQ ID NO: 430 - protein, 119 aa, Homo sapiens
SFEVLDELGKHMLLRRDCGPVDTKVTDDKNETLSSVLPLLNKEPLPQDFSVKMASIFKEFVTTYNRTYESKEETQWRMSVFSNNMMRAQKIQALDRGTAQYGVTKFSDLTEEEFHTIYL

SEQ ID NO: 431 - protein, 11 aa, Homo sapiens
TSHPLGGGVER

SEQ ID NO: 432 - protein, 9 aa, Homo sapiens
ACCCLEWAG

SEQ ID NO: 433 - protein, 43 aa, Homo sapiens
SAEQKTRLHLLYKTELYFSFIISRVAVLLVLIHWRGGIRTDVS

SEQ ID NO: 434 - protein, 23 aa, Homo sapiens
TLQNIYPLLIDASLYICVYIHTY

SEQ ID NO: 435 - protein, 31 aa, Homo sapiens
DVLLPLLYLLVRKHINRAGIGNTFQGGANCI

SEQ ID NO: 436 - protein, 99 aa, Homo sapiens
MCCCLCCTSWSGSTSTERVSGTRFREVPTASCSSSAPAPSELGSSLSVAAAALLSLPPRARLALPRLPRLPSQENLRNPKGPQGNFQAPGAFVLSSSVA

SEQ ID NO: 437 - protein, 216 aa, Homo sapiens
  SITE (108): Xaa equals any of the naturally occurring L-amino acids
CAAASAVPPGPEAHQQSGYREHVSGRCQLHHVRPLHPRRPNSALLSLLLLLLFSASHQEPGWHSQGSRAFQARRISGIPRDPRGTSKHLELLSFLVLWHRCCLPGGRXFCESLXQGRSACLLHQKPPLLMLSAPLGEQLPTQLLLPPRSSGSKFXRYQRPGPRVGVHLHKGSSEIREAGGPQLWPQCPHPVDLDVLRTTQHCLQSEGPTSVHLSSV

SEQ ID NO: 438 - protein, 147 aa, Homo sapiens
  SITE (34): Xaa equals any of the naturally occurring L-amino acids
EVEEAELAAALPMEPRASIAGASGAADMHFCPAXGTHRXAYPQEGSTYATELERTKAPGAWKFPWGPLGFLRFSWLGRRGSLGSASRALGGRLRRAAAATEREEPSSDGAGAEDEHDAVGTSLKRVPDTRSVDVLPDQEVQQRQQHI

SEQ ID NO: 439 - protein, 31 aa, Homo sapiens
RRISGIPRDPRGTSKHLELLSFLVLWHRCCL

SEQ ID NO: 440 - protein, 29 aa, Homo sapiens
RTKAPGAWKFPWGPLGFLRFSWLGRRGSL

SEQ ID NO: 441 - protein, 31 aa, Homo sapiens; identical to SEQ ID NO: 435

SEQ ID NO: 442 - protein, 11 aa, Homo sapiens
PRLAQLRLLSL

SEQ ID NO: 443 - protein, 178 aa, Homo sapiens
QSDFREMNQTNSTSNAAKAREAQQGRGRDREAIFSSSALEHLVCYLQAYKHTLLFIRSLNEHGLQQLLFQWRDGLFGNWYFRIPILLFFTGFHCYHLSCPHLPCAQRQSSRGTVPYVLCPHPHHHLHHYSWFPFLIPVLHTLPKLQPKFHGRPEQPLNLLQVKPTSGTIASAEQVWVK

SEQ ID NO: 444 - protein, 29 aa, Homo sapiens
VCYLQAYKHTLLFIRSLNEHGLQQLLFQW

SEQ ID NO: 445 - protein, 32 aa, Homo sapiens
VPYVLCPHPHHHLHHYSWFPFLIPVLHTLPKL

SEQ ID NO: 446 - protein, 31 aa, Homo sapiens
ESERAVVYLITGALFIVSSCVLCFLPSSRRE

SEQ ID NO: 447 - protein, 146 aa, Homo sapiens
  SITE (108): Xaa equals any of the naturally occurring L-amino acids
HEARQGVSRGVKAAMNRVLCAPAAGAVRALRLIGWASRSLHPLPGSRDRAHPAAEEEDDPDRPIEFSSSKANPHRWSVGHTMGKGHQRPWWKVLPLSCFLVALIIWCXLREESEADQWLRQVWGEVPEPSDRSEEPETPAAYRART

SEQ ID NO: 448 - protein, 249 aa, Homo sapiens
  SITE (4): Xaa equals any of the naturally occurring L-amino acids
MWVXGEEVLGSHAASPAFLHRCFSEESCVSIPEVEGYVVVLQPDAPQILLSGTAHFARPAVDFEGTNGVPLFPDLQITCSISHQVEAKKDESWQGTVTDTRMSDEIVHNLDGCEISLVGDDLDPERESLLLDTTSLQQRGLELTNTSAYLTIAGVESITVYEEILRQARYRLRHGAALYTRKFRLSCSEMNGRYSSNEFIVEVNVLHSMNRVAHPSHVLSXQQFLHRGHQPPPEMAGHSLASSHRNSST

SEQ ID NO: 449 - protein, 23 aa, Homo sapiens
LGSHAASPAFLHRCFSEESCVSI

SEQ ID NO: 450 - protein, 29 aa, Homo sapiens
GYVVVLQPDAPQILLSGTAHFARPAVDFE

SEQ ID NO: 451 - protein, 26 aa, Homo sapiens
ITCSISHQVEAKKDESWQGTVTDTRM

SEQ ID NO: 452 - protein, 29 aa, Homo sapiens
NLDGCEISLVGDDLDPERESLLLDTTSLQ

SEQ ID NO: 453 - protein, 23 aa, Homo sapiens
SAYLTIAGVESITVYEEILRQAR

SEQ ID NO: 454 - protein, 26 aa, Homo sapiens
RLSCSEMNGRYSSNEFIVEVNVLHSM

SEQ ID NO: 455 - protein, 25 aa, Homo sapiens
QQFLHRGHQPPPEMAGHSLASSHRN

SEQ ID NO: 456 - protein, 299 aa, Homo sapiens
  SITE (52): Xaa equals any of the naturally occurring L-amino acids
MADSETFISLEECRGHKRARKRTSMETALALEKLFPKQCQVLGIVTPGIVVXPMGSGSNRPQEIEIGESGFALLFPQIEGIKIQPFHFIKDPKNLTLERHQLTEVGLLDNPELRVVLVFGYNCCKVGASNYLQQVVSTFSDMNIILAGGQVDNLSSLTSEKNPLDIDASGVVGLSFSGHRIQSATVLLNEDVSDEKTAEAAMQRLKAANIPEHNTIGFMFACVGRGFQYYRAKGNVEADAFRKFFPSVPLFGFFGNGEIGCDRIVTGNFILRKCNEVKDDDLFHSYTTIMALIHLGSSK

SEQ ID NO: 457 - protein, 21 aa, Homo sapiens
HKRARKRTSMETALALEKLFP

SEQ ID NO: 458 - protein, 24 aa, Homo sapiens
MGSGSNRPQEIEIGESGFALLFPQ

SEQ ID NO: 459 - protein, 22 aa, Homo sapiens
FHFIKDPKNLTLERHQLTEVGL

SEQ ID NO: 460 - protein, 23 aa, Homo sapiens
FGYNCCKVGASNYLQQVVSTFSD

SEQ ID NO: 461 - protein, 20 aa, Homo sapiens
TSEKNPLDIDASGVVGLSFS

SEQ ID NO: 462 - protein, 26 aa, Homo sapiens
NEDVSDEKTAEAAMQRLKAANIPEHN

SEQ ID NO: 463 - protein, 25 aa, Homo sapiens
YYRAKGNVEADAFRKFFPSVPLFGF

SEQ ID NO: 464 - protein, 26 aa, Homo sapiens
IGCDRIVTGNFILRKCNEVKDDDLFH

SEQ ID NO: 465 - protein, 65 aa, Homo sapiens
GTRYFLMELVWFRFLHLNLLPRGVCCGICVCVRRGMVLSEPTSCGQRALSCEGGCHSGRVQFRRP

SEQ ID NO: 466 - protein, 341 aa, Homo sapiens
MPKRKVTFQGVGDEEDEDEIIVPKKKLVDPVAGSGGPGSRFKGKHSLDSDEEEDDDDGGSSKYDILASEDVEGQEAATLPSEGGVRITPFNLQEEMEEGHFDADGNYFLNRDAQIRDSWLDNIDWVKIRERPPGQRQASDSEEEDSLGQTSMSAQALLEGLLELLLPRETVAGALRRLGARGGGKGRKGPGQPSSPQRLDRLSGLADQMVARGNLGVYQETRERLAMRLKGLGCQTLGPHNPTPPPSLDMFAEELAEEELETPTPTQRGEAESRGDGLVDVMWEYKWENTGDAELYGPFTSAQMQTWVSEGYFPDGVYCRKLDPPGGQFYNSKRIDFDLYT

SEQ ID NO: 467 - protein, 24 aa, Homo sapiens
TFQGVGDEEDEDEIIVPKKKLVDP

SEQ ID NO: 468 - protein, 27 aa, Homo sapiens
PGSRFKGKHSLDSDEEEDDDDGGSSKY

SEQ ID NO: 469 - protein, 25 aa, Homo sapiens
EAATLPSEGGVRITPFNLQEEMEEG

SEQ ID NO: 470 - protein, 29 aa, Homo sapiens
FLNRDAQIRDSWLDNIDWVKIRERPPGQR

SEQ ID NO: 471 - protein, 26 aa, Homo sapiens
SLGQTSMSAQALLEGLLELLLPRETV

SEQ ID NO: 472 - protein, 28 aa, Homo sapiens
RGGGKGRKGPGQPSSPQRLDRLSGLADQ

SEQ ID NO: 473 - protein, 24 aa, Homo sapiens
QETRERLAMRLKGLGCQTLGPHNP

SEQ ID NO: 474 - protein, 28 aa, Homo sapiens
DMFAEELAEEELETPTPTQRGEAESRGD

SEQ ID NO: 475 - protein, 30 aa, Homo sapiens
ELYGPFTSAQMQTWVSEGYFPDGVYCRKLD

SEQ ID NO: 476 - protein, 14 aa, Homo sapiens
PHSSRVSFLQSLSF

SEQ ID NO: 477 - protein, 141 aa, Homo sapiens [sequence omitted]

SEQ ID NO: 478 - protein, 133 aa, Homo sapiens [sequence omitted]

SEQ ID NO: 479 - protein, 25 aa, Homo sapiens
VCLSPHSRFWECCSFYLQGLPALRC

SEQ ID NO: 480 - protein, 27 aa, Homo sapiens
QFSRALWVSTCLVLAITPGKWLLPEDR

SEQ ID NO: 481 - protein, 27 aa, Homo sapiens
SLSLLRAQTGTDCAVSPGLAGPCHQRG

SEQ ID NO: 482 - protein, 28 aa, Homo sapiens
SGRSHFPGVMAKTKHVDTHNARENWIRT

SEQ ID NO: 483 - protein, 91 aa, Homo sapiens
ARGWECEEGSPGPVFRGCASPRTPVSGNAVPSTFRACPPCGVAALLPGVISSESFLHALFPPHVPPRALPTSVPWFGSSSPVRYGYPRVWS

SEQ ID NO: 484 - protein, 20 aa, Homo sapiens
ARVEVQGQGPGAKVDAGEGQ

SEQ ID NO: 485 - protein, 121 aa, Homo sapiens
  SITE (46): Xaa equals any of the naturally occurring L-amino acids
WVVLSQLQAQGVAGMMCSYPEGQKKGKEATRSHRWVPRSLPGMGSXLAAPHSNPWLAPLALLEIPXPVLCEWKRKLIALEEVSECRPGVGGGGGFLSXCRRGHLSFLSGAPYPLFPISPLX

SEQ ID NO: 486 - protein, 206 aa, Homo sapiens
  SITE (105): Xaa equals any of the naturally occurring L-amino acids
ELRHGGPRQVKDSFLDYMGYPDEDRAGPPSRWFPRERFLSPPTVVPLCVELRLGFESGMGWGVPGSSHSEGGPEARWPLIAPMYTVTQWFQRPNSGRGPQPPPQXRGEIGKRGYGAPERKLRWPLLXWERXPPPPPTPGRHSETSSSAISFLFHSQRTGWGISSSANGASQGLLWGAARXLPIPGRDLGTHLWDLVASFPFFCPSG

SEQ ID NO: 487 - protein, 24 aa, Homo sapiens
PEGQKKGKEATRSHRWVPRSLPGM

SEQ ID NO: 488 - protein, 26 aa, Homo sapiens
LRLGFESGMGWGVPGSSHSEGGPEAR

SEQ ID NO: 489 - protein, 24 aa, Homo sapiens
HSQRTGWGISSSANGASQGLLWGA

SEQ ID NO: 490 - protein, 20 aa, Homo sapiens
DSLTIKSGSQPQYSPAITLW

SEQ ID NO: 491 - protein, 54 aa, Homo sapiens
FIMKLLYQLLMLTTSSSYSLITHLCYSIFLCSFYFHFPCNVSLFVLISEEFIYD

SEQ ID NO: 492 - protein, 21 aa, Homo sapiens
LMLTTSSSYSLITHLCYSIFL

SEQ ID NO: 493 - protein, 21 aa, Homo sapiens
LCSFYFHFPCNVSLFVLISEE

SEQ ID NO: 494 - protein, 53 aa, Homo sapiens [sequence omitted]

SEQ ID NO: 495 - protein, 21 aa, Homo sapiens
DKMLTCLIINELFRNQYKETN

SEQ ID NO: 496 - protein, 21 aa, Homo sapiens
NITREVKIKGTEENGIAQMSY

SEQ ID NO: 497 - protein, 7 aa, Homo sapiens
GISERKP

SEQ ID NO: 498 - protein, 25 aa, Homo sapiens [sequence omitted]

SEQ ID NO: 499 - protein, 100 aa, Homo sapiens
  SITE (96): Xaa equals any of the naturally occurring L-amino acids
LPSHPLRPLTFSSAMCMHLPPPLCRRAALSAPFATQHRPWSVAAACLPRIHQNPLDAEYPSGCCRMSFLPAACSNIYSQECHYTLMSHSEASTLQXAQLL

SEQ ID NO: 500 - protein, 76 aa, Homo sapiens [sequence omitted]

SEQ ID NO: 501 - protein, 25 aa, Homo sapiens
LCRRAALSAPFATQHRPWSVAAACL

SEQ ID NO: 502 - protein, 24 aa, Homo sapiens
RGFWWMRGRQAAATLHGRCWVAKG

SEQ ID NO: 503 - protein, 23 aa, Homo sapiens
QRGGGRCMHIADEKVRGLSGCDG

SEQ ID NO: 504 - protein, 106 aa, Homo sapiens
THPSHPSIVIQSTVSLCLTASSRRKKSDCLSLCQVSCSQRPGSHKTNVAWGFLMSRVHFSVRWVSGGRGITGAICKESSLPCKEIQGKACYFCHHPAQQSTPFSHI

SEQ ID NO: 505 - protein, 27 aa, Homo sapiens
VIQSTVSLCLTASSRRKKSDCLSLCQV

SEQ ID NO: 506 - protein, 26 aa, Homo sapiens
ICKESSLPCKEIQGKACYFCHHPAQQ

SEQ ID NO: 507 - protein, 11 aa, Homo sapiens
PTRPPTRPAGK

SEQ ID NO: 508 - protein, 35 aa, Homo sapiens
SITKYCQGCRKIGALLPWWECNMVPDTTSILKLIC

SEQ ID NO: 509 - protein, 188 aa, Homo sapiens
  SITE (140): Xaa equals any of the naturally occurring L-amino acids
SLQVLRTLGSKCGDFLRSRFCKDVLPKLAGSLVTQAPISARAGPVYSHTLAFKLQLAVLQGLGPLCERLDLGEGDLNKVADACLIYLSVKQPVKLQEAARSVFLHLMKVDPDSTWFLLNELYCPVQFTPPHPSLHPVQLXGASGQQNPXHDQRAPAAQGAAVTLLPHHRGHRSLPYCQPEAGLTPPRP

SEQ ID NO: 510 - protein, 138 aa, Homo sapiens
GADGNVSDFDNEEEEQSVPPKVDENDTRPDVEPPLPLQIQIAMDVMERCIHLLSDKNLQIRLKVLDVLDLCVVVLQSHKNQLLPLAHQAWPSLVHRLTRDAPLAVLRAFKFYVPWEASVVTFFAAGSAKMSCQSWLAP

SEQ ID NO: 511 - protein, 26 aa, Homo sapiens
TLGSKCGDFLRSRFCKDVLPKLAGSL

SEQ ID NO: 512 - protein, 29 aa, Homo sapiens
PVYSHTLAFKLQLAVLQGLGPLCERLDLG

SEQ ID NO: 513 - protein, 27 aa, Homo sapiens
SVPPKVDENDTRPDVEPPLPLQIQIAM

SEQ ID NO: 514 - protein, 26 aa, Homo sapiens
WPSLVHRLTRDAPLAVLRAFKFYVPW

SEQ ID NO: 515 - protein, 58 aa, Homo sapiens
SLGISTFGIMVFSVYFGGIMISIPYSGISFGNKKELNIDSCYNMVNLKNIMFSERSQT

SEQ ID NO: 516 - protein, 15 aa, Homo sapiens
HASGNNDPLWFLTYL

SEQ ID NO: 517 - protein, 21 aa, Homo sapiens
MVFSVYFGGIMISIPYSGISF

SEQ ID NO: 518 - protein, 20 aa, Homo sapiens
FGNKKELNIDSCYNMVNLKN

SEQ ID NO: 519 - protein, 143 aa, Homo sapiens
  SITE (139): Xaa equals any of the naturally occurring L-amino acids
MHQQKRQPELVEGNLPVFVFPTELIFYADDQSTHKQVLTLYNPYEFALKFKVLCTTPNKYVVVDAAGAVKPQCCVDIVIRHRDVRSCHYGVIDKFRLQVSEQSQRKALGKKRGCCYSSPISKRTTKGRRGKKIKGTFNXXFIF

SEQ ID NO: 520 - protein, 75 aa, Homo sapiens
  SITE (48): Xaa equals any of the naturally occurring L-amino acids
MNSFSVIASIVVLLPFPGLSVSACLPSHSHQCKTFILLFLPSSEKTLXXXPPSHSSTLGGQGGQIMRSGDRXHXG

SEQ ID NO: 521 - protein, 81 aa, Homo sapiens
  SITE (5): Xaa equals any of the naturally occurring L-amino acids
VVFFXXFFEMESHSVAQAGVQWRNLGSLQALPPGFMPFSCLSLPGSWDYRRPPPSPANLXCIFSRDGGHHVSQXGLDLLTS

SEQ ID NO: 522 - protein, 28 aa, Homo sapiens
IVVLLPFPGLSVSACLPSHSHQCKTFIL

SEQ ID NO: 523 - protein, 26 aa, Homo sapiens
PGFMPFSCLSLPGSWDYRRPPPSPAN

SEQ ID NO: 524 - protein, 16 aa, Homo sapiens
YRFKNPKCRLFSVPCR

SEQ ID NO: 525 - protein, 128 aa, Homo sapiens
TQNRELLAWKPKGTDDICTSHNTTHIQKMPGEANSCCPRGAKSYHIDCWPPALFPRCVAYLFLNKPATLRKKYYCKPYHTQLHPAWHREKSAFWIFETVSQSKQSLTSLVYSVNELLVLSNLAQWALG

SEQ ID NO: 526 - protein, 23 aa, Homo sapiens
AWKPKGTDDICTSHNTTHIQKMP

SEQ ID NO: 527 - protein, 25 aa, Homo sapiens
CPRGAKSYHIDCWPPALFPRCVAYL

SEQ ID NO: 528 - protein, 26 aa, Homo sapiens
SYHIDCWPPALFPRCVAYLFLNKPAT

SEQ ID NO: 529 - protein, 29 aa, Homo sapiens
RKKYYCKPYHTQLHPAWHREKSAFWIFET

SEQ ID NO: 530 - protein, 28 aa, Homo sapiens
ICLDSCSQVSVTSLWSFLRVHSLVQTLW

SEQ ID NO: 531 - protein, 75 aa, Homo sapiens
HYCCDFGTSLLGFYVPFHYYVHMVNIILTTIDFYHYKFCCSQNANKHCFKHFQIMTTVPYLNINKENLRFKNIFK

SEQ ID NO: 532 - protein, 27 aa, Homo sapiens
TSLLGFYVPFHYYVHMVNIILTTIDFY

SEQ ID NO: 533 - protein, 22 aa, Homo sapiens
FQIMTTVPYLNINKENLRFKNI

SEQ ID NO: 534 - protein, 106 aa, Homo sapiens
ISESMSLVRSLQFYRGKNRAERTVISSSSHSCHLIDLEFQPRSDGEVSISFLEKGVELRWGMGLEDLIGLGLGVSTRRSTVRRKEPTKAGMHTACSEEMEPENREN

SEQ ID NO: 535 - protein, 143 aa, Homo sapiens [sequence omitted]

SEQ ID NO: 536 - protein, 27 aa, Homo sapiens
RGKNRAERTVISSSSHSCHLIDLEFQP

SEQ ID NO: 537 - protein, 32 aa, Homo sapiens
LGLGVSTRRSTVRRKEPTKAGMHTACSEEMEP

SEQ ID NO: 538 - protein, 24 aa, Homo sapiens
GDPPALASQSAGITGVSHSTRPKL

SEQ ID NO: 539 - protein, 25 aa, Homo sapiens
ALYSIQWALLANSLYFQVPSPLSML

SEQ ID NO: 540 - protein, 35 aa, Homo sapiens
DRILLFYSRDGQTTSKGPNPACCLFLLKKFYWNTA

SEQ ID NO: 541 - protein, 21 aa, Homo sapiens
DGQTTSKGPNPACCLFLLKKF

SEQ ID NO: 542 - protein, 24 aa, Homo sapiens
DPRVRRTLDLGITLYLFLYIFLSL

SEQ ID NO: 543 - protein, 244 aa, Homo sapiens
PALGECCLDAFLFLLGKQLKKSGEKPLLGGSLMEYAILSAIAAMNEPKTCSTTALKKYVLENHPGTNSNYQMHLLKKTLQKCEKNGWMEQISGKGFSGTFQLCFPYYPSPGVLFPKKEPDDSRDEDEDEDESSEEDSEDEEPPPKRRLQKKTPAKSPGKAASVKQRGSKPAPKVSAAQRGKARPLPKKAPPKAKTPAKKTRPSSTVIKKPSGGSSKKPATSARKEVKLPGKGKSTMKKSFRVKK

SEQ ID NO: 544 - protein, 152 aa, Homo sapiens
DFEFHHDTLFSYKIYFFTLKDFFMVDLPLPGNFTSFLALVAGFFEEPPLGFLMTVDEGLVFLAGVLALGGAFLGKGLAFPRWAAETLGAGLDPLCFTDAAFPGDLAGVFFCNLLLGGGSSSSESSSDDSSSSSSSSLESSGSFFGNRTPGLG

SEQ ID NO: 545 - protein, 28 aa, Homo sapiens
CLDAFLFLLGKQLKKSGEKPLLGGSLME

SEQ ID NO: 546 - protein, 30 aa, Homo sapiens
YQMHLLKKTLQKCEKNGWMEQISGKGFSGT

SEQ ID NO: 547 - protein, 28 aa, Homo sapiens
KTPAKSPGKAASVKQRGSKPAPKVSAAQ

SEQ ID NO: 548 - protein, 28 aa, Homo sapiens
SSKKPATSARKEVKLPGKGKSTMKKSFR

SEQ ID NO: 549 - protein, 23 aa, Homo sapiens
VDEGLVFLAGVLALGGAFLGKGL

SEQ ID NO: 550 - protein, 25 aa, Homo sapiens
GLDPLCFTDAAFPGDLAGVFFCNLL

SEQ ID NO: 551 - protein, 59 aa, Homo sapiens
TMLFYLSSQPDWQLDFFRVSFNGPVFFIIIFNDRAGFRMQALVSQAACRRSRYKLSVVY

SEQ ID NO: 552 - protein, 23 aa, Homo sapiens
DRAGFRMQALVSQAACRRSRYKL

SEQ ID NO: 553 - protein, 438 aa, Homo sapiens
  SITE (84): Xaa equals any of the naturally occurring L-amino acids
MAMGFPGYDLSADDIAGKFQFSRGMRRSYDAGFKLMVVEYAESTNNCQAAKQFGVLEKNVRDWRKVKPQLQNAHAMRRAFRGPXNGRFALVDQRVAEYVRYMQAKGDPITREAMQLKALEIAQEMNIPEKGFKASLGWCRRMMRRYDLSLRHKVPVPQHLPEDLTEKLVTYQRSVLALRRAHDYEVAXMGNADETPICLEVPSRVTVDNQGEKPVLVKTPGREKLKITAMLGVLADGRKLPPYIILRGTYIPPGKFPSGMEIRCHRYGWMTEDLMQDWLEVVWRRRTGAVPKQRGMLILNGFRGHATDSVKNSMESMNTDMVIXPGGLTSQLQVLDVVVYKPLNDSVRAQYSNWLLAGNLALSPTGNAKKPPLGLFLEWVMVAWNSISSESIVQGFKKCHISSNLEEEDDVLWEIESELPGGGEPPKDCDTESMAESN

SEQ ID NO: 554 - protein, 30 aa, Homo sapiens
GQEEWTNSRHKAPSARTAKGVYRDQPYGRY

SEQ ID NO: 555 - protein, 26 aa, Homo sapiens
ILAISLAQNFTPSWKGGERECSDLYL

SEQ ID NO: 556 - protein, 11 aa, Homo sapiens
LQTYLSPYKLF

SEQ ID NO: 557 - protein, 22 aa, Homo sapiens
LAAGILNSSLPALYHSVEEISQ

SEQ ID NO: 558 - protein, 45 aa, Homo sapiens
XYRMNTKFLESYKMSTTLSRRHQNVSLCKDMKTPAGTDTKIAFLE

SEQ ID NO: 559 - protein, 21 aa, Homo sapiens
SYKMSTTLSRRHQNVSLCKDM

SEQ ID NO: 560 - protein, 57 aa, Homo sapiens
ICIESLMLHYIALVFEMAFMFPLVYHEMGSDSIRFHLCQVDSCLPSMMRFFFSFPFL

SEQ ID NO: 561 - protein, 21 aa, Homo sapiens
YIALVFEMAFMFPLVYHEMGS

SEQ ID NO: 562 - protein, 21 aa, Homo sapiens
SDSIRFHLCQVDSCLPSMMRF

SEQ ID NO: 563 - protein, 115 aa, Homo sapiens [sequence omitted]

SEQ ID NO: 564 - protein, 111 aa, Homo sapiens
  SITE (82): Xaa equals any of the naturally occurring L-amino acids
FTNKSCSKMSSTHLYKGSDVLCYARSSESMSLSCGDVANAGRLTPRLHLARSASQGPPTLPRVPPRGSRPPTAGESPAPRTXSLENHKNIDHLSSNSHGKFRIYGQNDIKI

SEQ ID NO: 565 - protein, 80 aa, Homo sapiens
QDVIYTFVQRFRRPMLCTILRKYEPVVRGRRKRWQAHPSSAFGKKRLPRPPHPAQGAPQREQASHSWREPGPQNTFPRKP

SEQ ID NO: 566 - protein, 22 aa, Homo sapiens
REETDVGEGGRPRGGQSEGARV

SEQ ID NO: 567 - protein, 27 aa, Homo sapiens
GPGLAPVVFVSTLPSLQPSSFCGWDLP

SEQ ID NO: 568 - protein, 24 aa, Homo sapiens
MSSTHLYKGSDVLCYARSSESMSL

SEQ ID NO: 569 - protein, 28 aa, Homo sapiens
SQGPPTLPRVPPRGSRPPTAGESPAPRT

SEQ ID NO: 570 - protein, 25 aa, Homo sapiens
RFRRPMLCTILRKYEPVVRGRRKRW

SEQ ID NO: 571 - protein, 24 aa, Homo sapiens
RLPRPPHPAQGAPQREQASHSWRE

SEQ ID NO: 572 - protein, 81 aa, Homo sapiens
  SITE (43): Xaa equals any of the naturally occurring L-amino acids
RGMRGRWLVSSGAAFPIPLNGFCESREFFPDSGSVLLHWRPNXVLIEIKVFGSRSQSLISSKNLKTSLTFIYGKVEEVLNN

SEQ ID NO: 573 - protein, 81 aa, Homo sapiens
  SITE (62): Xaa equals any of the naturally occurring L-amino acids
LKLSSADSQAIMNIFSADCMPRLHIALQTEMIPNRAPQGGAAANLWHEAQYRRLPFSRAPEXTDAHQASAQRGAAQLPREQ

SEQ ID NO: 574 - protein, 28 aa, Homo sapiens
  SITE (28): Xaa equals any of the naturally occurring L-amino acids
PIPLNGFCESREFFPDSGSVLLHWRPNX

SEQ ID NO: 575 - protein, 29 aa, Homo sapiens
NIFSADCMPRLHIALQTEMIPNRAPQGGA

SEQ ID NO: 576 - protein, 37 aa, Homo sapiens
TFRLVSAHLKTRKLINPEAAERRWRDWDSRQGWLSVK

SEQ ID NO: 577 - protein, 21 aa, Homo sapiens
KTRKLINPEAAERRWRDWDSR

SEQ ID NO: 578 - protein, 83 aa, Homo sapiens
WNYTVNNLYLFSFSIVSMKFMHVLSINIFFGRARWLTPVIPALLEAEAGGSLGQEFKTSLGKDGETPSLLKIQKLAGHGGRRL

SEQ ID NO: 579 - protein, 76 aa, Homo sapiens [sequence omitted]

SEQ ID NO: 580 - protein, 28 aa, Homo sapiens
VIPALLEAEAGGSLGQEFKTSLGKDGET

SEQ ID NO: 581 - protein, 29 aa, Homo sapiens
NRLNLGDRGCTELLHSSLGNRVRLSKKKE

SEQ ID NO: 582 - protein, 8 aa, Homo sapiens
HEIFGQVF

SEQ ID NO: 583 - protein, 17 aa, Homo sapiens
HASEHLAALPVNVKIGK

SEQ ID NO: 584 - protein, 77 aa, Homo sapiens
LVCILLVHWIPPLGAWGLSLMLFLILEQRCGKGKWRNALLSVSFSVPQLQMQKVSLDSTPLNVNHDKMDIWKLTPKL

SEQ ID NO: 585 - protein, 57 aa, Homo sapiens
IMIKWIFGNLLLSCDLGCISTSGLPQYQGLRLLNFEYSLGFMLRSLWSRSAIQCFFS

SEQ ID NO: 586 - protein, 21 aa, Homo sapiens
LLLSCDLGCISTSGLPQYQGL

SEQ ID NO: 587 - protein, 21 aa, Homo sapiens
LRLLNFEYSLGFMLRSLWSRS

SEQ ID NO: 588 - protein, 78 aa, Homo sapiens
ASPHLFIEKWGRAFILRKLLLVPVISKRIINIMAHQVKPPIFCAMIMCNLFCSGYEHLLFTLMRFFSFEQIFDEVVFH

SEQ ID NO: 589 - protein, 25 aa, Homo sapiens
KLLLVPVISKRIINIMAHQVKPPIF

SEQ ID NO: 590 - protein, 7 aa, Homo sapiens
PEQKRLH

SEQ ID NO: 591 - protein, 358 aa, Homo sapiens
  SITE (352): Xaa equals any of the naturally occurring L-amino acids
FAVIRFESIIHEFDPWFNYRSTHHLASHGFYEFLNWFDERAWYPLGRIVGGTVYPGLMITAGLIHWILNTLNITVHIRDVCVFLAPTFSGLTSISTFLLTRELWNQGAGLLAACFIAIVPGYISRSVAGSFDNEGIAIFALQFTYYLWVKSVKTGSVFWTMCCCLSYFYMVSAWGGYVFIINLIPLHVFVLLLMQRYSKRVYIAYSTFYIVGLILSMQIPFVGFQPIRTSEHMAAAGVFALLQAYAFLQYLRDRLTKQEFQTLFFLGVSLAAGAVFLSVIYLTYTGYIAPWSGRFYSLWDTGYAKIHIPIIASVSEHQPTTWVSFFFDLHILVCTFPAGLWFCIKNINDERXFGKXGF

SEQ ID NO: 592 - protein, 27 aa, Homo sapiens
EFDPWFNYRSTHHLASHGFYEFLNWFD

SEQ ID NO: 593 - protein, 23 aa, Homo sapiens
TRELWNQGAGLLAACFIAIVPGY

SEQ ID NO: 594 - protein, 22 aa, Homo sapiens
TYYLWVKSVKTGSVFWTMCCCL

SEQ ID NO: 595 - protein, 25 aa, Homo sapiens
GVFALLQAYAFLQYLRDRLTKQEFQ

SEQ ID NO: 596 - protein, 27 aa, Homo sapiens
YSLWDTGYAKIHIPIIASVSEHQPTTW

SEQ ID NO: 597 - protein, 408 aa, Homo sapiens
  SITE (20): Xaa equals any of the naturally occurring L-amino acids
MGHMLYLLGNINKRTMHKYXQESKKAGKASFAYAWVLDETGEERERGVTMDVGMTKFETTTKVITLMDAPGHKDFIPNMITGAAQADVAVLVVDASRGEFEAGFETGGQTREHGLLVRSLGVTQLAVAVNKMDQVNWQQERFQEITGKLGHFLKQAGFKESDVGFIPTSGLSGENLITRSQSSELTKWYKGLCLLEQIDSFKPPQRSIDKPFRLCVSDVFKDQGSGFCITGKIEAGYIQTGDRLLAMPPNETCTVKGITLHDEPVDWAAAGDHVSLTLVGMDIIKINVGCIFCGPKVPIKACTRFRARILIFNIEIPITKGFPVLLHYQTVSEPAVIKRLISVLNKSTGEVTKKKPKFLTKGQNALVELQTQRPIALELYKDFKELGRFMLRYGGSTIAAGVVTEIKE

SEQ ID NO: 598 - protein, 21 aa, Homo sapiens
  SITE (16): Xaa equals any of the naturally occurring L-amino acids
LYLLGNINKRTMHKYXQESKK

SEQ ID NO: 599 - protein, 23 aa, Homo sapiens
LDETGEERERGVTMDVGMTKFET

SEQ ID NO: 600 - protein, 22 aa, Homo sapiens
GHKDFIPNMITGAAQADVAVLV

SEQ ID NO: 601 - protein, 23 aa, Homo sapiens
GFETGGQTREHGLLVRSLGVTQL

SEQ ID NO: 602 - protein, 23 aa, Homo sapiens
WQQERFQEITGKLGHFLKQAGFK

SEQ ID NO: 603 - protein, 22 aa, Homo sapiens
TSGLSGENLITRSQSSELTKWY

SEQ ID NO: 604 - protein, 23 aa, Homo sapiens
PQRSIDKPFRLCVSDVFKDQGSG

SEQ ID NO: 605 - protein, 22 aa, Homo sapiens
LISVLNKSTGEVTKKKPKFLTK

SEQ ID NO: 606 - protein, 25 aa, Homo sapiens
QRPIALELYKDFKELGRFMLRYGGS

SEQ ID NO: 607 - protein, 83 aa, Homo sapiens
QKGPPIEDAIASSDVLETASKSANPPHTIQASEEQSSTPAPVKKSGKLRQQIDVKAELEKRQGGKQLLNLVVIGHVDAGKSTL

SEQ ID NO: 608 - protein, 120 aa, Homo sapiens
NGFFSFSMYIILCQTFFSVAALRWTGDSIGFINLSFSHLFIPQTFVEGHQALGRGKWFYKLVLSGIKEIYNLYYLIVATSHMWFSNKISITSPTTFSSLVRSRPRETVPFIVFSAFYKLR

SEQ ID NO: 609 - protein, 21 aa, Homo sapiens
IILCQTFFSVAALRWTGDSIG

SEQ ID NO: 610 - protein, 21 aa, Homo sapiens
GFINLSFSHLFIPQTFVEGHQ

SEQ ID NO: 611 - protein, 20 aa, Homo sapiens
QALGRGKWFYKLVLSGIKEI

SEQ ID NO: 612 - protein, 21 aa, Homo sapiens
IYNLYYLIVATSHMWFSNKIS